(19) United States
(12) Patent Application Publication
(10) Pub. No.: US 2026/0164917 A1
(43) Pub. Date: Jun. 11, 2026

(54) HETEROCYCLIC COMPOUND AND ORGANIC LIGHT EMITTING DEVICE COMPRISING THE SAME

(71) Applicant: LT Materials Co., Ltd., Yongin-si (KR)

(72) Inventors: Hae Won JUNG, Yongin-si (KR); Nam Jin LEE, Yongin-si (KR); Won Jang JEONG, Yongin-si (KR); Dong Jun KIM, Yongin-si (KR); Dae Hyuk CHOI, Yongin-si (KR)

(73) Assignee: LT Materials Co., Ltd., Yongin-si (KR)

(21) Appl. No.: 19/030,620

(22) Filed: Jan. 17, 2025

(30) Foreign Application Priority Data
Feb. 21, 2024 (KR) 10-2024-0024858

(51) Int. Cl. H10K 50/11; C07D 209/86; C07D 403/14; C09K 11/06; H10K 50/16; H10K 50/17; H10K 50/18; H10K 50/19; H10K 85/30; H10K 85/60

(52) U.S. Cl. CPC H10K 50/11; C07D 209/86; C07D 403/14; C09K 11/06; H10K 50/16; H10K 50/171; H10K 50/181; H10K 50/19; H10K 85/342; H10K 85/622; H10K 85/626; H10K 85/633; H10K 85/654; H10K 85/6572; H10K 85/6574; H10K 85/6576; C09K 2211/1059

(57) ABSTRACT

The present specification relates to a heterocyclic compound represented by Chemical Formula 1 and an organic light emitting device including the same.

### TECHNICAL FIELD

This application claims priority to Korean Patent Application No. 10-2024-0024858, filed on Feb. 21, 2024, the disclosure of which is incorporated herein by reference in its entirety.

The present specification relates to a heterocyclic compound and an organic light emitting device including the same.

### DESCRIPTION OF THE RELATED ART

An organic light emitting device is one type of self-emissive display devices, and has advantages of having a wide viewing angle and a high response speed as well as having an excellent contrast.

The organic light emitting device has a structure of disposing an organic thin film between two electrodes. When a voltage is applied to the organic light emitting device having such a structure, electrons and holes injected from the two electrodes bind and pair in the organic thin film, and then light is emitted as these annihilate. The organic thin film may be formed in a single layer or a multilayer as necessary.

A material of the organic thin film may have a light emitting function as necessary. For example, as a material of the organic thin film, compounds each capable of forming a light emitting layer themselves alone may be used, or compounds each capable of serving as a host or a dopant of a host-dopant-based light emitting layer may also be used. In addition thereto, compounds capable of performing roles of hole injection, hole transport, electron blocking, hole blocking, electron transport, electron injection and the like may also be used as a material of the organic thin film.

Development of an organic thin film material has been continuously required for enhancing performance, lifetime or efficiency of an organic light emitting device.

### PRIOR ART DOCUMENTS

### Patent Documents

(Patent Document 1) U.S. Pat. No. 4,356,429

### SUMMARY

The present specification is directed to providing a heterocyclic compound and an organic light emitting device including the same.

One embodiment of the present application provides a heterocyclic compound represented by the following Chemical Formula 1.

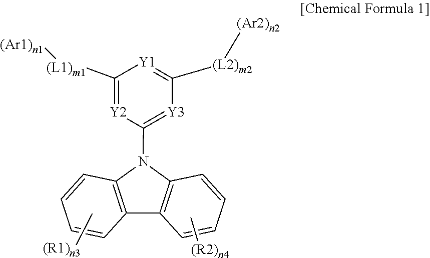

In Chemical Formula 1,
Y1 to Y3 are the same as or different from each other and each independently CH or N, and at least one of Y1 to Y3 is N,
L1 and L2 are the same as or different from each other, and each independently a single bond; a substituted or unsubstituted C6 to C60 arylene group; or a substituted or unsubstituted C2 to C60 heteroarylene group,
Ar1 and Ar2 are the same as or different from each other and each independently a substituted or unsubstituted C6 to C60 aryl group; or a substituted or unsubstituted C2 to C60 heteroaryl group, and at least one of Ar1 and Ar2 is a group represented by the following Chemical Formula 2 or Chemical Formula 3,
R1 and R2 are the same as or different from each other, and each independently selected from the group consisting of hydrogen; deuterium; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; and a substituted or unsubstituted C2 to C60 heteroaryl group, or two or more groups adjacent to each other bond to each other to form a substituted or unsubstituted C6 to C60 aromatic hydrocarbon ring or a substituted or unsubstituted C2 to C60 heteroring,
m1 and m2 are the same as or different from each other, and each independently an integer of 0 to 4, and
n1 to n4 are the same as or different from each other, and each independently an integer of 0 to 4,

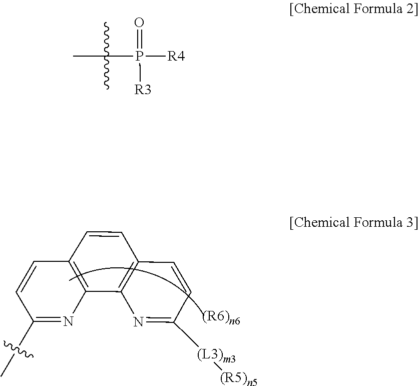

in Chemical Formulae 2 and 3,
R3 to R6 are the same as or different from each other, and each independently selected from the group consisting of hydrogen; deuterium; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; and a substituted or unsubstituted C2 to C60 heteroaryl group, or two or more groups adjacent to each other bond to each other to form a substituted or unsubstituted C6 to C60 aromatic hydrocarbon ring or a substituted or unsubstituted C2 to C60 heteroring,
L3 is a single bond; a substituted or unsubstituted C6 to C60 arylene group; or a substituted or unsubstituted C2 to C60 heteroarylene group,
m3 is an integer of 0 to 4,
n5 is an integer of 0 to 4, and
n6 is an integer of 0 to 6.

In addition, one embodiment of the present application provides an organic light emitting device including: a first electrode; a second electrode provided opposite to the first electrode; and one or more organic material layers provided between the first electrode and the second electrode, wherein the organic material layers include one or more types of the heterocyclic compound.

The compound described in the present specification can be used as an organic material layer material of an organic light emitting device. The compound is capable of performing roles of a hole injection material, a hole transport material, a light emitting material, an electron transport material, an electron injection material and the like in an organic light emitting device. Particularly, the compound can be used as an electron transport layer material or a charge generation layer material of an organic light emitting device.

Specifically, when the compound represented by Chemical Formula 1 is used in an organic material layer, it is possible to lower a driving voltage of a device, enhance light efficiency thereof, and enhance lifetime properties of the device by thermal stability of the compound.

### BRIEF DESCRIPTION OF THE DRAWINGS

FIGS. 1 to 4 are diagrams each schematically illustrating a lamination structure of an organic light emitting device according to one embodiment of the present application.

### DESCRIPTION OF SPECIFIC EMBODIMENTS

Hereinafter, the present disclosure will be described in more detail.

In the present specification, a term “substitution” means that a hydrogen atom bonding to a carbon atom of a compound is changed to another substituent, and the position of substitution is not limited as long as it is a position at which the hydrogen atom is substituted, that is, a position at which a substituent is capable of substituting, and when two or more substituents substitute, the two or more substituents may be the same as or different from each other.

In the present specification, “substituted or unsubstituted” means being substituted with one or more substituents selected from the group consisting of deuterium; a cyano group; a C1 to C60 linear or branched alkyl group; a C2 to C60 linear or branched alkenyl group; a C2 to C60 linear or branched alkynyl group; a C3 to C60 monocyclic or polycyclic cycloalkyl group; a C2 to C60 monocyclic or polycyclic heterocycloalkyl group; a C6 to C60 monocyclic or polycyclic aryl group; a C2 to C60 monocyclic or polycyclic heteroaryl group; —SiRR′R″; —P(═O)RR′; a C1 to C20 alkylamine group; a C6 to C60 monocyclic or polycyclic arylamine group; and a C2 to C60 monocyclic or polycyclic heteroarylamine group or being unsubstituted, or being substituted with a substituent in which two or more substituents selected from among the substituents exemplified above are linked or being unsubstituted.

In the present specification, “the number of protons” means the number of substituents that a specific compound may have, and specifically, the number of protons may mean the number of hydrogens.

For example, unsubstituted benzene may be expressed to have the number of protons of 5, an unsubstituted naphthyl group may be expressed to have the number of protons of 7, a naphthyl group substituted with a phenyl group may be expressed to have the number of protons of 6, and an unsubstituted biphenyl group may be expressed to have the number of protons of 9.

In the present specification, the alkyl group includes a linear or branched form having 1 to 60 carbon atoms, and may be further substituted with other substituents. The number of carbon atoms of the alkyl group may be from 1 to 60, specifically from 1 to 40 and more specifically from 1 to 20. Specific examples of the alkyl group may include a methyl group, an ethyl group, a propyl group, an n-propyl group, an isopropyl group, a butyl group, an n-butyl group, an isobutyl group, a tert-butyl group, a sec-butyl group, a 1-methyl-butyl group, a 1-ethyl-butyl group, a pentyl group, an n-pentyl group, an isopentyl group, a neopentyl group, a tert-pentyl group, a hexyl group, an n-hexyl group, a 1-methylpentyl group, a 2-methylpentyl group, a 4-methyl-2-pentyl group, a 3,3-dimethylbutyl group, a 2-ethylbutyl group, a heptyl group, an n-heptyl group, a 1-methylhexyl group, a cyclopentylmethyl group, a cyclohexylmethyl group, an octyl group, an n-octyl group, a tert-octyl group, a 1-methylheptyl group, a 2-ethylhexyl group, a 2-propylpentyl group, an n-nonyl group, a 2,2-dimethylheptyl group, a 1-ethyl-propyl group, a 1,1-dimethyl-propyl group, an isohexyl group, a 4-methylhexyl group, a 5-methylhexyl group and the like, but are not limited thereto.

In the present specification, the alkenyl group includes a linear or branched form having 2 to 60 carbon atoms, and may be further substituted with other substituents. The number of carbon atoms of the alkenyl group may be from 2 to 60, specifically from 2 to 40 and more specifically from 2 to 20. Specific examples of the alkenyl group may include a vinyl group, a 1-propenyl group, an isopropenyl group, a 1-butenyl group, a 2-butenyl group, a 3-butenyl group, a 1-pentenyl group, a 2-pentenyl group, a 3-pentenyl group, a 3-methyl-1-butenyl group, a 1,3-butadienyl group, an allyl group, a 1-phenylvinyl-1-yl group, a 2-phenylvinyl-1-yl group, a 2,2-diphenylvinyl-1-yl group, a 2-phenyl-2-(naphthyl-1-yl) vinyl-1-yl group, a 2,2-bis(diphenyl-1-yl) vinyl-1-yl group, a stilbenyl group, a styrenyl group and the like, but are not limited thereto.

In the present specification, the alkynyl group includes a linear or branched form having 2 to 60 carbon atoms, and may be further substituted with other substituents. The number of carbon atoms of the alkynyl group may be from 2 to 60, specifically from 2 to 40 and more specifically from 2 to 20.

In the present specification, the alkoxy group may be linear, branched or cyclic. The number of carbon atoms of the alkoxy group is not particularly limited, but is preferably from 1 to 20. Specific examples of the alkoxy group may include a methoxy group, an ethoxy group, an n-propoxy group, an isopropoxy group, an n-butoxy group, an isobutoxy group, a tert-butoxy group, a sec-butoxy group, an n-pentyloxy group, a neopentyloxy group, an isopentyloxy an group, n-hexyloxy group, a 3,3-dimethylbutyloxy group, a 2-ethylbutyloxy group, an n-octyloxy group, an n-nonyloxy group, an n-decyloxy group, a benzyloxy group, a p-methylbenzyloxy group and the like, but are not limited thereto.

In the present specification, the cycloalkyl group includes a monocyclic or polycyclic group having 3 to 60 carbon atoms, and may be further substituted with other substituents. Herein, the polycyclic group means a group in which the cycloalkyl group is directly linked to or fused with another cyclic group. Herein, the another cyclic group may be a cycloalkyl group, but may also be different types of cyclic groups such as a heterocycloalkyl group, an aryl group and a heteroaryl group. The number of carbon atoms of the cycloalkyl group may be from 3 to 60, specifically from 3 to 40 and more specifically from 5 to 20. Specific examples of the cycloalkyl group may include a cyclopropyl group, a cyclobutyl group, a cyclopentyl group, a 3-methylcyclopentyl group, a 2,3-dimethylcyclopentyl group, a cyclohexyl group, a 3-methylcyclohexyl group, a 4-methylcyclohexyl group, a 2,3-dimethylcyclohexyl group, a 3,4,5-trimethylcyclohexyl group, a 4-tert-butylcyclohexyl group, a cycloheptyl group, a cyclooctyl group and the like, but are not limited thereto.

In the present specification, the heterocycloalkyl group includes O, S, Se, N or Si as a heteroatom, includes a monocyclic or polycyclic group having 2 to 60 carbon atoms, and may be further substituted with other substituents. Herein, the polycyclic group means a group in which the heterocycloalkyl group is directly linked to or fused with another cyclic group. Herein, the another cyclic group may be a heterocycloalkyl group, but may also be different types of cyclic groups such as a cycloalkyl group, an aryl group and a heteroaryl group. The number of carbon atoms of the heterocycloalkyl group may be from 2 to 60, specifically from 2 to 40 and more specifically from 3 to 20.

In the present specification, the aryl group includes a monocyclic or polycyclic group having 6 to 60 carbon atoms, and may be further substituted with other substituents. Herein, the polycyclic group means a group in which the aryl group is directly linked to or fused with another cyclic group. Herein, the another cyclic group may be an aryl group, but may also be different types of cyclic groups such as a cycloalkyl group, a heterocycloalkyl group and a heteroaryl group. The aryl group may include a spiro group. The number of carbon atoms of the aryl group may be from 6 to 60, specifically from 6 to 40 and more specifically from 6 to 25. Specific examples of the aryl group may include a phenyl group, a biphenyl group, a terphenyl group, a naphthyl group, an anthryl group, a chrysenyl group, a phenanthrenyl group, a perylenyl group, a fluoranthenyl group, a triphenylenyl group, a phenalenyl group, a pyrenyl group, a tetracenyl group, a pentacenyl group, a fluorenyl group, an indenyl group, an acenaphthylenyl group, a benzofluorenyl group, a spirobifluorenyl group, a 2,3-dihydro-1H-indenyl group, a fused ring group thereof, and the like, but are not limited thereto.

In the present specification, the heteroaryl group includes S, O, Se, N or Si as a heteroatom, includes a monocyclic or polycyclic group having 2 to 60 carbon atoms, and may be further substituted with other substituents. Herein, the polycyclic group means a group in which the heteroaryl group is directly linked to or fused with another cyclic group. Herein, the another cyclic group may be a heteroaryl group, but may also be different types of cyclic groups such as a cycloalkyl group, a heterocycloalkyl group and an aryl group. The number of carbon atoms of the heteroaryl group may be from 2 to 60, specifically from 2 to 40 and more specifically from 3 to 25. Specific examples of the heteroaryl group may include a pyridyl group, a pyrrolyl group, a pyrimidyl group, a pyridazinyl group, a furanyl group, a thiophenyl group, an imidazolyl group, a pyrazolyl group, an oxazolyl group, an isoxazolyl group, a thiazolyl group, an isothiazolyl group, a triazolyl group, a furazanyl group, an oxadiazolyl group, a thiadiazolyl group, a dithiazolyl group, a tetrazolyl group, a pyranyl group, a thiopyranyl group, a diazinyl group, an oxazinyl group, a thiazinyl group, a dioxynyl group, a triazinyl group, a tetrazinyl group, a quinolyl group, an isoquinolyl group, a quinazolinyl group, an isoquinazolinyl group, a quinozolinyl group, a naphthyridyl group, an acridinyl group, a phenanthridinyl group, an imidazopyridinyl group, a diazanaphthalenyl group, a triazaindenyl group, a 2-indolyl group, an indolizinyl group, a benzothiazolyl group, a benzoxazolyl group, a benzimidazolyl group, a benzothiophenyl group, a benzofuranyl group, a dibenzothiophenyl group, a dibenzofuranyl group, a carbazolyl group, a benzocarbazolyl a phenazinyl group, a group, a dibenzocarbazolyl group, dibenzosilole group, a spirobi (dibenzosilole) group, a dihydrophenazinyl group, a phenoxazinyl group, a phenanthridyl group, a thienyl group, an indolo[2,3-a]carbazolyl group, an indolo[2,3-b]carbazolyl group, an indolinyl group, a 10,11-dihydro-dibenzo[b,f]azepinyl group, a 9,10-dihydroacridinyl group, a phenanthrazinyl group, a phenothiazinyl group, a phthalazinyl group, a naphthylidinyl group, a phenanthrolinyl group, a benzo[c][1,2,5]thiadiazolyl group, a 5,10-dihydrodibenzo[b,e][1,4]azasilinyl group, a pyrazolo[1,5-c]quinazolinyl group, a pyrido[1,2-b]indazolyl group, a pyrido[1,2-a]imidazo[1,2-e]indolinyl group, a 5,11-dihydroindeno[1,2-b]carbazolyl group and the like, but are not limited thereto.

In the present specification, the phosphine oxide group is represented by —P(═O)R101R102, and R101 and R102 are the same as or different from each other and may be each independently a substituent formed with at least one of hydrogen; deuterium; a halogen group; an alkyl group; an alkenyl group; an alkoxy group; a cycloalkyl group; an aryl group; and a heterocyclic group. Specifically, the phosphine oxide group may be substituted with an aryl group, and as the aryl group, the examples described above may be applied. Examples of the phosphine oxide group may include a diphenylphosphine oxide group, a dinaphthylphosphine oxide group and the like, but are not limited thereto.

In the present specification, the silyl group is a substituent including Si and having the Si atom directly linked as a radical, and is represented by —SiR101R102R103. R101 to R103 are the same as or different from each other, and may be each independently a substituent formed with at least one of hydrogen; deuterium; a halogen group; an alkyl group; an alkenyl group; an alkoxy group; a cycloalkyl group; an aryl group; and a heterocyclic group. Specific examples of the silyl group may include a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group and the like, but are not limited thereto.

In the present specification, the fluorenyl group may be substituted, and adjacent substituents may bond to each other to form a ring.

When the fluorenyl group is substituted, the following structural formulae and the like may be included, however, the structure is not limited thereto.

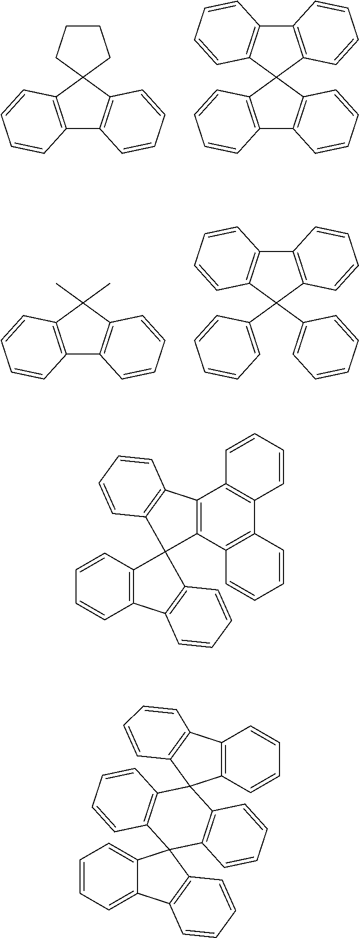

In the present specification, the spiro group is a group including a spiro structure, and may have 15 to 60 carbon atoms. For example, the spiro group may include a structure in which a 2,3-dihydro-1H-indene group or a cyclohexane group spiro bonds to a fluorenyl group. Specifically, the spiro group may include any one of groups of the following structural formulae.

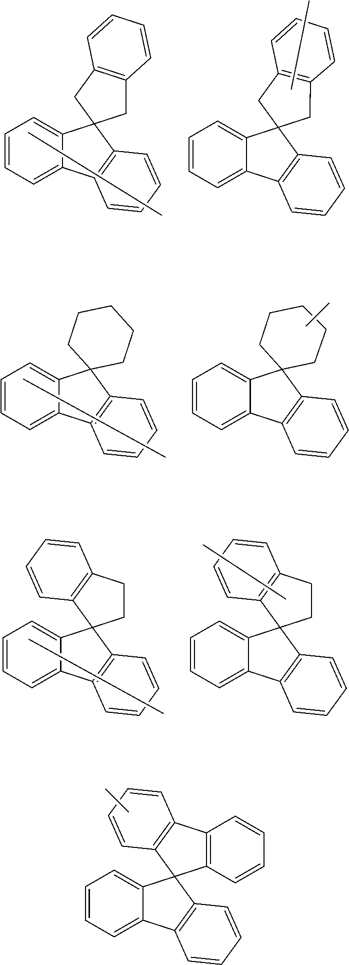

In the present specification, the amine group may be selected from the group consisting of a monoalkylamine group; a monoarylamine group; a monoheteroarylamine group; —NH₂; a dialkylamine group; a diarylamine group; a diheteroarylamine group; an alkylarylamine group; an alkylheteroarylamine group; and an arylheteroarylamine group, and the number of carbon atoms is not particularly limited, but preferably from 1 to 30. Specific examples of the amine group may include a methylamine group, a dimethylamine group, an ethylamine group, a diethylamine group, a phenylamine group, a naphthylamine group, a biphenylamine group, a dibiphenylamine group, an anthracenylamine group, a 9-methyl-anthracenylamine group, a diphenylamine group, a phenylnaphthylamine group, a ditolylamine group, a phenyltolylamine group, a triphenylamine group, a biphenylnaphthylamine group, a phenylbiphenylamine group, a biphenylfluorenylamine group, a phenyltriphenylenylamine group, a biphenyltriphenylenylamine group and the like, but are not limited thereto.

In the present specification, the arylene group means the aryl group having two bonding sites, that is, a divalent group. The descriptions on the aryl group provided above may be applied thereto except for those that are each a divalent group. In addition, the heteroarylene group means the heteroaryl group having two bonding sites, that is, a divalent group. The descriptions on the heteroaryl group provided above may be applied thereto except for those that are each a divalent group.

In the present specification, an “adjacent” group may mean a substituent substituting an atom directly linked to an atom substituted by the corresponding substituent, a substituent sterically most closely positioned to the corresponding substituent, or another substituent substituting an atom substituted by the corresponding substituent. For example, two substituents substituting at ortho positions in a benzene ring, and two substituents substituting at the same carbon in an aliphatic ring may be interpreted as groups “adjacent” to each other.

In the present disclosure, a “case of a substituent being not indicated in a chemical formula or compound structure” means that a hydrogen atom bonds to a carbon atom. However, since deuterium (²H) is an isotope of hydrogen, some hydrogen atoms may be deuterium.

In one embodiment of the present disclosure, a “case of a substituent being not indicated in a chemical formula or compound structure” may mean that positions to which substituents may come are all hydrogen or deuterium. In other words, since deuterium is an isotope of hydrogen, some hydrogen atoms may be deuterium that is an isotope, and herein, a content of the deuterium may be from 0% to 100%.

In one embodiment of the present disclosure, in a “case of a substituent being not indicated in a chemical formula or compound structure”, hydrogen and deuterium may be used interchangeably in compounds when deuterium is not explicitly excluded such as “a deuterium content being 0%”, “a hydrogen content being 100%” or “substituents being all hydrogen”.

In one embodiment of the present disclosure, deuterium is one of isotopes of hydrogen, is an element having deuteron formed with one proton and one neutron as a nucleus, and may be expressed as hydrogen-2, and the elemental symbol thereof may also be written as D or ²H.

In one embodiment of the present disclosure, an isotope means an atom with the same atomic number (Z) but with a different mass number (A), and may also be interpreted as an element with the same number of protons but with a different number of neutrons.

In one embodiment of the present disclosure, a content T % of a specific substituent may be defined as T2/T1×100=T % when the total number of substituents that a basic compound may have is defined as T1, and the number of specific substituents among these is defined as T2.

In other words, in one example, having a deuterium content of 20% in a phenyl group represented by

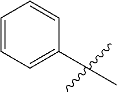 [SMILES: *c1ccccc1]

may mean that the total number of substituents that the phenyl group may have is 5 (T1 in the formula), and the number of deuterium atoms among these is 1 (T2 in the formula). In other words, having a deuterium content of 20% in a phenyl group may be represented by the following structural formulae.

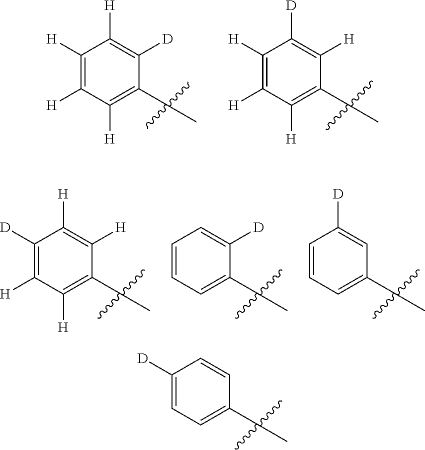 [SMILES: *c1ccccc1[2H] ; *c1cccc([2H])c1 ; *c1ccc([2H])cc1 ; *c1ccccc1[2H] ; *c1cccc([2H])c1 ; *c1ccc([2H])cc1]

In addition, in one embodiment of the present disclosure, “a phenyl group having a deuterium content of 0%” may mean a phenyl group that does not include a deuterium atom, that is, a phenyl group that has 5 hydrogen atoms.

In the present disclosure, the C6 to C60 aromatic hydrocarbon ring means a compound including an aromatic ring formed with C6 to C60 carbons and hydrogens. Examples thereof may include benzene, biphenyl, terphenyl, triphenylene, naphthalene, anthracene, phenalene, phenanthrene, fluorene, pyrene, chrysene, perylene, azulene and the like, but are not limited thereto, and include all aromatic hydrocarbon ring compounds known in the art and satisfying the above-mentioned number of carbon atoms.

One embodiment of the present application provides a heterocyclic compound represented by the following Chemical Formula 1.

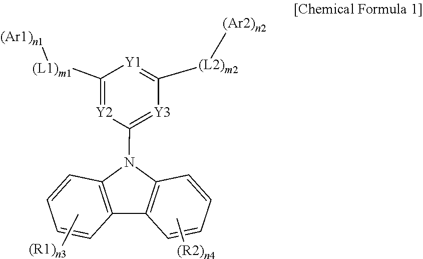

In Chemical Formula 1,
Y1 to Y3 are the same as or different from each other and each independently CH or N, and at least one of Y1 to Y3 is N,
L1 and L2 are the same as or different from each other, and each independently a single bond; a substituted or unsubstituted C6 to C60 arylene group; or a substituted or unsubstituted C2 to C60 heteroarylene group,
Ar1 and Ar2 are the same as or different from each other and each independently a substituted or unsubstituted C6 to C60 aryl group; or a substituted or unsubstituted C2 to C60 heteroaryl group, and at least one of Ar1 and Ar2 is a group represented by the following Chemical Formula 2 or Chemical Formula 3,
R1 and R2 are the same as or different from each other, and each independently selected from the group consisting of hydrogen; deuterium; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; and a substituted or unsubstituted C2 to C60 heteroaryl group, or two or more groups adjacent to each other bond to each other to form a substituted or unsubstituted C6 to C60 aromatic hydrocarbon ring or a substituted or unsubstituted C2 to C60 heteroring,
m1 and m2 are the same as or different from each other, and each independently an integer of 0 to 4, and
n1 to n4 are the same as or different from each other, and each independently an integer of 0 to 4,

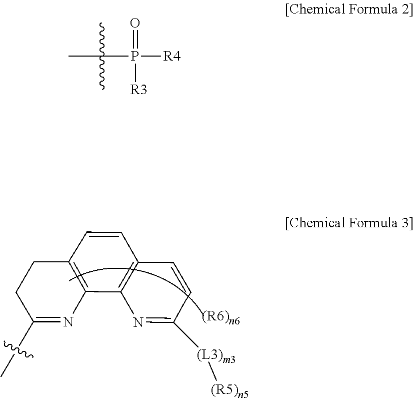

in Chemical Formulae 2 and 3,
R3 to R6 are the same as or different from each other, and each independently selected from the group consisting of hydrogen; deuterium; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; and a substituted or unsubstituted C2 to C60 heteroaryl group, or two or more groups adjacent to each other bond to each other to form a substituted or unsubstituted C6 to C60 aromatic hydrocarbon ring or a substituted or unsubstituted C2 to C60 heteroring,
L3 is a single bond; a substituted or unsubstituted C6 to C60 arylene group; or a substituted or unsubstituted C2 to C60 heteroarylene group,
m3 is an integer of 0 to 4,
n5 is an integer of 0 to 4, and
n6 is an integer of 0 to 6.

In one embodiment of the present application, Y1 of Chemical Formula 1 is N, and Y2 and Y3 thereof may be CH.

In another embodiment, Y1 and Y2 of Chemical Formula 1 are N, and Y3 thereof may be CH.

In another embodiment, Y1 and Y3 of Chemical Formula 1 are N, and Y2 thereof may be CH.

In another embodiment, Y1 of Chemical Formula 1 is CH, and Y2 and Y3 thereof may be N.

In another embodiment, Y1 and Y2 of Chemical Formula 1 are CH, and Y3 thereof may be N.

In another embodiment, Y1 and Y3 of Chemical Formula 1 are CH, and Y2 thereof may be N.

In another embodiment, Y1 to Y3 of Chemical Formula 1 may be N.

In one embodiment of the present application, L1 and L2 of Chemical Formula 1 are the same as or different from each other, and may be each independently a single bond; a substituted or unsubstituted C6 to C40 arylene group; or a substituted or unsubstituted C2 to C40 heteroarylene group.

In one embodiment of the present application, L1 and L2 of Chemical Formula 1 are the same as or different from each other, and may be each independently a single bond; a substituted or unsubstituted C6 to C30 arylene group; or a substituted or unsubstituted C2 to C30 heteroarylene group.

In one embodiment of the present application, L1 and L2 of Chemical Formula 1 are the same as or different from each other, and may be each independently a single bond; a substituted or unsubstituted C6 to C20 arylene group; or a substituted or unsubstituted C2 to C20 heteroarylene group.

In one embodiment of the present application, L1 and L2 of Chemical Formula 1 are the same as or different from each other, and may be each independently a single bond; a substituted or unsubstituted C6 to C10 arylene group; or a substituted or unsubstituted C2 to C10 heteroarylene group.

In one embodiment of the present application, L1 and L2 of Chemical Formula 1 are the same as or different from each other, and may be each independently a single bond; a substituted or unsubstituted phenylene group; a substituted or unsubstituted biphenylene group; a substituted or unsubstituted terphenylene group; a substituted or unsubstituted naphthylene group; a substituted or unsubstituted anthracenylene group; a substituted or unsubstituted phenanthrylene group; a substituted or unsubstituted dibenzofuranylene group; or a substituted or unsubstituted dibenzothiophenylene group.

In one embodiment of the present application, L1 and L2 of Chemical Formula 1 are the same as or different from each other, and may be each independently a single bond; a substituted or unsubstituted phenylene group; or a substituted or unsubstituted biphenylene group.

In one embodiment of the present application, Ar1 and Ar2 of Chemical Formula 1 are the same as or different from each other and each independently a substituted or unsubstituted C6 to C40 aryl group; or a substituted or unsubstituted C2 to C40 heteroaryl group, and at least one of Ar1 and Ar2 may be the group represented by Chemical Formula 2 or Chemical Formula 3.

In one embodiment of the present application, Ar1 and Ar2 of Chemical Formula 1 are the same as or different from each other and each independently a substituted or unsubstituted C6 to C30 aryl group; or a substituted or unsubstituted C2 to C30 heteroaryl group, and at least one of Ar1 and Ar2 may be the group represented by Chemical Formula 2 or Chemical Formula 3.

In one embodiment of the present application, Ar1 and Ar2 of Chemical Formula 1 are the same as or different from each other and each independently a substituted or unsubstituted C6 to C20 aryl group; or a substituted or unsubstituted C2 to C20 heteroaryl group, and at least one of Ar1 and Ar2 may be the group represented by Chemical Formula 2 or Chemical Formula 3.

In one embodiment of the present application, Ar1 and Ar2 of Chemical Formula 1 are the same as or different from each other and each independently a substituted or unsubstituted C6 to C10 aryl group; or a substituted or unsubstituted C2 to C10 heteroaryl group, and at least one of Ar1 and Ar2 may be the group represented by Chemical Formula 2 or Chemical Formula 3.

In one embodiment of the present application, Ar1 and Ar2 of Chemical Formula 1 are the same as or different from each other and each independently a substituted or unsubstituted phenyl group; a substituted or unsubstituted biphenyl group; a substituted or unsubstituted terphenyl group; a substituted or unsubstituted naphthyl group; a substituted or unsubstituted anthracenyl group; a substituted or unsubstituted phenanthryl group; a substituted or unsubstituted triphenylenyl group; a substituted or unsubstituted pyridyl group; a substituted or unsubstituted pyrimidyl group; a substituted or unsubstituted quinolinyl group; a substituted or unsubstituted isoquinolinyl group; a substituted or unsubstituted dibenzofuranyl group; a substituted or unsubstituted dibenzothiophenyl group; a substituted or unsubstituted carbazolyl group; a substituted or unsubstituted benzofuropyridyl or a substituted or unsubstituted benzothiolopyridyl group, and at least one of Ar1 and Ar2 may be the group represented by Chemical Formula 2 or Chemical Formula 3.

In one embodiment of the present application, Ar1 and Ar2 of Chemical Formula 1 are the same as or different from each other and each independently a substituted or unsubstituted phenyl group; a substituted or unsubstituted biphenyl group; a substituted or unsubstituted naphthyl group; a substituted or unsubstituted phenanthryl group; a substituted or unsubstituted triphenylenyl group; a substituted or unsubstituted dibenzofuranyl group; a substituted or unsubstituted dibenzothiophenyl group; or a substituted or unsubstituted carbazolyl group, and at least one of Ar1 and Ar2 may be the group represented by Chemical Formula 2 or Chemical Formula 3.

In one embodiment of the present application, Ar1 of Chemical Formula 1 may be the group represented by Chemical Formula 2.

In one embodiment of the present application, Ar1 of Chemical Formula 1 may be the group represented by Chemical Formula 3.

In one embodiment of the present application, Ar2 of Chemical Formula 1 may be the group represented by Chemical Formula 2.

In one embodiment of the present application, Ar3 of Chemical Formula 1 may be the group represented by Chemical Formula 3.

In one embodiment of the present application, R1 and R2 of Chemical Formula 1 are the same as or different from each other, and each independently selected from the group consisting of hydrogen; deuterium; a cyano group; a substituted or unsubstituted C1 to C40 alkyl group; a substituted or unsubstituted C2 to C40 alkenyl group; a substituted or unsubstituted C2 to C40 alkynyl group; a substituted or unsubstituted C1 to C40 alkoxy group; a substituted or unsubstituted C3 to C40 cycloalkyl group; a substituted or unsubstituted C2 to C40 heterocycloalkyl group; a substituted or unsubstituted C6 to C40 aryl group; and a substituted or unsubstituted C2 to C40 heteroaryl group, or two or more groups adjacent to each other may bond to each other to form a substituted or unsubstituted C6 to C40 aromatic hydrocarbon ring or a substituted or unsubstituted C2 to C40 heteroring.

In one embodiment of the present application, R1 and R2 of Chemical Formula 1 are the same as or different from each other, and each independently selected from the group consisting of hydrogen; deuterium; a cyano group; a substituted or unsubstituted C1 to C30 alkyl group; a substituted or unsubstituted C2 to C30 alkenyl group; a substituted or unsubstituted C2 to C30 alkynyl group; a substituted or unsubstituted C1 to C30 alkoxy group; a substituted or unsubstituted C3 to C30 cycloalkyl group; a substituted or unsubstituted C2 to C30 heterocycloalkyl group; a substituted or unsubstituted C6 to C30 aryl group; and a substituted or unsubstituted C2 to C30 heteroaryl group, or two or more groups adjacent to each other may bond to each other to form a substituted or unsubstituted C6 to C30 aromatic hydrocarbon ring or a substituted or unsubstituted C2 to C30 heteroring.

In one embodiment of the present application, R1 and R2 of Chemical Formula 1 are the same as or different from each other, and each independently selected from the group consisting of hydrogen; deuterium; a cyano group; a substituted or unsubstituted C1 to C20 alkyl group; a substituted or unsubstituted C2 to C20 alkenyl group; a substituted or unsubstituted C2 to C20 alkynyl group; a substituted or unsubstituted C1 to C20 alkoxy group; a substituted or unsubstituted C3 to C20 cycloalkyl group; a substituted or unsubstituted C2 to C20 heterocycloalkyl group; a substituted or unsubstituted C6 to C20 aryl group; and a substituted or unsubstituted C2 to C20 heteroaryl group, or two or more groups adjacent to each other may bond to each other to form a substituted or unsubstituted C6 to C20 aromatic hydrocarbon ring or a substituted or unsubstituted C2 to C20 heteroring.

In one embodiment of the present application, R1 and R2 of Chemical Formula 1 are the same as or different from each other, and each independently selected from the group consisting of hydrogen; or deuterium; a cyano group; a substituted unsubstituted C1 to C10 alkyl group; a substituted or unsubstituted C2 to C10 alkenyl group; a substituted or unsubstituted C2 to C10 alkynyl group; a substituted or unsubstituted C1 to C10 alkoxy group; a substituted or unsubstituted C3 to C10 cycloalkyl group; a substituted or unsubstituted C2 to C10 heterocycloalkyl group; a substituted or unsubstituted C6 to C10 aryl group; and a substituted or unsubstituted C2 to C10 heteroaryl group, or two or more groups adjacent to each other may bond to each other to form a substituted or unsubstituted C6 to C10 aromatic hydrocarbon ring or a substituted or unsubstituted C2 to C10 heteroring.

In one embodiment of the present application, R1 and R2 of Chemical Formula 1 are the same as or different from each other, and may be each independently hydrogen; or deuterium.

In one embodiment of the present application, R3 to R6 of Chemical Formulae 2 and 3 are the same as or different from each other, and each independently selected from the group consisting of hydrogen; deuterium; a cyano group; a substituted or unsubstituted C1 to C40 alkyl group; a substituted or unsubstituted C2 to C40 alkenyl group; a substituted or unsubstituted C2 to C40 alkynyl group; a substituted or unsubstituted C1 to C40 alkoxy group; a substituted or unsubstituted C3 to C40 cycloalkyl group; a substituted or unsubstituted C2 to C40 heterocycloalkyl group; a substituted or unsubstituted C6 to C40 aryl group; and a substituted or unsubstituted C2 to C40 heteroaryl group, or two or more groups adjacent to each other may bond to each other to form a substituted or unsubstituted C6 to C40 aromatic hydrocarbon ring or a substituted or unsubstituted C2 to C40 heteroring.

In one embodiment of the present application, R3 to R6 Chemical Formulae 2 and 3 are the same as or different from each other, and each independently selected from the group consisting of hydrogen; deuterium; a cyano group; a substituted or unsubstituted C1 to C30 alkyl group; a substituted or unsubstituted C2 to C30 alkenyl group; a substituted or unsubstituted C2 to C30 alkynyl group; a substituted or unsubstituted C1 to C30 alkoxy group; a substituted or unsubstituted C3 to C30 cycloalkyl group; a substituted or unsubstituted C2 to C30 heterocycloalkyl group; a substituted or unsubstituted C6 to C30 aryl group; and a substituted or unsubstituted C2 to C30 heteroaryl group, or two or more groups adjacent to each other may bond to each other to form a substituted or unsubstituted C6 to C30 aromatic hydrocarbon ring or a substituted or unsubstituted C2 to C30 heteroring.

In one embodiment of the present application, R3 to R6 of Chemical Formulae 2 and 3 are the same as or different from each other, and each independently selected from the group consisting of hydrogen; deuterium; a cyano group; a substituted or group; a substituted or unsubstituted C1 to C20 alkyl unsubstituted C2 to C20 alkenyl group; a substituted or unsubstituted C2 to C20 alkynyl group; a substituted or unsubstituted C1 to C20 alkoxy group; a substituted or unsubstituted C3 to C20 cycloalkyl group; a substituted or unsubstituted C2 to C20 heterocycloalkyl group; a substituted or unsubstituted C6 to C20 aryl group; and a substituted or unsubstituted C2 to C20 heteroaryl group, or two or more groups adjacent to each other may bond to each other to form a substituted or unsubstituted C6 to C20 aromatic hydrocarbon ring or a substituted or unsubstituted C2 to C20 heteroring.

In one embodiment of the present application, R3 to R6 of Chemical Formulae 2 and 3 are the same as or different from each other, and each independently selected from the group consisting of hydrogen; deuterium; a cyano group; a substituted or unsubstituted C1 to C10 alkyl group; a substituted or unsubstituted C2 to C10 alkenyl group; a substituted or unsubstituted C2 to C10 alkynyl group; a substituted or unsubstituted C1 to C10 alkoxy group; a substituted or unsubstituted C3 to C10 cycloalkyl group; a substituted or unsubstituted C2 to C10 heterocycloalkyl group; a substituted or unsubstituted C6 to C10 aryl group; and a substituted or unsubstituted C2 to C10 heteroaryl group, or two or more groups adjacent to each other may bond to each other to form a substituted or unsubstituted C6 to C10 aromatic hydrocarbon ring or a substituted or unsubstituted C2 to C10 heteroring.

In one embodiment of the present application, R3 to R6 of Chemical Formulae 2 and 3 are the same as or different from each other, and may be each independently hydrogen; deuterium; a cyano group; a substituted or unsubstituted methyl group; a substituted or unsubstituted ethyl group; a substituted or unsubstituted propyl group; a substituted or unsubstituted phenyl group; a substituted or unsubstituted biphenyl group; a substituted or unsubstituted terphenyl group; a substituted or unsubstituted naphthyl group; a substituted or unsubstituted anthracenyl group, a substituted or unsubstituted phenanthryl group, a substituted or unsubstituted pyridyl group; a substituted or unsubstituted pyrimidyl group; a substituted or unsubstituted quinolinyl group; a substituted or unsubstituted isoquinolinyl group; a substituted or unsubstituted dibenzofuranyl group; a substituted or unsubstituted dibenzothiophenyl group; a substituted or unsubstituted benzofuropyridyl group; or a substituted or unsubstituted benzothiolopyridyl group.

In one embodiment of the present application, R3 and R4 of Chemical Formula 2 are the same as or different from each other, and may be each independently a substituted or unsubstituted methyl group; or a substituted or unsubstituted phenyl group.

In one embodiment of the present application, R5 of Chemical Formula 3 may be a substituted or unsubstituted phenyl group.

In one embodiment of the present application, R6 of Chemical Formula 3 may be hydrogen; or deuterium.

In one embodiment of the present application, L3 of Chemical Formula 3 may be a single bond; a substituted or unsubstituted C6 to C40 arylene group; or a substituted or unsubstituted C2 to C40 heteroarylene group.

In one embodiment of the present application, L3 of Chemical Formula 3 may be a single bond; a substituted or unsubstituted C6 to C30 arylene group; or a substituted or unsubstituted C2 to C30 heteroarylene group.

In one embodiment of the present application, L3 of Chemical Formula 3 may be a single bond; a substituted or unsubstituted C6 to C20 arylene group; or a substituted or unsubstituted C2 to C20 heteroarylene group.

In one embodiment of the present application, L3 of Chemical Formula 3 may be a single bond; a substituted or unsubstituted C6 to C10 arylene group; or a substituted or unsubstituted C2 to C10 heteroarylene group.

In one embodiment of the present application, L3 of Chemical Formula 3 may be a single bond; a substituted or unsubstituted phenylene group; a substituted or unsubstituted biphenylene group; a substituted or unsubstituted terphenylene group; a substituted or unsubstituted naphthylene group; a substituted or unsubstituted anthracenylene group; a substituted or unsubstituted phenanthrylene group; a substituted or unsubstituted dibenzofuranylene group; or a substituted or unsubstituted dibenzothiophenylene group.

In one embodiment of the present application, L3 of Chemical Formula 3 may be a single bond.

In one embodiment of the present application, Chemical Formula 1 may be represented by any one of the following Chemical Formula 1-1 or Chemical Formula 1-2.

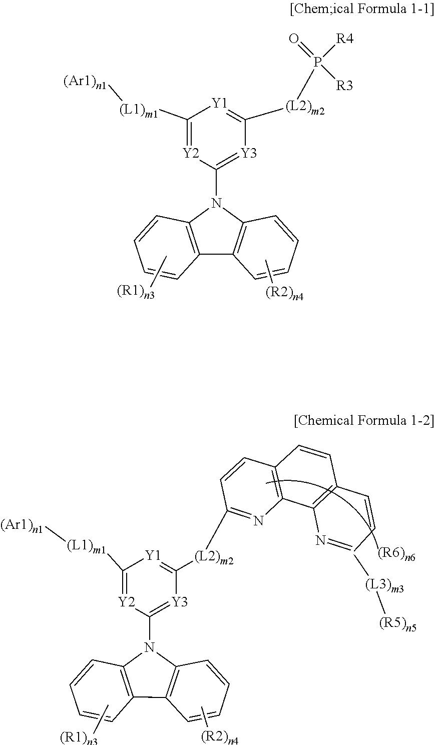

In Chemical Formula 1-1 and Chemical Formula 1-2, each substituent has the same definition as in Chemical Formula 1.

In one embodiment of the present application, L1 and L2 of Chemical Formula 1 are the same as or different from each other, and may be each independently represented by any one of a single bond; the following Chemical Formula 4; or the following Chemical Formula 5.

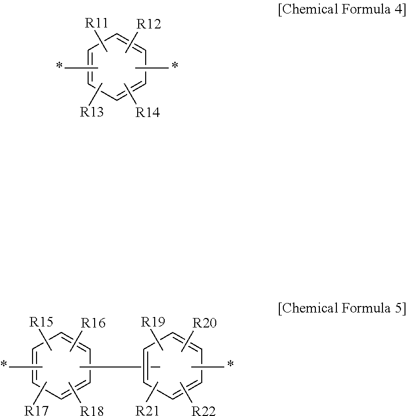

In Chemical Formulae 4 and 5,
R11 to R22 are the same as or different from each other, and each independently selected from the group consisting of hydrogen; deuterium; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; and a substituted or unsubstituted C2 to C60 heteroaryl group, or two or more groups adjacent to each other bond to each other to form a substituted or unsubstituted C6 to C60 aromatic hydrocarbon ring or a substituted or unsubstituted C2 to C60 heteroring, and
*s each indicate a point connected to carbon between Y1 and Y3 and Ar2 of Chemical Formula 1.

In one embodiment of the present application, R11 to R22 of Chemical Formulae 4 and 5 are the same as or different from each other, and each independently selected from the group consisting of hydrogen; deuterium; a cyano group; a substituted or unsubstituted C1 to C40 alkyl group; a substituted or unsubstituted C2 to C40 alkenyl group; a substituted or unsubstituted C2 to C40 alkynyl group; a substituted or unsubstituted C1 to C40 alkoxy group; a substituted or unsubstituted C3 to C40 cycloalkyl group; a substituted or unsubstituted C2 to C40 heterocycloalkyl group; a substituted or unsubstituted C6 to C40 aryl group; and a substituted or unsubstituted C2 to C40 heteroaryl group, or two or more groups adjacent to each other may bond to each other to form a substituted or unsubstituted C6 to C40 aromatic hydrocarbon ring or a substituted or unsubstituted C2 to C40 heteroring.

In one embodiment of the present application, R11 to R22 of Chemical Formulae 4 and 5 are the same as or different from each other, and each independently selected from the group consisting of hydrogen; deuterium; a cyano group; a substituted or unsubstituted C1 to C30 alkyl group; a substituted or unsubstituted C2 to C30 alkenyl group; a substituted or unsubstituted C2 to C30 alkynyl group; a substituted or unsubstituted C1 to C30 alkoxy group; a substituted or unsubstituted C3 to C30 cycloalkyl group; a substituted or unsubstituted C2 to C30 heterocycloalkyl group; a substituted or unsubstituted C6 to C30 aryl group; and a substituted or unsubstituted C2 to C30 heteroaryl group, or two or more groups adjacent to each other may bond to each other to form a substituted or unsubstituted C6 to C30 aromatic hydrocarbon ring or a substituted or unsubstituted C2 to C30 heteroring.

In one embodiment of the present application, R11 to R22 of Chemical Formulae 4 and 5 are the same as or different from each other, and each independently selected from the group consisting of hydrogen; deuterium; a cyano group; a substituted or unsubstituted C1 to C20 alkyl group; a substituted or unsubstituted C2 to C20 alkenyl group; a substituted or unsubstituted C2 to C20 alkynyl group; a substituted or unsubstituted C1 to C20 alkoxy group; a substituted or unsubstituted C3 to C20 cycloalkyl group; a substituted or unsubstituted C2 to C20 heterocycloalkyl group; a substituted or unsubstituted C6 to C20 aryl group; and a substituted or unsubstituted C2 to C20 heteroaryl group, or two or more groups adjacent to each other may bond to each other to form a substituted or unsubstituted C6 to C20 aromatic hydrocarbon ring or a substituted or unsubstituted C2 to C20 heteroring.

In one embodiment of the present application, R11 to R22 of Chemical Formulae 4 and 5 are the same as or different from each other, and each independently selected from the group consisting of hydrogen; deuterium; a cyano group; a substituted or unsubstituted C1 to C10 alkyl group; a substituted or unsubstituted C2 to C10 alkenyl group; a substituted or unsubstituted C2 to C10 alkynyl group; a substituted or unsubstituted C1 to C10 alkoxy group; a substituted or unsubstituted C3 to C10 cycloalkyl group; a substituted or unsubstituted C2 to C10 heterocycloalkyl group; a substituted or unsubstituted C6 to C10 aryl group; and a substituted or unsubstituted C2 to C10 heteroaryl group, or two or more groups adjacent to each other may bond to each other to form a substituted or unsubstituted C6 to C10 aromatic hydrocarbon ring or a substituted or unsubstituted C2 to C10 heteroring.

In one embodiment of the present application, R11 to R22 of Chemical Formulae 4 and 5 are the same as or different from each other, and may be each independently hydrogen; deuterium; a cyano group; a substituted or unsubstituted methyl group; a substituted or unsubstituted ethyl group; a substituted or unsubstituted propyl group; a substituted or unsubstituted phenyl group; a substituted or unsubstituted biphenyl group; a substituted or unsubstituted terphenyl group; a substituted or unsubstituted naphthyl group; a substituted or unsubstituted anthracenyl group, a substituted or unsubstituted phenanthryl group, a substituted or unsubstituted pyridyl group; a substituted or unsubstituted pyrimidyl group; a substituted or unsubstituted quinolinyl group; a substituted or unsubstituted isoquinolinyl group; a substituted or unsubstituted carbazolyl group; a substituted or unsubstituted dibenzofuranyl group; a substituted or unsubstituted dibenzothiophenyl group; a substituted or unsubstituted benzofuropyridyl group; or a substituted or unsubstituted benzothiolopyridyl group.

In one embodiment of the present application, R11 to R22 of Chemical Formulae 4 and 5 are the same as or different from each other, and may be each independently hydrogen; deuterium; a substituted or unsubstituted phenyl group; a substituted or unsubstituted carbazolyl group; a substituted or unsubstituted dibenzofuranyl group; or a substituted or unsubstituted dibenzothiophenyl group.

In one embodiment of the present application, R11 to R14 of Chemical Formula 4 are the same as or different from each other, and may be each independently hydrogen; deuterium; or a substituted or unsubstituted carbazolyl group.

In one embodiment of the present application, R15 to R22 of Chemical Formula 5 are the same as or different from each other, and may be each independently hydrogen; deuterium; a substituted or unsubstituted carbazolyl group; a substituted or unsubstituted dibenzofuranyl group; a or substituted or unsubstituted dibenzothiophenyl group.

In one embodiment of the present application, the heterocyclic compound represented by Chemical Formula 1 may not include deuterium as a substituent, or may have a deuterium content of, for example, 1% or greater, 5% or greater, 10% or greater, 15% or greater, 20% or greater, 25% or greater, 30% or greater, 35% or greater, 40% or greater, 45% or greater or 50% or greater, and 100% or less, 95% or less, 90% or less, 85% or less, 80% or less, 75% or less, 70% or less, 65% or less or 60% or less based on the total number of hydrogen atoms and deuterium atoms.

In one embodiment of the present application, the heterocyclic compound represented by Chemical Formula 1 may not include deuterium, or may have a deuterium content of 1% to 100% based on the total number of hydrogen atoms and deuterium atoms.

In one embodiment of the present application, the heterocyclic compound represented by Chemical Formula 1 may not include deuterium, or may have a deuterium content of 10% to 100% based on the total number of hydrogen atoms and deuterium atoms.

In one embodiment of the present application, the heterocyclic compound represented by Chemical Formula 1 may not include deuterium, or may have a deuterium content of 20% to 90% based on the total number of hydrogen atoms and deuterium atoms.

In one embodiment of the present application, the heterocyclic compound represented by Chemical Formula 1 may not include deuterium, or may have a deuterium content of 30% to 80% based on the total number of hydrogen atoms and deuterium atoms.

In one embodiment of the present application, the heterocyclic compound represented by Chemical Formula 1 may not include deuterium, or may have a deuterium content of 40% to 70% based on the total number of hydrogen atoms and deuterium atoms.

In one embodiment of the present application, the heterocyclic compound represented by Chemical Formula 1 may not include deuterium, or may have a deuterium content of 50% to 60% based on the total number of hydrogen atoms and deuterium atoms.

One embodiment of the present application provides a heterocyclic compound, in which Chemical Formula 1 is represented by any one of the following compounds. In addition, in one embodiment of the present application, the following compounds are just one example, and the present application is not limited thereto and may include other compounds included in Chemical Formula 1 including additional substituents.

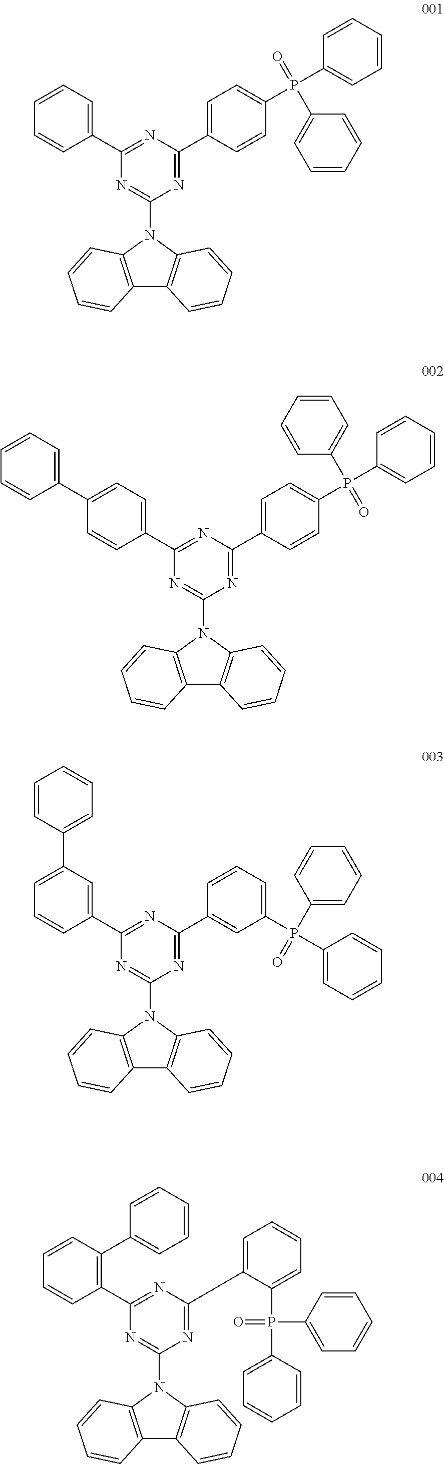 [SMILES: O=P(c1ccccc1)(c1ccccc1)c1ccc(-c2nc(-c3ccccc3)nc(-n3c4ccccc4c4ccccc43)n2)cc1 ; O=P(c1ccccc1)(c1ccccc1)c1ccc(-c2nc(-c3ccc(-c4ccccc4)cc3)nc(-n3c4ccccc4c4ccccc43)n2)cc1 ; O=P(c1ccccc1)(c1ccccc1)c1cccc(-c2nc(-c3cccc(-c4ccccc4)c3)nc(-n3c4ccccc4c4ccccc43)n2)c1 ; O=P(c1ccccc1)(c1ccccc1)c1ccccc1-c1nc(-c2ccccc2-c2ccccc2)nc(-n2c3ccccc3c3ccccc32)n1]

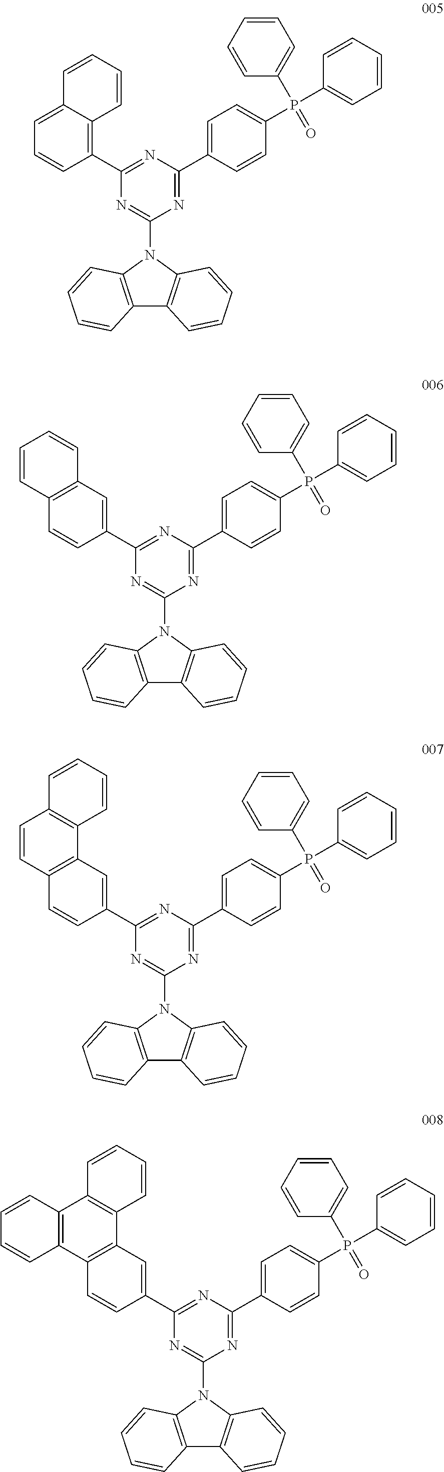 [SMILES: O=P(c1ccccc1)(c1ccccc1)c1ccc(-c2nc(-c3cccc4ccccc34)nc(-n3c4ccccc4c4ccccc43)n2)cc1 ; O=P(c1ccccc1)(c1ccccc1)c1ccc(-c2nc(-c3ccc4ccccc4c3)nc(-n3c4ccccc4c4ccccc43)n2)cc1 ; O=P(c1ccccc1)(c1ccccc1)c1ccc(-c2nc(-c3ccc4ccc5ccccc5c4c3)nc(-n3c4ccccc4c4ccccc43)n2)cc1 ; O=P(c1ccccc1)(c1ccccc1)c1ccc(-c2nc(-c3ccc4c5ccccc5c5ccccc5c4c3)nc(-n3c4ccccc4c4ccccc43)n2)cc1]

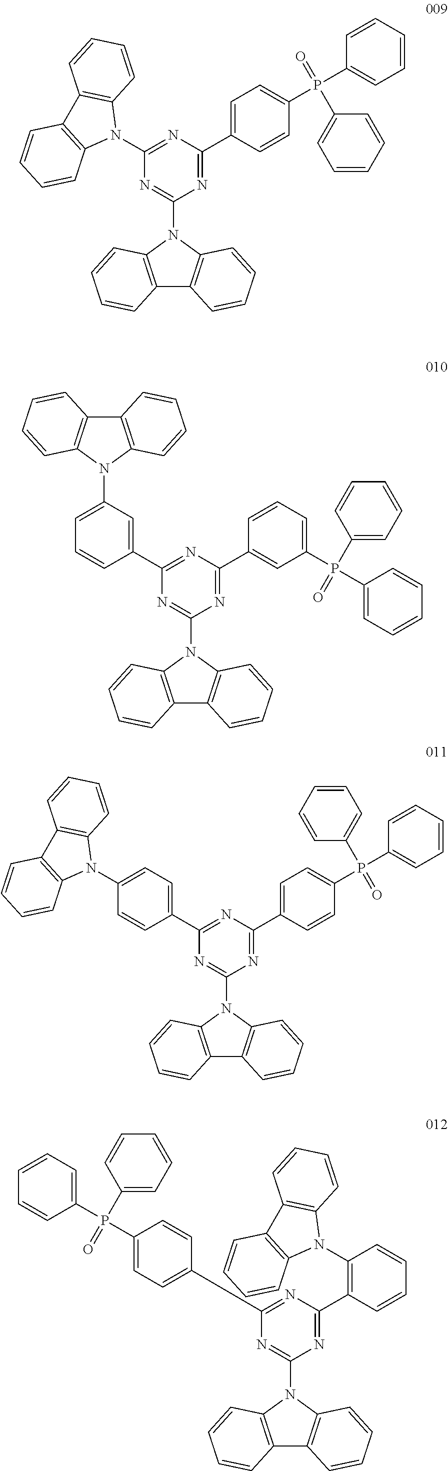 [SMILES: O=P(c1ccccc1)(c1ccccc1)c1ccc(-c2nc(-n3c4ccccc4c4ccccc43)nc(-n3c4ccccc4c4ccccc43)n2)cc1 ; O=P(c1ccccc1)(c1ccccc1)c1cccc(-c2nc(-c3cccc(-n4c5ccccc5c5ccccc54)c3)nc(-n3c4ccccc4c4ccccc43)n2)c1 ; O=P(c1ccccc1)(c1ccccc1)c1ccc(-c2nc(-c3ccc(-n4c5ccccc5c5ccccc54)cc3)nc(-n3c4ccccc4c4ccccc43)n2)cc1 ; O=P(c1ccccc1)(c1ccccc1)c1ccc(-c2nc(-c3ccccc3-n3c4ccccc4c4ccccc43)nc(-n3c4ccccc4c4ccccc43)n2)cc1]

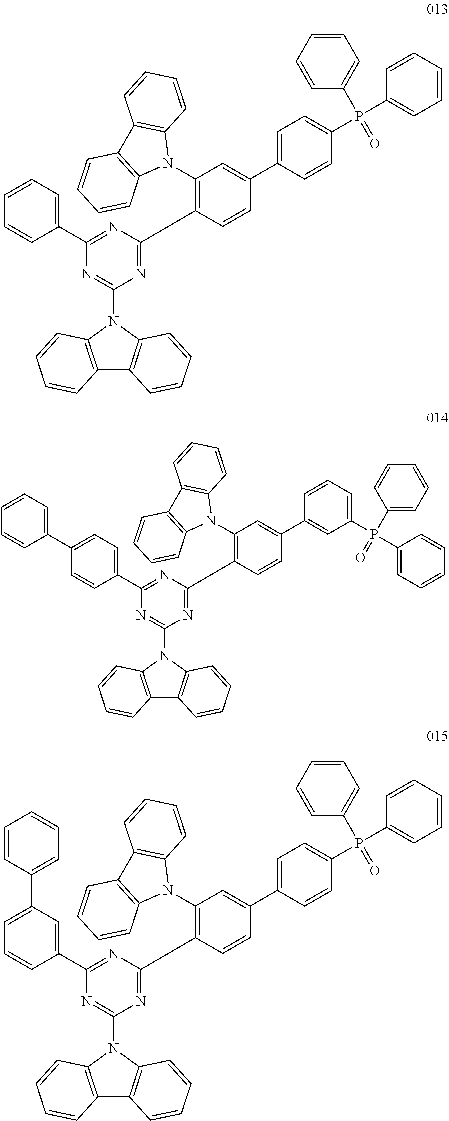 [SMILES: O=P(c1ccccc1)(c1ccccc1)c1ccc(-c2ccc(-c3nc(-c4ccccc4)nc(-n4c5ccccc5c5ccccc54)n3)c(-n3c4ccccc4c4ccccc43)c2)cc1 ; O=P(c1ccccc1)(c1ccccc1)c1cccc(-c2ccc(-c3nc(-c4ccc(-c5ccccc5)cc4)nc(-n4c5ccccc5c5ccccc54)n3)c(-n3c4ccccc4c4ccccc43)c2)c1 ; O=P(c1ccccc1)(c1ccccc1)c1ccc(-c2ccc(-c3nc(-c4cccc(-c5ccccc5)c4)nc(-n4c5ccccc5c5ccccc54)n3)c(-n3c4ccccc4c4ccccc43)c2)cc1]

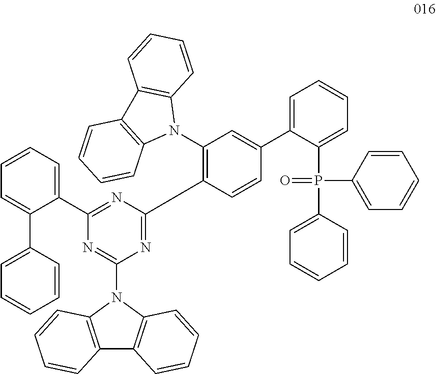 [SMILES: O=P(c1ccccc1)(c1ccccc1)c1ccccc1-c1ccc(-c2nc(-c3ccccc3-c3ccccc3)nc(-n3c4ccccc4c4ccccc43)n2)c(-n2c3ccccc3c3ccccc32)c1]

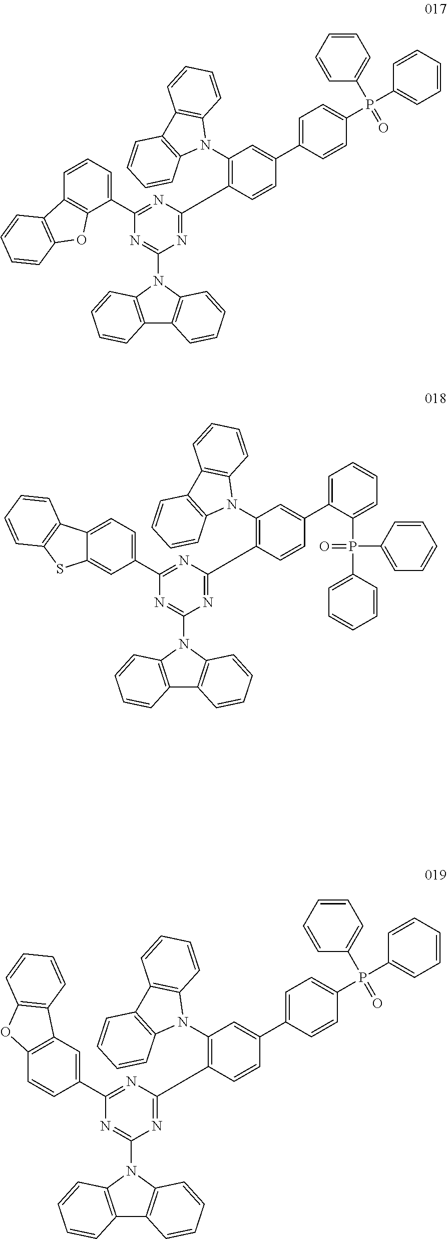 [SMILES: O=P(c1ccccc1)(c1ccccc1)c1ccc(-c2ccc(-c3nc(-c4cccc5c4oc4ccccc45)nc(-n4c5ccccc5c5ccccc54)n3)c(-n3c4ccccc4c4ccccc43)c2)cc1 ; O=P(c1ccccc1)(c1ccccc1)c1ccccc1-c1ccc(-c2nc(-c3ccc4c(c3)sc3ccccc34)nc(-n3c4ccccc4c4ccccc43)n2)c(-n2c3ccccc3c3ccccc32)c1 ; O=P(c1ccccc1)(c1ccccc1)c1ccc(-c2ccc(-c3nc(-c4ccc5oc6ccccc6c5c4)nc(-n4c5ccccc5c5ccccc54)n3)c(-n3c4ccccc4c4ccccc43)c2)cc1]

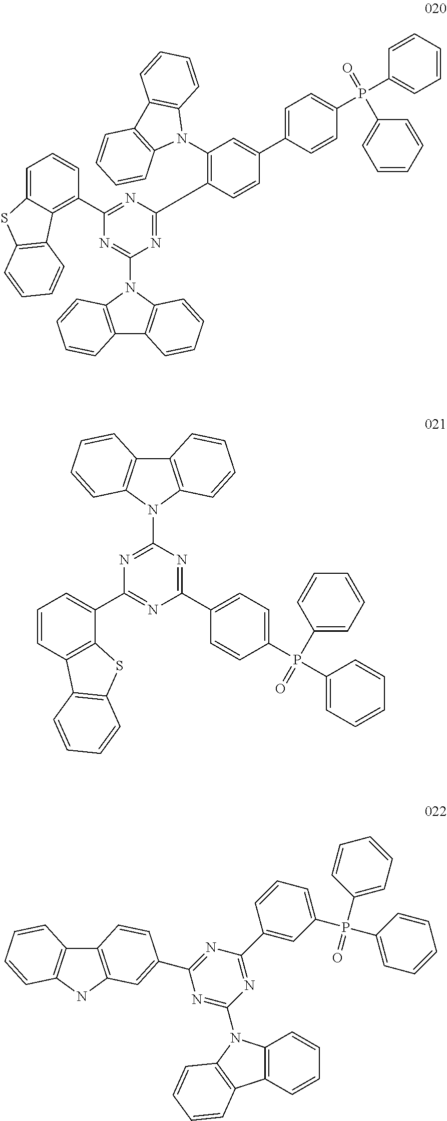 [SMILES: O=P(c1ccccc1)(c1ccccc1)c1ccc(-c2ccc(-c3nc(-c4cccc5sc6ccccc6c45)nc(-n4c5ccccc5c5ccccc54)n3)c(-n3c4ccccc4c4ccccc43)c2)cc1 ; O=P(c1ccccc1)(c1ccccc1)c1ccc(-c2nc(-c3cccc4c3sc3ccccc34)nc(-n3c4ccccc4c4ccccc43)n2)cc1 ; O=P(c1ccccc1)(c1ccccc1)c1cccc(-c2nc(-c3ccc4c(c3)[nH]c3ccccc34)nc(-n3c4ccccc4c4ccccc43)n2)c1]

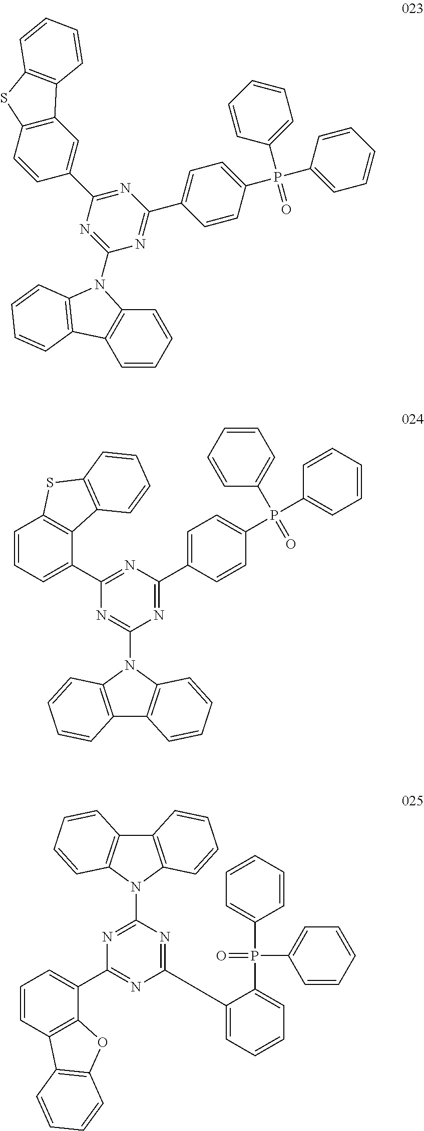 [SMILES: O=P(c1ccccc1)(c1ccccc1)c1ccc(-c2nc(-c3ccc4sc5ccccc5c4c3)nc(-n3c4ccccc4c4ccccc43)n2)cc1 ; O=P(c1ccccc1)(c1ccccc1)c1ccc(-c2nc(-c3cccc4sc5ccccc5c34)nc(-n3c4ccccc4c4ccccc43)n2)cc1 ; O=P(c1ccccc1)(c1ccccc1)c1ccccc1-c1nc(-c2cccc3c2oc2ccccc23)nc(-n2c3ccccc3c3ccccc32)n1]

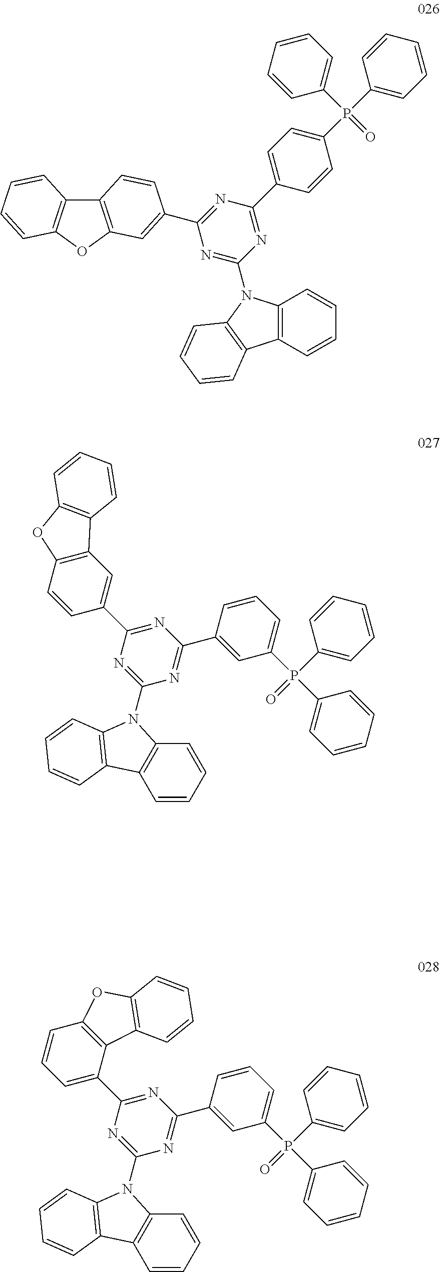 [SMILES: O=P(c1ccccc1)(c1ccccc1)c1ccc(-c2nc(-c3ccc4c(c3)oc3ccccc34)nc(-n3c4ccccc4c4ccccc43)n2)cc1 ; O=P(c1ccccc1)(c1ccccc1)c1cccc(-c2nc(-c3ccc4oc5ccccc5c4c3)nc(-n3c4ccccc4c4ccccc43)n2)c1 ; O=P(c1ccccc1)(c1ccccc1)c1cccc(-c2nc(-c3cccc4oc5ccccc5c34)nc(-n3c4ccccc4c4ccccc43)n2)c1]

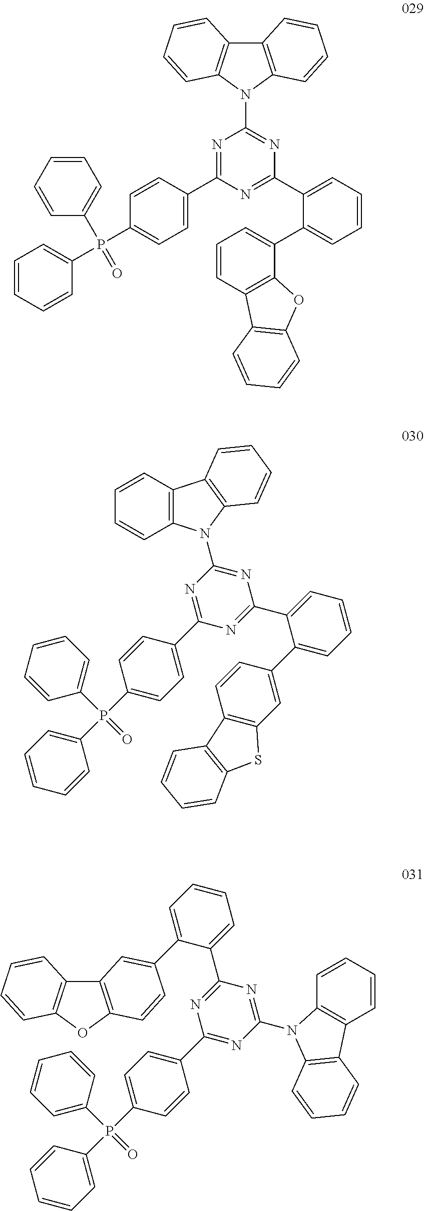 [SMILES: O=P(c1ccccc1)(c1ccccc1)c1ccc(-c2nc(-c3ccccc3-c3cccc4c3oc3ccccc34)nc(-n3c4ccccc4c4ccccc43)n2)cc1 ; O=P(c1ccccc1)(c1ccccc1)c1ccc(-c2nc(-c3ccccc3-c3ccc4c(c3)sc3ccccc34)nc(-n3c4ccccc4c4ccccc43)n2)cc1 ; O=P(c1ccccc1)(c1ccccc1)c1ccc(-c2nc(-c3ccccc3-c3ccc4oc5ccccc5c4c3)nc(-n3c4ccccc4c4ccccc43)n2)cc1]

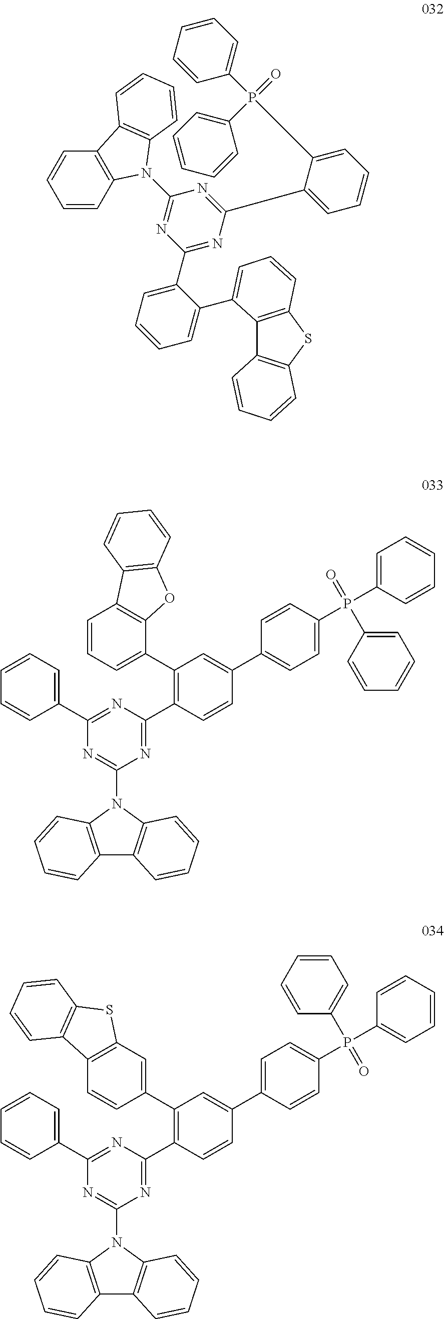 [SMILES: O=P(c1ccccc1)(c1ccccc1)c1ccccc1-c1nc(-c2ccccc2-c2cccc3sc4ccccc4c23)nc(-n2c3ccccc3c3ccccc32)n1 ; O=P(c1ccccc1)(c1ccccc1)c1ccc(-c2ccc(-c3nc(-c4ccccc4)nc(-n4c5ccccc5c5ccccc54)n3)c(-c3cccc4c3oc3ccccc34)c2)cc1 ; O=P(c1ccccc1)(c1ccccc1)c1ccc(-c2ccc(-c3nc(-c4ccccc4)nc(-n4c5ccccc5c5ccccc54)n3)c(-c3ccc4c(c3)sc3ccccc34)c2)cc1]

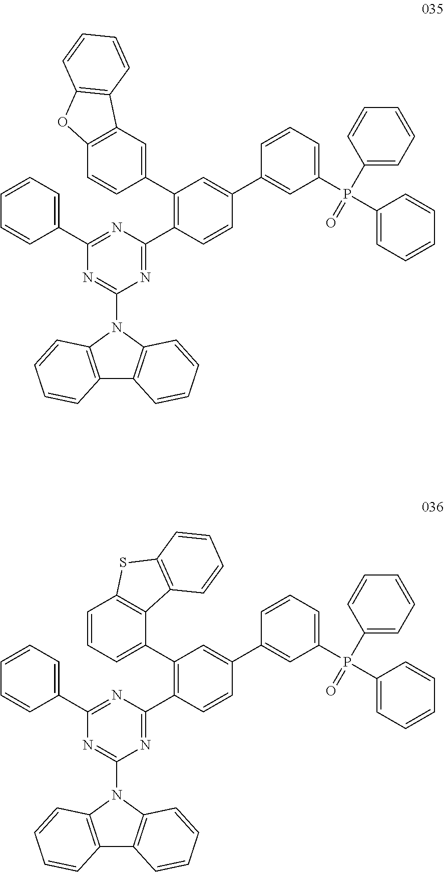 [SMILES: O=P(c1ccccc1)(c1ccccc1)c1cccc(-c2ccc(-c3nc(-c4ccccc4)nc(-n4c5ccccc5c5ccccc54)n3)c(-c3ccc4oc5ccccc5c4c3)c2)c1 ; O=P(c1ccccc1)(c1ccccc1)c1cccc(-c2ccc(-c3nc(-c4ccccc4)nc(-n4c5ccccc5c5ccccc54)n3)c(-c3cccc4sc5ccccc5c34)c2)c1]

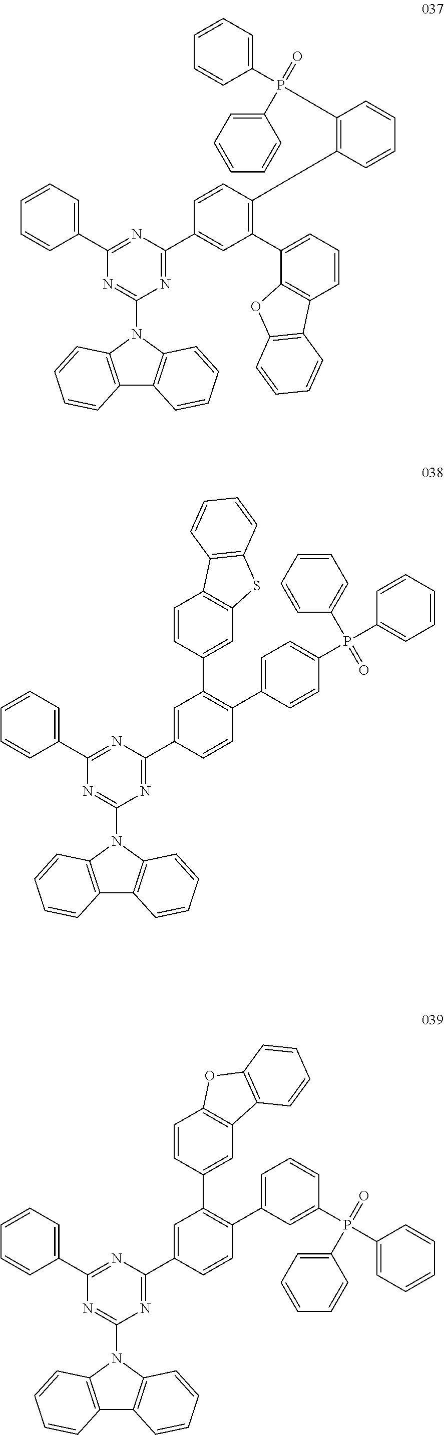 [SMILES: O=P(c1ccccc1)(c1ccccc1)c1ccccc1-c1ccc(-c2nc(-c3ccccc3)nc(-n3c4ccccc4c4ccccc43)n2)cc1-c1cccc2c1oc1ccccc12 ; O=P(c1ccccc1)(c1ccccc1)c1ccc(-c2ccc(-c3nc(-c4ccccc4)nc(-n4c5ccccc5c5ccccc54)n3)cc2-c2ccc3c(c2)sc2ccccc23)cc1 ; O=P(c1ccccc1)(c1ccccc1)c1cccc(-c2ccc(-c3nc(-c4ccccc4)nc(-n4c5ccccc5c5ccccc54)n3)cc2-c2ccc3oc4ccccc4c3c2)c1]

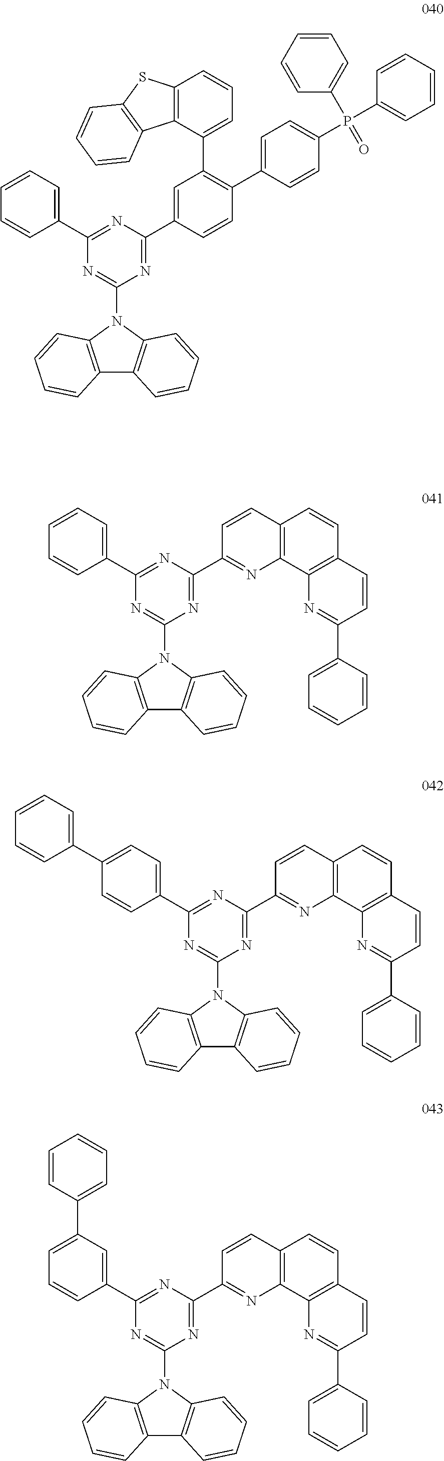 [SMILES: O=P(c1ccccc1)(c1ccccc1)c1ccc(-c2ccc(-c3nc(-c4ccccc4)nc(-n4c5ccccc5c5ccccc54)n3)cc2-c2cccc3sc4ccccc4c23)cc1 ; c1ccc(-c2ccc3ccc4ccc(-c5nc(-c6ccccc6)nc(-n6c7ccccc7c7ccccc76)n5)nc4c3n2)cc1 ; c1ccc(-c2ccc(-c3nc(-c4ccc5ccc6ccc(-c7ccccc7)nc6c5n4)nc(-n4c5ccccc5c5ccccc54)n3)cc2)cc1 ; c1ccc(-c2cccc(-c3nc(-c4ccc5ccc6ccc(-c7ccccc7)nc6c5n4)nc(-n4c5ccccc5c5ccccc54)n3)c2)cc1]

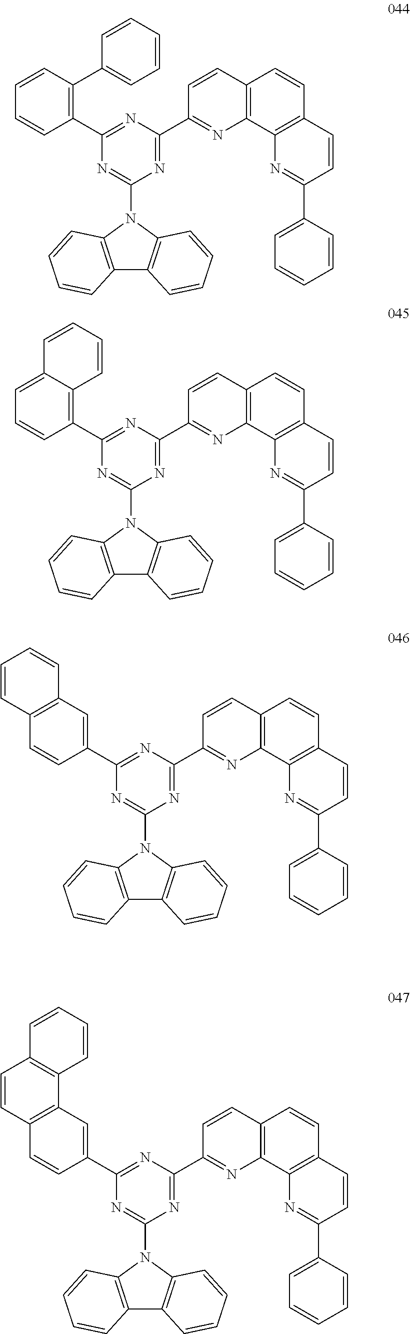

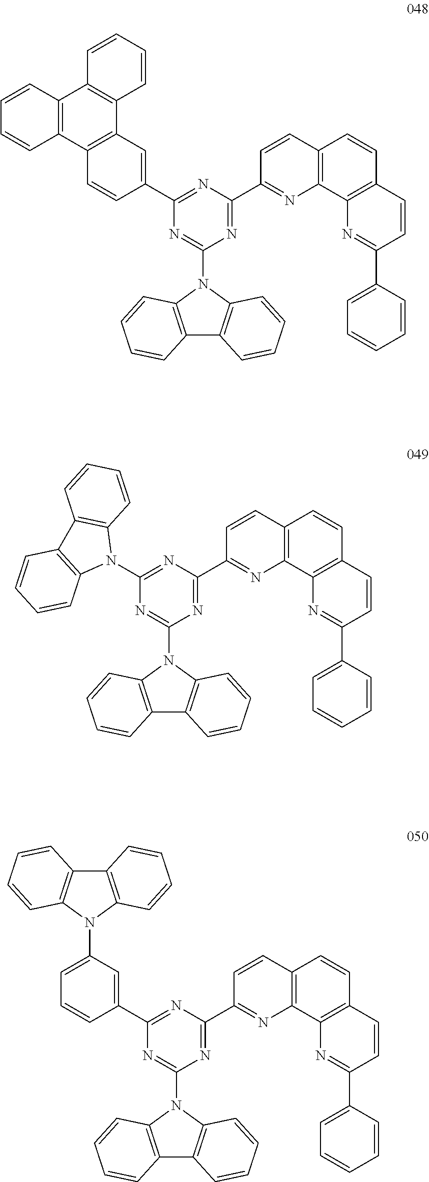 [SMILES: c1ccc(-c2ccc3ccc4ccc(-c5nc(-c6ccc7c8ccccc8c8ccccc8c7c6)nc(-n6c7ccccc7c7ccccc76)n5)nc4c3n2)cc1 ; c1ccc(-c2ccc3ccc4ccc(-c5nc(-n6c7ccccc7c7ccccc76)nc(-n6c7ccccc7c7ccccc76)n5)nc4c3n2)cc1 ; c1ccc(-c2ccc3ccc4ccc(-c5nc(-c6cccc(-n7c8ccccc8c8ccccc87)c6)nc(-n6c7ccccc7c7ccccc76)n5)nc4c3n2)cc1]

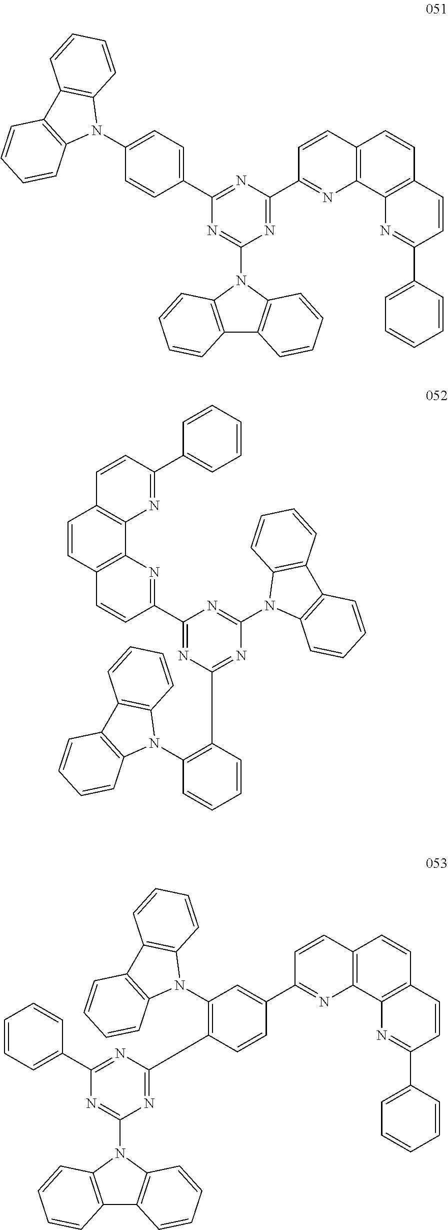 [SMILES: c1ccc(-c2ccc3ccc4ccc(-c5nc(-c6ccc(-n7c8ccccc8c8ccccc87)cc6)nc(-n6c7ccccc7c7ccccc76)n5)nc4c3n2)cc1 ; c1ccc(-c2ccc3ccc4ccc(-c5nc(-c6ccccc6-n6c7ccccc7c7ccccc76)nc(-n6c7ccccc7c7ccccc76)n5)nc4c3n2)cc1 ; c1ccc(-c2ccc3ccc4ccc(-c5ccc(-c6nc(-c7ccccc7)nc(-n7c8ccccc8c8ccccc87)n6)c(-n6c7ccccc7c7ccccc76)c5)nc4c3n2)cc1]

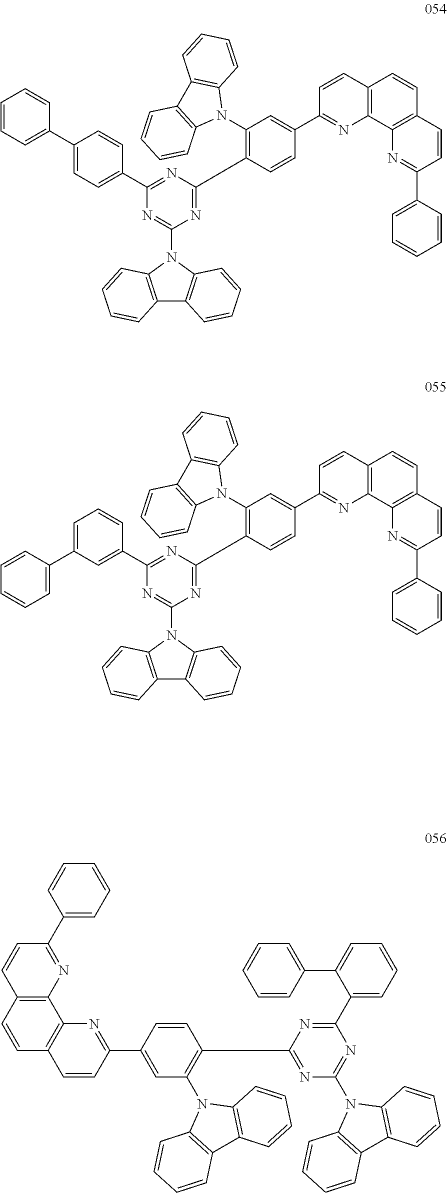 [SMILES: c1ccc(-c2ccc(-c3nc(-c4ccc(-c5ccc6ccc7ccc(-c8ccccc8)nc7c6n5)cc4-n4c5ccccc5c5ccccc54)nc(-n4c5ccccc5c5ccccc54)n3)cc2)cc1 ; c1ccc(-c2cccc(-c3nc(-c4ccc(-c5ccc6ccc7ccc(-c8ccccc8)nc7c6n5)cc4-n4c5ccccc5c5ccccc54)nc(-n4c5ccccc5c5ccccc54)n3)c2)cc1 ; c1ccc(-c2ccc3ccc4ccc(-c5ccc(-c6nc(-c7ccccc7-c7ccccc7)nc(-n7c8ccccc8c8ccccc87)n6)c(-n6c7ccccc7c7ccccc76)c5)nc4c3n2)cc1]

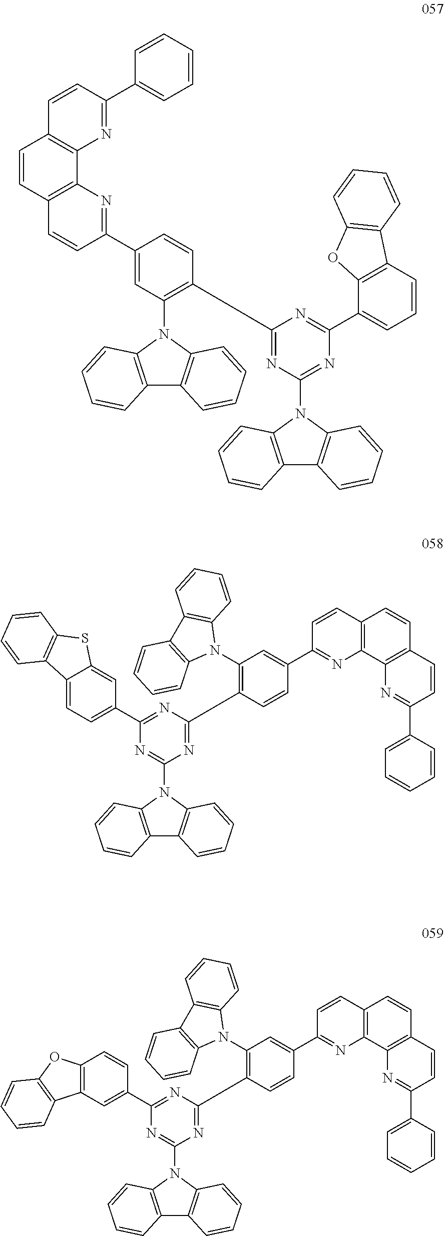 [SMILES: c1ccc(-c2ccc3ccc4ccc(-c5ccc(-c6nc(-c7cccc8c7oc7ccccc78)nc(-n7c8ccccc8c8ccccc87)n6)c(-n6c7ccccc7c7ccccc76)c5)nc4c3n2)cc1 ; c1ccc(-c2ccc3ccc4ccc(-c5ccc(-c6nc(-c7ccc8c(c7)sc7ccccc78)nc(-n7c8ccccc8c8ccccc87)n6)c(-n6c7ccccc7c7ccccc76)c5)nc4c3n2)cc1 ; c1ccc(-c2ccc3ccc4ccc(-c5ccc(-c6nc(-c7ccc8oc9ccccc9c8c7)nc(-n7c8ccccc8c8ccccc87)n6)c(-n6c7ccccc7c7ccccc76)c5)nc4c3n2)cc1]

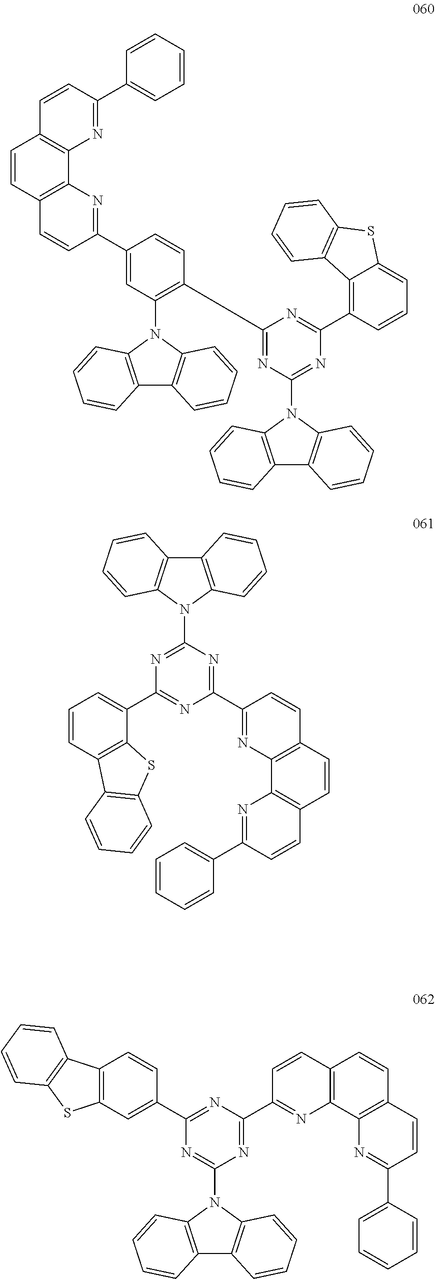 [SMILES: c1ccc(-c2ccc3ccc4ccc(-c5ccc(-c6nc(-c7cccc8sc9ccccc9c78)nc(-n7c8ccccc8c8ccccc87)n6)c(-n6c7ccccc7c7ccccc76)c5)nc4c3n2)cc1 ; c1ccc(-c2ccc3ccc4ccc(-c5nc(-c6cccc7c6sc6ccccc67)nc(-n6c7ccccc7c7ccccc76)n5)nc4c3n2)cc1 ; c1ccc(-c2ccc3ccc4ccc(-c5nc(-c6ccc7c(c6)sc6ccccc67)nc(-n6c7ccccc7c7ccccc76)n5)nc4c3n2)cc1]

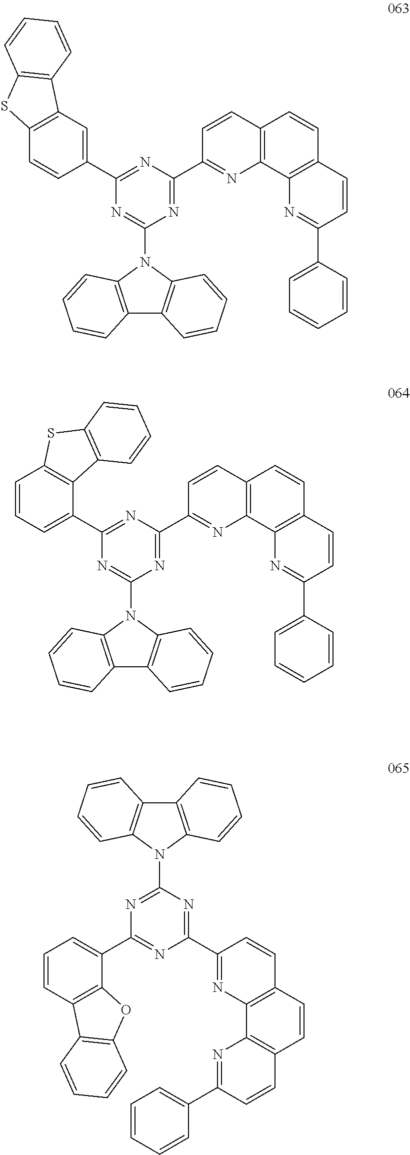 [SMILES: c1ccc(-c2ccc3ccc4ccc(-c5nc(-c6ccc7sc8ccccc8c7c6)nc(-n6c7ccccc7c7ccccc76)n5)nc4c3n2)cc1 ; c1ccc(-c2ccc3ccc4ccc(-c5nc(-c6cccc7sc8ccccc8c67)nc(-n6c7ccccc7c7ccccc76)n5)nc4c3n2)cc1 ; c1ccc(-c2ccc3ccc4ccc(-c5nc(-c6cccc7c6oc6ccccc67)nc(-n6c7ccccc7c7ccccc76)n5)nc4c3n2)cc1]

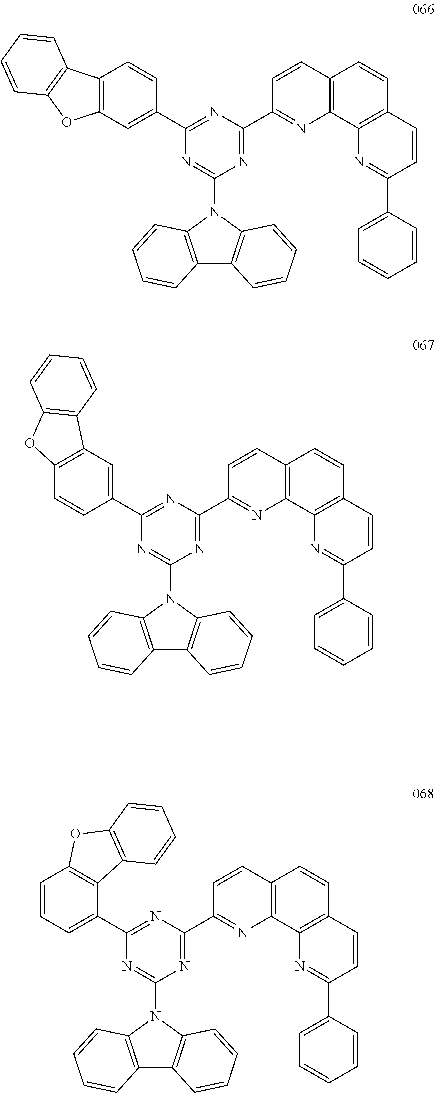 [SMILES: c1ccc(-c2ccc3ccc4ccc(-c5nc(-c6ccc7c(c6)oc6ccccc67)nc(-n6c7ccccc7c7ccccc76)n5)nc4c3n2)cc1 ; c1ccc(-c2ccc3ccc4ccc(-c5nc(-c6ccc7oc8ccccc8c7c6)nc(-n6c7ccccc7c7ccccc76)n5)nc4c3n2)cc1 ; c1ccc(-c2ccc3ccc4ccc(-c5nc(-c6cccc7oc8ccccc8c67)nc(-n6c7ccccc7c7ccccc76)n5)nc4c3n2)cc1]

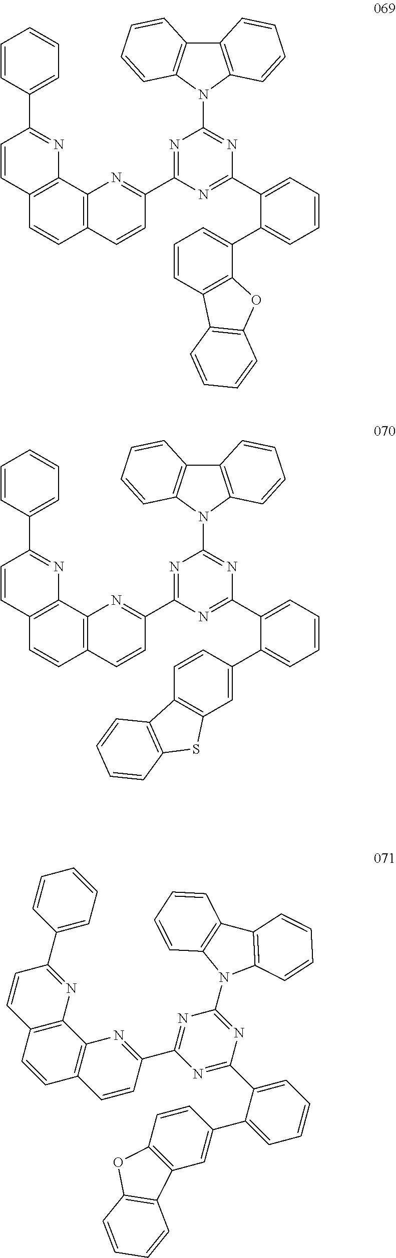 [SMILES: c1ccc(-c2ccc3ccc4ccc(-c5nc(-c6ccccc6-c6cccc7c6oc6ccccc67)nc(-n6c7ccccc7c7ccccc76)n5)nc4c3n2)cc1 ; c1ccc(-c2ccc3ccc4ccc(-c5nc(-c6ccccc6-c6ccc7c(c6)sc6ccccc67)nc(-n6c7ccccc7c7ccccc76)n5)nc4c3n2)cc1 ; c1ccc(-c2ccc3ccc4ccc(-c5nc(-c6ccccc6-c6ccc7oc8ccccc8c7c6)nc(-n6c7ccccc7c7ccccc76)n5)nc4c3n2)cc1]

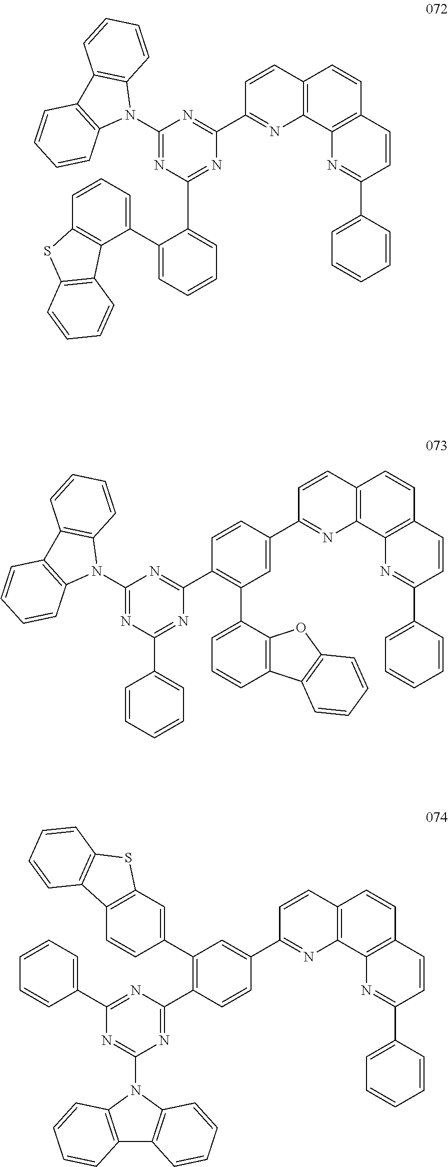 [SMILES: c1ccc(-c2ccc3ccc4ccc(-c5nc(-c6ccccc6-c6cccc7sc8ccccc8c67)nc(-n6c7ccccc7c7ccccc76)n5)nc4c3n2)cc1 ; c1ccc(-c2ccc3ccc4ccc(-c5ccc(-c6nc(-c7ccccc7)nc(-n7c8ccccc8c8ccccc87)n6)c(-c6cccc7c6oc6ccccc67)c5)nc4c3n2)cc1 ; c1ccc(-c2ccc3ccc4ccc(-c5ccc(-c6nc(-c7ccccc7)nc(-n7c8ccccc8c8ccccc87)n6)c(-c6ccc7c(c6)sc6ccccc67)c5)nc4c3n2)cc1]

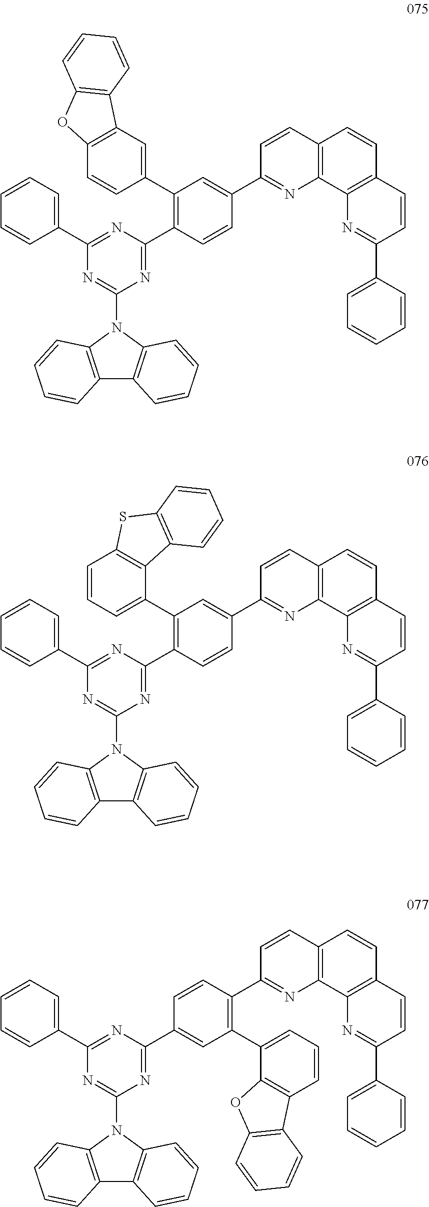 [SMILES: c1ccc(-c2ccc3ccc4ccc(-c5ccc(-c6nc(-c7ccccc7)nc(-n7c8ccccc8c8ccccc87)n6)c(-c6ccc7oc8ccccc8c7c6)c5)nc4c3n2)cc1 ; c1ccc(-c2ccc3ccc4ccc(-c5ccc(-c6nc(-c7ccccc7)nc(-n7c8ccccc8c8ccccc87)n6)c(-c6cccc7sc8ccccc8c67)c5)nc4c3n2)cc1 ; c1ccc(-c2ccc3ccc4ccc(-c5ccc(-c6nc(-c7ccccc7)nc(-n7c8ccccc8c8ccccc87)n6)cc5-c5cccc6c5oc5ccccc56)nc4c3n2)cc1]

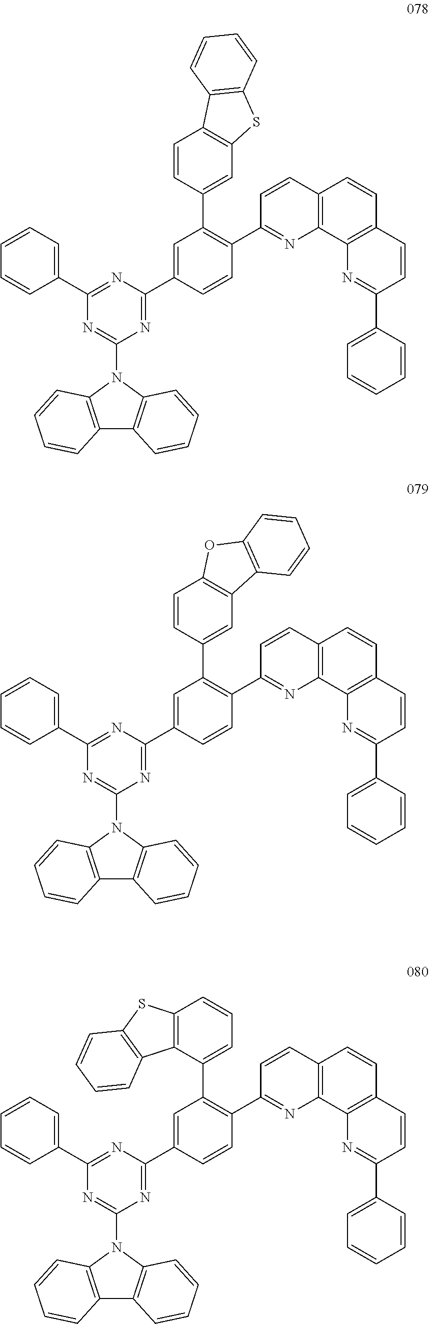 [SMILES: c1ccc(-c2ccc3ccc4ccc(-c5ccc(-c6nc(-c7ccccc7)nc(-n7c8ccccc8c8ccccc87)n6)cc5-c5ccc6c(c5)sc5ccccc56)nc4c3n2)cc1 ; c1ccc(-c2ccc3ccc4ccc(-c5ccc(-c6nc(-c7ccccc7)nc(-n7c8ccccc8c8ccccc87)n6)cc5-c5ccc6oc7ccccc7c6c5)nc4c3n2)cc1 ; c1ccc(-c2ccc3ccc4ccc(-c5ccc(-c6nc(-c7ccccc7)nc(-n7c8ccccc8c8ccccc87)n6)cc5-c5cccc6sc7ccccc7c56)nc4c3n2)cc1]

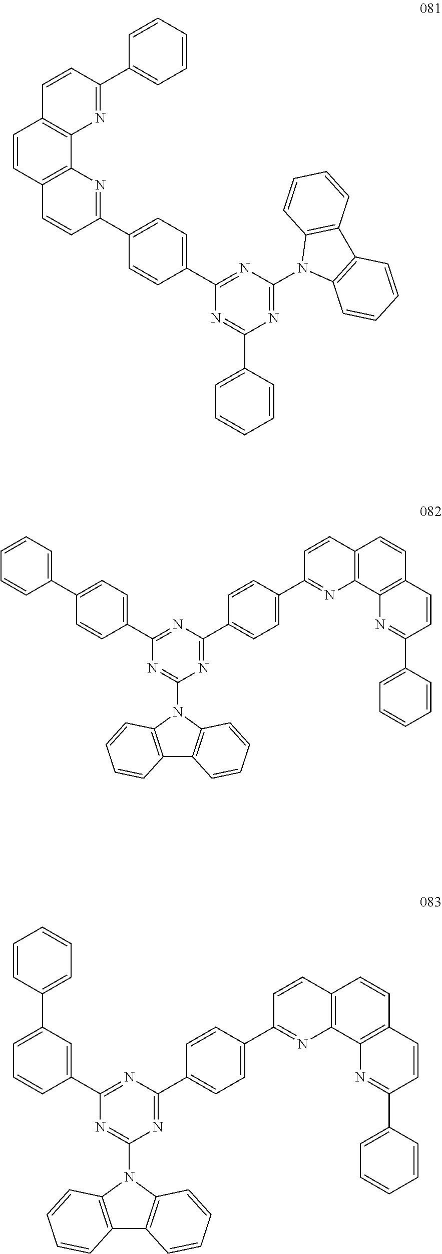 [SMILES: c1ccc(-c2ccc3ccc4ccc(-c5ccc(-c6nc(-c7ccccc7)nc(-n7c8ccccc8c8ccccc87)n6)cc5)nc4c3n2)cc1 ; c1ccc(-c2ccc(-c3nc(-c4ccc(-c5ccc6ccc7ccc(-c8ccccc8)nc7c6n5)cc4)nc(-n4c5ccccc5c5ccccc54)n3)cc2)cc1 ; c1ccc(-c2cccc(-c3nc(-c4ccc(-c5ccc6ccc7ccc(-c8ccccc8)nc7c6n5)cc4)nc(-n4c5ccccc5c5ccccc54)n3)c2)cc1]

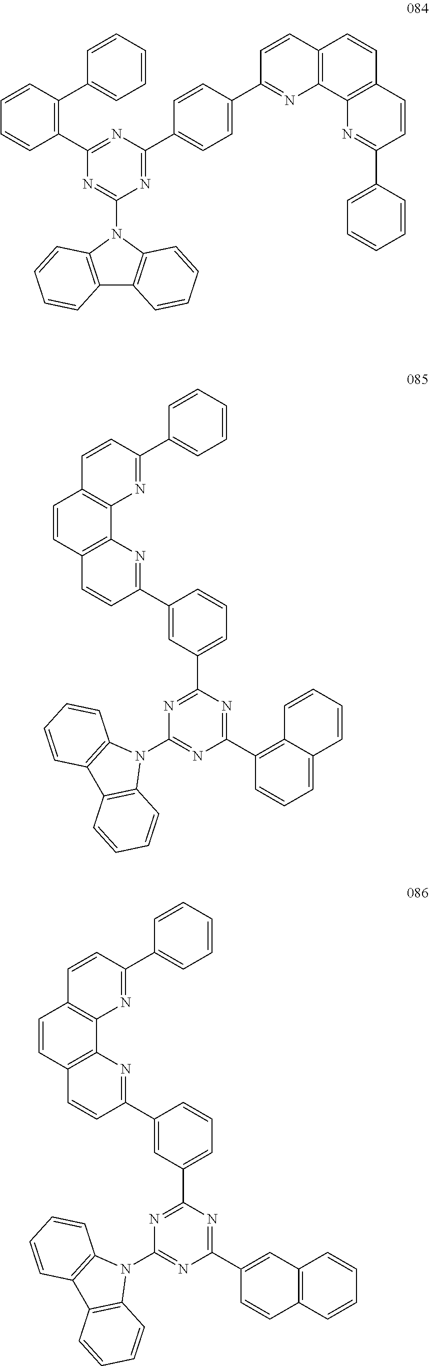 [SMILES: c1ccc(-c2ccc3ccc4ccc(-c5ccc(-c6nc(-c7ccccc7-c7ccccc7)nc(-n7c8ccccc8c8ccccc87)n6)cc5)nc4c3n2)cc1 ; c1ccc(-c2ccc3ccc4ccc(-c5cccc(-c6nc(-c7cccc8ccccc78)nc(-n7c8ccccc8c8ccccc87)n6)c5)nc4c3n2)cc1 ; c1ccc(-c2ccc3ccc4ccc(-c5cccc(-c6nc(-c7ccc8ccccc8c7)nc(-n7c8ccccc8c8ccccc87)n6)c5)nc4c3n2)cc1]

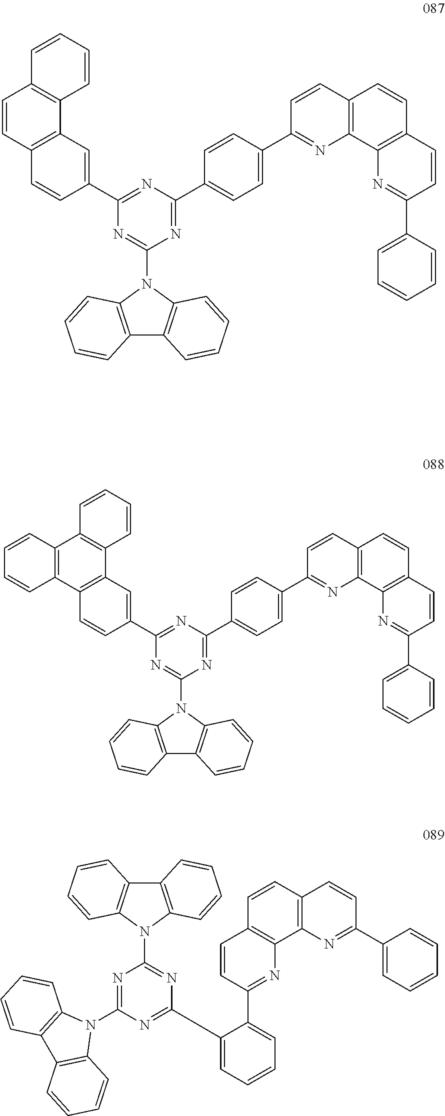 [SMILES: c1ccc(-c2ccc3ccc4ccc(-c5ccc(-c6nc(-c7ccc8ccc9ccccc9c8c7)nc(-n7c8ccccc8c8ccccc87)n6)cc5)nc4c3n2)cc1 ; c1ccc(-c2ccc3ccc4ccc(-c5ccc(-c6nc(-c7ccc8c9ccccc9c9ccccc9c8c7)nc(-n7c8ccccc8c8ccccc87)n6)cc5)nc4c3n2)cc1 ; c1ccc(-c2ccc3ccc4ccc(-c5ccccc5-c5nc(-n6c7ccccc7c7ccccc76)nc(-n6c7ccccc7c7ccccc76)n5)nc4c3n2)cc1]

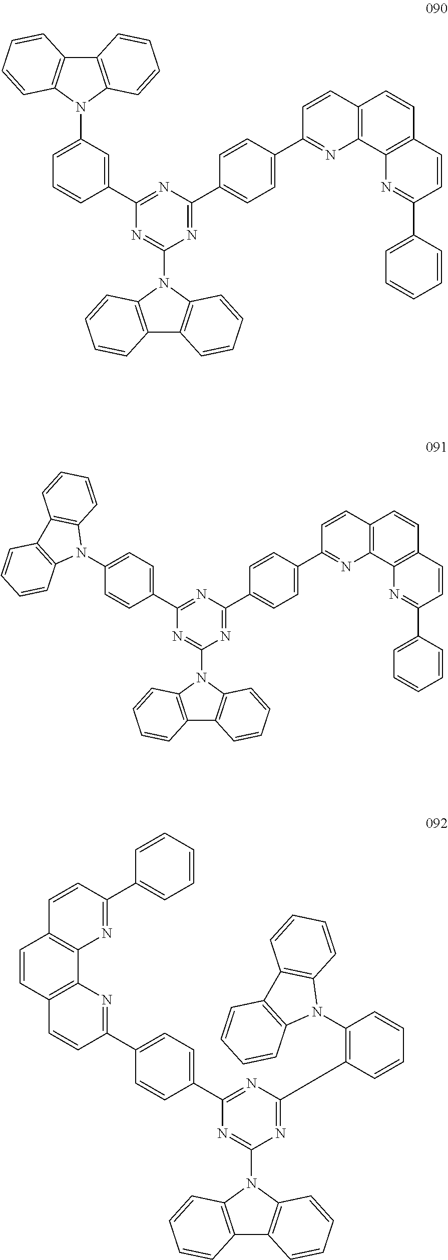 [SMILES: c1ccc(-c2ccc3ccc4ccc(-c5ccc(-c6nc(-c7cccc(-n8c9ccccc9c9ccccc98)c7)nc(-n7c8ccccc8c8ccccc87)n6)cc5)nc4c3n2)cc1 ; c1ccc(-c2ccc3ccc4ccc(-c5ccc(-c6nc(-c7ccc(-n8c9ccccc9c9ccccc98)cc7)nc(-n7c8ccccc8c8ccccc87)n6)cc5)nc4c3n2)cc1 ; c1ccc(-c2ccc3ccc4ccc(-c5ccc(-c6nc(-c7ccccc7-n7c8ccccc8c8ccccc87)nc(-n7c8ccccc8c8ccccc87)n6)cc5)nc4c3n2)cc1]

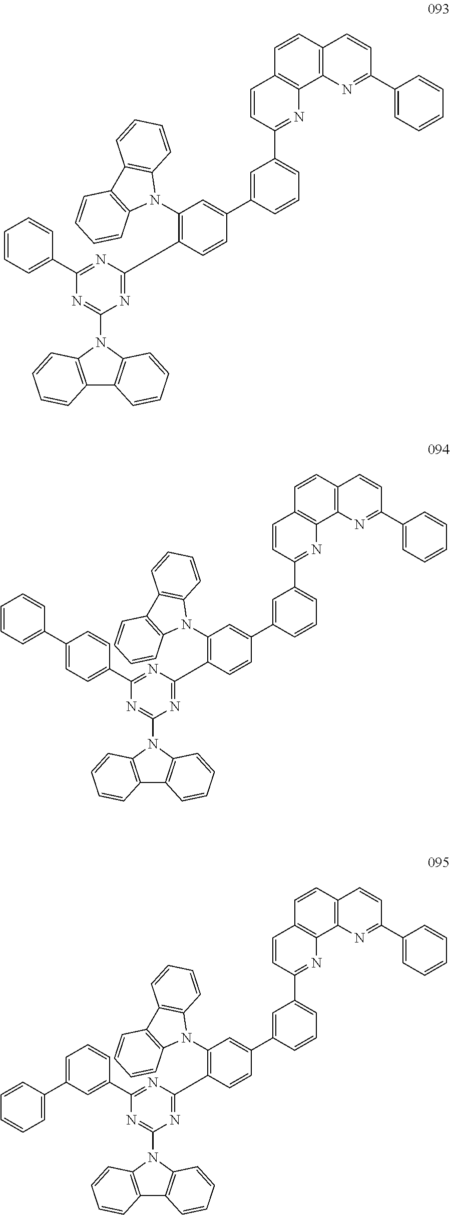

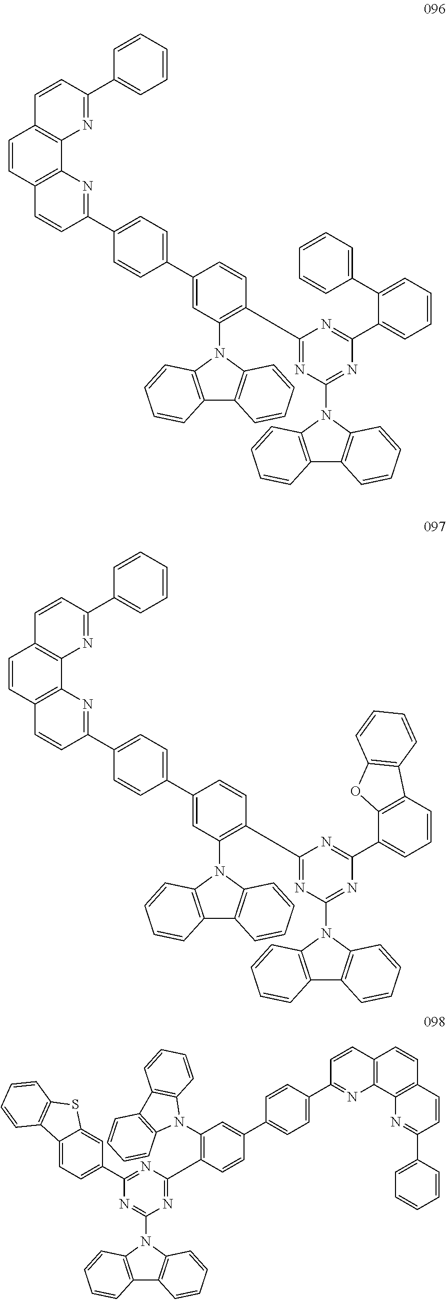

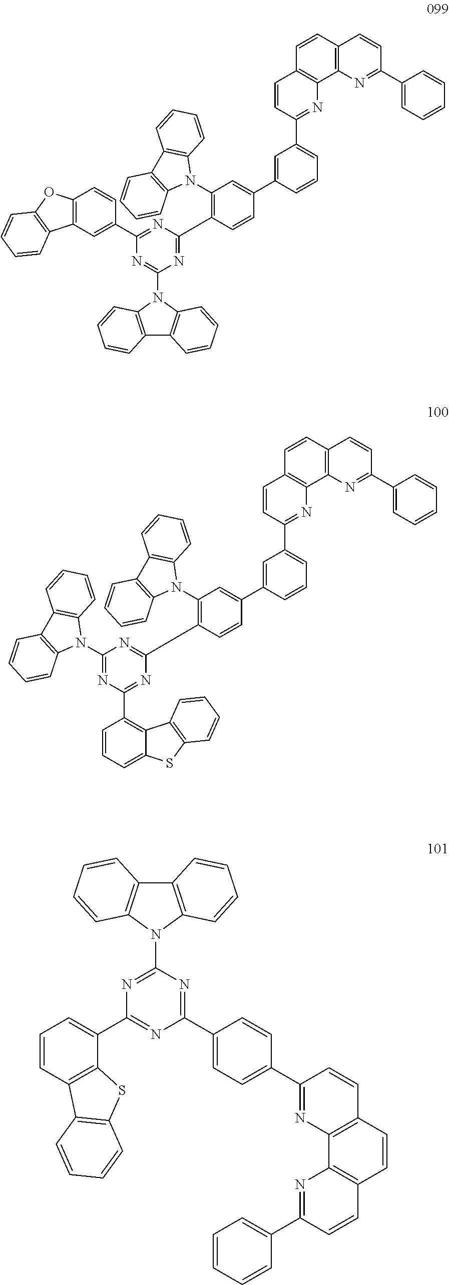

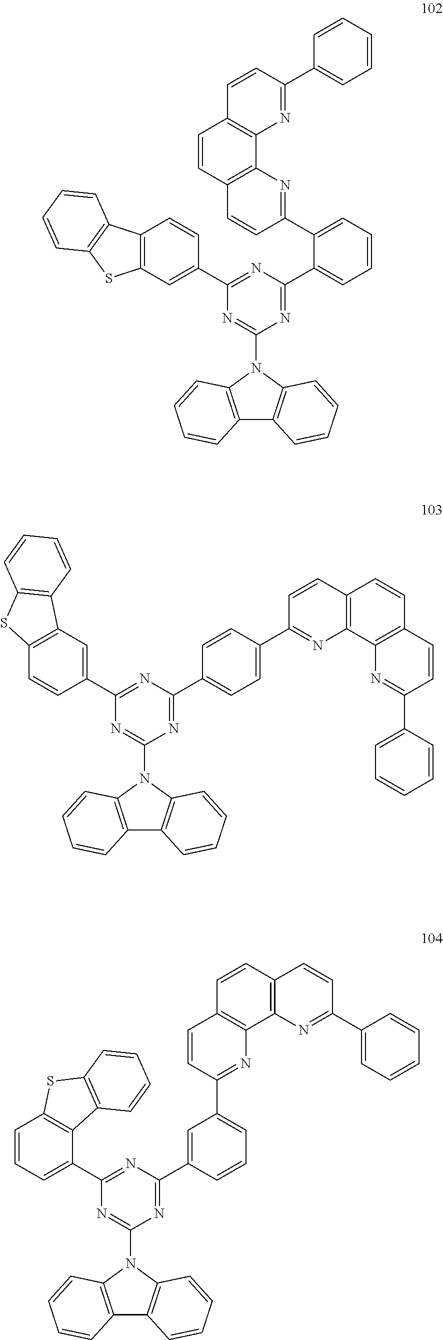 [SMILES: c1ccc(-c2ccc3ccc4ccc(-c5ccccc5-c5nc(-c6ccc7c(c6)sc6ccccc67)nc(-n6c7ccccc7c7ccccc76)n5)nc4c3n2)cc1 ; c1ccc(-c2ccc3ccc4ccc(-c5ccc(-c6nc(-c7ccc8sc9ccccc9c8c7)nc(-n7c8ccccc8c8ccccc87)n6)cc5)nc4c3n2)cc1 ; c1ccc(-c2ccc3ccc4ccc(-c5cccc(-c6nc(-c7cccc8sc9ccccc9c78)nc(-n7c8ccccc8c8ccccc87)n6)c5)nc4c3n2)cc1]

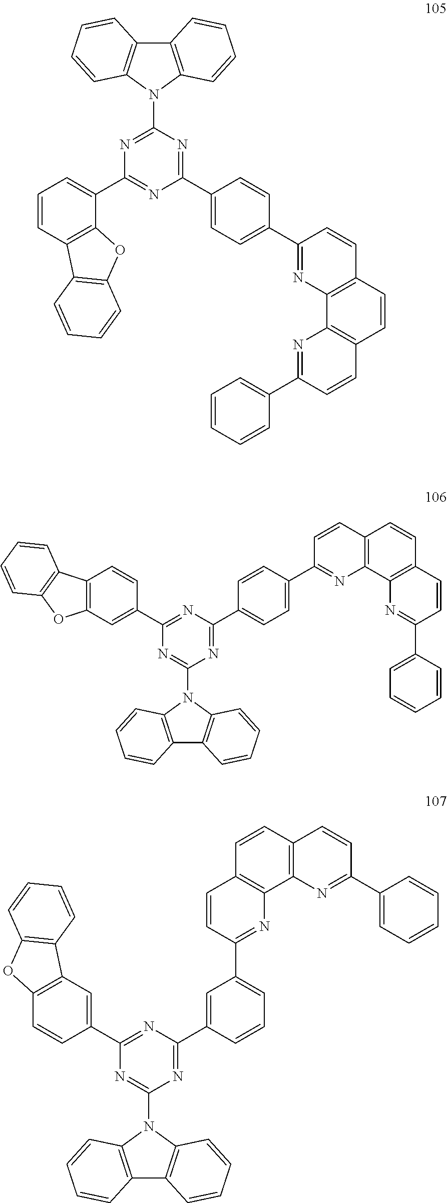

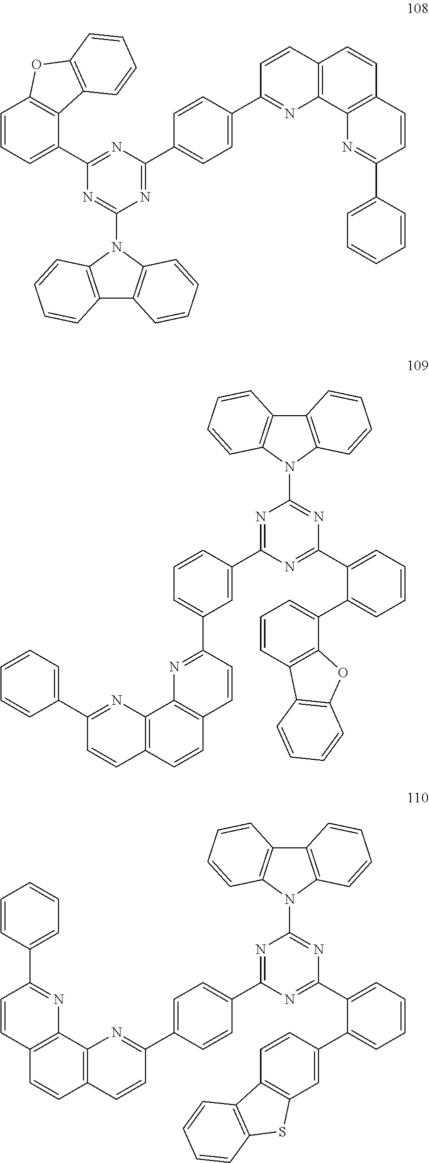 [SMILES: c1ccc(-c2ccc3ccc4ccc(-c5ccc(-c6nc(-c7cccc8oc9ccccc9c78)nc(-n7c8ccccc8c8ccccc87)n6)cc5)nc4c3n2)cc1 ; c1ccc(-c2ccc3ccc4ccc(-c5cccc(-c6nc(-c7ccccc7-c7cccc8c7oc7ccccc78)nc(-n7c8ccccc8c8ccccc87)n6)c5)nc4c3n2)cc1 ; c1ccc(-c2ccc3ccc4ccc(-c5ccc(-c6nc(-c7ccccc7-c7ccc8c(c7)sc7ccccc78)nc(-n7c8ccccc8c8ccccc87)n6)cc5)nc4c3n2)cc1]

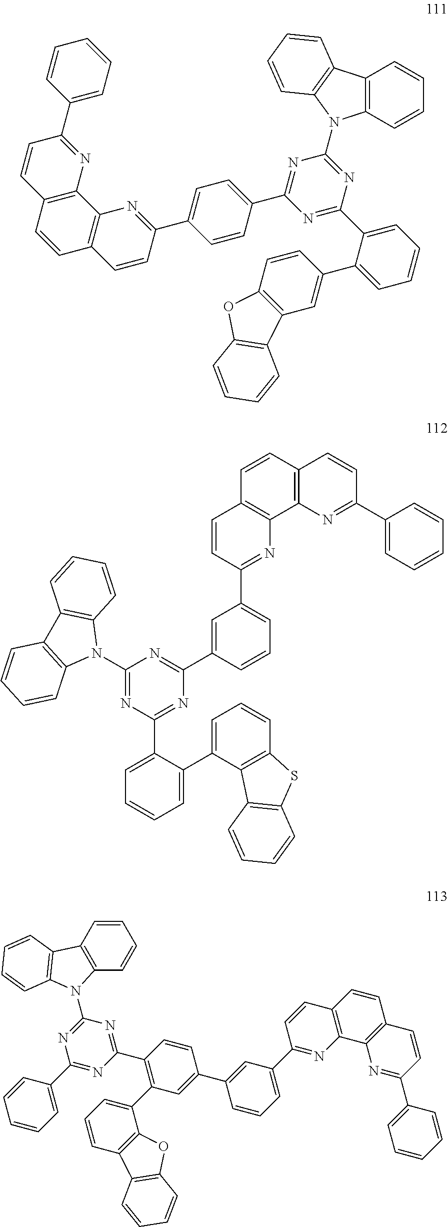 [SMILES: c1ccc(-c2ccc3ccc4ccc(-c5ccc(-c6nc(-c7ccccc7-c7ccc8oc9ccccc9c8c7)nc(-n7c8ccccc8c8ccccc87)n6)cc5)nc4c3n2)cc1 ; c1ccc(-c2ccc3ccc4ccc(-c5cccc(-c6nc(-c7ccccc7-c7cccc8sc9ccccc9c78)nc(-n7c8ccccc8c8ccccc87)n6)c5)nc4c3n2)cc1 ; c1ccc(-c2ccc3ccc4ccc(-c5cccc(-c6ccc(-c7nc(-c8ccccc8)nc(-n8c9ccccc9c9ccccc98)n7)c(-c7cccc8c7oc7ccccc78)c6)c5)nc4c3n2)cc1]

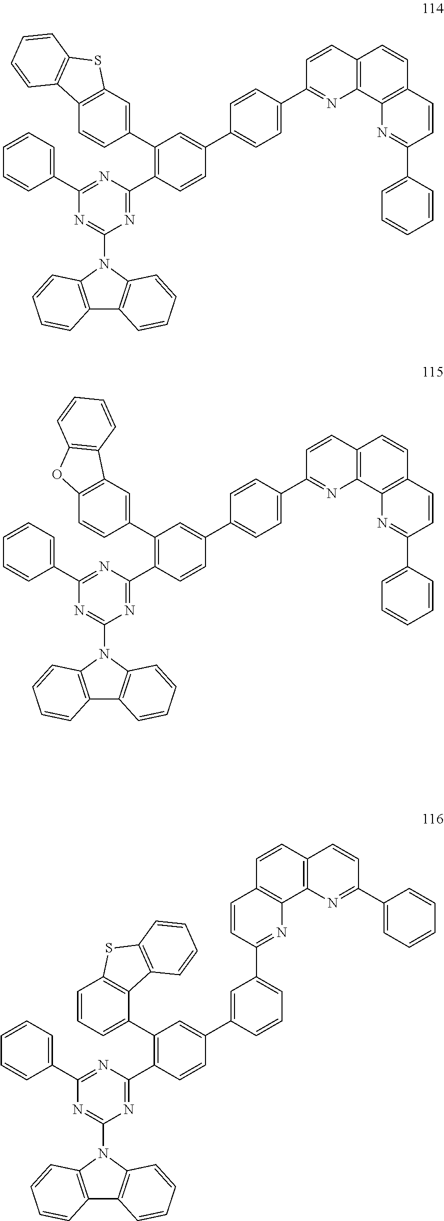 [SMILES: c1ccc(-c2ccc3ccc4ccc(-c5ccc(-c6ccc(-c7nc(-c8ccccc8)nc(-n8c9ccccc9c9ccccc98)n7)c(-c7ccc8c(c7)sc7ccccc78)c6)cc5)nc4c3n2)cc1 ; c1ccc(-c2ccc3ccc4ccc(-c5ccc(-c6ccc(-c7nc(-c8ccccc8)nc(-n8c9ccccc9c9ccccc98)n7)c(-c7ccc8oc9ccccc9c8c7)c6)cc5)nc4c3n2)cc1 ; c1ccc(-c2ccc3ccc4ccc(-c5cccc(-c6ccc(-c7nc(-c8ccccc8)nc(-n8c9ccccc9c9ccccc98)n7)c(-c7cccc8sc9ccccc9c78)c6)c5)nc4c3n2)cc1]

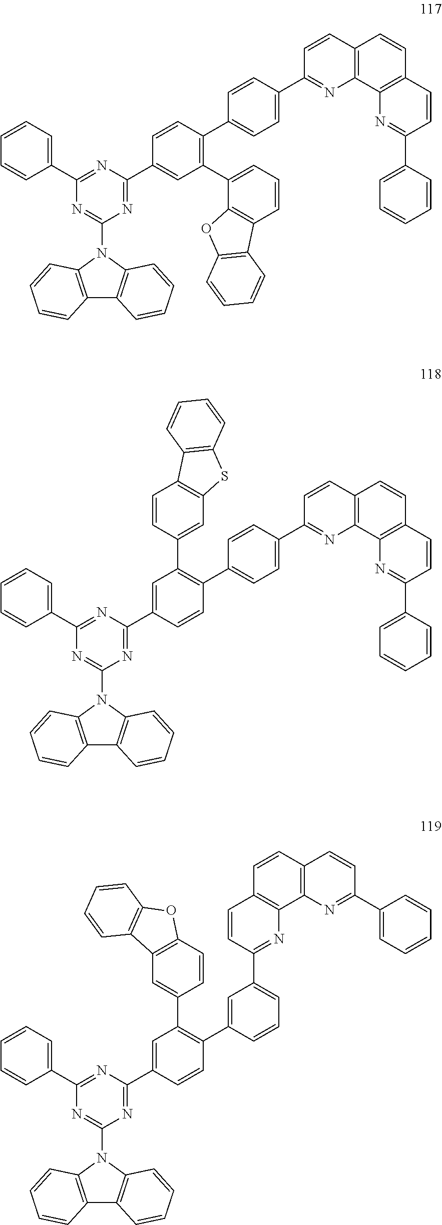 [SMILES: c1ccc(-c2ccc3ccc4ccc(-c5ccc(-c6ccc(-c7nc(-c8ccccc8)nc(-n8c9ccccc9c9ccccc98)n7)cc6-c6cccc7c6oc6ccccc67)cc5)nc4c3n2)cc1 ; c1ccc(-c2ccc3ccc4ccc(-c5ccc(-c6ccc(-c7nc(-c8ccccc8)nc(-n8c9ccccc9c9ccccc98)n7)cc6-c6ccc7c(c6)sc6ccccc67)cc5)nc4c3n2)cc1 ; c1ccc(-c2ccc3ccc4ccc(-c5cccc(-c6ccc(-c7nc(-c8ccccc8)nc(-n8c9ccccc9c9ccccc98)n7)cc6-c6ccc7oc8ccccc8c7c6)c5)nc4c3n2)cc1]

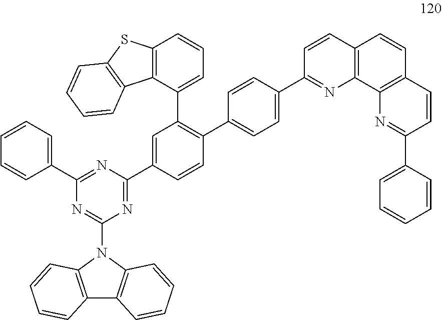 [SMILES: c1ccc(-c2ccc3ccc4ccc(-c5ccc(-c6ccc(-c7nc(-c8ccccc8)nc(-n8c9ccccc9c9ccccc98)n7)cc6-c6cccc7sc8ccccc8c67)cc5)nc4c3n2)cc1]

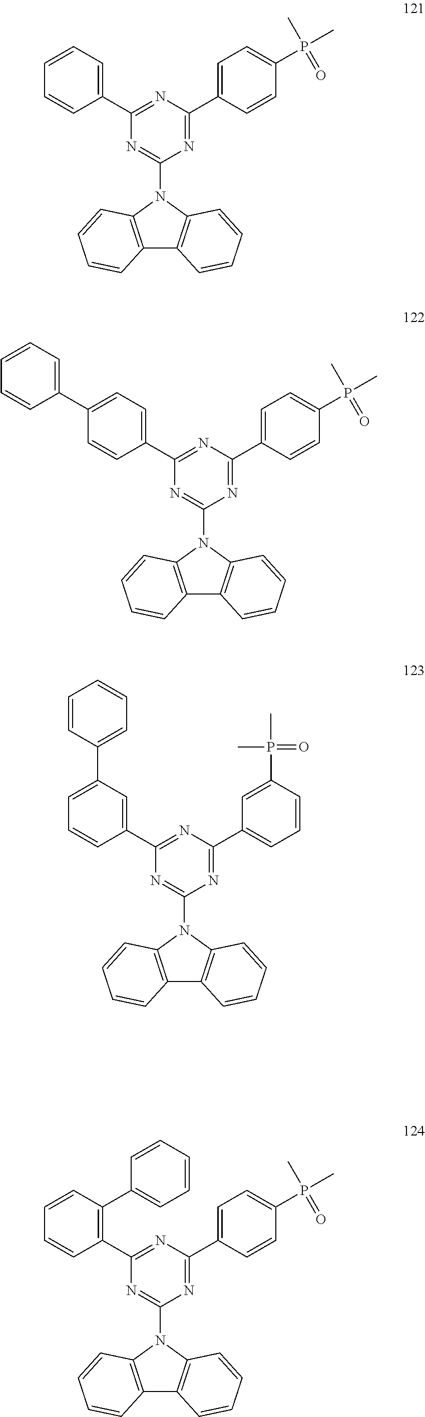 [SMILES: CP(C)(=O)c1ccc(-c2nc(-c3ccccc3)nc(-n3c4ccccc4c4ccccc43)n2)cc1 ; CP(C)(=O)c1ccc(-c2nc(-c3ccc(-c4ccccc4)cc3)nc(-n3c4ccccc4c4ccccc43)n2)cc1 ; CP(C)(=O)c1cccc(-c2nc(-c3cccc(-c4ccccc4)c3)nc(-n3c4ccccc4c4ccccc43)n2)c1 ; CP(C)(=O)c1ccc(-c2nc(-c3ccccc3-c3ccccc3)nc(-n3c4ccccc4c4ccccc43)n2)cc1]

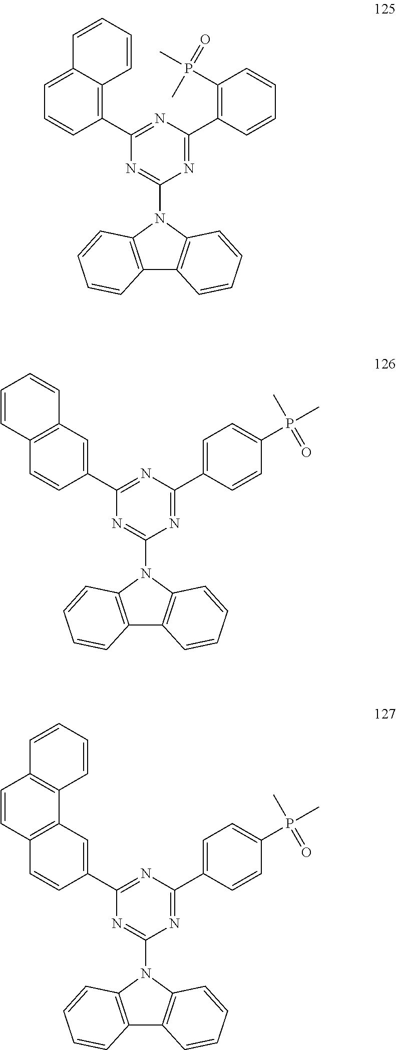 [SMILES: CP(C)(=O)c1ccccc1-c1nc(-c2cccc3ccccc23)nc(-n2c3ccccc3c3ccccc32)n1 ; CP(C)(=O)c1ccc(-c2nc(-c3ccc4ccccc4c3)nc(-n3c4ccccc4c4ccccc43)n2)cc1 ; CP(C)(=O)c1ccc(-c2nc(-c3ccc4ccc5ccccc5c4c3)nc(-n3c4ccccc4c4ccccc43)n2)cc1]

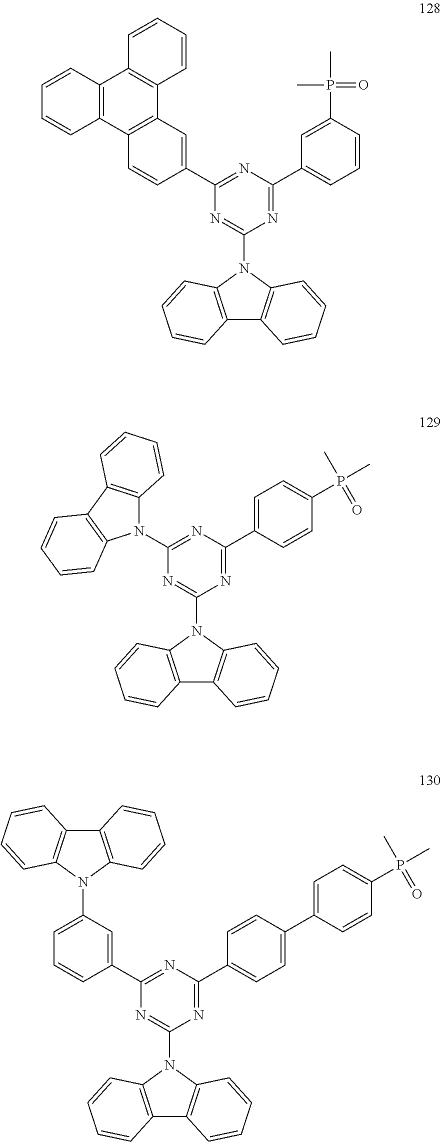 [SMILES: CP(C)(=O)c1cccc(-c2nc(-c3ccc4c5ccccc5c5ccccc5c4c3)nc(-n3c4ccccc4c4ccccc43)n2)c1 ; CP(C)(=O)c1ccc(-c2nc(-n3c4ccccc4c4ccccc43)nc(-n3c4ccccc4c4ccccc43)n2)cc1 ; CP(C)(=O)c1ccc(-c2ccc(-c3nc(-c4cccc(-n5c6ccccc6c6ccccc65)c4)nc(-n4c5ccccc5c5ccccc54)n3)cc2)cc1]

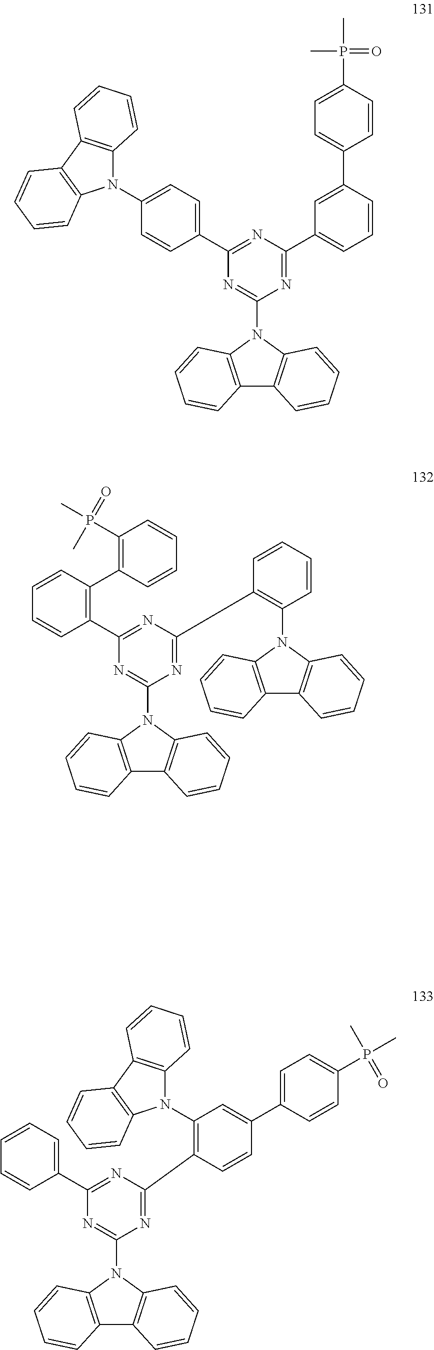 [SMILES: CP(C)(=O)c1ccc(-c2cccc(-c3nc(-c4ccc(-n5c6ccccc6c6ccccc65)cc4)nc(-n4c5ccccc5c5ccccc54)n3)c2)cc1 ; CP(C)(=O)c1ccccc1-c1ccccc1-c1nc(-c2ccccc2-n2c3ccccc3c3ccccc32)nc(-n2c3ccccc3c3ccccc32)n1 ; CP(C)(=O)c1ccc(-c2ccc(-c3nc(-c4ccccc4)nc(-n4c5ccccc5c5ccccc54)n3)c(-n3c4ccccc4c4ccccc43)c2)cc1]

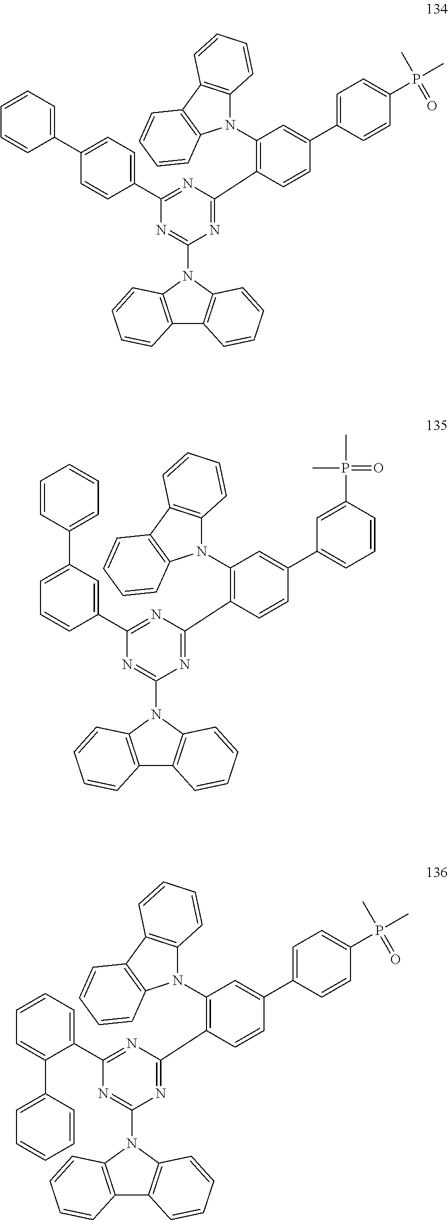 [SMILES: CP(C)(=O)c1ccc(-c2ccc(-c3nc(-c4ccc(-c5ccccc5)cc4)nc(-n4c5ccccc5c5ccccc54)n3)c(-n3c4ccccc4c4ccccc43)c2)cc1 ; CP(C)(=O)c1cccc(-c2ccc(-c3nc(-c4cccc(-c5ccccc5)c4)nc(-n4c5ccccc5c5ccccc54)n3)c(-n3c4ccccc4c4ccccc43)c2)c1 ; CP(C)(=O)c1ccc(-c2ccc(-c3nc(-c4ccccc4-c4ccccc4)nc(-n4c5ccccc5c5ccccc54)n3)c(-n3c4ccccc4c4ccccc43)c2)cc1]

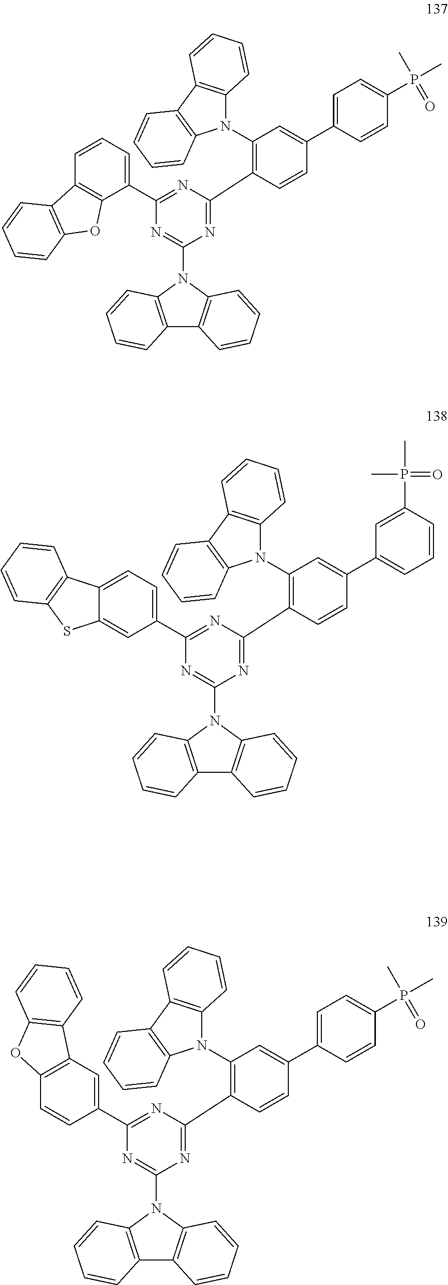 [SMILES: CP(C)(=O)c1ccc(-c2ccc(-c3nc(-c4cccc5c4oc4ccccc45)nc(-n4c5ccccc5c5ccccc54)n3)c(-n3c4ccccc4c4ccccc43)c2)cc1 ; CP(C)(=O)c1cccc(-c2ccc(-c3nc(-c4ccc5c(c4)sc4ccccc45)nc(-n4c5ccccc5c5ccccc54)n3)c(-n3c4ccccc4c4ccccc43)c2)c1 ; CP(C)(=O)c1ccc(-c2ccc(-c3nc(-c4ccc5oc6ccccc6c5c4)nc(-n4c5ccccc5c5ccccc54)n3)c(-n3c4ccccc4c4ccccc43)c2)cc1]

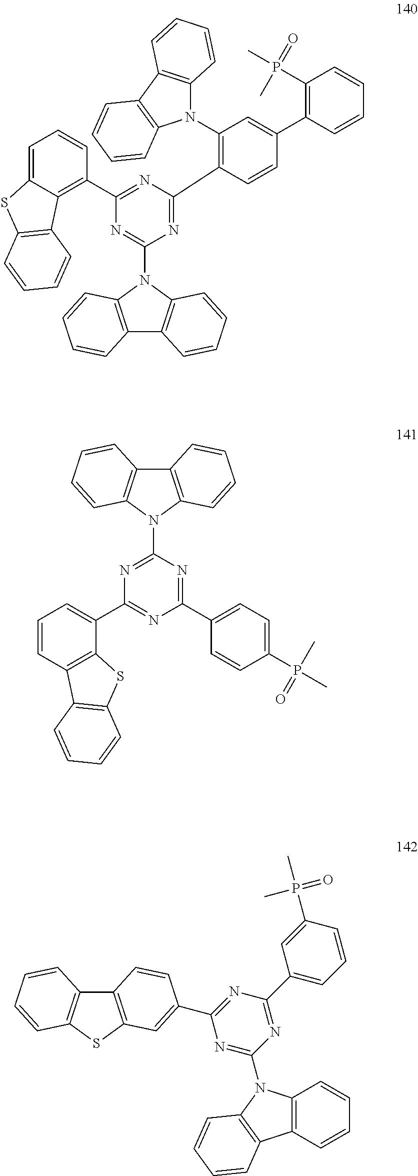 [SMILES: CP(C)(=O)c1ccccc1-c1ccc(-c2nc(-c3cccc4sc5ccccc5c34)nc(-n3c4ccccc4c4ccccc43)n2)c(-n2c3ccccc3c3ccccc32)c1 ; CP(C)(=O)c1ccc(-c2nc(-c3cccc4c3sc3ccccc34)nc(-n3c4ccccc4c4ccccc43)n2)cc1 ; CP(C)(=O)c1cccc(-c2nc(-c3ccc4c(c3)sc3ccccc34)nc(-n3c4ccccc4c4ccccc43)n2)c1]

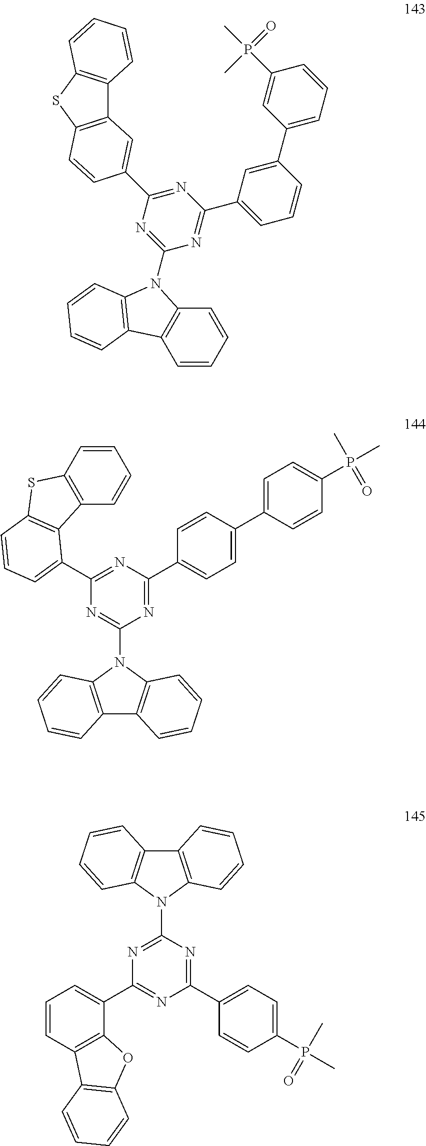 [SMILES: CP(C)(=O)c1cccc(-c2cccc(-c3nc(-c4ccc5sc6ccccc6c5c4)nc(-n4c5ccccc5c5ccccc54)n3)c2)c1 ; CP(C)(=O)c1ccc(-c2ccc(-c3nc(-c4cccc5sc6ccccc6c45)nc(-n4c5ccccc5c5ccccc54)n3)cc2)cc1 ; CP(C)(=O)c1ccc(-c2nc(-c3cccc4c3oc3ccccc34)nc(-n3c4ccccc4c4ccccc43)n2)cc1]

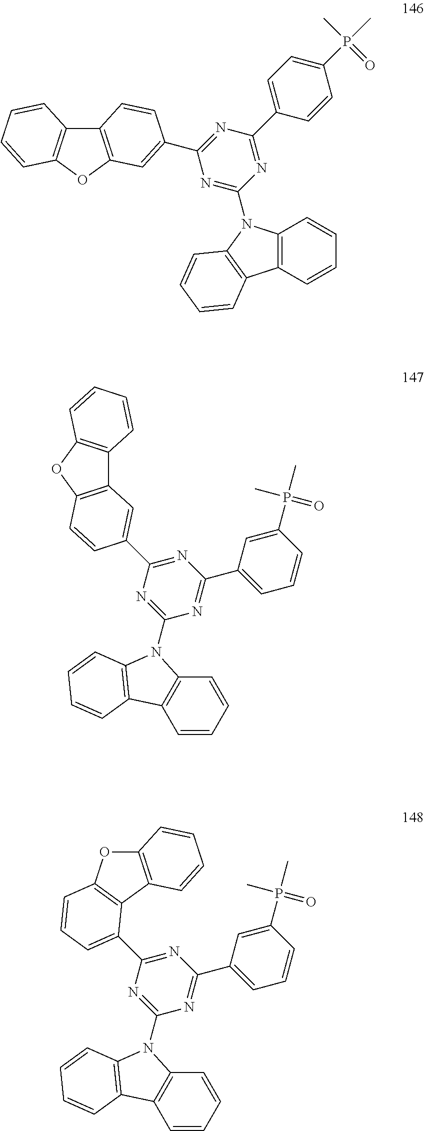 [SMILES: CP(C)(=O)c1ccc(-c2nc(-c3ccc4c(c3)oc3ccccc34)nc(-n3c4ccccc4c4ccccc43)n2)cc1 ; CP(C)(=O)c1cccc(-c2nc(-c3ccc4oc5ccccc5c4c3)nc(-n3c4ccccc4c4ccccc43)n2)c1 ; CP(C)(=O)c1cccc(-c2nc(-c3cccc4oc5ccccc5c34)nc(-n3c4ccccc4c4ccccc43)n2)c1]

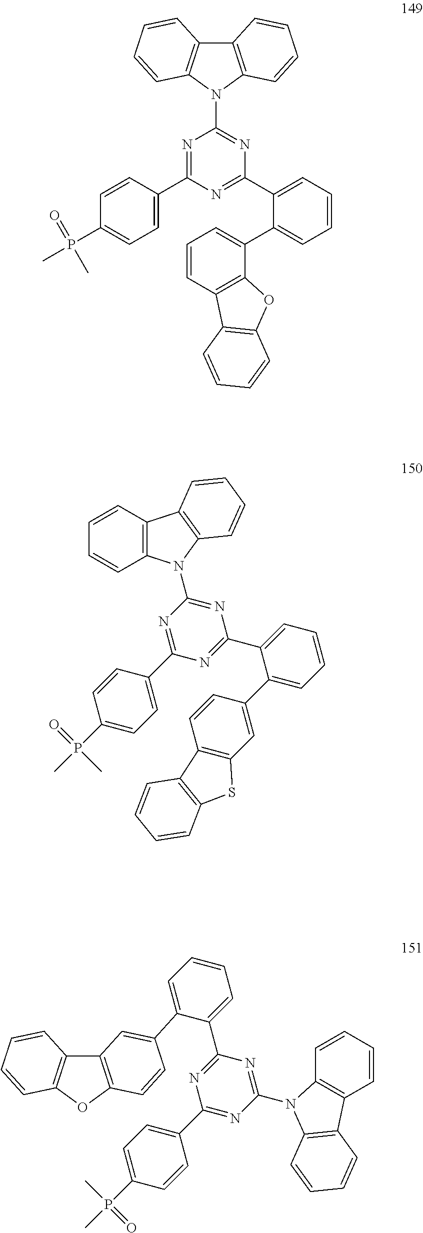 [SMILES: CP(C)(=O)c1ccc(-c2nc(-c3ccccc3-c3cccc4c3oc3ccccc34)nc(-n3c4ccccc4c4ccccc43)n2)cc1 ; CP(C)(=O)c1ccc(-c2nc(-c3ccccc3-c3ccc4c(c3)sc3ccccc34)nc(-n3c4ccccc4c4ccccc43)n2)cc1 ; CP(C)(=O)c1ccc(-c2nc(-c3ccccc3-c3ccc4oc5ccccc5c4c3)nc(-n3c4ccccc4c4ccccc43)n2)cc1]

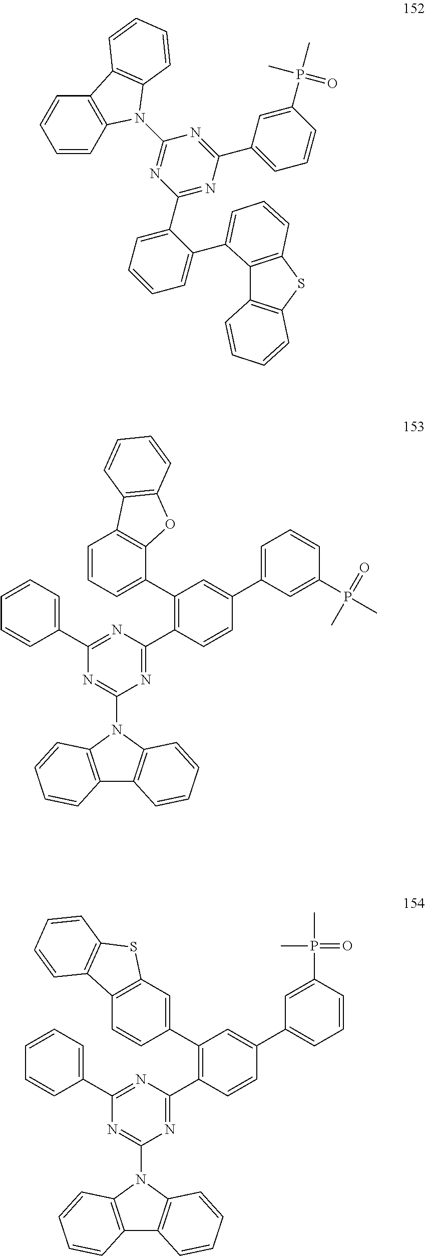 [SMILES: CP(C)(=O)c1cccc(-c2nc(-c3ccccc3-c3cccc4sc5ccccc5c34)nc(-n3c4ccccc4c4ccccc43)n2)c1 ; CP(C)(=O)c1cccc(-c2ccc(-c3nc(-c4ccccc4)nc(-n4c5ccccc5c5ccccc54)n3)c(-c3cccc4c3oc3ccccc34)c2)c1 ; CP(C)(=O)c1cccc(-c2ccc(-c3nc(-c4ccccc4)nc(-n4c5ccccc5c5ccccc54)n3)c(-c3ccc4c(c3)sc3ccccc34)c2)c1]

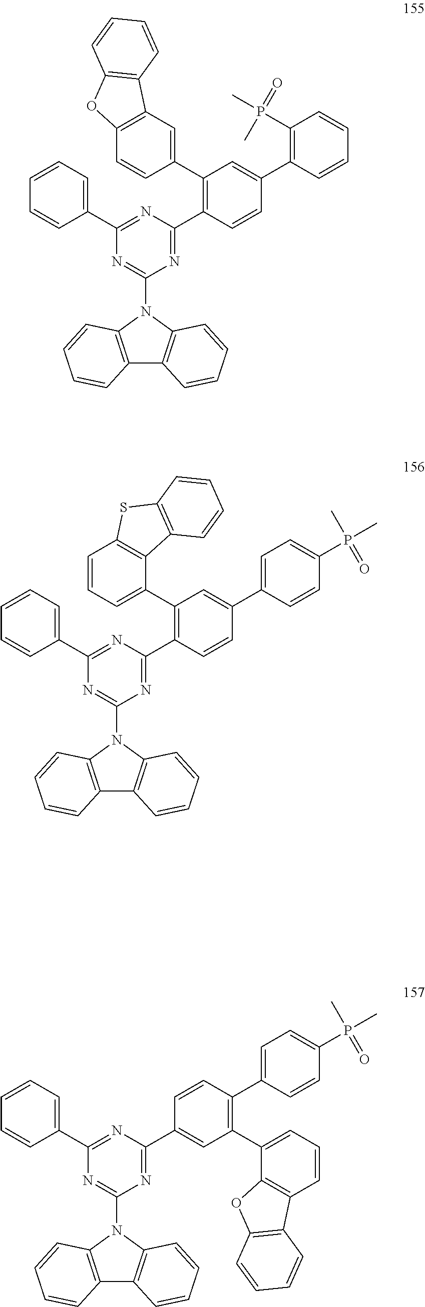 [SMILES: CP(C)(=O)c1ccccc1-c1ccc(-c2nc(-c3ccccc3)nc(-n3c4ccccc4c4ccccc43)n2)c(-c2ccc3oc4ccccc4c3c2)c1 ; CP(C)(=O)c1ccc(-c2ccc(-c3nc(-c4ccccc4)nc(-n4c5ccccc5c5ccccc54)n3)c(-c3cccc4sc5ccccc5c34)c2)cc1 ; CP(C)(=O)c1ccc(-c2ccc(-c3nc(-c4ccccc4)nc(-n4c5ccccc5c5ccccc54)n3)cc2-c2cccc3c2oc2ccccc23)cc1]

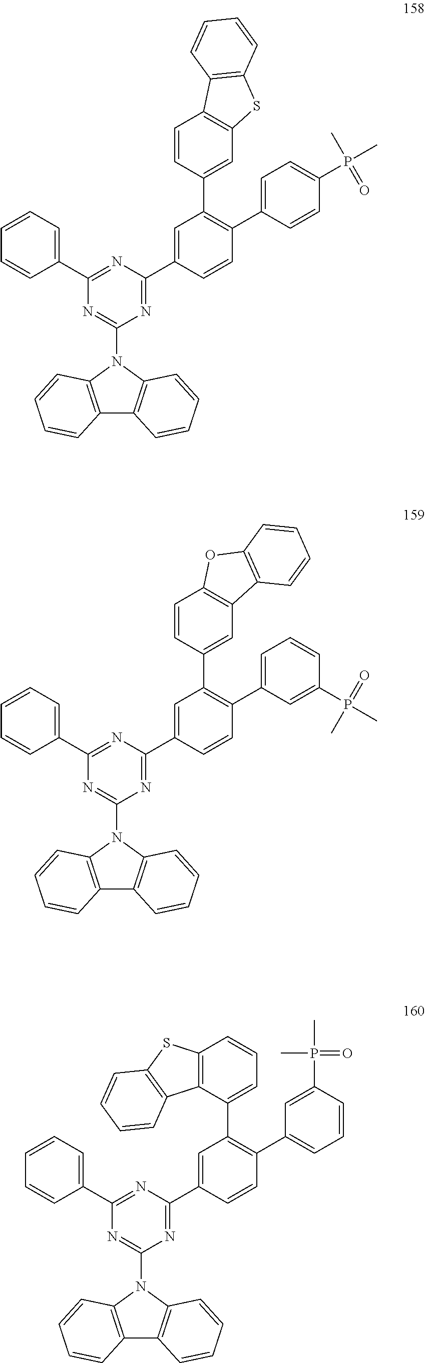 [SMILES: CP(C)(=O)c1ccc(-c2ccc(-c3nc(-c4ccccc4)nc(-n4c5ccccc5c5ccccc54)n3)cc2-c2ccc3c(c2)sc2ccccc23)cc1 ; CP(C)(=O)c1cccc(-c2ccc(-c3nc(-c4ccccc4)nc(-n4c5ccccc5c5ccccc54)n3)cc2-c2ccc3oc4ccccc4c3c2)c1 ; CP(C)(=O)c1cccc(-c2ccc(-c3nc(-c4ccccc4)nc(-n4c5ccccc5c5ccccc54)n3)cc2-c2cccc3sc4ccccc4c23)c1]

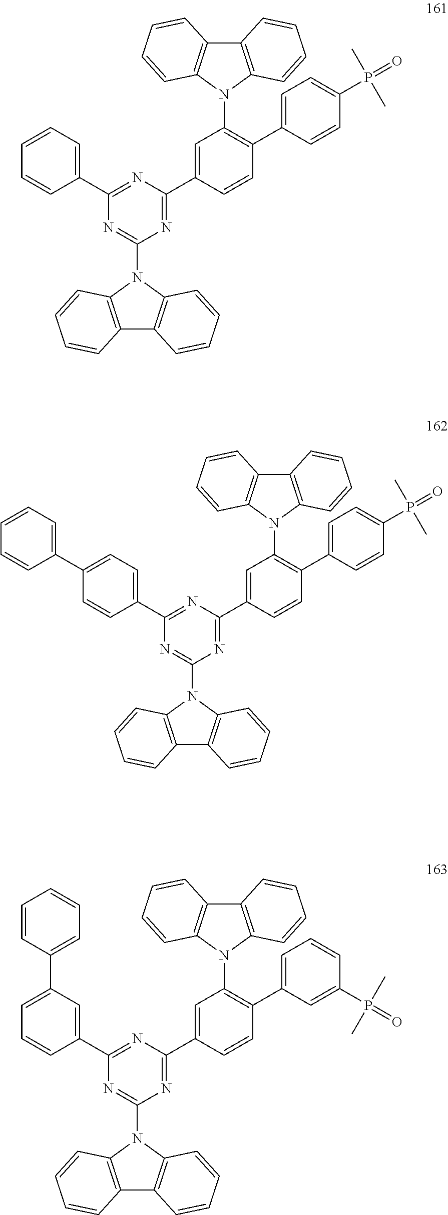 [SMILES: CP(C)(=O)c1ccc(-c2ccc(-c3nc(-c4ccccc4)nc(-n4c5ccccc5c5ccccc54)n3)cc2-n2c3ccccc3c3ccccc32)cc1 ; CP(C)(=O)c1ccc(-c2ccc(-c3nc(-c4ccc(-c5ccccc5)cc4)nc(-n4c5ccccc5c5ccccc54)n3)cc2-n2c3ccccc3c3ccccc32)cc1 ; CP(C)(=O)c1cccc(-c2ccc(-c3nc(-c4cccc(-c5ccccc5)c4)nc(-n4c5ccccc5c5ccccc54)n3)cc2-n2c3ccccc3c3ccccc32)c1]

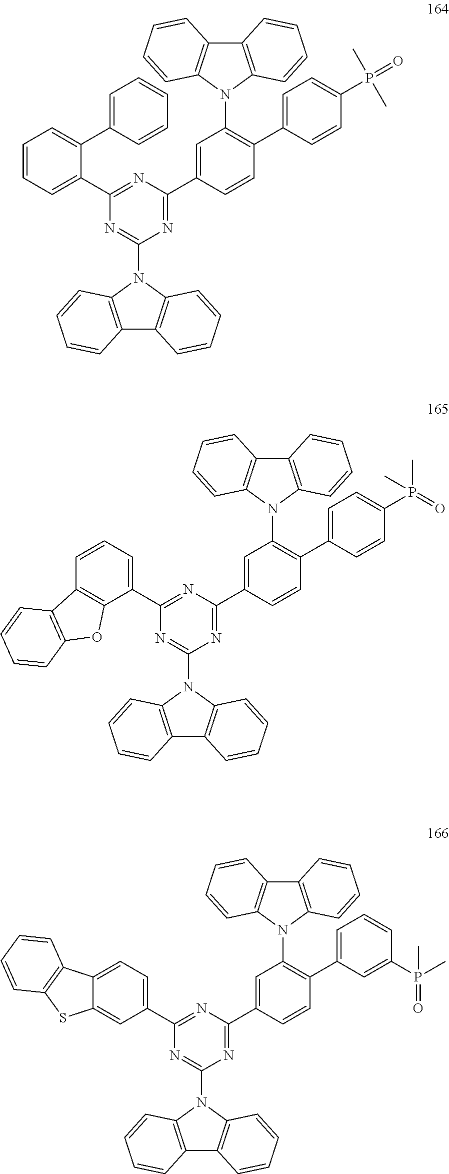 [SMILES: CP(C)(=O)c1ccc(-c2ccc(-c3nc(-c4ccccc4-c4ccccc4)nc(-n4c5ccccc5c5ccccc54)n3)cc2-n2c3ccccc3c3ccccc32)cc1 ; CP(C)(=O)c1ccc(-c2ccc(-c3nc(-c4cccc5c4oc4ccccc45)nc(-n4c5ccccc5c5ccccc54)n3)cc2-n2c3ccccc3c3ccccc32)cc1 ; CP(C)(=O)c1cccc(-c2ccc(-c3nc(-c4ccc5c(c4)sc4ccccc45)nc(-n4c5ccccc5c5ccccc54)n3)cc2-n2c3ccccc3c3ccccc32)c1]

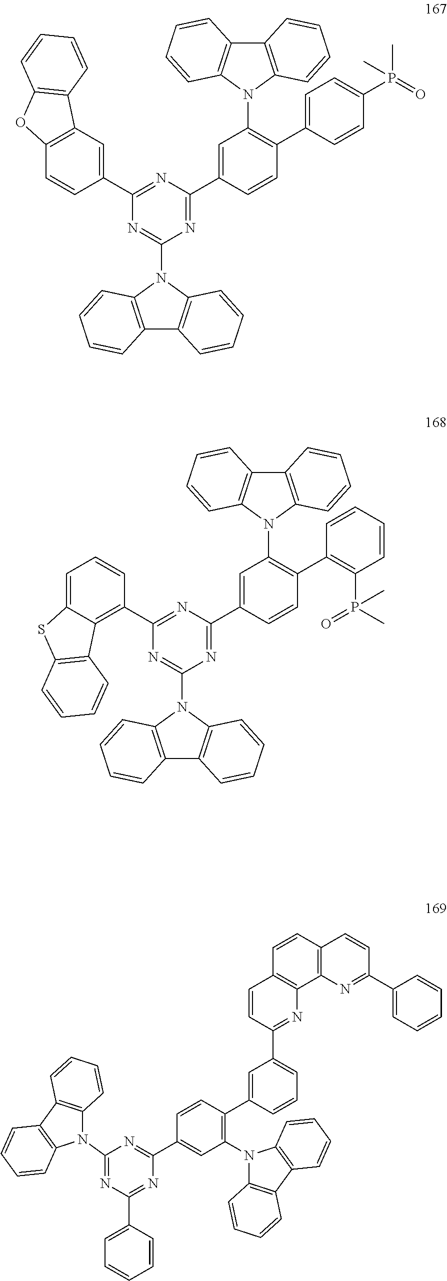 [SMILES: CP(C)(=O)c1ccc(-c2ccc(-c3nc(-c4ccc5oc6ccccc6c5c4)nc(-n4c5ccccc5c5ccccc54)n3)cc2-n2c3ccccc3c3ccccc32)cc1 ; CP(C)(=O)c1ccccc1-c1ccc(-c2nc(-c3cccc4sc5ccccc5c34)nc(-n3c4ccccc4c4ccccc43)n2)cc1-n1c2ccccc2c2ccccc21 ; c1ccc(-c2ccc3ccc4ccc(-c5cccc(-c6ccc(-c7nc(-c8ccccc8)nc(-n8c9ccccc9c9ccccc98)n7)cc6-n6c7ccccc7c7ccccc76)c5)nc4c3n2)cc1]

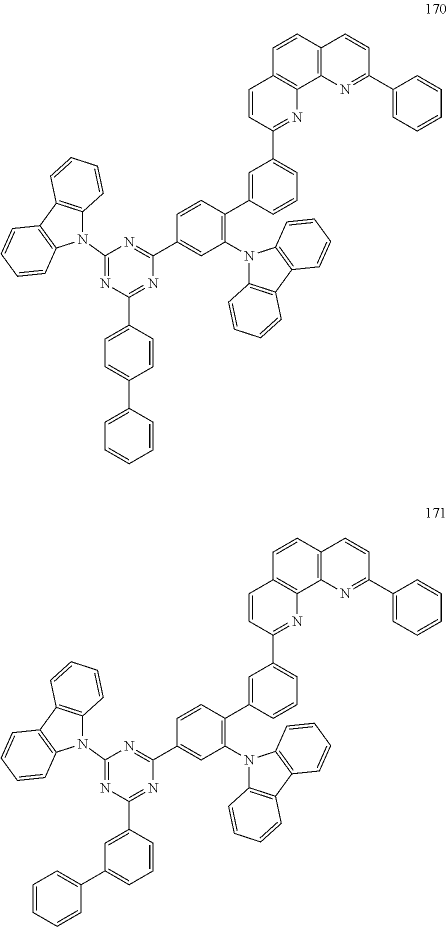 [SMILES: c1ccc(-c2ccc(-c3nc(-c4ccc(-c5cccc(-c6ccc7ccc8ccc(-c9ccccc9)nc8c7n6)c5)c(-n5c6ccccc6c6ccccc65)c4)nc(-n4c5ccccc5c5ccccc54)n3)cc2)cc1 ; c1ccc(-c2cccc(-c3nc(-c4ccc(-c5cccc(-c6ccc7ccc8ccc(-c9ccccc9)nc8c7n6)c5)c(-n5c6ccccc6c6ccccc65)c4)nc(-n4c5ccccc5c5ccccc54)n3)c2)cc1]

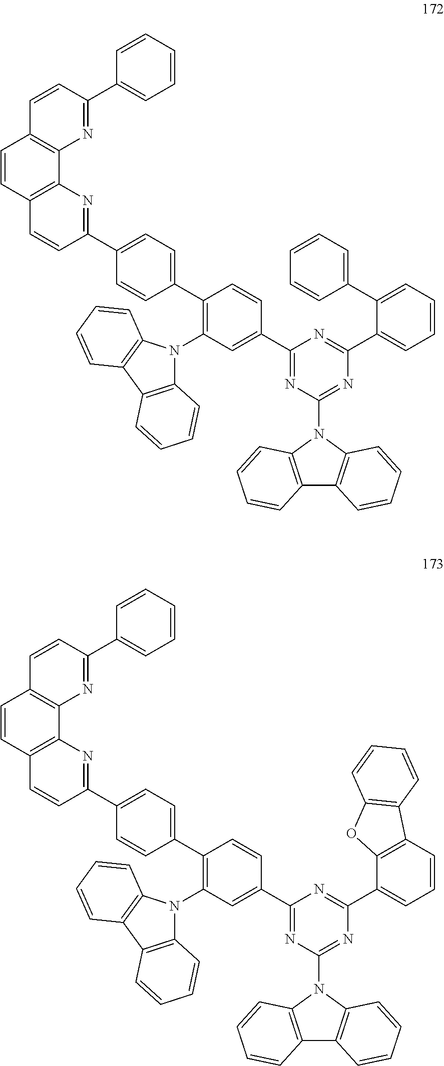 [SMILES: c1ccc(-c2ccc3ccc4ccc(-c5ccc(-c6ccc(-c7nc(-c8ccccc8-c8ccccc8)nc(-n8c9ccccc9c9ccccc98)n7)cc6-n6c7ccccc7c7ccccc76)cc5)nc4c3n2)cc1 ; c1ccc(-c2ccc3ccc4ccc(-c5ccc(-c6ccc(-c7nc(-c8cccc9c8oc8ccccc89)nc(-n8c9ccccc9c9ccccc98)n7)cc6-n6c7ccccc7c7ccccc76)cc5)nc4c3n2)cc1]

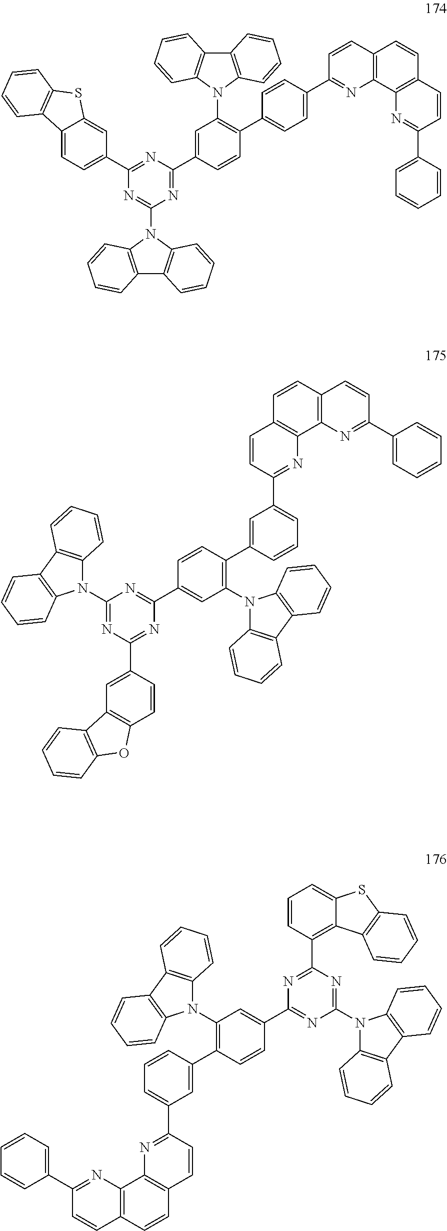 [SMILES: c1ccc(-c2ccc3ccc4ccc(-c5ccc(-c6ccc(-c7nc(-c8ccc9c(c8)sc8ccccc89)nc(-n8c9ccccc9c9ccccc98)n7)cc6-n6c7ccccc7c7ccccc76)cc5)nc4c3n2)cc1 ; c1ccc(-c2ccc3ccc4ccc(-c5cccc(-c6ccc(-c7nc(-c8ccc9oc%10ccccc%10c9c8)nc(-n8c9ccccc9c9ccccc98)n7)cc6-n6c7ccccc7c7ccccc76)c5)nc4c3n2)cc1 ; c1ccc(-c2ccc3ccc4ccc(-c5cccc(-c6ccc(-c7nc(-c8cccc9sc%10ccccc%10c89)nc(-n8c9ccccc9c9ccccc98)n7)cc6-n6c7ccccc7c7ccccc76)c5)nc4c3n2)cc1]

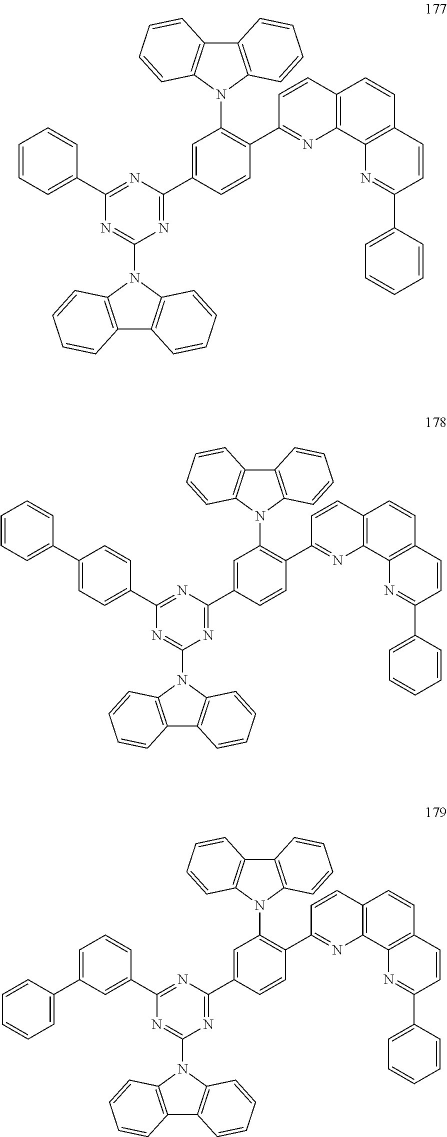 [SMILES: c1ccc(-c2ccc3ccc4ccc(-c5ccc(-c6nc(-c7ccccc7)nc(-n7c8ccccc8c8ccccc87)n6)cc5-n5c6ccccc6c6ccccc65)nc4c3n2)cc1 ; c1ccc(-c2ccc(-c3nc(-c4ccc(-c5ccc6ccc7ccc(-c8ccccc8)nc7c6n5)c(-n5c6ccccc6c6ccccc65)c4)nc(-n4c5ccccc5c5ccccc54)n3)cc2)cc1 ; c1ccc(-c2cccc(-c3nc(-c4ccc(-c5ccc6ccc7ccc(-c8ccccc8)nc7c6n5)c(-n5c6ccccc6c6ccccc65)c4)nc(-n4c5ccccc5c5ccccc54)n3)c2)cc1]

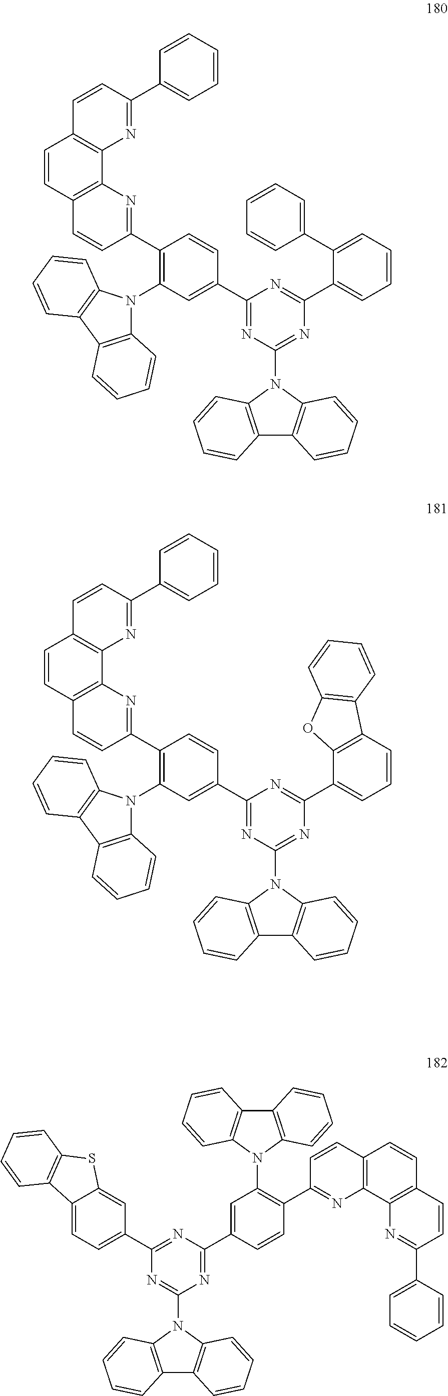 [SMILES: c1ccc(-c2ccc3ccc4ccc(-c5ccc(-c6nc(-c7ccccc7-c7ccccc7)nc(-n7c8ccccc8c8ccccc87)n6)cc5-n5c6ccccc6c6ccccc65)nc4c3n2)cc1 ; c1ccc(-c2ccc3ccc4ccc(-c5ccc(-c6nc(-c7cccc8c7oc7ccccc78)nc(-n7c8ccccc8c8ccccc87)n6)cc5-n5c6ccccc6c6ccccc65)nc4c3n2)cc1 ; c1ccc(-c2ccc3ccc4ccc(-c5ccc(-c6nc(-c7ccc8c(c7)sc7ccccc78)nc(-n7c8ccccc8c8ccccc87)n6)cc5-n5c6ccccc6c6ccccc65)nc4c3n2)cc1]

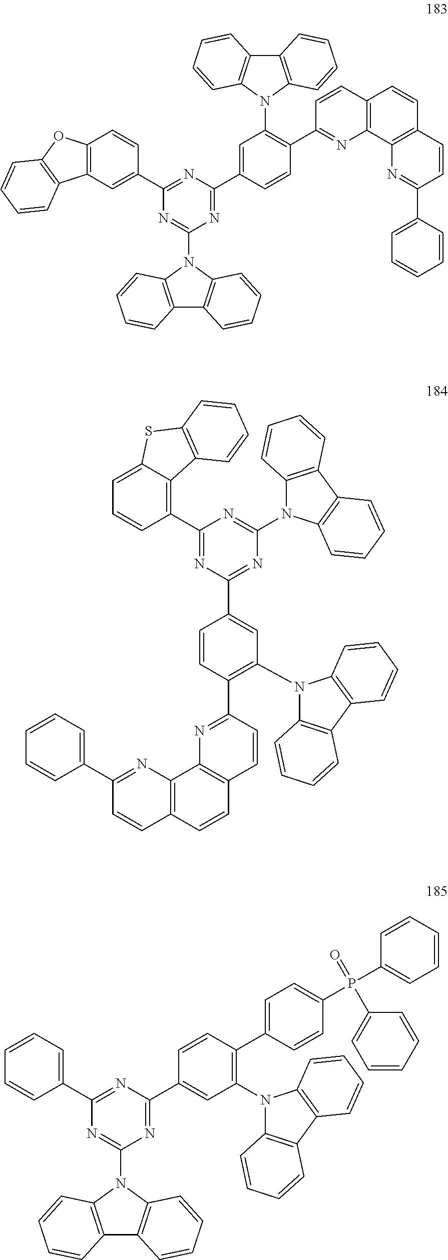 [SMILES: c1ccc(-c2ccc3ccc4ccc(-c5ccc(-c6nc(-c7ccc8oc9ccccc9c8c7)nc(-n7c8ccccc8c8ccccc87)n6)cc5-n5c6ccccc6c6ccccc65)nc4c3n2)cc1 ; c1ccc(-c2ccc3ccc4ccc(-c5ccc(-c6nc(-c7cccc8sc9ccccc9c78)nc(-n7c8ccccc8c8ccccc87)n6)cc5-n5c6ccccc6c6ccccc65)nc4c3n2)cc1 ; O=P(c1ccccc1)(c1ccccc1)c1ccc(-c2ccc(-c3nc(-c4ccccc4)nc(-n4c5ccccc5c5ccccc54)n3)cc2-n2c3ccccc3c3ccccc32)cc1]

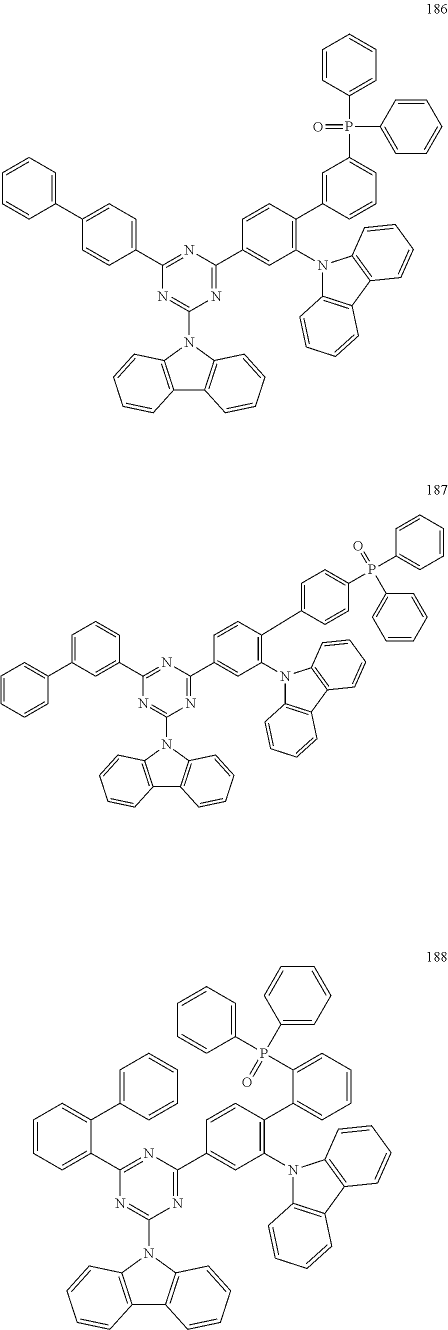 [SMILES: O=P(c1ccccc1)(c1ccccc1)c1cccc(-c2ccc(-c3nc(-c4ccc(-c5ccccc5)cc4)nc(-n4c5ccccc5c5ccccc54)n3)cc2-n2c3ccccc3c3ccccc32)c1 ; O=P(c1ccccc1)(c1ccccc1)c1ccc(-c2ccc(-c3nc(-c4cccc(-c5ccccc5)c4)nc(-n4c5ccccc5c5ccccc54)n3)cc2-n2c3ccccc3c3ccccc32)cc1 ; O=P(c1ccccc1)(c1ccccc1)c1ccccc1-c1ccc(-c2nc(-c3ccccc3-c3ccccc3)nc(-n3c4ccccc4c4ccccc43)n2)cc1-n1c2ccccc2c2ccccc21]

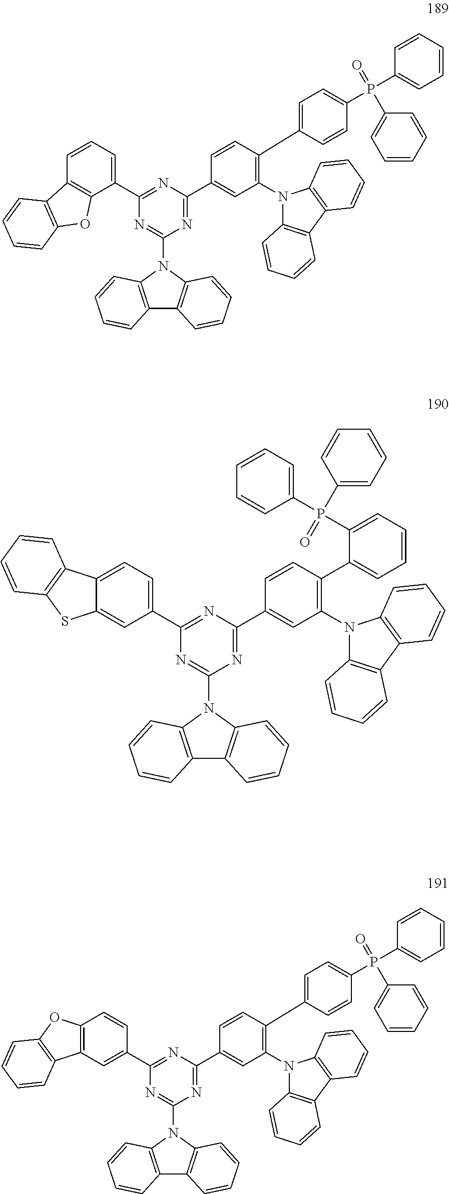 [SMILES: O=P(c1ccccc1)(c1ccccc1)c1ccc(-c2ccc(-c3nc(-c4cccc5c4oc4ccccc45)nc(-n4c5ccccc5c5ccccc54)n3)cc2-n2c3ccccc3c3ccccc32)cc1 ; O=P(c1ccccc1)(c1ccccc1)c1ccccc1-c1ccc(-c2nc(-c3ccc4c(c3)sc3ccccc34)nc(-n3c4ccccc4c4ccccc43)n2)cc1-n1c2ccccc2c2ccccc21 ; O=P(c1ccccc1)(c1ccccc1)c1ccc(-c2ccc(-c3nc(-c4ccc5oc6ccccc6c5c4)nc(-n4c5ccccc5c5ccccc54)n3)cc2-n2c3ccccc3c3ccccc32)cc1]

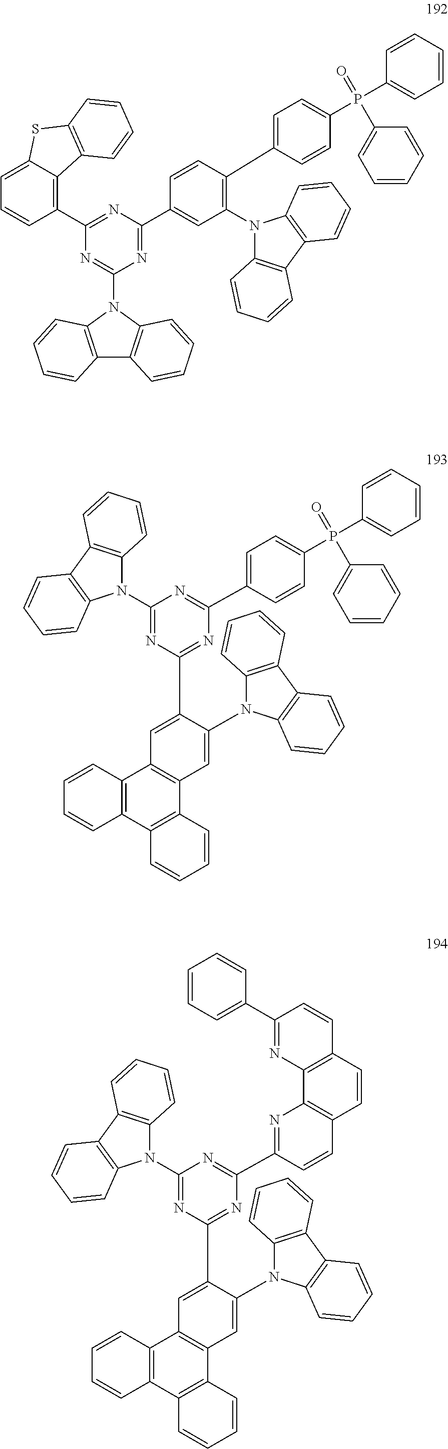 [SMILES: O=P(c1ccccc1)(c1ccccc1)c1ccc(-c2ccc(-c3nc(-c4cccc5sc6ccccc6c45)nc(-n4c5ccccc5c5ccccc54)n3)cc2-n2c3ccccc3c3ccccc32)cc1 ; O=P(c1ccccc1)(c1ccccc1)c1ccc(-c2nc(-c3cc4c5ccccc5c5ccccc5c4cc3-n3c4ccccc4c4ccccc43)nc(-n3c4ccccc4c4ccccc43)n2)cc1 ; c1ccc(-c2ccc3ccc4ccc(-c5nc(-c6cc7c8ccccc8c8ccccc8c7cc6-n6c7ccccc7c7ccccc76)nc(-n6c7ccccc7c7ccccc76)n5)nc4c3n2)cc1]

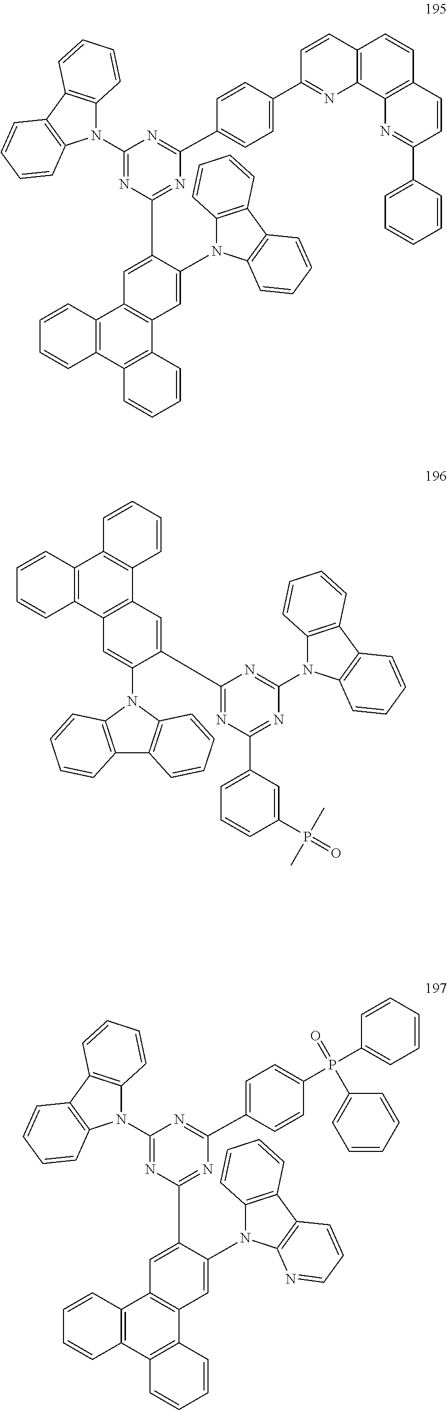 [SMILES: c1ccc(-c2ccc3ccc4ccc(-c5ccc(-c6nc(-c7cc8c9ccccc9c9ccccc9c8cc7-n7c8ccccc8c8ccccc87)nc(-n7c8ccccc8c8ccccc87)n6)cc5)nc4c3n2)cc1 ; CP(C)(=O)c1cccc(-c2nc(-c3cc4c5ccccc5c5ccccc5c4cc3-n3c4ccccc4c4ccccc43)nc(-n3c4ccccc4c4ccccc43)n2)c1 ; O=P(c1ccccc1)(c1ccccc1)c1ccc(-c2nc(-c3cc4c5ccccc5c5ccccc5c4cc3-n3c4ccccc4c4cccnc43)nc(-n3c4ccccc4c4ccccc43)n2)cc1]

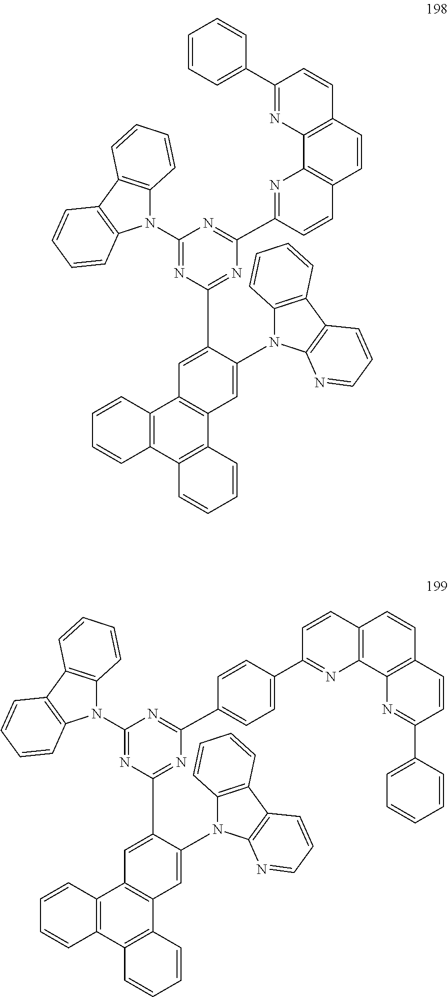 [SMILES: c1ccc(-c2ccc3ccc4ccc(-c5nc(-c6cc7c8ccccc8c8ccccc8c7cc6-n6c7ccccc7c7cccnc76)nc(-n6c7ccccc7c7ccccc76)n5)nc4c3n2)cc1 ; c1ccc(-c2ccc3ccc4ccc(-c5ccc(-c6nc(-c7cc8c9ccccc9c9ccccc9c8cc7-n7c8ccccc8c8cccnc87)nc(-n7c8ccccc8c8ccccc87)n6)cc5)nc4c3n2)cc1]

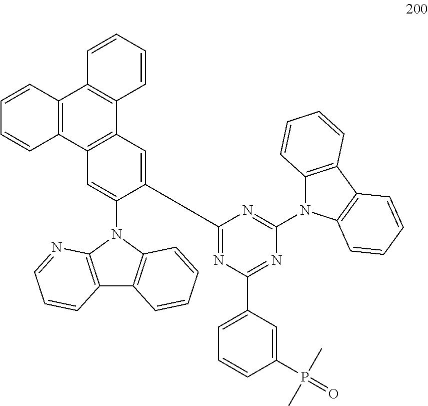 [SMILES: CP(C)(=O)c1cccc(-c2nc(-c3cc4c5ccccc5c5ccccc5c4cc3-n3c4ccccc4c4cccnc43)nc(-n3c4ccccc4c4ccccc43)n2)c1]

In addition, by introducing various substituents to the structure of Chemical Formula 1, compounds having unique properties of the introduced substituents may be synthesized. For example, by introducing substituents normally used for a hole injection layer material, a hole transport layer material, a light emitting layer material, an electron transport layer material and a charge generation layer material used for manufacturing an organic light emitting device to the core structure, materials satisfying conditions required for each organic material layer may be synthesized.

In addition, by introducing various substituents to the structure of Chemical Formula 1, the energy band gap may be finely controlled, and meanwhile, properties at interfaces between organic materials may be enhanced, and material applications may become diverse.

Another embodiment of the present disclosure provides an organic light emitting device including the heterocyclic compound represented by Chemical Formula 1. The “organic light emitting device” may be expressed in terms such as an “organic light emitting diode”, an “OLED”, an “OLED device” and an “organic electroluminescent device”.

One embodiment of the present application provides an organic light emitting device including: a first electrode; a second electrode provided opposite to the first electrode; and one or more organic material layers provided between the first electrode and the second electrode, wherein one or more layers of the organic material layers include the heterocyclic compound represented by Chemical Formula 1.

In one embodiment of the present application, the first electrode may be a positive electrode, and the second electrode may be a negative electrode.

In another embodiment, the first electrode may be a negative electrode, and the second electrode may be a positive electrode.

In one embodiment of the present application, the organic light emitting device may be a blue organic light emitting device, and the heterocyclic compound according to Chemical Formula 1 may be used as a material of the blue organic light emitting device.

In one embodiment of the present application, the organic light emitting device may be a green organic light emitting device, and the heterocyclic compound represented by Chemical Formula 1 may be used as a material of the green organic light emitting device.

In one embodiment of the present application, the organic light emitting device may be a red organic light emitting device, and the heterocyclic compound represented by Chemical Formula 1 may be used as a material of the red organic light emitting device.

Specific descriptions on the heterocyclic compound represented by Chemical Formula 1 are the same as the descriptions provided above.

The organic light emitting device of the present disclosure may be manufactured using common organic light emitting device manufacturing methods and materials except that one or more organic material layers are formed using the heterocyclic compound described above.

The heterocyclic compound may be formed into the organic material layer using a solution coating method as well as a vacuum deposition method when the organic light emitting device is manufactured. Herein, the solution coating method means spin coating, dip coating, inkjet printing, screen printing, a spray method, roll coating and the like, but is not limited thereto.

The organic material layer of the organic light emitting device of the present disclosure may be formed in a single layer structure, but may also be formed in a multilayer structure in which two or more organic material layers are laminated. For example, the organic light emitting device of the present disclosure may have a structure including a hole injection layer, a hole transport layer, a light emitting layer, an electron transport layer, an electron injection layer and the like as the organic material layer. However, the structure of the organic light emitting device is not limited thereto, and may include a smaller number of organic material layers.

In the organic light emitting device of the present disclosure, the organic material layer includes an electron injection layer or an electron transport layer, and the electron injection layer or the electron transport layer may include the heterocyclic compound.

In the organic light emitting device of the present disclosure, the organic material layer includes an electron transport layer, and the electron transport layer may include the heterocyclic compound.

In another organic light emitting device, the organic material layer includes an electron blocking layer or a hole blocking layer, and the electron blocking layer or the hole blocking layer may include the heterocyclic compound.

In another organic light emitting device, the organic material layer includes a hole blocking layer, and the hole blocking layer may include the heterocyclic compound.

In another organic light emitting device, the organic material layer includes an electron transport layer, a light emitting layer or a hole blocking layer, and the electron transport layer, the light emitting layer or the hole blocking layer may include the heterocyclic compound.

The organic light emitting device of the present disclosure may further include, one, or two or more layers selected from the group consisting of a light emitting layer, a hole injection layer, a hole transport layer, an electron injection layer, an electron transport layer, an electron blocking layer and a hole blocking layer.

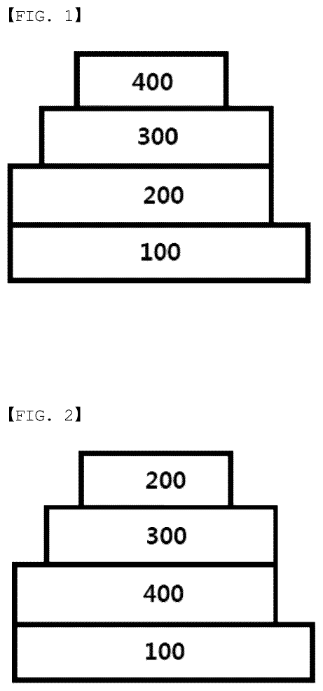

FIGS. 1 to 3 illustrate a lamination order of electrodes and organic material layers of the organic light emitting device according to one embodiment of the present disclosure. However, it is not intended that the scope of the present application be limited by these drawings, and structures of organic light emitting devices known in the art may also be applied to the present application.

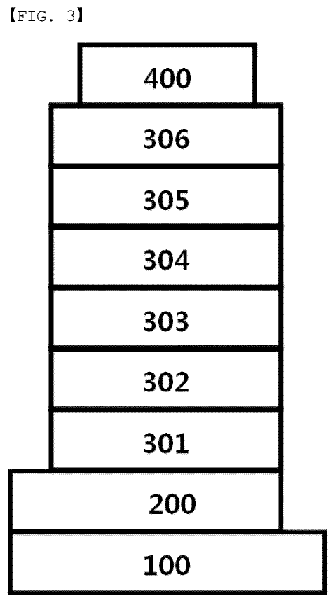

FIG. 1 illustrates an organic light emitting device in which a positive electrode 200, an organic material layer 300 and a negative electrode 400 are sequentially laminated on a substrate 100. However, the structure is not limited only to such a structure, and as illustrated in FIG. 2, an organic light emitting device in which a negative electrode, an organic material layer and a positive electrode are sequentially laminated on a substrate may also be obtained.

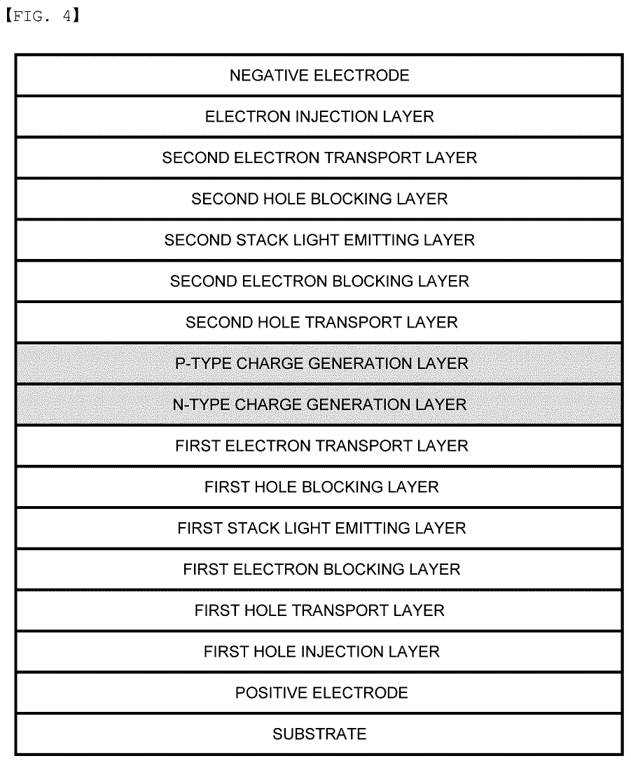

FIG. 3 illustrates a case of the organic material layer being a multilayer. An organic light emitting device according to FIG. 3 includes a hole injection layer 301, a hole transport layer 302, a light emitting layer 303, a hole blocking layer 304, an electron transport layer 305 and an electron injection layer 306. However, the scope of the present application is not limited by such a lamination structure, and as necessary, the layers other than the light emitting layer may not be included, and other necessary functional layers may be further added.

The organic material layer including Chemical Formula 1 may further include other materials as necessary.

In addition, the organic light emitting device according to one embodiment of the present application includes a positive electrode, a negative electrode and two or more stacks provided between the positive electrode and the negative electrode, wherein the two or more stacks each independently include a light emitting layer, a charge generation layer is included between the two or more stacks, and the charge generation layer includes the heterocyclic compound represented by Chemical Formula 1.

In addition, the organic light emitting device according to one embodiment of the present application includes a positive electrode, a first stack provided on the positive electrode and including a first light emitting layer, a charge generation layer provided on the first stack, a second stack provided on the charge generation layer and including a second light emitting layer, and a negative electrode provided on the second stack. Herein, the charge generation layer may include the heterocyclic compound represented by Chemical Formula 1. In addition, the first stack and the second stack may each independently further include one or more of the hole injection layer, the hole transport layer, the hole blocking layer, the electron transport layer, the electron injection layer and the like described above.

The charge generation layer may be an N-type charge generation layer, and the charge generation layer may further include a dopant known in the art in addition to the heterocyclic compound represented by Chemical Formula 1.

As the organic light emitting device according to one embodiment of the present application, an organic light emitting device having a 2-stack tandem structure is schematically illustrated in FIG. 4.

Herein, the first electron blocking layer, the first hole blocking layer, the second hole blocking layer and the like described in FIG. 4 may not be included depending on the case.

In the organic light emitting device according to one embodiment of the present application, materials other than the compound of Chemical Formula 1 are illustrated below, however, these are for illustrative purposes only and not for limiting the scope of the present application, and these materials may be replaced by materials known in the art.

As the positive electrode material, materials each having a relatively large work function may be used, and transparent conductive oxides, metals, conductive polymers or the like may be used. Specific examples of the positive electrode material include metals such as vanadium, chromium, copper, zinc and gold, or alloys thereof; metal oxides such as zinc oxide, indium oxide, indium tin oxide (ITO) and indium zinc oxide (IZO); combinations of metals and oxides such as ZnO:Al or SnO₂:Sb; conductive polymers such as poly(3-methylthiophene), poly[3,4-(ethylene-1,2-dioxy)thiophene] (PEDOT), polypyrrole and polyaniline, and the like, but are not limited thereto.

As the negative electrode material, materials each having a relatively small work function may be used, and metals, metal oxides, conductive polymers or the like may be used. Specific examples of the negative electrode material include metals such as magnesium, calcium, sodium, potassium, titanium, indium, yttrium, lithium, gadolinium, aluminum, silver, tin and lead, or alloys thereof; multilayer structure materials such as LiF/Al or LiO₂/Al, and the like, but are not limited thereto.

As the hole injection material, known hole injection materials may be used, and for example, phthalocyanine compounds such as copper phthalocyanine disclosed in U.S. Pat. No. 4,356,429, or starburst-type amine derivatives such as tris(4-carbazoyl-9-ylphenyl)amine (TCTA), 4,4′,4″-tri[phenyl(m-tolyl)amino]triphenylamine (m-MTDATA) or 1,3,5-tris[4-(3-methylphenylphenylamino)phenyl]benzene (m-MTDAPB) described in the literature [Advanced Material, 6, p. 677 (1994)], conductive polymers having solubility such as polyaniline/dodecylbenzenesulfonic acid or poly(3,4-ethylenedioxythiophene)/poly(4-styrenesulfonate), polyaniline/camphor sulfonic acid or polyaniline/poly(4-styrene-sulfonate), and the like, may be used.

As the hole transport material, pyrazoline derivatives, arylamine-based derivatives, stilbene derivatives, triphenyldiamine derivatives and the like may be used, and low molecular or high molecular materials may also be used.

As the electron transport material, metal complexes of oxadiazole derivatives, anthraquinodimethane and derivatives thereof, benzoquinone and derivatives thereof, naphthoquinone and derivatives thereof, anthraquinone and derivatives thereof, tetracyanoanthraquinodimethane and derivatives thereof, fluorenone derivatives, diphenyldicyanoethylene and derivatives thereof, diphenoquinone derivatives, 8-hydroxyquinoline and derivatives thereof, and the like, may be used, and high molecular materials as well as low molecular materials may also be used.

As examples of the electron injection material, LiF is typically used in the art, however, the present application is not limited thereto.

As the light emitting material, red, green or blue light emitting materials may be used, and as necessary, two or more light emitting materials may be mixed and used. Herein, the two or more light emitting materials may be deposited as individual sources of supply or pre-mixed and deposited as one source of supply when used. In addition, fluorescent materials may also be used as the light emitting material, however, phosphorescent materials may also be used. As the light emitting material, materials emitting light alone by binding holes and electrons injected from a positive electrode and a negative electrode, respectively, may be used, however, materials having a host material and a dopant material involved in light emission together may also be used.

When hosts of the light emitting material are mixed and used, same series hosts may be mixed and used, or different series hosts may be mixed and used. For example, any two or more types of materials among n-type host materials and p-type host materials may be selected and used as a host material of a light emitting layer.

The organic light emitting device according to one embodiment of the present application may be a top-emission type, a bottom-emission type or a dual-emission type depending on the materials used.

The heterocyclic compound according to one embodiment of the present application may also be used in an organic electronic device including an organic solar cell, an organic photo conductor, an organic transistor and the like under a principle similar to that in the organic light emitting device.

Hereinafter, preferred examples are provided to help to understand the present disclosure, however, the following examples are only provided to more readily understand the present disclosure, and the present disclosure is not limited thereto.

### PREPARATION EXAMPLE

### <Preparation Example 1> Preparation of Compound 009

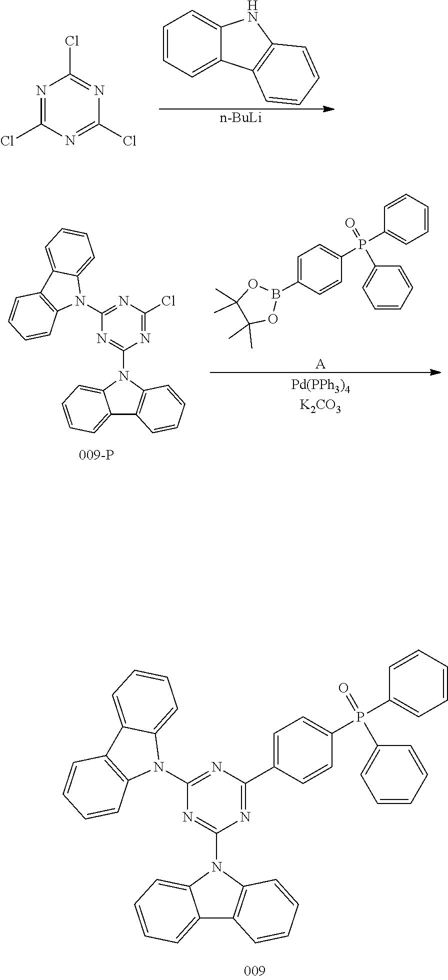

### 1) Preparation of Compound 009-P

2.5 M n-BuLi (44 ml, 0.11 mol, 2 eq.) was introduced to 9H-carbazole (20 g, 0.12 mol, 2.2 eq.) and THE (200 ml) at −30° C., and the mixture was stirred for 1 hour. Cyanuric chloride (10 g, 0.054 mol, 1 eq.) and THE (200 ml) were introduced thereto, and the result was stirred for 2 hours at room temperature. Water was introduced thereto to terminate the reaction, and then a produced solid was filtered. Compound 009-P (16.8 g) was obtained in a yield of 70%.

### 2) Preparation of Compound 009

Compound 009-P (16.8 g, 0.038 mol, 1 eq.), diphenyl(4-(4,4,5,5-tetramethyl-1,3,2-dioxaborolan-2-yl)phenyl)phosphine oxide (A) (16.9 g, 0.042 mol, 1.1 eq.), Pd(PPh₃)₄ (2.2 g, 0.002 mol, 0.05 eq.), K₂CO₃ (10.5 g, 0.076 mol, 2 eq.), 1,4-dioxane (200 ml) and water (50 ml) were introduced, and stirred for 4 hours at 100° C. Compound 009 (23.5 g) was obtained in a yield of 90%.

### <Preparation Example 2> Preparation of Compounds 049, 089 and 129

Compounds 049, 089 and 129 were synthesized in the same manner as in Preparation Example 1, except that Intermediate A of the following Table 1 was used instead of diphenyl(4-(4,4,5,5-5 tetramethyl-1,3,2-dioxaborolan-2-yl)phenyl)phosphine oxide (A).

TABLE 1

| Compound | | |
|---|---|---|
| No. | Intermediate A | Yield |
| 049 | 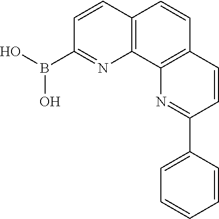 [SMILES: OB(O)c1ccc2ccc3ccc(-c4ccccc4)nc3c2n1] | 70% |
| 089 | 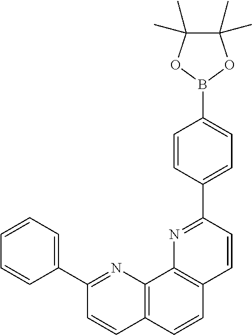 [SMILES: CC1(C)OB(c2ccc(-c3ccc4ccc5ccc(-c6ccccc6)nc5c4n3)cc2)OC1(C)C] | 75% |
| 129 | 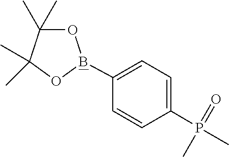 | 74% |

### <Preparation Example 3> Preparation of Compound 023

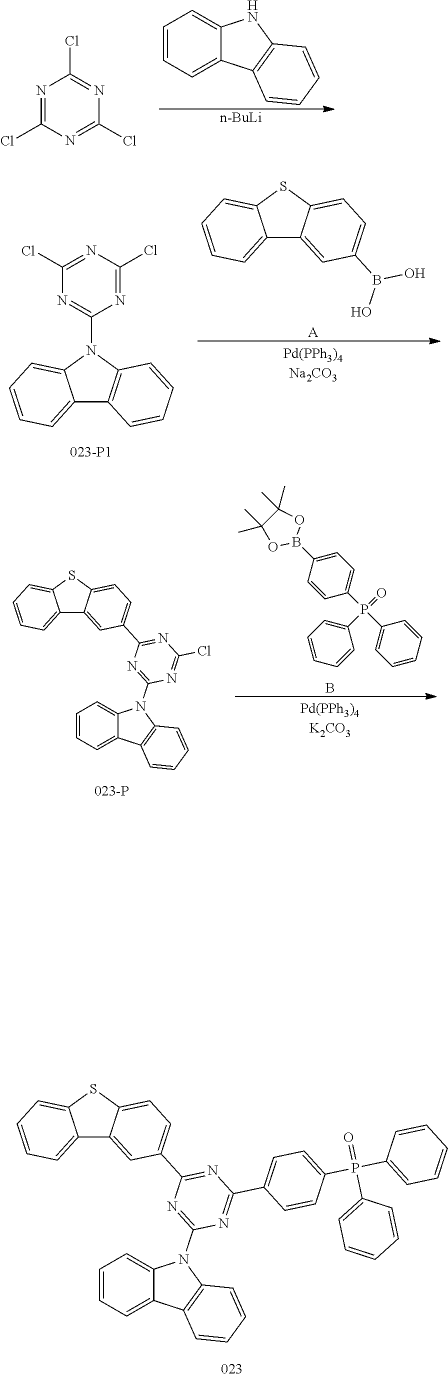

### 1) Preparation of Compound 023-P1

2.5 M n-BuLi (44 ml, 0.11 mol, 1 eq.) was introduced to 9H-carbazole (20 g, 0.12 mol, 1.1 eq.) and THE (200 ml) at −30° C., and the mixture was stirred for 1 hour. Cyanuric chloride (20 g, 0.11 mol, 1 eq.) and THE (200 ml) were introduced thereto, and the result was stirred for 2 hours at room temperature. Water was introduced thereto to terminate the reaction, and then a produced solid was filtered. Compound 023-P1 (24.3 g) was obtained in a yield of 70%.

### 2) Preparation of Compound 023-P

Compound 023-P1 (15 g, 0.047 mol, 1 eq.), dibenzo[b,d]thiophen-2-ylboronic acid (A) (11.8 g, 0.052 mol, 1.1 eq.), Pd(PPh₃)₄ (2.7 g, 0.002 mol, 0.05 eq.), Na₂CO₃ (10 g, 0.094 mol, 2 eq.), 1,4-dioxane (200 ml) and water (50 ml) were introduced, and stirred for 4 hours at 100° C. Compound 023-P (15.2 g) was obtained in a yield of 70%.

### 3) Preparation of Compound 023

Compound 023-P (15 g, 0.032 mol, 1 eq.), diphenyl(4-(4,4,5,5-tetramethyl-1,3,2-dioxaborolan-2-yl)phenyl)phosphine oxide (B) (14.4 g, 0.035 mol, 1.1 eq.), Pd(PPh₃)₄ (1.8 g, 0.0016 mol, 0.05 eq.), K₂CO₃ (8.8 g, 0.064 mol, 2 eq.), 1,4-dioxane (200 ml) and water (50 ml) were introduced, and stirred for 4 hours at 100° C. Compound 023 (16.9 g) was obtained in a yield of 75%.

### <Preparation Example 4> Preparation of Compounds 023, 031, 041, 048, 052, 070, 103, 105, 124, 152, 195 and 197

Compounds 023, 031, 041, 048, 052, 070, 103, 105, 124, 152, 195 and 197 were synthesized in the same manner as in Preparation Example 3, except that Intermediate A of the following Table 2 was used instead of dibenzo[b,d]thiophen-2-ylboronic acid (A), and Intermediate B of the following Table 2 was used instead of diphenyl(4-(4,4,5,5-tetramethyl-1,3,2-dioxaborolan-2-yl)phenyl)phosphine oxide (B).

TABLE 2

| Compound | | | |
|---|---|---|---|
| No. | Intermediate A | Intermediate B | Yield |
| 023 | 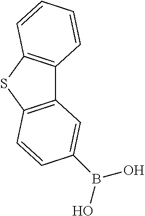 [SMILES: OB(O)c1ccc2sc3ccccc3c2c1] | 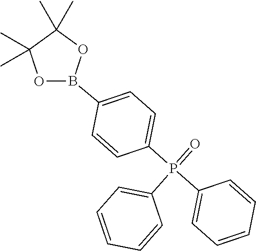 [SMILES: CC1(C)OB(c2ccc(P(=O)(c3ccccc3)c3ccccc3)cc2)OC1(C)C] | 90% |
| 031 | 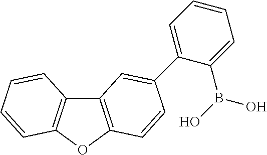 [SMILES: OB(O)c1ccccc1-c1ccc2oc3ccccc3c2c1] | 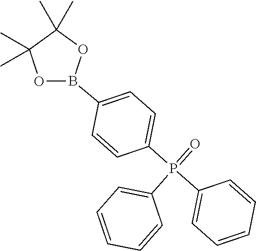 [SMILES: CC1(C)OB(c2ccc(P(=O)(c3ccccc3)c3ccccc3)cc2)OC1(C)C] | 86% |
| 041 | 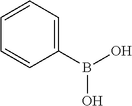 | 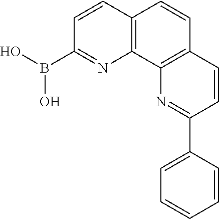 [SMILES: OB(O)c1ccc2ccc3ccc(-c4ccccc4)nc3c2n1] | 83% |
| 048 | 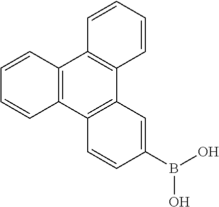 [SMILES: OB(O)c1ccc2c3ccccc3c3ccccc3c2c1] | 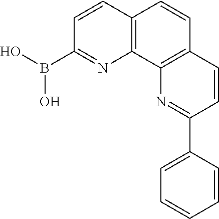 [SMILES: OB(O)c1ccc2ccc3ccc(-c4ccccc4)nc3c2n1] | 88% |
| 052 | 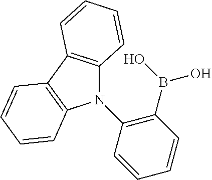 [SMILES: OB(O)c1ccccc1-n1c2ccccc2c2ccccc21] | 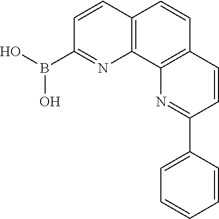 [SMILES: OB(O)c1ccc2ccc3ccc(-c4ccccc4)nc3c2n1] | 88% |
| 070 | 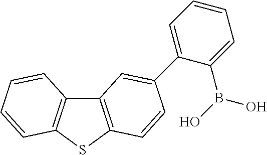 [SMILES: OB(O)c1ccccc1-c1ccc2sc3ccccc3c2c1] | 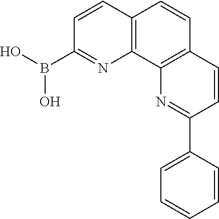 [SMILES: OB(O)c1ccc2ccc3ccc(-c4ccccc4)nc3c2n1] | 77% |
| 103 | 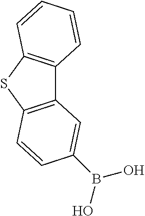 [SMILES: OB(O)c1ccc2sc3ccccc3c2c1] | 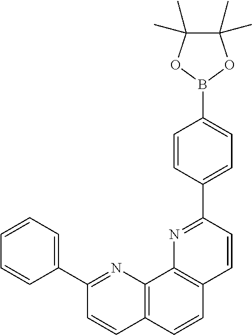 [SMILES: CC1(C)OB(c2ccc(-c3ccc4ccc5ccc(-c6ccccc6)nc5c4n3)cc2)OC1(C)C] | 88 |
| 105 | 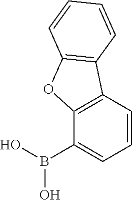 [SMILES: OB(O)c1cccc2c1oc1ccccc12] | 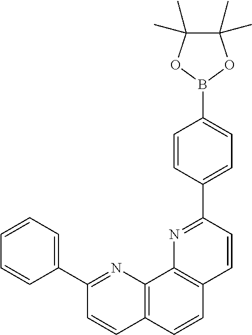 [SMILES: CC1(C)OB(c2ccc(-c3ccc4ccc5ccc(-c6ccccc6)nc5c4n3)cc2)OC1(C)C] | 86 |
| 124 | 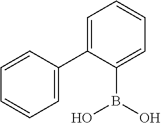 [SMILES: OB(O)c1ccccc1-c1ccccc1] | 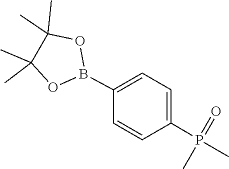 | 73 |
| 152 | 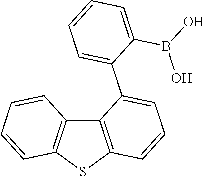 [SMILES: OB(O)c1ccccc1-c1cccc2sc3ccccc3c12] | 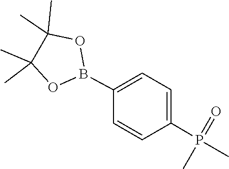 | 80 |
| 195 | 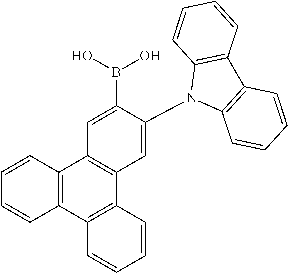 [SMILES: OB(O)c1cc2c3ccccc3c3ccccc3c2cc1-n1c2ccccc2c2ccccc21] | 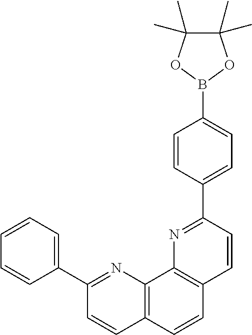 [SMILES: CC1(C)OB(c2ccc(-c3ccc4ccc5ccc(-c6ccccc6)nc5c4n3)cc2)OC1(C)C] | |
| 197 | 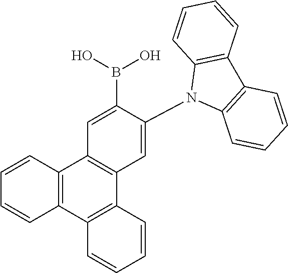 [SMILES: OB(O)c1cc2c3ccccc3c3ccccc3c2cc1-n1c2ccccc2c2ccccc21] | 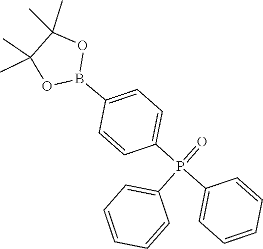 [SMILES: CC1(C)OB(c2ccc(P(=O)(c3ccccc3)c3ccccc3)cc2)OC1(C)C] | |

### <Preparation Example 5> Preparation of Compound 014

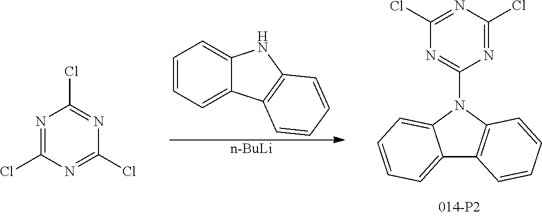

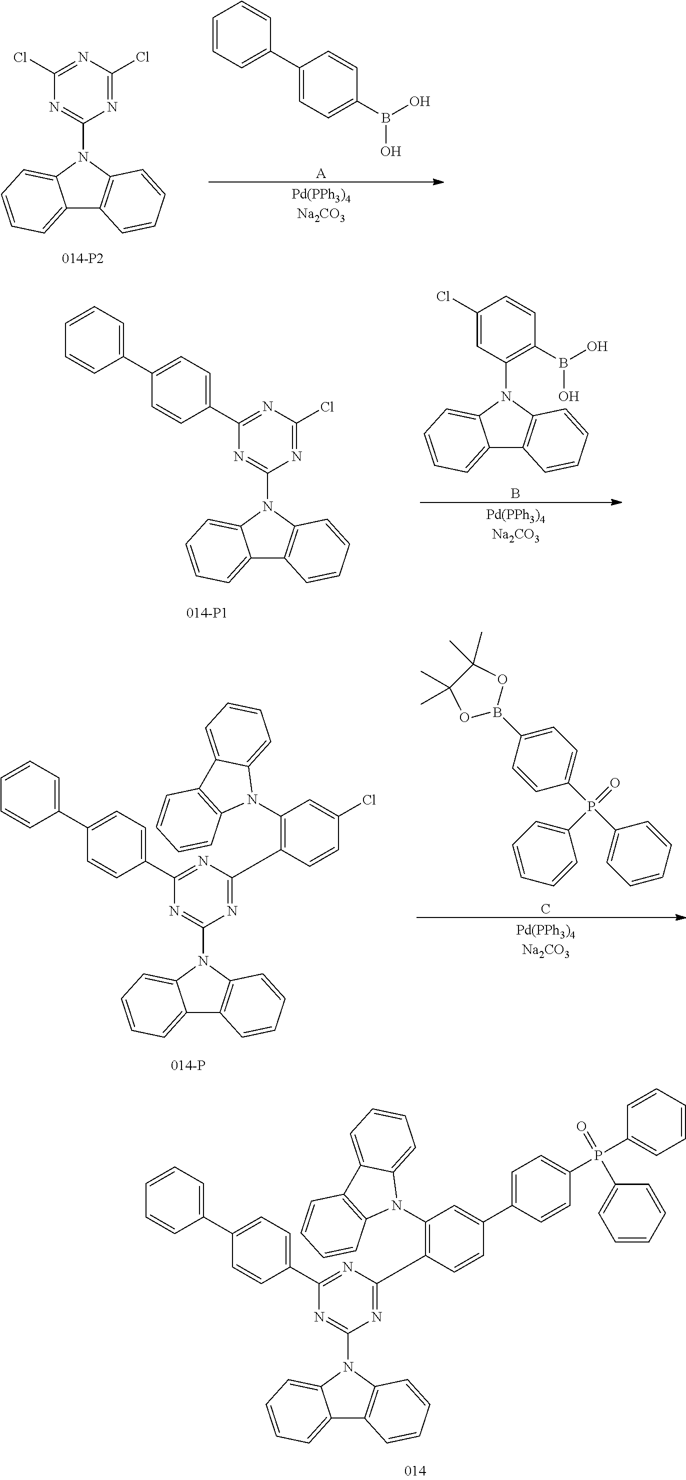

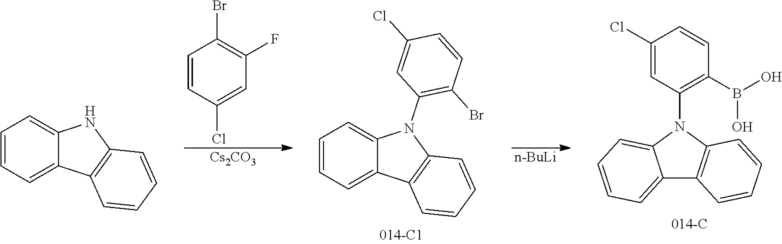

### 1) Preparation of Compound 014-C1

Compound 9H-carbazole (20 g, 0.12 mol, 1 eq.), 1-bromo-4-chloro-2-fluorobenzene (25.3 g, 0.12 mol, 1 eq.), Cs₂CO₃ (78 g, 0.24 mol, 2 eq.) and dimethylacetamide (400 ml) were introduced, and stirred for 2 hours at 120° C. Water was introduced thereto to terminate the reaction, and then a produced solid was filtered. Compound 014-C1 (38.5 g) was obtained in a yield of 90%.

### 2) Preparation of Compound 014-C

2.5 M n-BuLi (20 ml, 0.05 mol, 2 eq.) was introduced to Compound 014-C1 (15 g, 0.042 mol, 1 eq.) and THE (200 ml) at −78° C., the mixture was stirred for 1 hour, then triethylborane (6.2 g, 0.063 mol, 1.5 eq.) was introduced thereto, and the result was stirred for 4 hours at room temperature. Ethyl acetate (100 ml) and 1 N HCl (100 ml) were introduced thereto, and a produced solid was filtered to obtain Compound 014-C (10.8 g) in a yield of 80%.

### 3) Preparation of Compound 014-P2

2.5 M n-BuLi (44 ml, 0.11 mol, 1 eq.) was introduced to Compound 9H-carbazole (20 g, 0.12 mol, 1.1 eq.) and THF (200 ml) at −30° C., and the mixture was stirred for 1 hour. Cyanuric chloride (20 g, 0.11 mol, 1 eq.) and THE (200 ml) were introduced thereto, and the result was stirred for 2 hours at room temperature. Water was introduced thereto to terminate the reaction, and then a produced solid was filtered. Compound 014-P2 (24.3 g) was obtained in a yield of 70%.

### 4) Preparation of Compound 014-P1

Compound 014-P2 (15 g, 0.047 mol, 1 eq.), [1,1′-biphenyl]-4-ylboronic acid (A) (10.2 g, 0.052 mol, 1.1 eq.), Pd(PPh₃)₄ (2.7 g, 0.002 mol, 0.05 eq.), Na₂CO₃ (10 g, 0.094 mol, 2 eq.), 1,4-dioxane (200 ml) and water (50 ml) were introduced, and stirred for 4 hours at 100° C. Compound 014-P1 (13.6 g) was obtained in a yield of 67%.

### 5) Preparation of Compound 014-P

Compound 014-P1 (13.6 g, 0.031 mol, 1 eq.), Compound 014-C (B) (11 g, 0.034 mol, 1.1 eq.), Pd(PPh₃)₄ (1.8 g, 0.0015 mol, 0.05 eq.), K₂CO₃ (8.5 g, 0.062 mol, 2 eq.), 1,4-dioxane (200 ml) and water (50 ml) were introduced, and stirred for 4 hours at 100° C. Compound 014-P (17.7 g) was obtained in a yield of 85%.

### 6) Preparation of Compound 014

Compound 014-P (15 g, 0.026 mol, 1 eq.), diphenyl(4-(4,4,5,5-tetramethyl-1,3,2-dioxaborolan-2-yl)phenyl)phosphine oxide (C) (11.5 g, 0.029 mol, 1.1 eq.), Pd₂(dba)₃ (1.2 g, 0.0013 mol, 0.05 eq.), XPhos (1.2 g, 0.0026 mol, 0.1 eq.), K₂CO₃ (10.5 g, 0.076 mol, 2 eq.), 1,4-dioxane (200 ml) and water (50 ml) were introduced, and stirred for 4 hours at 100° C. Compound 014 (15.5 g) was obtained in a yield of 65%.

### <Preparation Example 6> Preparation of Compounds 055, 079, 098, 115, 119, 133, 157, 163, 170, 176, 178, 182 and 183

Compounds 055, 079, 098, 115, 119, 133, 157, 163, 170, 176, 178, 182 and 183 were synthesized in the same manner as in Preparation Example 5, except that Intermediate A of the following Table 3 was used instead of [1,1′-biphenyl]-4-ylboronic acid (A), Intermediate B of the following Table 3 was used instead of (2-(9H-carbazol-9-yl)-4-chlorophenyl) boronic acid (B), and Intermediate C of the following Table 3 was used instead of diphenyl(4-(4,4,5,5-tetramethyl-1,3,2-dioxaborolan-2-yl)phenyl)phosphine oxide (C).

TABLE 3

| Compound | Intermediate | Intermediate | | |
|---|---|---|---|---|
| No. | A | B | Intermediate C | Yield |
| 055 | 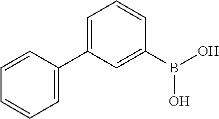 | 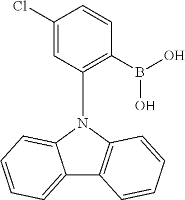 | 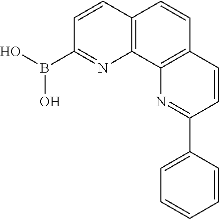 [SMILES: OB(O)c1ccc2ccc3ccc(-c4ccccc4)nc3c2n1] | 78% |
| 079 | 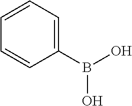 | 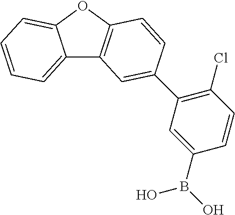 | 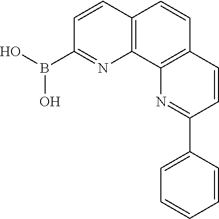 [SMILES: OB(O)c1ccc2ccc3ccc(-c4ccccc4)nc3c2n1] | 70% |
| 098 | 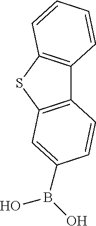 | 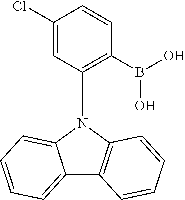 | 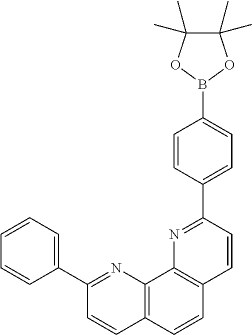 [SMILES: CC1(C)OB(c2ccc(-c3ccc4ccc5ccc(-c6ccccc6)nc5c4n3)cc2)OC1(C)C] | 92 |
| 115 | 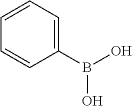 | 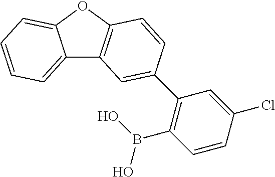 | 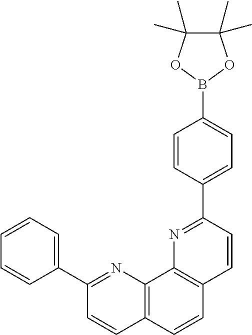 [SMILES: CC1(C)OB(c2ccc(-c3ccc4ccc5ccc(-c6ccccc6)nc5c4n3)cc2)OC1(C)C] | 78 |
| 119 | 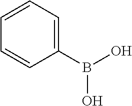 | 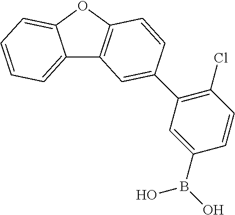 | 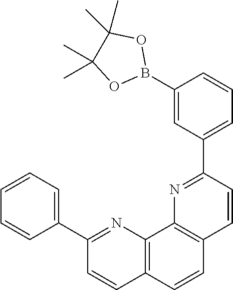 [SMILES: CC1(C)OB(c2cccc(-c3ccc4ccc5ccc(-c6ccccc6)nc5c4n3)c2)OC1(C)C] | 72 |
| 133 | 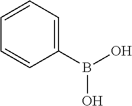 | 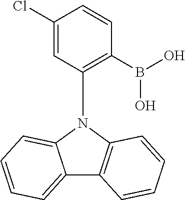 | 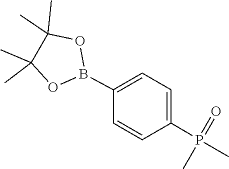 | 83 |
| 157 | 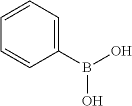 | 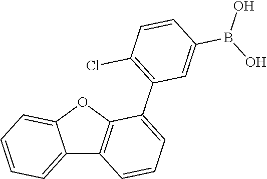 | 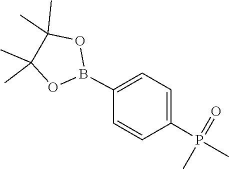 | 70 |
| 163 | 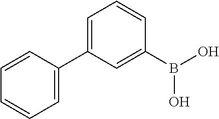 | 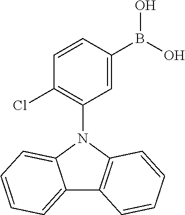 | 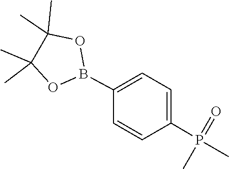 | 71 |
| 170 | 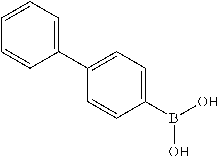 | 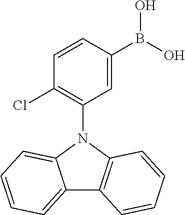 | 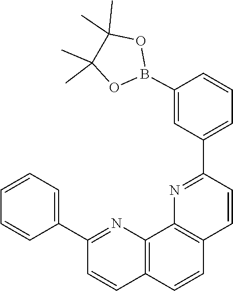 [SMILES: CC1(C)OB(c2cccc(-c3ccc4ccc5ccc(-c6ccccc6)nc5c4n3)c2)OC1(C)C] | 77 |
| 176 | 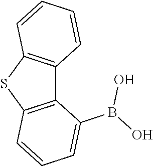 | 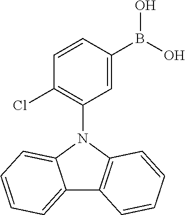 | 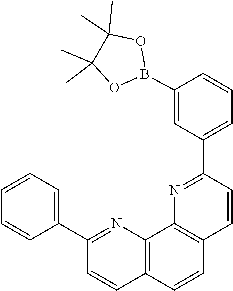 [SMILES: CC1(C)OB(c2cccc(-c3ccc4ccc5ccc(-c6ccccc6)nc5c4n3)c2)OC1(C)C] | |
| 178 | 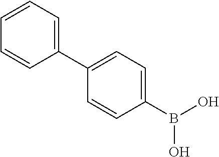 | 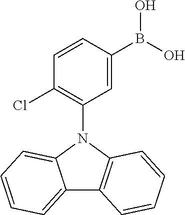 | 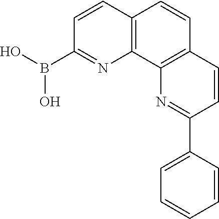 [SMILES: OB(O)c1ccc2ccc3ccc(-c4ccccc4)nc3c2n1] | |
| 182 | 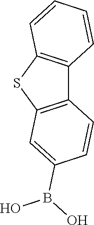 | 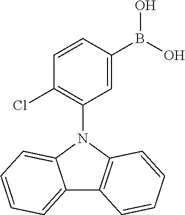 | 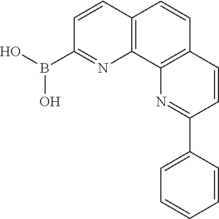 [SMILES: OB(O)c1ccc2ccc3ccc(-c4ccccc4)nc3c2n1] | |
| 183 | 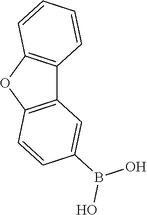 | 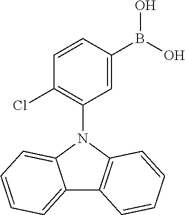 | 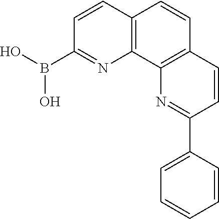 [SMILES: OB(O)c1ccc2ccc3ccc(-c4ccccc4)nc3c2n1] | |

The rest of compounds other than the compounds described in Preparation Examples 1 to 6, and Tables 1 to 3 were also prepared in the same manner as in the preparation examples described above, and the synthesis identification results are shown in the following Table 4 and Table 5. Table 4 shows measurement values of 1H NMR (DMSO, 500 MHz), and Table 5 shows measurement values of field desorption mass spectrometry (FD-MS).

TABLE 4

| Com- | |
|---|---|
| pound | ¹H NMR (DMSO, 500 MHz) |
| 009 | 8.55(2H, s), 8.19(2H, d), 7.97(2H, d), 7.96(2H, d), 7.94(2H, |
| | d), 7.77(4H, d), 7.58(2H, d), 7.51(6H, d), 7.5(4H, d), |
| | 7.49(1H, dd), 7.41(2H, d), 7.35(2H, d), 7.2(2H, dd), |
| | 7.16(2H, dd) |
| 014 | 8.55(2H, s), 8.4(1H, d), 8.19(2H, d), 8.13(1H, d), 8.1(1H, |
| | d), 7.96(2H, d), 7.94(2H, d), 7.87(1H, d), 7.77(4H, d), |
| | 7.76(1H, dd), 7.75(2H, d), 7.74(1H, d), 7.58(2H, dd), |
| | 7.51(6H, dd), 7.5(2H, s), 7.49 (2H, t), 7.35 (2H, t), 7.25 |
| | (2H, dd), 7.21 (1H, dd), 7.2 (2H, t), 7.16 (2H, d) |
| 023 | 8.55(1H, s), 8.45(1H, d), 8.19(1H, d), 8.12(1H, d), 7.99(1H, |
| | d), 7.97(2H, d), 7.96(2H, d), 7.94(1H, d), 7.93(1H, d), |
| | 7.92(1H, dd), 7.77(4H, d), 7.58(1H, d), 7.56(1H, dd), |
| | 7.51(6H, dd), 7.5(1H, s), 7.49 (1H, t), 7.35 (1H, t), 7.2 |
| | (1H, dd), 7.16 (1H, dd) |
| 031 | 8.55(1H, s), 8.19(1H, d), 7.98(1H, d), 7.97(2H, d), 7.96(4H, |
| | d), 7.94(1H, d), 7.88(1H, d), 7.83(1H, d), 7.79(1H, d), |
| | 7.77(4H, dd), 7.6(2H, d), 7.58(1H, d), 7.54(1H, dd), |
| | 7.51(6H, dd), 7.5(1H, s), 7.39 (1H, t), 7.35 (1H, t), 7.31 |
| | (1H, dd), 7.2 (1H, dd), 7.16 (1H, t) |
| 041 | 8.71(1H, s), 8.65(1H, d), 8.55(1H, d), 8.36(2H, d), 8.33(2H, |
| | d), 8.19(1H, d), 8.14(1H, d), 7.94(1H, d), 7.89(1H, d), |
| | 7.63(1H, dd), 7.58(1H, d), 7.55(2H, d), 7.5(4H, dd), |
| | 7.49(1H, dd), 7.35(1H, s), 7.29 (1H, t), 7.2 (1H, t), 7.16 |
| | (1H, dd) |
| 048 | 9.6(1H, s), 9.27(1H, d), 8.71(1H, d), 8.65(1H, d), 8.55(1H, |
| | d), 8.33(3H, d), 8.3(1H, d), 8.19(1H, d), 8.15(1H, d), |
| | 8.14(1H, dd), 7.94(1H, d), 7.89(1H, d), 7.7(2H, dd), |
| | 7.64(2H, dd), 7.63(1H, s), 7.58 (1H, t), 7.55 (2H, t), 7.52 |
| | (2H, dd), 7.5 (1H, dd), 7.49 (1H, t), 7.35 (1H, d), 7.29 |
| | (1H, d), 7.2 (1H, d) |
| 049 | 8.71(1H, s), 8.65(1H, d), 8.55(2H, d), 8.33(2H, d), 8.19(2H, |
| | d), 8.14(1H, d), 7.94(2H, d), 7.89(1H, d), 7.63(1H, d), |
| | 7.58(2H, dd), 7.55(2H, d), 7.5(2H, d), 7.49(1H, dd), |
| | 7.35(2H, dd), 7.29(1H, s), 7.2 (2H, t), 7.16 (2H, t) |
| 052 | 8.71(1H, s), 8.65(1H, d), 8.55(2H, d), 8.33(2H, d), 8.19(2H, |
| | d), 8.14(1H, d), 7.94(2H, d), 7.92(1H, d), 7.91(1H, d), |
| | 7.89(1H, dd), 7.8(1H, d), 7.63(1H, d), 7.58(2H, dd), |
| | 7.55(2H, dd), 7.5(2H, s), 7.49 (1H, t), 7.46 (1H, t), 7.35 |
| | (2H, dd), 7.29 (1H, dd), 7.2 (2H, t), 7.16 (2H, d) |
| 055 | 8.79(1H, s), 8.71(2H, d), 8.65(1H, d), 8.55(2H, d), 8.38(1H, |
| | d), 8.33(2H, d), 8.2(1H, d), 8.19(2H, d), 8.1(1H, d), |
| | 7.94(3H, dd), 7.9(1H, d), 7.75(2H, d), 7.73(1H, dd), |
| | 7.61(1H, dd), 7.58(2H, s), 7.55 (2H, t), 7.5 (2H, t), 7.49 |
| | (3H, dd), 7.41 (1H, dd), 7.35 (2H, t), 7.29 (2H, d), 7.2 |
| | (2H, d), 7.16 (2H, d) |
| 070 | 8.71(1H, s), 8.65(1H, d), 8.55(1H, d), 8.45(1H, d), 8.33(2H, |
| | d), 8.24(1H, d), 8.2(1H, d), 8.19(1H, d), 8.17(1H, d), |
| | 8.14(1H, dd), 7.96(2H, d), 7.94(1H, d), 7.93(1H, dd), |
| | 7.89(1H, dd), 7.63(1H, s), 7.6 (2H, t), 7.58 (1H, t), 7.56 |
| | (1H, dd), 7.55 (2H, dd), 7.5 (1H, t), 7.49 (2H, d), 7.35 |
| | (1H, d), 7.29 (1H, d) |
| 079 | 8.71(2H, s), 8.55(1H, d), 8.54(1H, d), 8.36(2H, d), 8.33(2H, |
| | d), 8.2(1H, d), 8.19(1H, d), 8.13(1H, d), 8.06(1H, d), |
| | 7.98(1H, dd), 7.94(1H, d), 7.9(1H, d), 7.88(1H, dd), |
| | 7.83(1H, dd), 7.79(1H, s), 7.58 (1H, t), 7.55 (2H, t), 7.54 |
| | (1H, dd), 7.5 (4H, dd), 7.49 (1H, t), 7.39 (1H, d), 7.35 |
| | (1H, d), 7.31 (1H, d) |
| 089 | 8.71(2H, s), 8.55(2H, d), 8.35(1H, d), 8.33(2H, d), 8.2(1H, |
| | d), 8.19(2H, d), 7.96(1H, d), 7.94(2H, d), 7.9(1H, d), |
| | 7.6(2H, dd), 7.58(2H, d), 7.55(2H, d), 7.5(2H, dd), 7.49(1H, |
| | dd), 7.35(2H, s), 7.29 (2H, t), 7.2 (2H, t), 7.16 (2H, dd) |
| 098 | 8.71(2H, s), 8.69(2H, d), 8.55(2H, d), 8.45(1H, d), 8.4(1H, |
| | d), 8.33(2H, d), 8.24(1H, d), 8.2(2H, d), 8.19(2H, d), |
| | 8.1(1H, dd), 7.94(3H, d), 7.93(1H, d), 7.9(1H, dd), 7.85(2H, |
| | dd), 7.58(2H, s), 7.56 (1H, t), 7.55 (2H, t), 7.5 (2H, dd), |
| | 7.49 (2H, dd), 7.35 (2H, t), 7.29 (2H, d), 7.21 (1H, d), 7.2 |
| | (2H, d) |
| 103 | 8.71(2H, s), 8.69(2H, d), 8.55(1H, d), 8.45(1H, d), 8.33(2H, |
| | d), 8.2(1H, d), 8.19(1H, d), 8.12(1H, d), 7.99(1H, d), |
| | 7.96(2H, dd), 7.94(1H, d), 7.93(1H, d), 7.92(1H, dd), |
| | 7.9(1H, dd), 7.58(1H, s), 7.56 (1H, t), 7.55 (2H, t), 7.5 |
| | (1H, dd), 7.49 (2H, dd), 7.35 (1H, t), 7.29 (2H, d), 7.2 |
| | (1H, d), 7.16 (1H, d) |
| 105 | 8.71(2H, s), 8.69(2H, d), 8.55(1H, d), 8.33(2H, d), 8.2(1H, |
| | d), 8.19(1H, d), 8.08(1H, d), 7.98(1H, d), 7.96(2H, d), |
| | 7.94(1H, dd), 7.9(1H, d), 7.88(1H, d), 7.58(1H, dd), |
| | 7.55(2H, dd), 7.54(1H, s), 7.51 (1H, t), 7.5 (1H, t), 7.49 |
| | (1H, dd), 7.39 (1H, dd), 7.35 (1H, t), 7.31 (1H, d), 7.29 |
| | (2H, d), 7.2 (1H, d) |
| 115 | 8.71(2H, s), 8.69(2H, d), 8.55(1H, d), 8.36(2H, d), 8.33(2H, |
| | d), 8.2(1H, d), 8.19(1H, d), 8.15(1H, d), 8.13(1H, d), |
| | 7.98(1H, dd), 7.94(1H, d), 7.9(1H, d), 7.88(1H, dd), |
| | 7.85(2H, dd), 7.83(1H, s), 7.79 (1H, t), 7.58 (1H, t), 7.55 |
| | (2H, dd), 7.54 (1H, dd), 7.5 (4H, t), 7.49 (1H, d), 7.39 |
| | (1H, d), 7.35 (2H, d) |
| 119 | 8.71(2H, s), 8.55(1H, d), 8.36(2H, d), 8.33(4H, d), 8.2(1H, |
| | d), 8.19(1H, d), 8.15(1H, d), 8.13(1H, d), 8.06(1H, d), |
| | 7.98(1H, dd), 7.94(1H, d), 7.9(1H, d), 7.88(1H, dd), |
| | 7.83(1H, dd), 7.79(1H, s), 7.73 (1H, t), 7.61 (1H, t), 7.58 |
| | (1H, dd), 7.55 (2H, dd), 7.54 (1H, t), 7.5 (4H, d), 7.49 |
| | (1H, d), 7.39 (1H, d) |
| 124 | 8.55(1H, s), 8.19(1H, d), 7.96(2H, d), 7.94(1H, d), 7.79(2H, |
| | d), 7.77(2H, d), 7.6(2H, d), 7.58(1H, d), 7.5(1H, d), |
| | 7.46(2H, dd), 7.43(2H, d), 7.41(1H, d), 7.35(1H, dd), |
| | 7.2(1H, dd), 7.16(1H, s), 2.06 (2H, t) |
| 129 | 8.55(2H, s), 8.19(2H, d), 7.94(2H, d), 7.77(2H, d), 7.58(2H, |
| | d), 7.5(2H, d), 7.43(2H, d), 7.35(2H, d), 7.2(2H, d), |
| | 7.16(2H, dd), 2.06(2H, d) |
| 133 | 8.55(2H, s), 8.4(1H, d), 8.36(2H, d), 8.19(2H, d), 8.1(1H, |
| | d), 7.94(2H, d), 7.77(2H, d), 7.58(2H, d), 7.5(5H, d), |
| | 7.43(2H, dd), 7.35(2H, d), 7.21(1H, d), 7.2(2H, dd), |
| | 7.16(2H, dd), 2.06(2H, s) |
| 152 | 8.55(1H, s), 8.45(1H, d), 8.28(1H, d), 8.19(1H, d), 8.03(1H, |
| | d), 7.96(2H, d), 7.94(2H, d), 7.93(1H, d), 7.68(1H, d), |
| | 7.67(1H, dd), 7.6(2H, d), 7.58(1H, d), 7.56(1H, dd), |
| | 7.54(1H, dd), 7.5(1H, s), 7.49 (1H, t), 7.35 (1H, t), 7.34 |
| | (1H, dd), 7.2 (1H, dd), 7.16 (1H, t), 2.06 (2H, d) |
| 157 | 8.55(1H, s), 8.36(2H, d), 8.19(1H, d), 8.15(1H, d), 8.13(1H, |
| | d), 8.08(1H, d), 8.06(1H, d), 8.02(1H, d), 7.98(1H, d), |
| | 7.94(1H, dd), 7.77(2H, d), 7.58(1H, d), 7.54(1H, dd), |
| | 7.51(1H, dd), 7.5(4H, s), 7.43 (2H, t), 7.39 (1H, t), 7.35 |
| | (1H, dd), 7.31 (1H, dd), 7.2 (1H, t), 7.16 (1H, d), 2.06 |
| | (2H, d) |
| 163 | 8.55(2H, s), 8.4(1H, d), 8.38(1H, d), 8.19(2H, d), 8.1(1H, |
| | d), 7.94(3H, d), 7.92(1H, d), 7.75(2H, d), 7.73(1H, d), |
| | 7.67(1H, dd), 7.61(1H, d), 7.58(2H, d), 7.54(1H, dd), |
| | 7.51(1H, dd), 7.5(2H, s), 7.49 (2H, t), 7.41 (1H, t), 7.35 |
| | (2H, dd), 7.34 (1H, dd), 7.2 (2H, t), 7.16 (2H, d), 2.06 |
| | (2H, d) |
| 170 | 8.71(2H, s), 8.55(2H, d), 8.4(1H, d), 8.33(4H, d), 8.2(1H, |
| | d), 8.19(2H, d), 8.1(1H, d), 7.96(2H, d), 7.94(2H, d), |
| | 7.92(1H, dd), 7.9(1H, d), 7.75(2H, d), 7.73(1H, dd), |
| | 7.61(1H, dd), 7.58(2H, s), 7.55 (2H, t), 7.5 (2H, t), 7.49 |
| | (3H, dd), 7.35 (2H, dd), 7.29 (2H, t), 7.25 (2H, d), 7.2 |
| | (2H, d), 7.16 (2H, d) |
| 176 | 8.71(2H, s), 8.55(2H, d), 8.45(1H, d), 8.4(1H, d), 8.33(4H, |
| | d), 8.2(1H, d), 8.19(2H, d), 8.1(1H, d), 8.03(1H, d), |
| | 7.94(3H, dd), 7.93(1H, d), 7.92(1H, d), 7.9(1H, dd), |
| | 7.73(1H, dd), 7.68(1H, s), 7.61 (1H, t), 7.58 (2H, t), 7.56 |
| | (1H, dd), 7.55 (2H, dd), 7.5 (2H, t), 7.49 (2H, d), 7.35 |
| | (2H, d), 7.29 (2H, d) |
| 178 | 8.71(2H, s), 8.55(2H, d), 8.49(1H, d), 8.4(1H, d), 8.33(2H, |
| | d), 8.2(1H, d), 8.19(2H, d), 7.96(2H, d), 7.94(2H, d), |
| | 7.92(1H, dd), 7.9(1H, d), 7.75(2H, d), 7.58(2H, dd), |
| | 7.55(2H, dd), 7.5(2H, s), 7.49 (3H, t), 7.41 (1H, t), 7.35 |
| | (2H, dd), 7.29 (2H, dd), 7.25 (2H, t), 7.2 (2H, d), 7.16 |
| | (2H, d) |
| 182 | 8.71(2H, s), 8.55(2H, d), 8.49(1H, d), 8.45(1H, d), 8.4(1H, |
| | d), 8.33(2H, d), 8.24(1H, d), 8.2(2H, d), 8.19(2H, d), |
| | 7.94(3H, dd), 7.93(1H, d), 7.92(1H, d), 7.9(1H, dd), |
| | 7.58(2H, dd), 7.56(1H, s), 7.55 (2H, t), 7.5 (2H, t), 7.49 |
| | (2H, dd), 7.35 (2H, dd), 7.29 (2H, t), 7.2 (2H, d), 7.16 |
| | (2H, d) |
| 183 | 8.71(2H, s), 8.55(2H, d), 8.49(1H, d), 8.4(1H, d), 8.33(2H, |
| | d), 8.2(1H, d), 8.19(2H, d), 7.98(1H, d), 7.94(2H, d), |
| | 7.92(1H, dd), 7.9(1H, d), 7.88(1H, d), 7.83(1H, dd), |
| | 7.79(1H, dd), 7.58(2H, s), 7.55(2H, t), 7.54 (1H, t), 7.5 |
| | (2H, dd), 7.49 (1H, dd), 7.39 (1H, t), 7.35 (2H, d), 7.31 |
| | (1H, d), 7.29 (2H, d) |
| 195 | 9.6(1H, s), 9.24(1H, d), 9.08(1H, d), 8.71(2H, d), 8.69(2H, |
| | d), 8.6(1H, d), 8.55(2H, d), 8.33(2H, d), 8.2(1H, d), |
| | 8.19(2H, dd), 7.96(2H, d), 7.94(2H, d), 7.9(1H, dd), 7.7(2H, |
| | dd), 7.64(2H, s), 7.58 (2H, t), 7.55 (2H, t), 7.52 (2H, dd), |
| | 7.5 (2H, dd), 7.35 (2H, t), 7.29 (2H, d), 7.2 (2H, d), 7.16 |
| | (2H, d) |
| 197 | 9.6(1H, s), 9.24(1H, d), 9.08(1H, d), 8.6(1H, d), 8.55(2H, |
| | d), 8.51(1H, d), 8.39(1H, d), 8.19(1H, d), 7.97(2H, d), |
| | 7.96(2H, dd), 7.94(2H, d), 7.77(4H, d), 7.7(2H, dd), |
| | 7.64(2H, dd), 7.58(1H, s), 7.52 (2H, t), 7.51 (6H, t), 7.5 |
| | (1H, dd), 7.35 (2H, dd), 7.2 (1H, t), 7.17 (1H, d), 7.16 |
| | (2H, d) |

TABLE 5

| Compound | FD-MS | Compound | FD-MS |
|---|---|---|---|
| 009 | m/z = 687.22 | 014 | m/z = 915.31 |
| | (C45H30N5OP, 687.74) | | (C63H42N5OP = 916.04) |
| 023 | m/z = 704.18 | 031 | m/z = 764.23 |
| | (C45H29N4OPS = 704.79) | | (C51H33N4O2P = 764.82) |
| 041 | m/z = 576.21 | 048 | m/z = 726.25 |
| | (C39H24N6 = 576.66) | | (C51H30N6 = 726.84) |
| 049 | m/z = 665.23 | 052 | m/z = 741.26 |
| | (C45H27N7 = 665.76) | | (C51H31N7 = 741.86) |
| 055 | m/z = 893.33 | 070 | m/z = 758.23 |
| | (C63H39N7 = 894.05) | | (C51H30N6S = 758.90) |
| 079 | m/z = 818.28 | 089 | m/z = 741.26 |
| | (C57H34N6O = 818.94) | | (C51H31N7 = 741.86) |
| 098 | m/z = 999.31 | 103 | m/z = 758.23 |
| | (C69H41N7S = 1000.20) | | (C51H30N6S = 758.90) |
| 105 | m/z = 742.25 | 115 | m/z = 894.31 |
| | (C51H30N6O = 742.84) | | (C63H38N6O = 895.04) |
| 119 | m/z = 894.31 | 124 | m/z = 550.19 |
| | (C63H38N6O = 895.04) | | (C35H27N4OP = 550.60) |
| 129 | m/z = 563.19 | 133 | m/z = 715.25 |
| | (C35H26N5OP = 563.60) | | (C47H34N5OP = 715.80) |
| 152 | m/z = 656.18 | 157 | m/z = 716.23 |
| | (C41H29N4OPS = 656.74) | | (C47H33N4O2P = 716.78) |
| 163 | m/z = 791.28 | 170 | m/z = 969.36 |
| | (C53H38N5OP = 791.89) | | (C69H43N7 = 970.15) |
| 176 | m/z = 999.31 | 178 | m/z = 893.33 |
| | (C69H41N7S = 1000.20) | | (C63H39N7 = 894.05) |
| 182 | m/z = 923.28 | 183 | m/z = 907.31 |
| | (C63H37N7S = 924.10) | | (C63H37N7O = 908.04) |
| 195 | m/z = 967.34 | 197 | m/z = 914.29 |
| | (C69H41N7 = 968.14) | | (C62H39N6OP = 915.01) |

### Experimental Example 1

### 1) Manufacture of Organic Light Emitting Device

A transparent electrode indium tin oxide (ITO) thin film obtained from glass for an OLED (manufactured by Samsung Corning Advanced Glass) was ultrasonic cleaned using trichloroethylene, acetone, ethanol and distilled water sequentially for 5 minutes each, and then stored in isopropanol before use. Then, the ITO substrate installed in a substrate folder of a vacuum deposition apparatus, and the following 4,4′,4″-tris(N,N-(2-naphthyl)-phenylamino)triphenyl amine (2-TNATA) was introduced to a cell in the vacuum deposition apparatus.

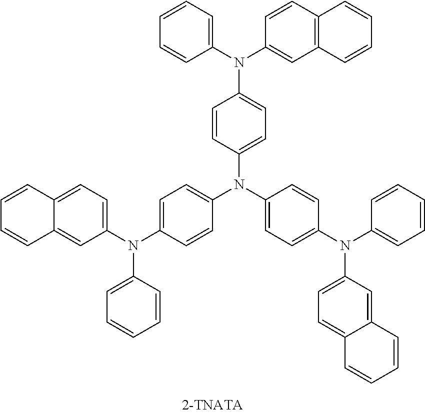 [SMILES: c1ccc(N(c2ccc(N(c3ccc(N(c4ccccc4)c4ccc5ccccc5c4)cc3)c3ccc(N(c4ccccc4)c4ccc5ccccc5c4)cc3)cc2)c2ccc3ccccc3c2)cc1]

Subsequently, the chamber was evacuated until the degree of vacuum therein reached 10⁻⁶ torr, and then the 2-TNATA was evaporated by applying a current to the cell to deposit a hole injection layer having a thickness of 600 Å on the ITO substrate. To another cell in the vacuum deposition apparatus, the following N,N′-bis(α-naphthyl)-N, N′-diphenyl-4,4′-diamine (NPB) was introduced, and evaporated by applying a current to the cell to deposit a hole transport layer having a thickness of 300 Å on the hole injection layer.

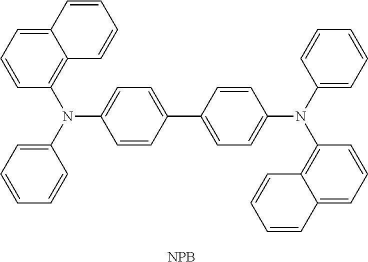 [SMILES: c1ccc(N(c2ccc(-c3ccc(N(c4ccccc4)c4cccc5ccccc45)cc3)cc2)c2cccc3ccccc23)cc1 ; "NPB"]

After forming the hole injection layer and the hole transport layer as above, a blue light emitting material having the following structure was deposited thereon as a light emitting layer. Specifically, on one cell in the vacuum deposition apparatus, H1 that is a blue light emitting host material was vacuum deposited to a thickness of 200 Å, and D1 that is a blue light emitting dopant material was vacuum deposited thereon by 5% with respect to the host material.

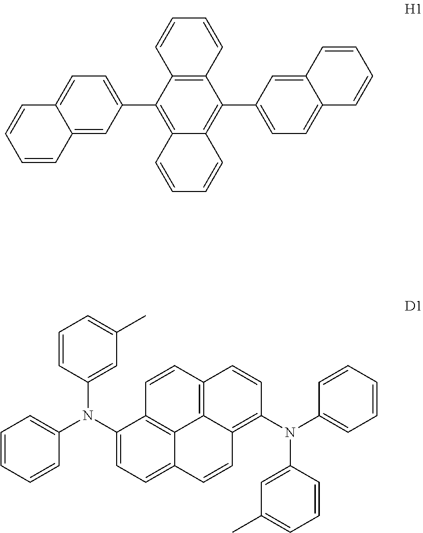

Subsequently, a compound of the following structural formula E1 was deposited to a thickness of 300 Å as an electron transport layer.

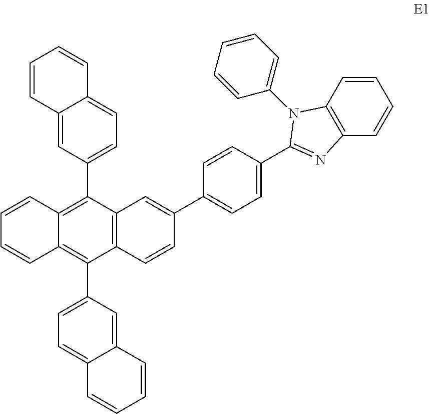 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1]

Lithium fluoride (LiF) was deposited to a thickness of 10 Å as an electron injection layer, and an Al negative electrode was employed to a thickness of 1,000 Å, and as a result, an OLED was manufactured. Meanwhile, all the organic compounds required to manufacture the OLED were vacuum sublimation purified under 10⁻⁸ torr to 10⁻⁶ torr for each material to be used in the manufacture of the OLED.

An organic electroluminescent device was manufactured in the same manner as in Comparative Example 1, except that a compound shown in the following Table 6 was used instead of E1 used when forming the electron transport layer in Comparative Example 1.

### 2) Driving Voltage and Light Emission Efficiency of Organic Electroluminescent Device

For each of the organic light emitting devices manufactured as above, electroluminescent (EL) properties were measured using M7000 manufactured by McScience Inc., and with the measurement results, lifetime T₉₅ (unit: h, hour), a time taken for luminance to become 95% with respect to initial luminance, was measured when standard luminance was 3,500 cd/m² through a lifetime measurement system (M6000) manufactured by McScience Inc. Results of measuring driving voltage, light emission efficiency and lifetime of the organic light emitting device of the present disclosure are shown in the following Table 6.

TABLE 6

| | | Driving | Light Emission | | Life |
|---|---|---|---|---|---|
| | | Voltage | Efficiency | | time |
| | Compound | (V) | (cd/A) | CIE (x, y) | (T₉₅) |
| Example 1 | 009 | 4.31 | 6.81 | (0.133, 0.100) | 88 |
| Example 2 | 014 | 4.33 | 6.78 | (0.134, 0.100) | 90 |
| Example 3 | 023 | 4.55 | 6.80 | (0.134, 0.100) | 89 |
| Example 4 | 031 | 4.48 | 6.83 | (0.134, 0.101) | 88 |
| Example 5 | 041 | 4.44 | 6.73 | (0.133, 0.101) | 85 |
| Example 6 | 048 | 4.42 | 6.85 | (0.134, 0.100) | 83 |
| Example 7 | 049 | 4.48 | 6.90 | (0.133, 0.101) | 89 |
| Example 8 | 052 | 4.38 | 6.74 | (0.134, 0.102) | 84 |
| Example 9 | 055 | 4.33 | 6.88 | (0.134, 0.101) | 86 |
| Example 10 | 070 | 4.45 | 6.80 | (0.134, 0.101) | 89 |
| Example 11 | 079 | 4.35 | 6.70 | (0.133, 0.100) | 82 |
| Example 12 | 089 | 4.36 | 6.85 | (0.134, 0.100) | 83 |
| Example 13 | 098 | 4.41 | 6.85 | (0.134, 0.100) | 85 |
| Example 14 | 103 | 4.42 | 6.84 | (0.133, 0.100) | 85 |
| Example 15 | 105 | 4.48 | 6.86 | (0.134, 0.101) | 88 |
| Example 16 | 115 | 4.47 | 6.87 | (0.134, 0.101) | 84 |
| Example 17 | 119 | 4.31 | 6.88 | (0.133, 0.100) | 84 |
| Example 18 | 124 | 4.33 | 6.85 | (0.133, 0.101) | 85 |
| Example 19 | 129 | 4.55 | 6.87 | (0.134, 0.100) | 85 |
| Example 20 | 133 | 4.48 | 6.71 | (0.134, 0.100) | 81 |
| Example 21 | 152 | 4.44 | 6.90 | (0.133, 0.101) | 85 |
| Example 22 | 157 | 4.42 | 6.88 | (0.134, 0.101) | 84 |
| Example 23 | 163 | 4.48 | 6.93 | (0.133, 0.101) | 86 |
| Example 24 | 170 | 4.38 | 6.95 | (0.134, 0.100) | 95 |
| Example 25 | 176 | 4.33 | 6.88 | (0.133, 0.101) | 87 |
| Example 26 | 178 | 4.37 | 6.90 | (0.133, 0.100) | 93 |
| Example 27 | 182 | 4.33 | 6.88 | (0.134, 0.101) | 88 |
| Example 28 | 183 | 4.45 | 6.80 | (0.134, 0.101) | 85 |
| Example 29 | 195 | 4.35 | 6.70 | (0.133, 0.100) | 83 |
| Example 30 | 197 | 4.36 | 6.85 | (0.134, 0.100) | 89 |
| Comparative | E1 | 5.51 | 6.17 | (0.134, 0.100) | 31 |
| Example 1 | | | | | |
| Comparative | Comparative | 5.28 | 6.35 | (0.134, 0.100) | 48 |
| Example 2 | Compound A | | | | |
| Comparative | Comparative | 5.31 | 6.38 | (0.134, 0.100) | 55 |
| Example 3 | Compound B | | | | |
| Comparative | Comparative | 5.44 | 6.35 | (0.134, 0.100) | 44 |
| Example 4 | Compound C | | | | |
| Comparative | Comparative | 5.80 | 6.34 | (0.134, 0.100) | 50 |
| Example 5 | Compound D | | | | |

Comparative compounds used in Table 6 are as follows.

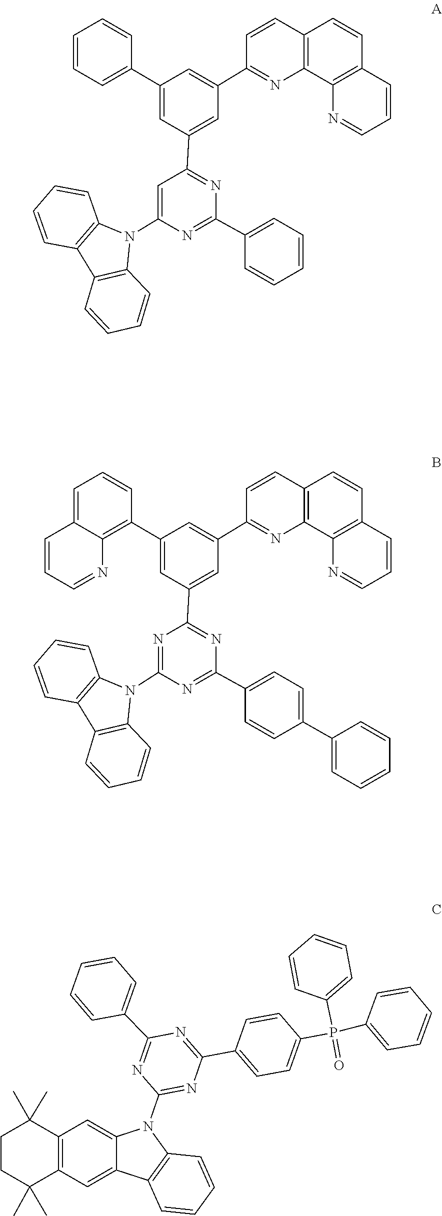 [SMILES: c1ccc(-c2cc(-c3cc(-n4c5ccccc5c5ccccc54)nc(-c4ccccc4)n3)cc(-c3ccc4ccc5cccnc5c4n3)c2)cc1 ; c1ccc(-c2ccc(-c3nc(-c4cc(-c5ccc6ccc7cccnc7c6n5)cc(-c5cccc6cccnc56)c4)nc(-n4c5ccccc5c5ccccc54)n3)cc2)cc1 ; CC1(C)CCC(C)(C)c2cc3c(cc21)c1ccccc1n3-c1nc(-c2ccccc2)nc(-c2ccc(P(=O)(c3ccccc3)c3ccccc3)cc2)n1]

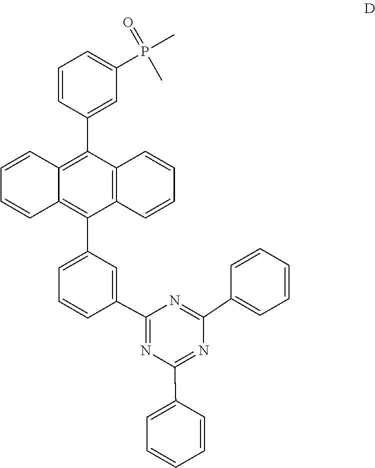 [SMILES: CP(C)(=O)c1cccc(-c2c3ccccc3c(-c3cccc(-c4nc(-c5ccccc5)nc(-c5ccccc5)n4)c3)c3ccccc23)c1]

As seen from the results of Table 6, the organic light emitting device using the electron transport layer material of the blue organic light emitting device of the present disclosure had lower driving voltage, and significantly improved light emission efficiency and lifetime compared to Comparative Examples 1 to 5.

The reason for such results may be understood to be obtained from the fact that the material of the compound represented by Chemical Formula 1 of the present disclosure has a unipolar structure in which a hole transport unit is added, and the structure of the compound facilitates electron transport.

In addition, unlike Comparative Examples 2 and 3, the compound represented by Chemical Formula 1 of the present disclosure has a phenyl group being fixed at the position No. 9 of the phenanthroline substituent represented by Chemical Formula 3, and accordingly, electrons may be efficiently transferred by increasing thermal stability and the band gap between HOMO and LUMO energy levels.

It may be seen that, in Comparative Compound C according to Comparative Example 4, an electron donating group (EDG) is further expanded in the carbazole group part, resulting in a stronger hole trap tendency, and as a result, electrons are not readily transported compared to in the heterocyclic compound according to the present disclosure.

It may be identified that, compared to Comparative Example 5, the heterocyclic compound according to the present disclosure has a structure in which a carbazole group and a heteroaryl group are added, lowering a HOMO energy level, and has an increased electron transport ability.

Accordingly, it is considered that the heterocyclic compound according to the present disclosure improves electron-transport properties or stability, thereby bringing excellences in all aspects of driving, efficiency and lifetime.

### Experimental Example 2

### 1) Manufacture of Organic Light Emitting Device

A glass substrate on which ITO was coated as a thin film to a thickness of 1,500 Å was ultrasonic cleaned with distilled water. When the cleaning with distilled water was finished, the substrate was ultrasonic cleaned with solvents such as acetone, methanol and isopropyl alcohol, then dried, and then subjected to UVO treatment for 5 minutes using UV in a UV cleaner. After that, the substrate was transferred to a plasma cleaner (PT), then subjected to plasma treatment under vacuum for ITO work function and residual film removal, and transferred to a thermal deposition apparatus for organic deposition.

On the transparent ITO electrode (positive electrode), organic materials were formed in a 2 stack WOLED (white organic light emitting device) structure. As for a first stack, TAPC was thermal vacuum deposited first to a thickness of 300 Å to form a hole transport layer. After forming the hole transport layer, a light emitting layer was thermal vacuum deposited thereon as follows. The light emitting layer was deposited to 300 Å by doping FIrpic, a blue phosphorescent dopant, to TCz1, a host, by 8 wt %. An electron transport layer was formed to 400 Å using TmPyPB, and then an N-type charge generation layer was formed to 100 Å by doping Cs₂CO₃ to a compound described in the following Table 7 by 20%.

As for a second stack, MoO₃ was thermal vacuum deposited first to a thickness of 50 Å to form a hole injection layer. As a hole transport layer that is a common layer, TAPC doped with MoO₃ by 20% was formed to 100 Å, and then TAPC was deposited to 300 Å. A light emitting layer was deposited to 300 Å thereon by doping Ir(ppy)₃, a green phosphorescent dopant, to TCz1, a host, by 8 wt %, and then an electron transport layer was formed to 600 Å using TmPyPB.

Lastly, lithium fluoride (LiF) was deposited on the electron transport layer to a thickness of 10 Å to form an electron injection layer, and then an aluminum (Al) negative electrode was deposited on the electron injection layer to a thickness of 1,200 Å to form a negative electrode, and as a result, an organic electroluminescent device was manufactured.

Meanwhile, all the organic compounds required to manufacture the OLED were vacuum sublimation purified under 10⁻⁸ torr to 10⁻⁶ torr for each material to be used in the manufacture of the OLED.

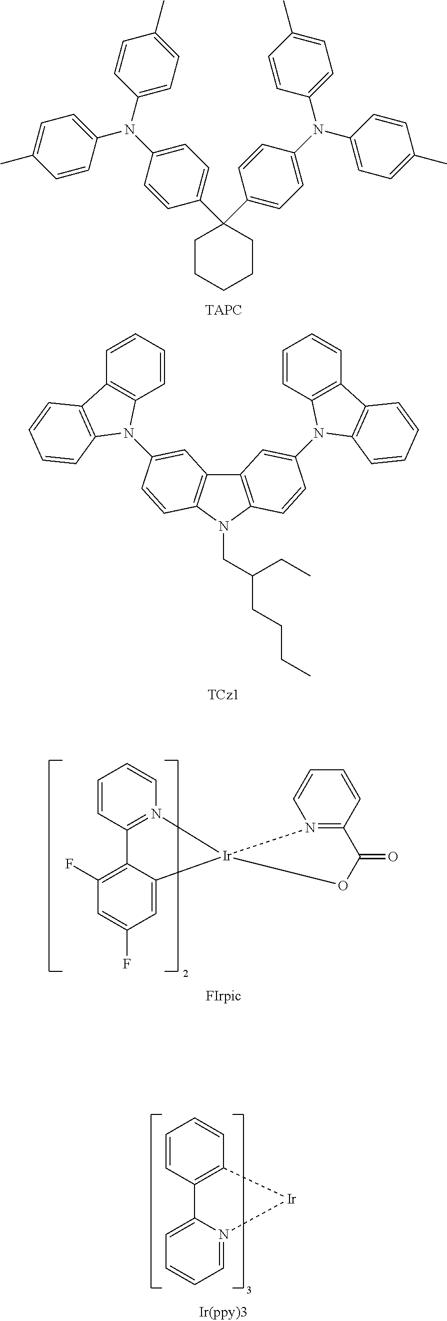

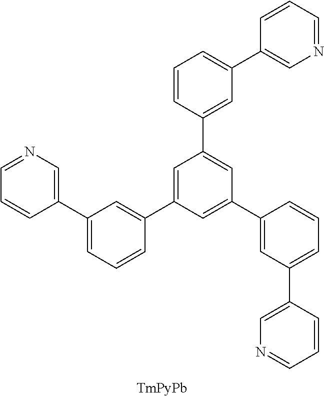 [SMILES: c1cncc(-c2cccc(-c3cc(-c4cccc(-c5cccnc5)c4)cc(-c4cccc(-c5cccnc5)c4)c3)c2)c1]

### 2) Driving Voltage and Light Emission Efficiency of Organic Electroluminescent Device

For each of the organic light emitting devices manufactured as above, electroluminescent (EL) properties were measured using M7000 manufactured by McScience Inc., and with the measurement results, lifetime T₉₅ (unit: h, hour), a time taken for luminance to become 95% with respect to initial luminance, was measured when standard luminance was 3,500 cd/m² through a lifetime measurement system (M6000) manufactured by McScience Inc. Results of measuring driving voltage, light emission efficiency and lifetime of the white organic light emitting device of the present disclosure are shown in the following Table 7.

TABLE 7

| | | Driving | Light Emission | | Life |
|---|---|---|---|---|---|
| | | Voltage | Efficiency | | time |
| | Compound | (V) | (cd/A) | CIE (x, y) | (T₉₅) |
| Example 31 | 009 | 7.03 | 67.58 | (0.214, 0.418) | 88 |
| Example 32 | 014 | 7.20 | 69.77 | (0.213, 0.417) | 84 |
| Example 33 | 023 | 7.44 | 68.68 | (0.213, 0.420) | 85 |
| Example 34 | 031 | 7.32 | 69.12 | (0.215, 0.420) | 86 |
| Example 35 | 041 | 7.35 | 69.15 | (0.215, 0.420) | 86 |
| Example 36 | 048 | 7.34 | 68.88 | (0.214, 0.420) | 85 |
| Example 37 | 049 | 7.33 | 69.02 | (0.214, 0.418) | 87 |
| Example 38 | 052 | 7.33 | 68.53 | (0.215, 0.419) | 86 |
| Example 39 | 055 | 7.40 | 70.03 | (0.216, 0,424) | 86 |
| Example 40 | 070 | 7.38 | 69.87 | (0.214, 0.425) | 85 |
| Example 41 | 079 | 7.24 | 68.13 | (0.219, 0.426) | 86 |
| Example 42 | 089 | 7.25 | 69.00 | (0.218, 0.421) | 85 |
| Example 43 | 098 | 7.30 | 69.50 | (0.215, 0.417) | 84 |
| Example 44 | 103 | 7.42 | 67.72 | (0.209, 0.422) | 85 |
| Example 45 | 105 | 7.33 | 68.58 | (0.210, 0.428) | 86 |
| Example 46 | 115 | 7.35 | 69.13 | (0.209, 0.428) | 87 |
| Example 47 | 119 | 7.31 | 68.43 | (0.210, 0.430) | 85 |
| Example 48 | 124 | 7.38 | 69.84 | (0.212, 0.428) | 83 |
| Example 49 | 129 | 7.42 | 70.18 | (0.219, 0.426) | 82 |
| Example 50 | 133 | 7.39 | 70.02 | (0.218, 0.424) | 84 |
| Example 51 | 152 | 7.30 | 70.42 | (0.215, 0.417) | 84 |
| Example 52 | 157 | 7.37 | 70.25 | (0.214, 0.420) | 85 |
| Example 53 | 163 | 7.33 | 69.39 | (0.214, 0.418) | 86 |
| Example 54 | 170 | 7.41 | 69.88 | (0.214, 0.418) | 95 |
| Example 55 | 176 | 7.31 | 69.98 | (0.215, 0.419) | 91 |
| Example 56 | 178 | 7.25 | 68.80 | (0.219, 0.426) | 96 |
| Example 57 | 182 | 7.30 | 69.22 | (0.220, 0.424) | 90 |
| Example 58 | 183 | 7.28 | 70.13 | (0.219, 0.422) | 92 |
| Example 59 | 195 | 7.41 | 69.88 | (0.214, 0.418) | 95 |
| Example 60 | 197 | 7.35 | 69.13 | (0.209, 0.428) | 87 |
| Comparative | TmPyPB | 8.20 | 47.71 | (0.211, 0.430) | 60 |
| Example 6 | | | | | |
| Comparative | Comparative | 7.98 | 55.84 | (0.134, 0.101) | 66 |
| Example 7 | Compound E | | | | |
| Comparative | Comparative | 8.53 | 58.43 | (0.133, 0.100) | 69 |
| Example 8 | Compound F | | | | |
| Comparative | Comparative | 8.01 | 47.44 | (0.134, 0.101) | 44 |
| Example 9 | Compound G | | | | |

Comparative compounds used in Table 7 are as follows.

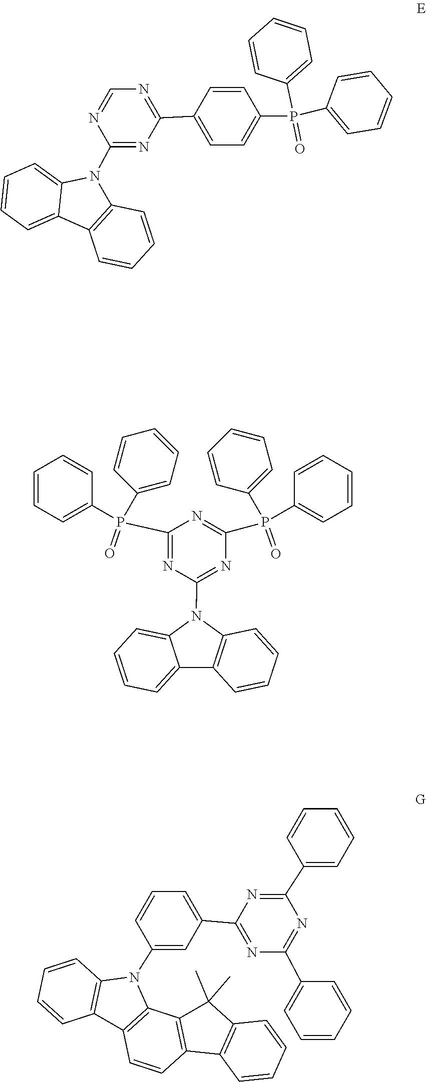 [SMILES: O=P(c1ccccc1)(c1ccccc1)c1ccc(-c2ncnc(-n3c4ccccc4c4ccccc43)n2)cc1 ; O=P(c1ccccc1)(c1ccccc1)c1nc(-n2c3ccccc3c3ccccc32)nc(P(=O)(c2ccccc2)c2ccccc2)n1 ; CC1(C)c2ccccc2-c2ccc3c4ccccc4n(-c4cccc(-c5nc(-c6ccccc6)nc(-c6ccccc6)n5)c4)c3c21]

As seen from the results of Table 7, the organic electroluminescent device using the N-type charge generation layer material of the 2-stack white organic electroluminescent device of the present disclosure had lower driving voltage, and improved lifetime and light emission efficiency compared to Comparative Examples 6 to 9.

The compound used in Comparative Example 7 has an sp³ orbital at the No. 5 carbon position with a structure in which the carbon of the triazine nucleus is disubstituted, showing a slow charge generation effect compared to the heterocyclic compound according to the present disclosure.

The compound used in Comparative Example 8 has a structure disubstituted with a metal acceptor compared to the heterocyclic compound according to the present disclosure, and, although binding with a metal readily occurs, a transport ability is reduced when charges are generated, greatly increasing a driving voltage.

The compound used in Comparative Example 9 has a bipolar structure, which has a greater deviation in electron migration compared to a unipolar structure, and shows lower lifetime properties compared to the organic light emitting device using the heterocyclic compound according to the present disclosure. In addition, the compound used in Comparative Example 9 does not have a metal binding unit unlike the heterocyclic compound of the present disclosure, thereby showing low efficiency and lifetime.

Accordingly, the heterocyclic compound represented by Chemical Formula 1 according to the present disclosure may be used as an efficient and stable N-type charge generation layer material. It may be understood that, due to the structural characteristics of the compound, a gap state is stably formed in the N-type charge generation layer when the compound used as the N-type charge generation layer is doped with a metal, and electrons generated from the P-type charge generation layer are readily injected into the electron transport layer through the gap state produced in the N-type charge generation layer.

[Reference Numeral]

100: Substrate
200: Positive Electrode
300: Organic Material Layer
301: Hole Injection Layer
302: Hole Transport Layer
303: Light Emitting Layer
304: Hole Blocking Layer
305: Electron Transport Layer
306: Electron Injection Layer
400: Negative Electrode

1. A heterocyclic compound represented by the following Chemical Formula 1: 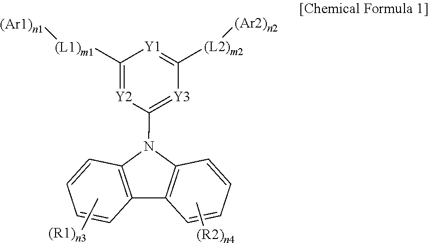
wherein, in Chemical Formula 1,
Y1 to Y3 are the same as or different from each other and each independently CH or N, and at least one of Y1 to Y3 is N;
L1 and L2 are the same as or different from each other, and each independently a single bond; a substituted or unsubstituted C6 to C60 arylene group; or a substituted or unsubstituted C2 to C60 heteroarylene group;
Ar1 and Ar2 are the same as or different from each other and each independently a substituted or unsubstituted C6 to C60 aryl group; or a substituted or unsubstituted C2 to C60 heteroaryl group, and at least one of Ar1 and Ar2 is a group represented by the following Chemical Formula 2 or Chemical Formula 3;
R1 and R2 are the same as or different from each other, and each independently selected from the group consisting of hydrogen; deuterium; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; and a substituted or unsubstituted C2 to C60 heteroaryl group, or two or more groups adjacent to each other bond to each other to form a substituted or unsubstituted C6 to C60 aromatic hydrocarbon ring or a substituted or unsubstituted C2 to C60 heteroring;
m1 and m2 are the same as or different from each other, and each independently an integer of 0 to 4; and
n1 to n4 are the same as or different from each other, and each independently an integer of 0 to 4, 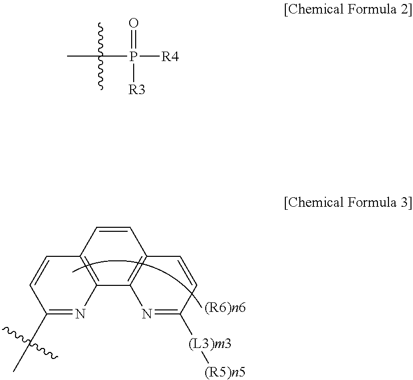
in Chemical Formulae 2 and 3,
R3 to R6 are the same as or different from each other, and each independently selected from the group consisting of hydrogen; deuterium; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; and a substituted or unsubstituted C2 to C60 heteroaryl group, or two or more groups adjacent to each other bond to each other to form a substituted or unsubstituted C6 to C60 aromatic hydrocarbon ring or a substituted or unsubstituted C2 to C60 heteroring;
L3 is a single bond; a substituted or unsubstituted C6 to C60 arylene group; or a substituted or unsubstituted C2 to C60 heteroarylene group;
m3 is an integer of 0 to 4;
n5 is an integer of 0 to 4; and
n6 is an integer of 0 to 6.

2. The heterocyclic compound of claim 1, wherein Chemical Formula 1 is represented by any one of the following Chemical Formula 1-1 or Chemical Formula 1-2: 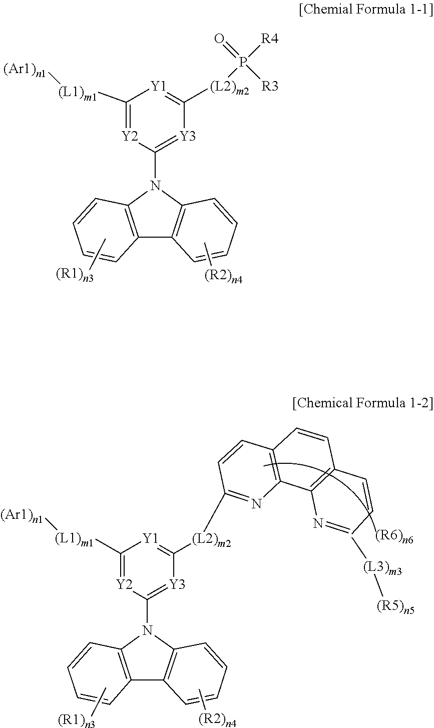
in Chemical Formula 1-1 and Chemical Formula 1-2,
each substituent has the same definition as in Chemical Formula 1.

3. The heterocyclic compound of claim 1, wherein L1 and L2 of Chemical Formula 1 are the same as or different from each other, and each independently represented by any one of a single bond; the following Chemical Formula 4; or the following Chemical Formula 5: 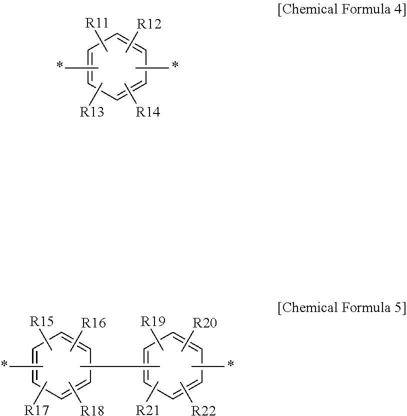
in Chemical Formulae 4 and 5,
R11 to R22 are the same as or different from each other, and each independently selected from the group consisting of hydrogen; deuterium; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; and a substituted or unsubstituted C2 to C60 heteroaryl group, or two or more groups adjacent to each other bond to each other to form a substituted or unsubstituted C6 to C60 aromatic hydrocarbon ring or a substituted or unsubstituted C2 to C60 heteroring; and
*s each indicate a point connected to carbon between Y1 and Y3 and Ar2 of Chemical Formula 1.

4. The heterocyclic compound of claim 1, wherein the heterocyclic compound represented by Chemical Formula 1 does not include deuterium as a substituent, or has a deuterium content of 1% to 100% based on a total number of hydrogen atoms and deuterium atoms.

5. The heterocyclic compound of claim 1, wherein the heterocyclic compound represented by Chemical Formula 1 is represented by any one of the following compounds:

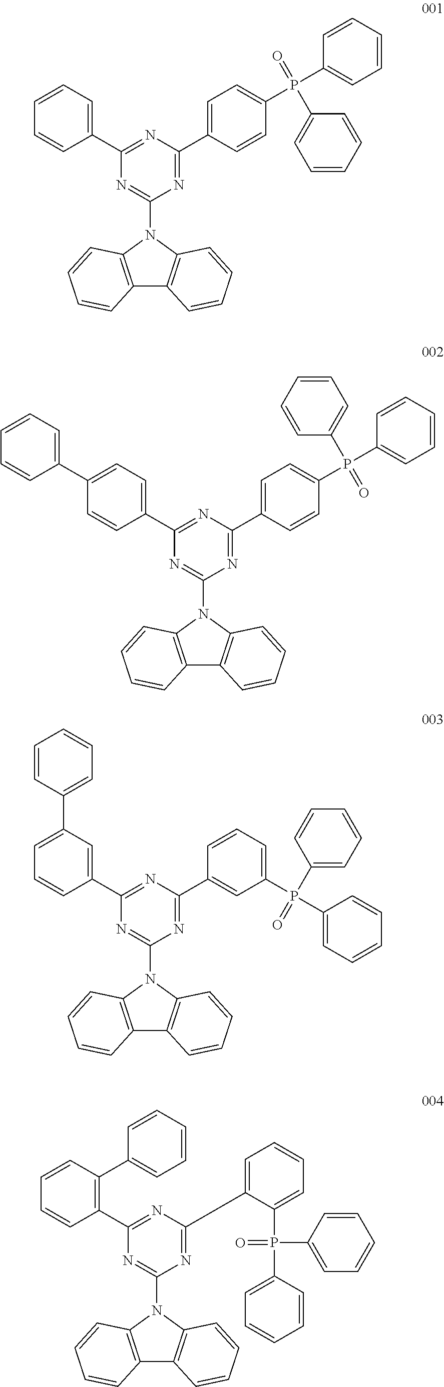 [SMILES: O=P(c1ccccc1)(c1ccccc1)c1ccc(-c2nc(-c3ccccc3)nc(-n3c4ccccc4c4ccccc43)n2)cc1 ; O=P(c1ccccc1)(c1ccccc1)c1ccc(-c2nc(-c3ccc(-c4ccccc4)cc3)nc(-n3c4ccccc4c4ccccc43)n2)cc1 ; O=P(c1ccccc1)(c1ccccc1)c1cccc(-c2nc(-c3cccc(-c4ccccc4)c3)nc(-n3c4ccccc4c4ccccc43)n2)c1 ; O=P(c1ccccc1)(c1ccccc1)c1ccccc1-c1nc(-c2ccccc2-c2ccccc2)nc(-n2c3ccccc3c3ccccc32)n1]

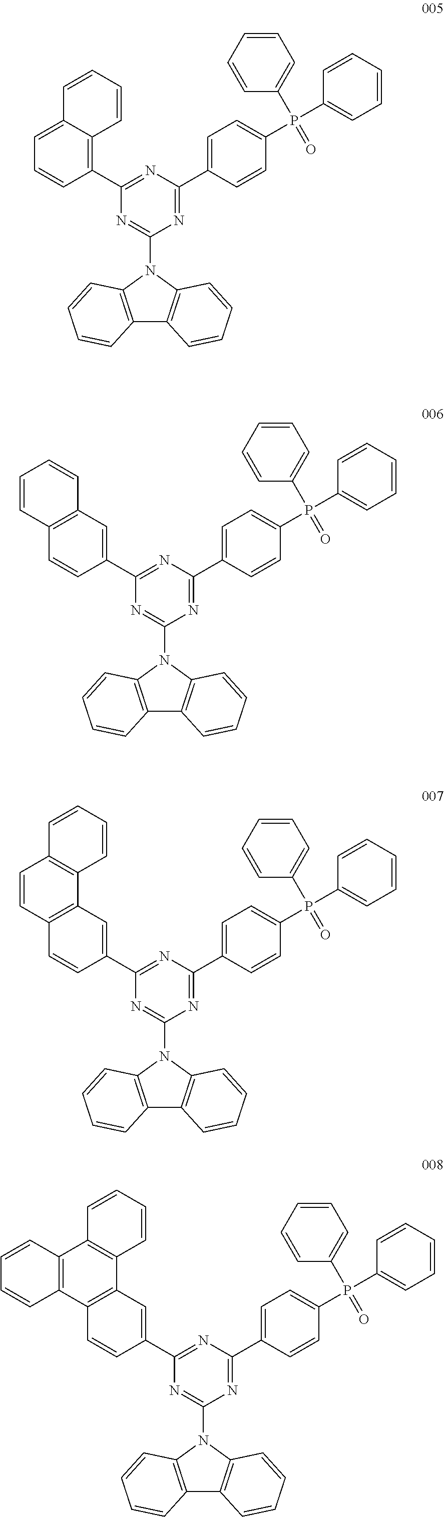 [SMILES: O=P(c1ccccc1)(c1ccccc1)c1ccc(-c2nc(-c3cccc4ccccc34)nc(-n3c4ccccc4c4ccccc43)n2)cc1 ; O=P(c1ccccc1)(c1ccccc1)c1ccc(-c2nc(-c3ccc4ccccc4c3)nc(-n3c4ccccc4c4ccccc43)n2)cc1 ; O=P(c1ccccc1)(c1ccccc1)c1ccc(-c2nc(-c3ccc4ccc5ccccc5c4c3)nc(-n3c4ccccc4c4ccccc43)n2)cc1 ; O=P(c1ccccc1)(c1ccccc1)c1ccc(-c2nc(-c3ccc4c5ccccc5c5ccccc5c4c3)nc(-n3c4ccccc4c4ccccc43)n2)cc1]

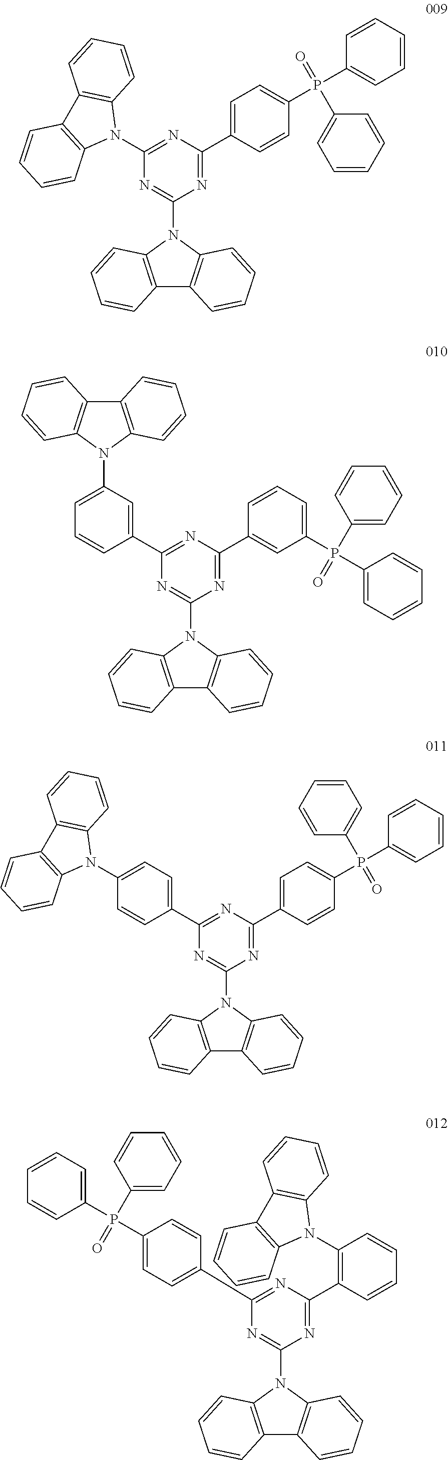 [SMILES: O=P(c1ccccc1)(c1ccccc1)c1ccc(-c2nc(-n3c4ccccc4c4ccccc43)nc(-n3c4ccccc4c4ccccc43)n2)cc1 ; O=P(c1ccccc1)(c1ccccc1)c1cccc(-c2nc(-c3cccc(-n4c5ccccc5c5ccccc54)c3)nc(-n3c4ccccc4c4ccccc43)n2)c1 ; O=P(c1ccccc1)(c1ccccc1)c1ccc(-c2nc(-c3ccc(-n4c5ccccc5c5ccccc54)cc3)nc(-n3c4ccccc4c4ccccc43)n2)cc1 ; O=P(c1ccccc1)(c1ccccc1)c1ccc(-c2nc(-c3ccccc3-n3c4ccccc4c4ccccc43)nc(-n3c4ccccc4c4ccccc43)n2)cc1]

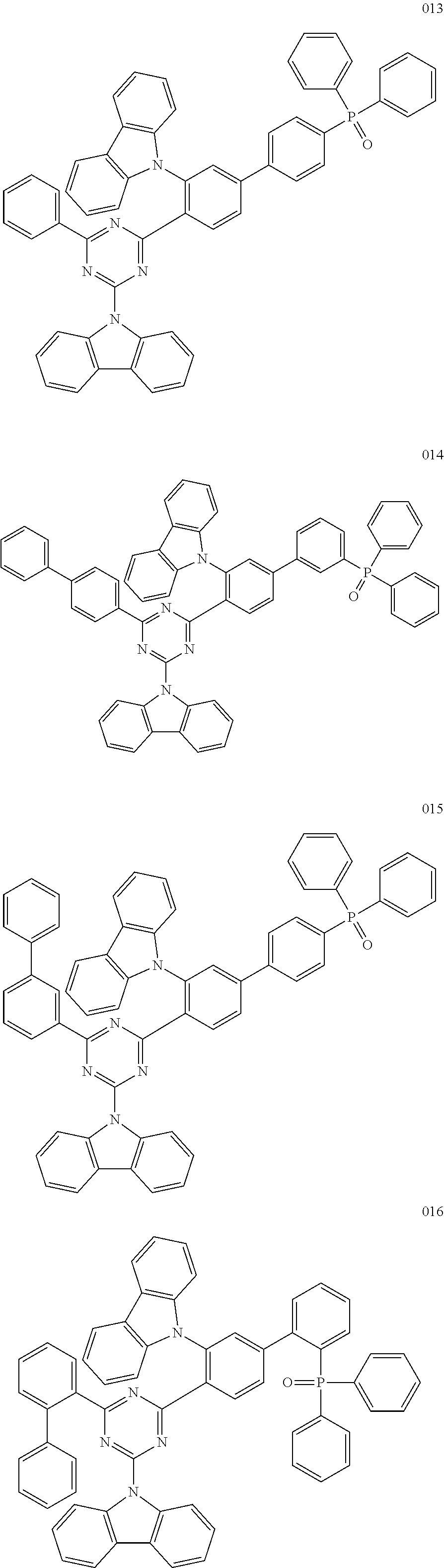

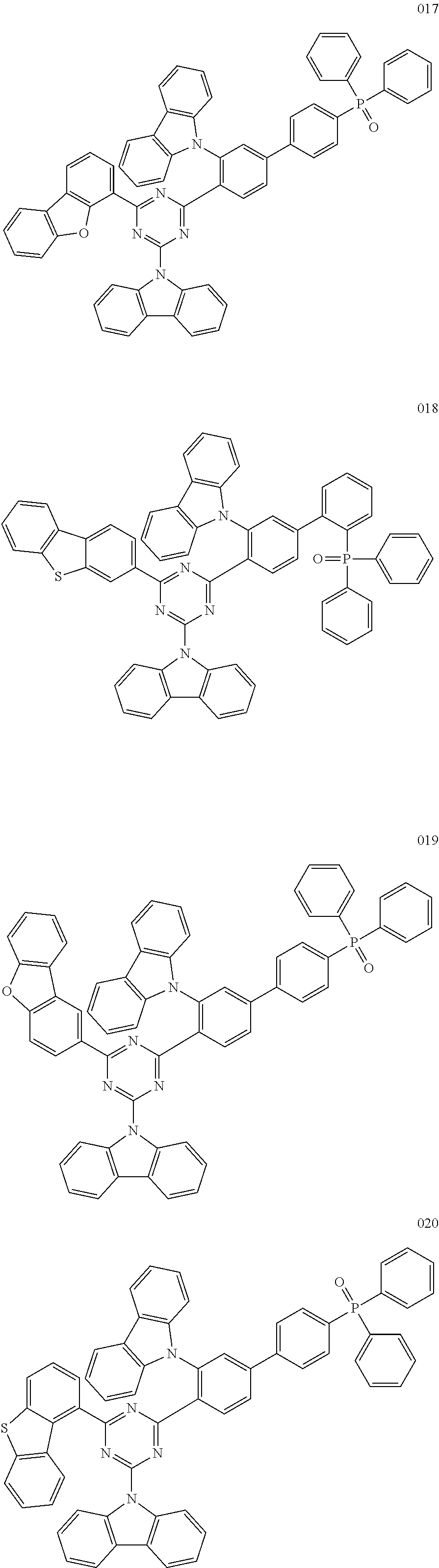 [SMILES: O=P(c1ccccc1)(c1ccccc1)c1ccc(-c2ccc(-c3nc(-c4cccc5c4oc4ccccc45)nc(-n4c5ccccc5c5ccccc54)n3)c(-n3c4ccccc4c4ccccc43)c2)cc1 ; O=P(c1ccccc1)(c1ccccc1)c1ccccc1-c1ccc(-c2nc(-c3ccc4c(c3)sc3ccccc34)nc(-n3c4ccccc4c4ccccc43)n2)c(-n2c3ccccc3c3ccccc32)c1 ; O=P(c1ccccc1)(c1ccccc1)c1ccc(-c2ccc(-c3nc(-c4ccc5oc6ccccc6c5c4)nc(-n4c5ccccc5c5ccccc54)n3)c(-n3c4ccccc4c4ccccc43)c2)cc1 ; O=P(c1ccccc1)(c1ccccc1)c1ccc(-c2ccc(-c3nc(-c4cccc5sc6ccccc6c45)nc(-n4c5ccccc5c5ccccc54)n3)c(-n3c4ccccc4c4ccccc43)c2)cc1]

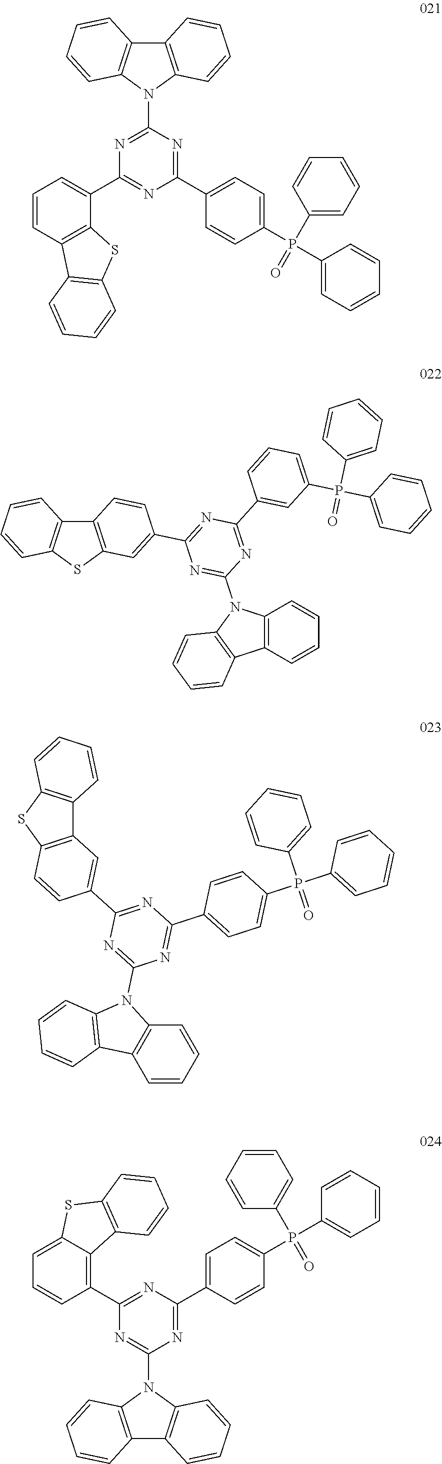 [SMILES: O=P(c1ccccc1)(c1ccccc1)c1ccc(-c2nc(-c3cccc4c3sc3ccccc34)nc(-n3c4ccccc4c4ccccc43)n2)cc1 ; O=P(c1ccccc1)(c1ccccc1)c1cccc(-c2nc(-c3ccc4c(c3)sc3ccccc34)nc(-n3c4ccccc4c4ccccc43)n2)c1 ; O=P(c1ccccc1)(c1ccccc1)c1ccc(-c2nc(-c3ccc4sc5ccccc5c4c3)nc(-n3c4ccccc4c4ccccc43)n2)cc1 ; O=P(c1ccccc1)(c1ccccc1)c1ccc(-c2nc(-c3cccc4sc5ccccc5c34)nc(-n3c4ccccc4c4ccccc43)n2)cc1]

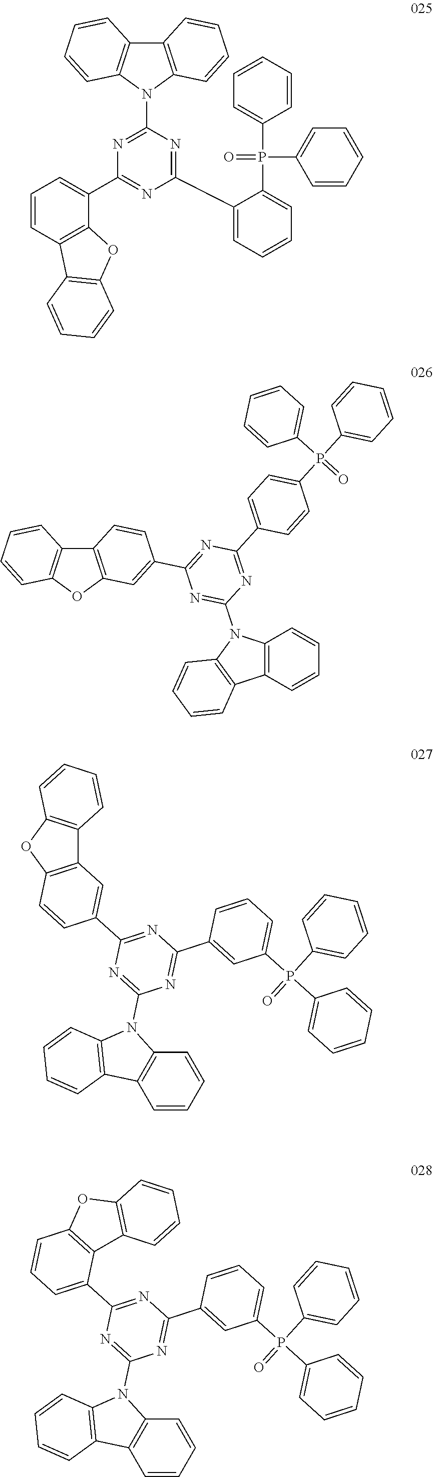 [SMILES: O=P(c1ccccc1)(c1ccccc1)c1ccccc1-c1nc(-c2cccc3c2oc2ccccc23)nc(-n2c3ccccc3c3ccccc32)n1 ; O=P(c1ccccc1)(c1ccccc1)c1ccc(-c2nc(-c3ccc4c(c3)oc3ccccc34)nc(-n3c4ccccc4c4ccccc43)n2)cc1 ; O=P(c1ccccc1)(c1ccccc1)c1cccc(-c2nc(-c3ccc4oc5ccccc5c4c3)nc(-n3c4ccccc4c4ccccc43)n2)c1 ; O=P(c1ccccc1)(c1ccccc1)c1cccc(-c2nc(-c3cccc4oc5ccccc5c34)nc(-n3c4ccccc4c4ccccc43)n2)c1]

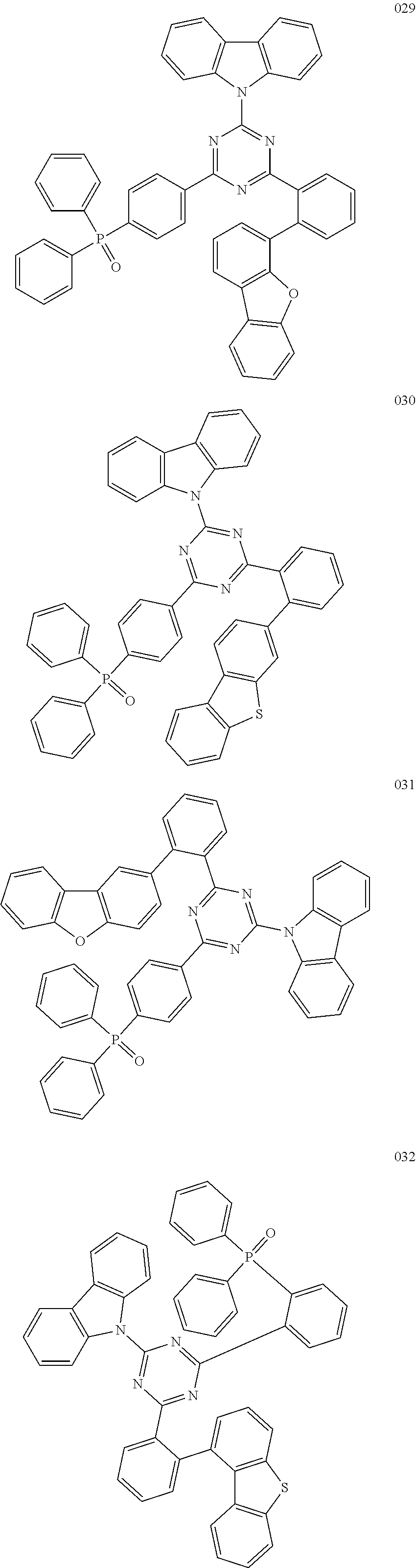

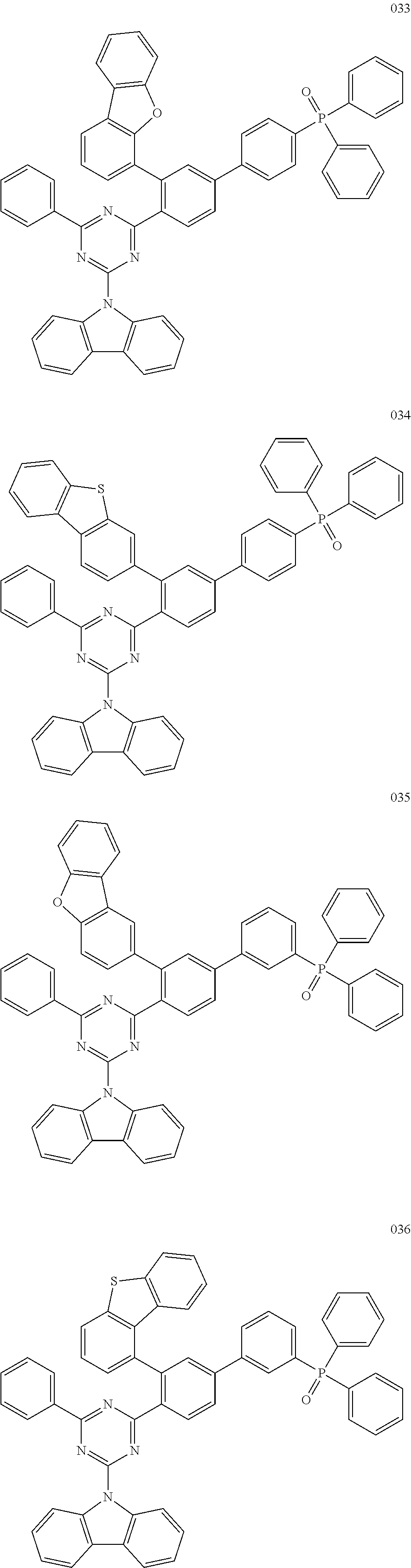 [SMILES: O=P(c1ccccc1)(c1ccccc1)c1ccc(-c2ccc(-c3nc(-c4ccccc4)nc(-n4c5ccccc5c5ccccc54)n3)c(-c3cccc4c3oc3ccccc34)c2)cc1 ; O=P(c1ccccc1)(c1ccccc1)c1ccc(-c2ccc(-c3nc(-c4ccccc4)nc(-n4c5ccccc5c5ccccc54)n3)c(-c3ccc4c(c3)sc3ccccc34)c2)cc1 ; O=P(c1ccccc1)(c1ccccc1)c1cccc(-c2ccc(-c3nc(-c4ccccc4)nc(-n4c5ccccc5c5ccccc54)n3)c(-c3ccc4oc5ccccc5c4c3)c2)c1 ; O=P(c1ccccc1)(c1ccccc1)c1cccc(-c2ccc(-c3nc(-c4ccccc4)nc(-n4c5ccccc5c5ccccc54)n3)c(-c3cccc4sc5ccccc5c34)c2)c1]

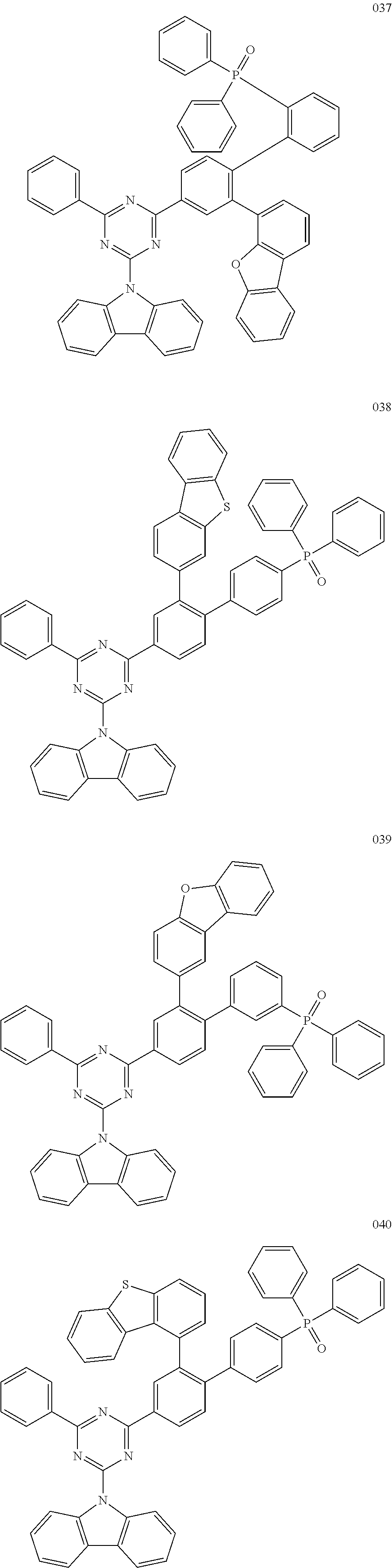 [SMILES: O=P(c1ccccc1)(c1ccccc1)c1ccccc1-c1ccc(-c2nc(-c3ccccc3)nc(-n3c4ccccc4c4ccccc43)n2)cc1-c1cccc2c1oc1ccccc12 ; O=P(c1ccccc1)(c1ccccc1)c1ccc(-c2ccc(-c3nc(-c4ccccc4)nc(-n4c5ccccc5c5ccccc54)n3)cc2-c2ccc3c(c2)sc2ccccc23)cc1 ; O=P(c1ccccc1)(c1ccccc1)c1cccc(-c2ccc(-c3nc(-c4ccccc4)nc(-n4c5ccccc5c5ccccc54)n3)cc2-c2ccc3oc4ccccc4c3c2)c1 ; O=P(c1ccccc1)(c1ccccc1)c1ccc(-c2ccc(-c3nc(-c4ccccc4)nc(-n4c5ccccc5c5ccccc54)n3)cc2-c2cccc3sc4ccccc4c23)cc1]

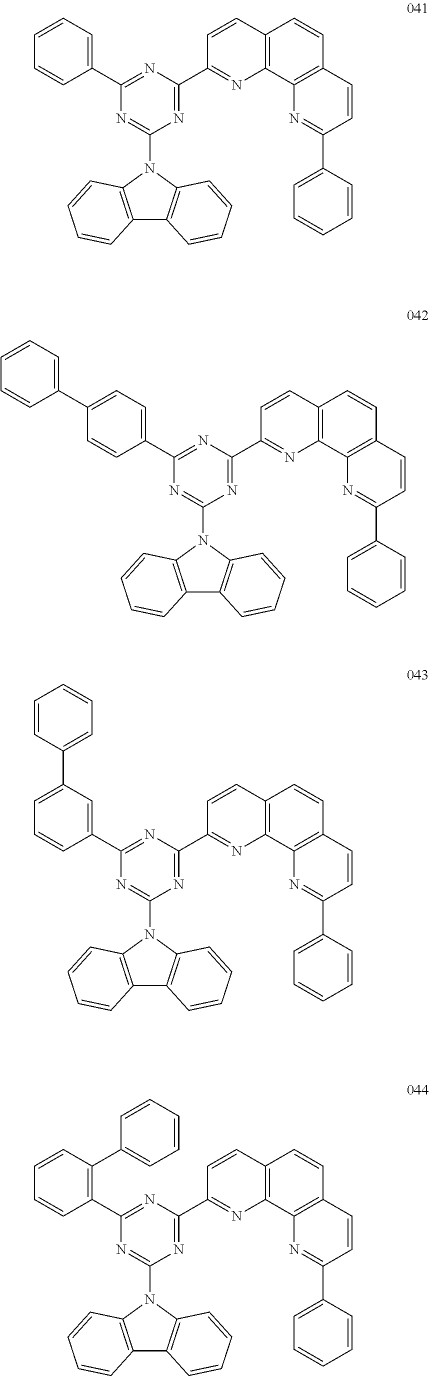 [SMILES: c1ccc(-c2ccc3ccc4ccc(-c5nc(-c6ccccc6)nc(-n6c7ccccc7c7ccccc76)n5)nc4c3n2)cc1 ; c1ccc(-c2ccc(-c3nc(-c4ccc5ccc6ccc(-c7ccccc7)nc6c5n4)nc(-n4c5ccccc5c5ccccc54)n3)cc2)cc1 ; c1ccc(-c2cccc(-c3nc(-c4ccc5ccc6ccc(-c7ccccc7)nc6c5n4)nc(-n4c5ccccc5c5ccccc54)n3)c2)cc1 ; c1ccc(-c2ccc3ccc4ccc(-c5nc(-c6ccccc6-c6ccccc6)nc(-n6c7ccccc7c7ccccc76)n5)nc4c3n2)cc1]

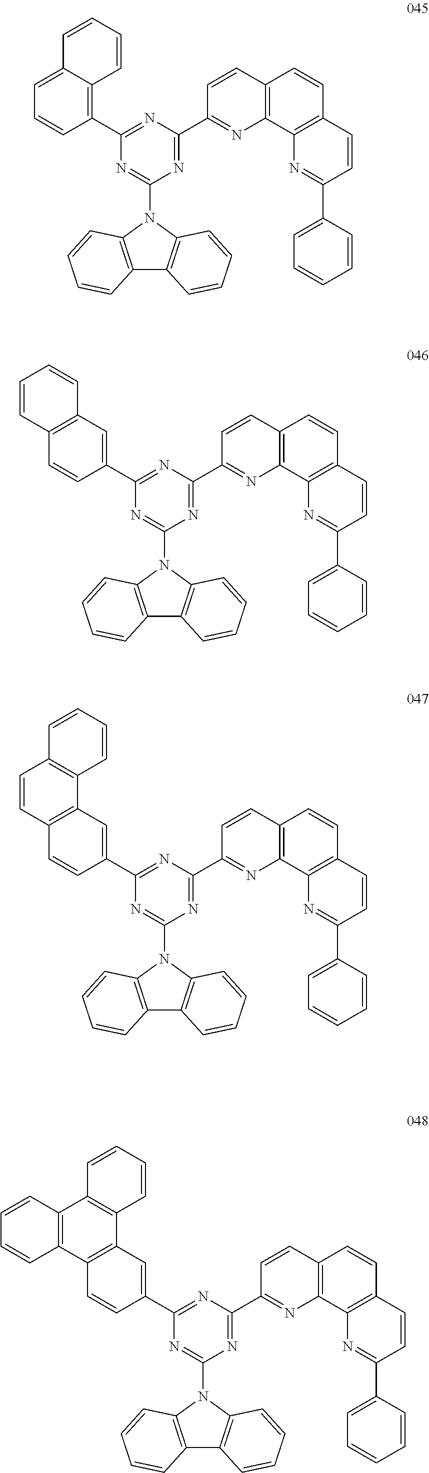 [SMILES: c1ccc(-c2ccc3ccc4ccc(-c5nc(-c6cccc7ccccc67)nc(-n6c7ccccc7c7ccccc76)n5)nc4c3n2)cc1 ; c1ccc(-c2ccc3ccc4ccc(-c5nc(-c6ccc7ccccc7c6)nc(-n6c7ccccc7c7ccccc76)n5)nc4c3n2)cc1 ; c1ccc(-c2ccc3ccc4ccc(-c5nc(-c6ccc7ccc8ccccc8c7c6)nc(-n6c7ccccc7c7ccccc76)n5)nc4c3n2)cc1 ; c1ccc(-c2ccc3ccc4ccc(-c5nc(-c6ccc7c8ccccc8c8ccccc8c7c6)nc(-n6c7ccccc7c7ccccc76)n5)nc4c3n2)cc1]

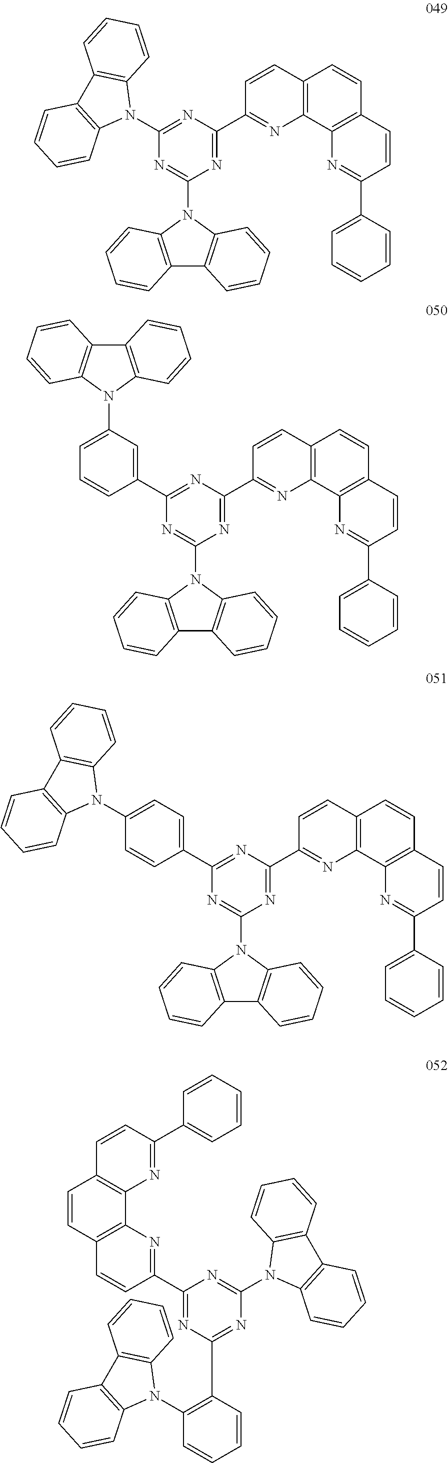

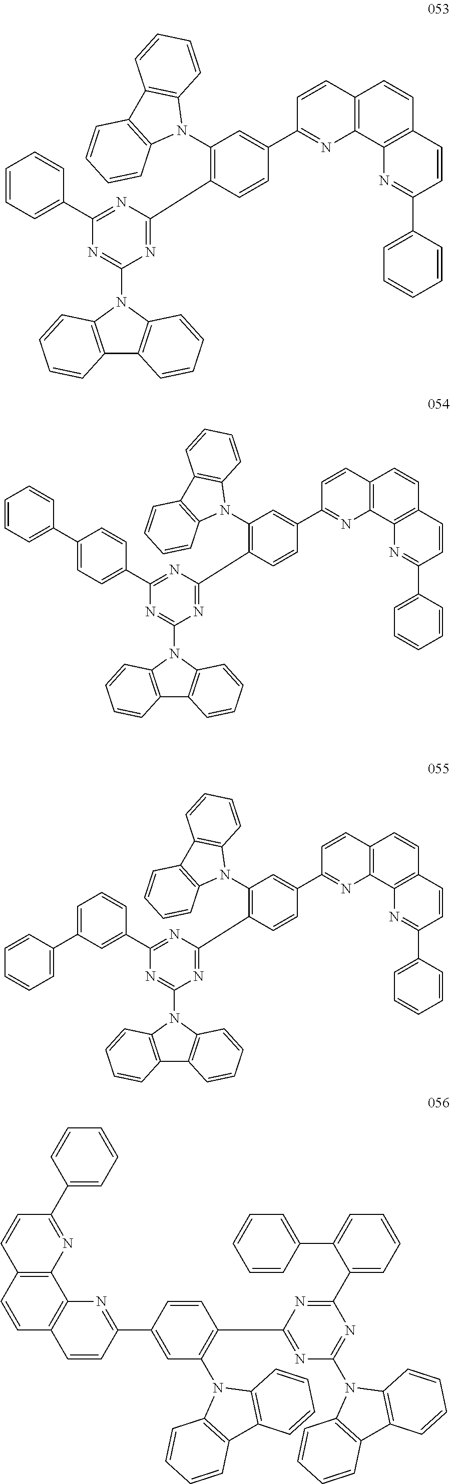 [SMILES: c1ccc(-c2ccc3ccc4ccc(-c5ccc(-c6nc(-c7ccccc7)nc(-n7c8ccccc8c8ccccc87)n6)c(-n6c7ccccc7c7ccccc76)c5)nc4c3n2)cc1 ; c1ccc(-c2ccc(-c3nc(-c4ccc(-c5ccc6ccc7ccc(-c8ccccc8)nc7c6n5)cc4-n4c5ccccc5c5ccccc54)nc(-n4c5ccccc5c5ccccc54)n3)cc2)cc1 ; c1ccc(-c2cccc(-c3nc(-c4ccc(-c5ccc6ccc7ccc(-c8ccccc8)nc7c6n5)cc4-n4c5ccccc5c5ccccc54)nc(-n4c5ccccc5c5ccccc54)n3)c2)cc1 ; c1ccc(-c2ccc3ccc4ccc(-c5ccc(-c6nc(-c7ccccc7-c7ccccc7)nc(-n7c8ccccc8c8ccccc87)n6)c(-n6c7ccccc7c7ccccc76)c5)nc4c3n2)cc1]

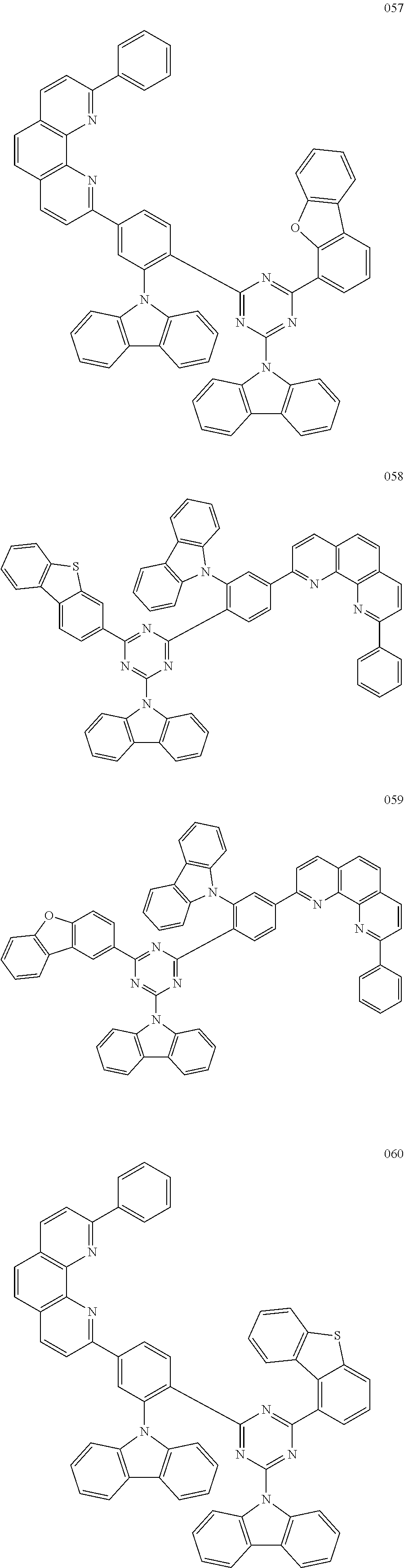 [SMILES: c1ccc(-c2ccc3ccc4ccc(-c5ccc(-c6nc(-c7cccc8c7oc7ccccc78)nc(-n7c8ccccc8c8ccccc87)n6)c(-n6c7ccccc7c7ccccc76)c5)nc4c3n2)cc1 ; c1ccc(-c2ccc3ccc4ccc(-c5ccc(-c6nc(-c7ccc8c(c7)sc7ccccc78)nc(-n7c8ccccc8c8ccccc87)n6)c(-n6c7ccccc7c7ccccc76)c5)nc4c3n2)cc1 ; c1ccc(-c2ccc3ccc4ccc(-c5ccc(-c6nc(-c7ccc8oc9ccccc9c8c7)nc(-n7c8ccccc8c8ccccc87)n6)c(-n6c7ccccc7c7ccccc76)c5)nc4c3n2)cc1 ; c1ccc(-c2ccc3ccc4ccc(-c5ccc(-c6nc(-c7cccc8sc9ccccc9c78)nc(-n7c8ccccc8c8ccccc87)n6)c(-n6c7ccccc7c7ccccc76)c5)nc4c3n2)cc1]

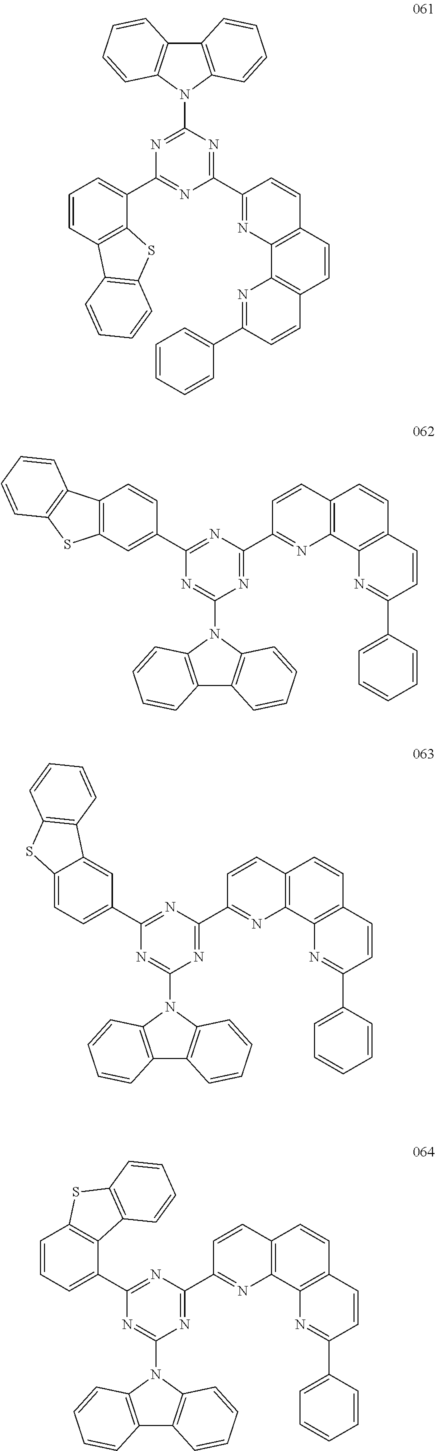 [SMILES: c1ccc(-c2ccc3ccc4ccc(-c5nc(-c6cccc7c6sc6ccccc67)nc(-n6c7ccccc7c7ccccc76)n5)nc4c3n2)cc1 ; c1ccc(-c2ccc3ccc4ccc(-c5nc(-c6ccc7c(c6)sc6ccccc67)nc(-n6c7ccccc7c7ccccc76)n5)nc4c3n2)cc1 ; c1ccc(-c2ccc3ccc4ccc(-c5nc(-c6ccc7sc8ccccc8c7c6)nc(-n6c7ccccc7c7ccccc76)n5)nc4c3n2)cc1 ; c1ccc(-c2ccc3ccc4ccc(-c5nc(-c6cccc7sc8ccccc8c67)nc(-n6c7ccccc7c7ccccc76)n5)nc4c3n2)cc1]

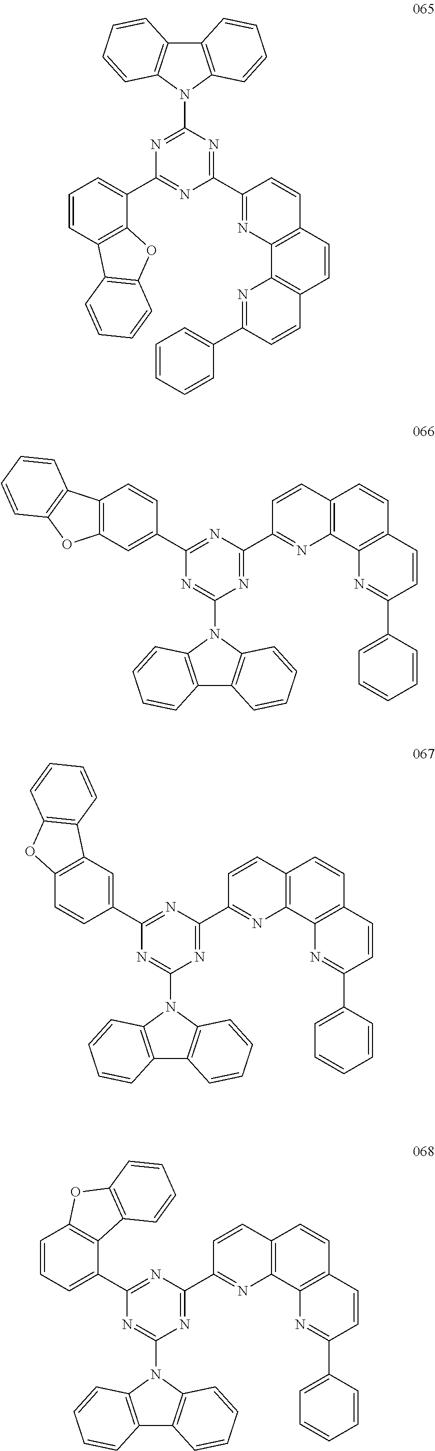 [SMILES: c1ccc(-c2ccc3ccc4ccc(-c5nc(-c6cccc7c6oc6ccccc67)nc(-n6c7ccccc7c7ccccc76)n5)nc4c3n2)cc1 ; c1ccc(-c2ccc3ccc4ccc(-c5nc(-c6ccc7c(c6)oc6ccccc67)nc(-n6c7ccccc7c7ccccc76)n5)nc4c3n2)cc1 ; c1ccc(-c2ccc3ccc4ccc(-c5nc(-c6ccc7oc8ccccc8c7c6)nc(-n6c7ccccc7c7ccccc76)n5)nc4c3n2)cc1 ; c1ccc(-c2ccc3ccc4ccc(-c5nc(-c6cccc7oc8ccccc8c67)nc(-n6c7ccccc7c7ccccc76)n5)nc4c3n2)cc1]

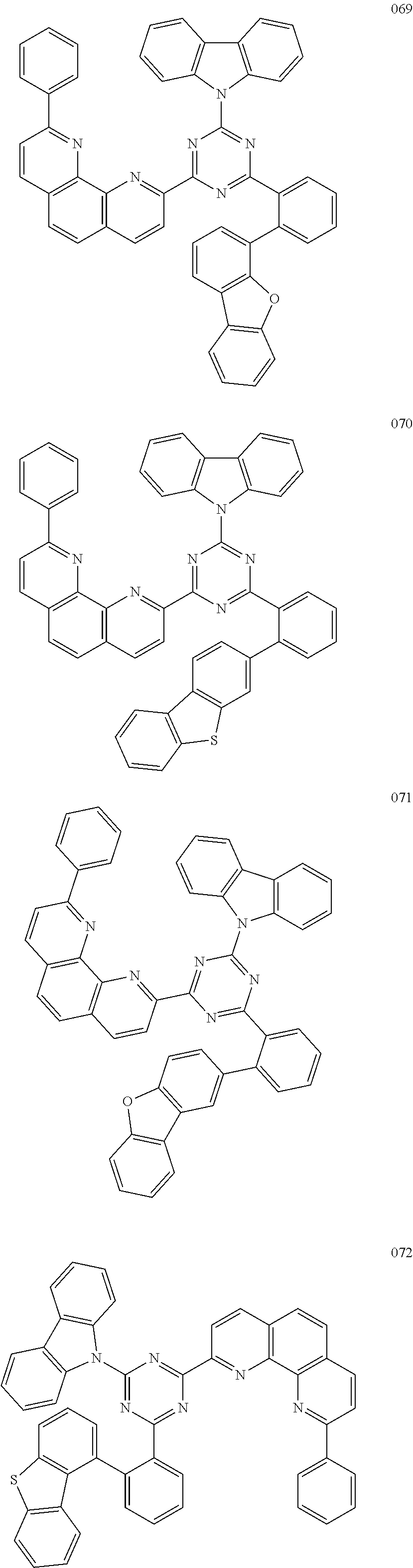 [SMILES: c1ccc(-c2ccc3ccc4ccc(-c5nc(-c6ccccc6-c6cccc7c6oc6ccccc67)nc(-n6c7ccccc7c7ccccc76)n5)nc4c3n2)cc1 ; c1ccc(-c2ccc3ccc4ccc(-c5nc(-c6ccccc6-c6ccc7c(c6)sc6ccccc67)nc(-n6c7ccccc7c7ccccc76)n5)nc4c3n2)cc1 ; c1ccc(-c2ccc3ccc4ccc(-c5nc(-c6ccccc6-c6ccc7oc8ccccc8c7c6)nc(-n6c7ccccc7c7ccccc76)n5)nc4c3n2)cc1 ; c1ccc(-c2ccc3ccc4ccc(-c5nc(-c6ccccc6-c6cccc7sc8ccccc8c67)nc(-n6c7ccccc7c7ccccc76)n5)nc4c3n2)cc1]

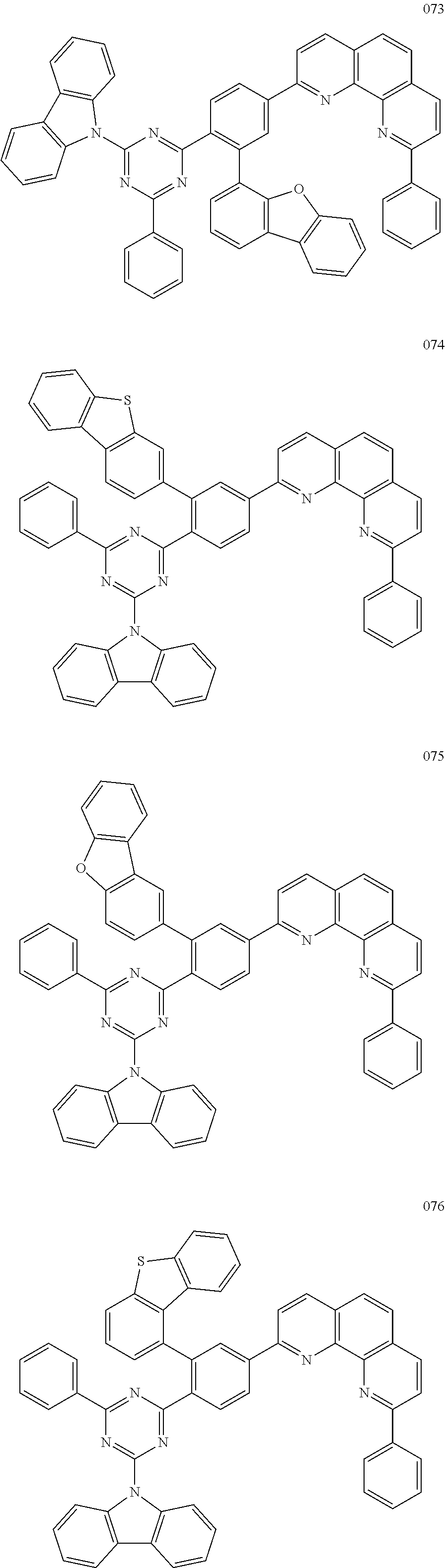 [SMILES: c1ccc(-c2ccc3ccc4ccc(-c5ccc(-c6nc(-c7ccccc7)nc(-n7c8ccccc8c8ccccc87)n6)c(-c6cccc7c6oc6ccccc67)c5)nc4c3n2)cc1 ; c1ccc(-c2ccc3ccc4ccc(-c5ccc(-c6nc(-c7ccccc7)nc(-n7c8ccccc8c8ccccc87)n6)c(-c6ccc7c(c6)sc6ccccc67)c5)nc4c3n2)cc1 ; c1ccc(-c2ccc3ccc4ccc(-c5ccc(-c6nc(-c7ccccc7)nc(-n7c8ccccc8c8ccccc87)n6)c(-c6ccc7oc8ccccc8c7c6)c5)nc4c3n2)cc1 ; c1ccc(-c2ccc3ccc4ccc(-c5ccc(-c6nc(-c7ccccc7)nc(-n7c8ccccc8c8ccccc87)n6)c(-c6cccc7sc8ccccc8c67)c5)nc4c3n2)cc1]

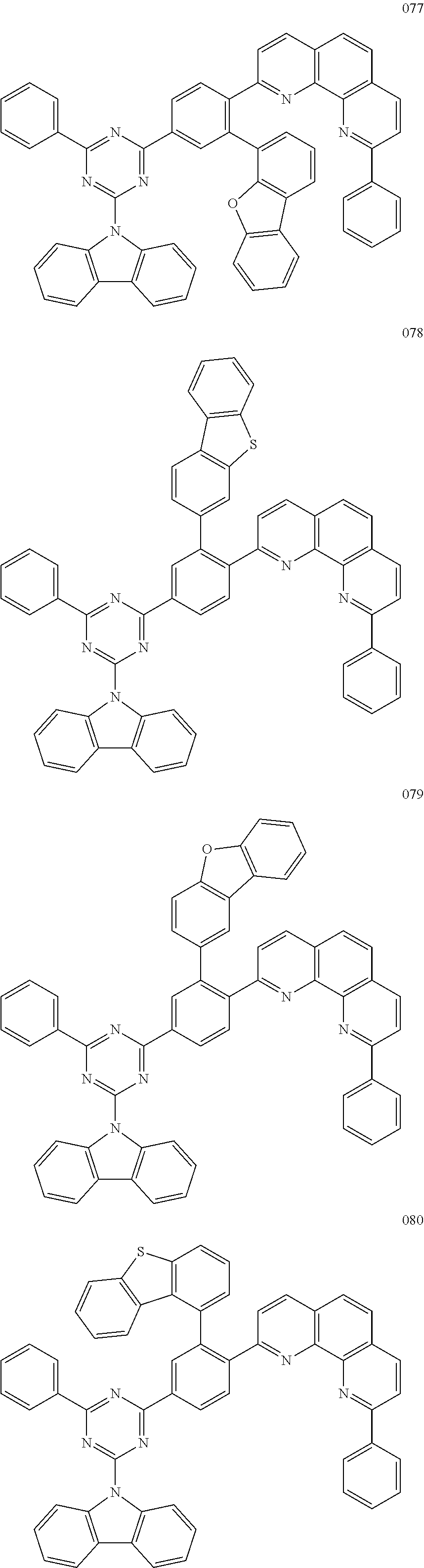 [SMILES: c1ccc(-c2ccc3ccc4ccc(-c5ccc(-c6nc(-c7ccccc7)nc(-n7c8ccccc8c8ccccc87)n6)cc5-c5cccc6c5oc5ccccc56)nc4c3n2)cc1 ; c1ccc(-c2ccc3ccc4ccc(-c5ccc(-c6nc(-c7ccccc7)nc(-n7c8ccccc8c8ccccc87)n6)cc5-c5ccc6c(c5)sc5ccccc56)nc4c3n2)cc1 ; c1ccc(-c2ccc3ccc4ccc(-c5ccc(-c6nc(-c7ccccc7)nc(-n7c8ccccc8c8ccccc87)n6)cc5-c5ccc6oc7ccccc7c6c5)nc4c3n2)cc1 ; c1ccc(-c2ccc3ccc4ccc(-c5ccc(-c6nc(-c7ccccc7)nc(-n7c8ccccc8c8ccccc87)n6)cc5-c5cccc6sc7ccccc7c56)nc4c3n2)cc1]

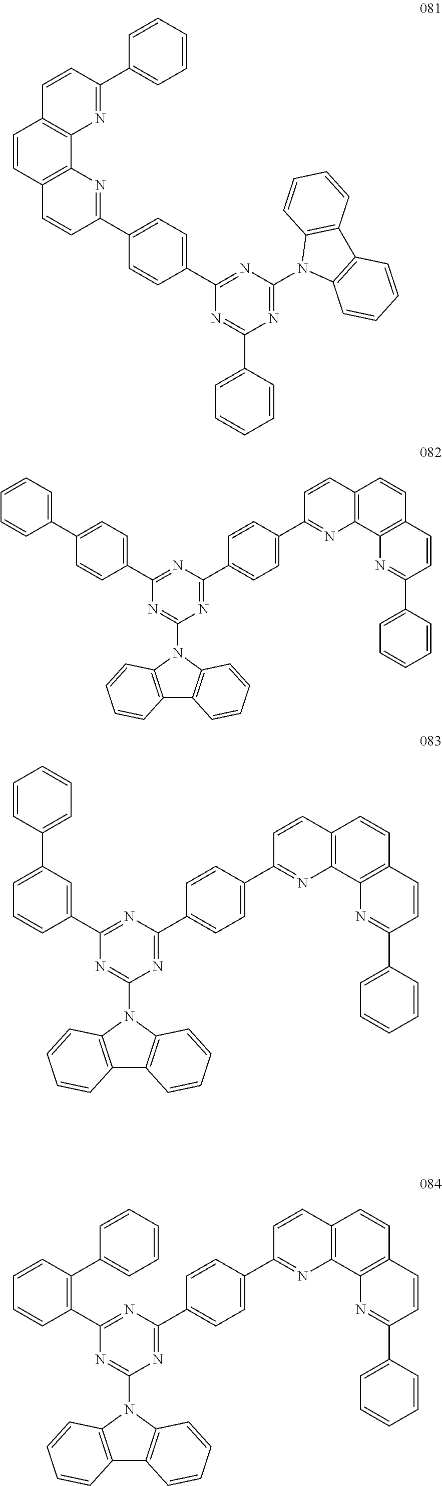 [SMILES: c1ccc(-c2ccc3ccc4ccc(-c5ccc(-c6nc(-c7ccccc7)nc(-n7c8ccccc8c8ccccc87)n6)cc5)nc4c3n2)cc1 ; c1ccc(-c2ccc(-c3nc(-c4ccc(-c5ccc6ccc7ccc(-c8ccccc8)nc7c6n5)cc4)nc(-n4c5ccccc5c5ccccc54)n3)cc2)cc1 ; c1ccc(-c2cccc(-c3nc(-c4ccc(-c5ccc6ccc7ccc(-c8ccccc8)nc7c6n5)cc4)nc(-n4c5ccccc5c5ccccc54)n3)c2)cc1 ; c1ccc(-c2ccc3ccc4ccc(-c5ccc(-c6nc(-c7ccccc7-c7ccccc7)nc(-n7c8ccccc8c8ccccc87)n6)cc5)nc4c3n2)cc1]

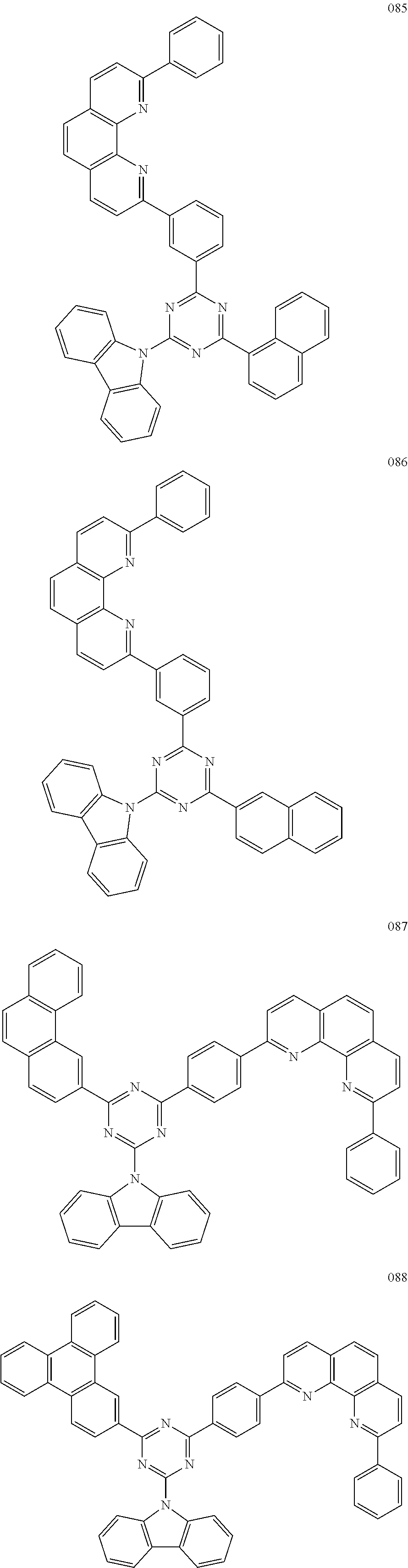 [SMILES: c1ccc(-c2ccc3ccc4ccc(-c5cccc(-c6nc(-c7cccc8ccccc78)nc(-n7c8ccccc8c8ccccc87)n6)c5)nc4c3n2)cc1 ; c1ccc(-c2ccc3ccc4ccc(-c5cccc(-c6nc(-c7ccc8ccccc8c7)nc(-n7c8ccccc8c8ccccc87)n6)c5)nc4c3n2)cc1 ; c1ccc(-c2ccc3ccc4ccc(-c5ccc(-c6nc(-c7ccc8ccc9ccccc9c8c7)nc(-n7c8ccccc8c8ccccc87)n6)cc5)nc4c3n2)cc1 ; c1ccc(-c2ccc3ccc4ccc(-c5ccc(-c6nc(-c7ccc8c9ccccc9c9ccccc9c8c7)nc(-n7c8ccccc8c8ccccc87)n6)cc5)nc4c3n2)cc1]

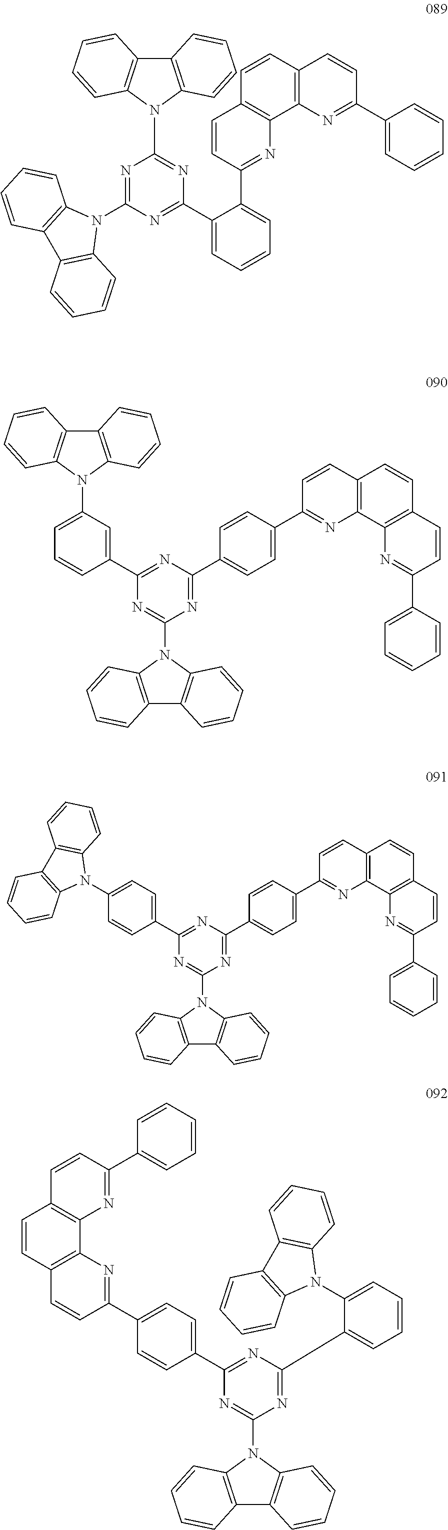

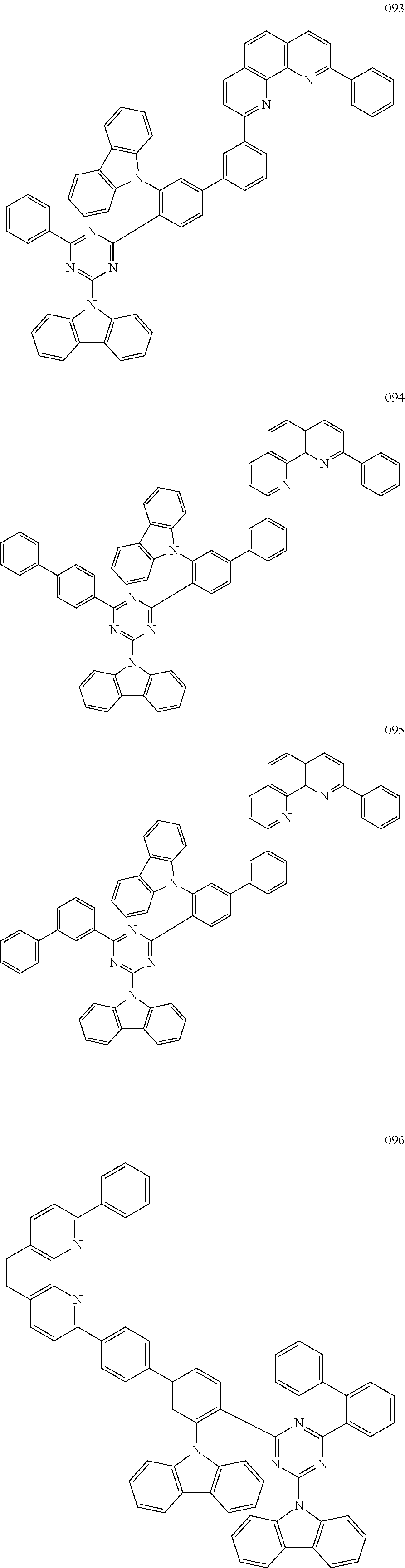

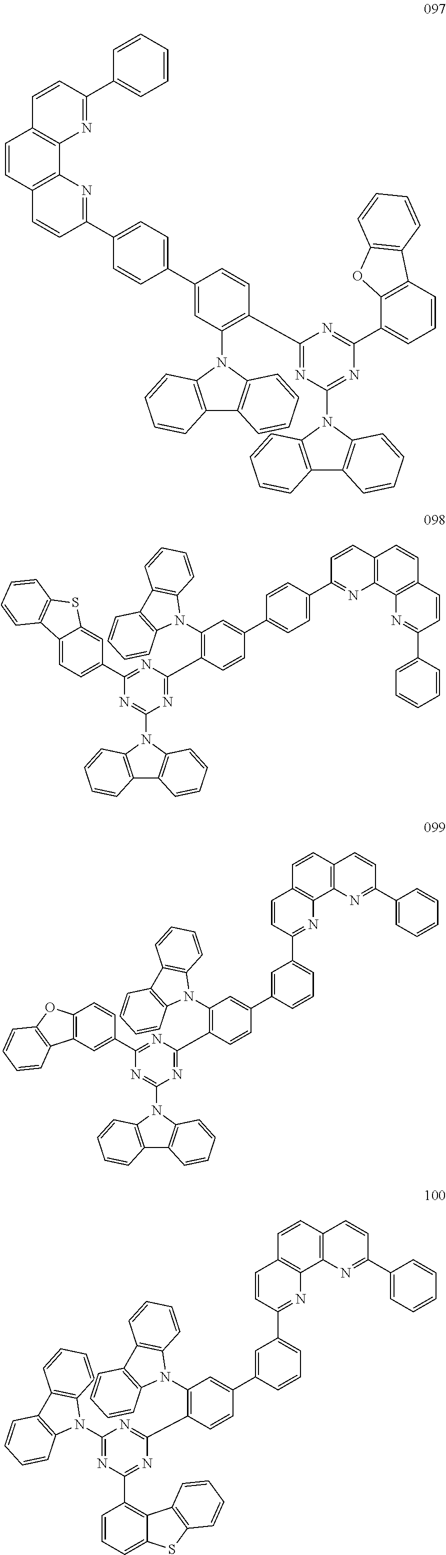

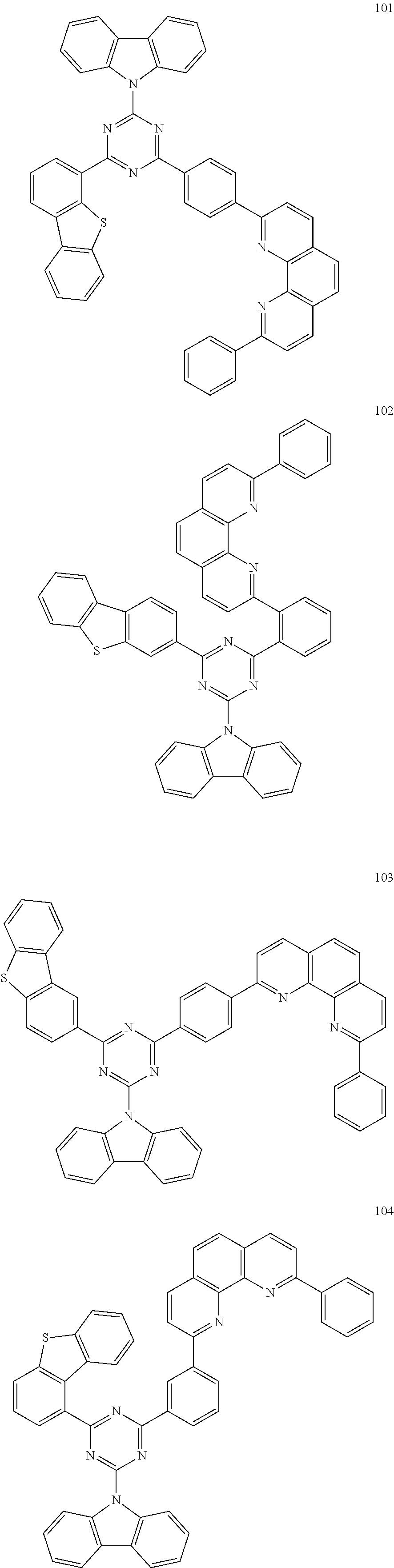 [SMILES: c1ccc(-c2ccc3ccc4ccc(-c5ccc(-c6nc(-c7cccc8c7sc7ccccc78)nc(-n7c8ccccc8c8ccccc87)n6)cc5)nc4c3n2)cc1 ; c1ccc(-c2ccc3ccc4ccc(-c5ccccc5-c5nc(-c6ccc7c(c6)sc6ccccc67)nc(-n6c7ccccc7c7ccccc76)n5)nc4c3n2)cc1 ; c1ccc(-c2ccc3ccc4ccc(-c5ccc(-c6nc(-c7ccc8sc9ccccc9c8c7)nc(-n7c8ccccc8c8ccccc87)n6)cc5)nc4c3n2)cc1 ; c1ccc(-c2ccc3ccc4ccc(-c5cccc(-c6nc(-c7cccc8sc9ccccc9c78)nc(-n7c8ccccc8c8ccccc87)n6)c5)nc4c3n2)cc1]

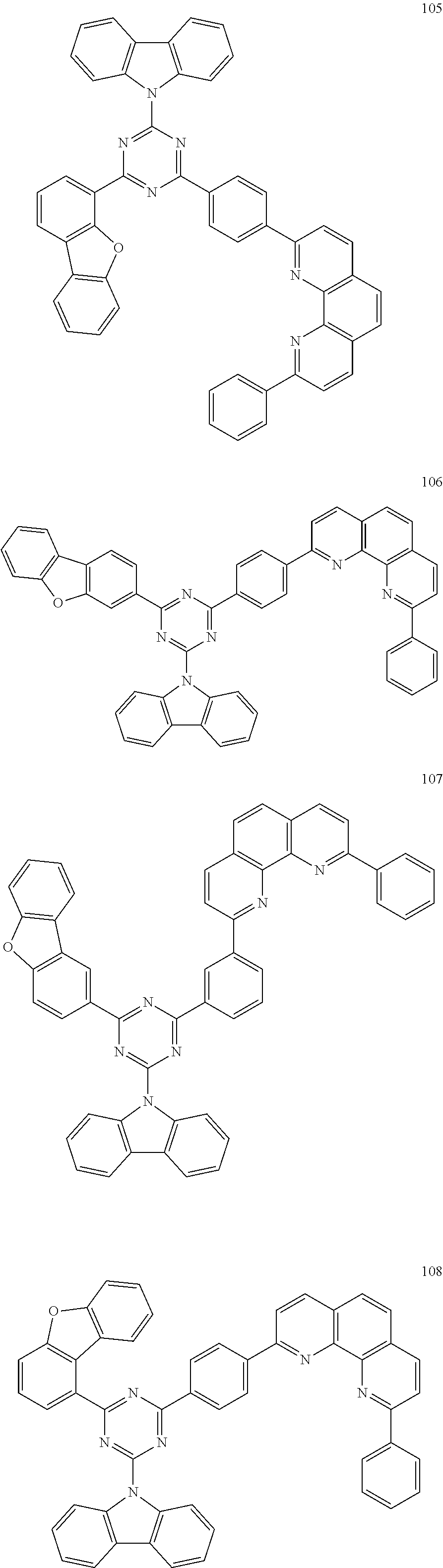 [SMILES: c1ccc(-c2ccc3ccc4ccc(-c5ccc(-c6nc(-c7cccc8c7oc7ccccc78)nc(-n7c8ccccc8c8ccccc87)n6)cc5)nc4c3n2)cc1 ; c1ccc(-c2ccc3ccc4ccc(-c5ccc(-c6nc(-c7ccc8c(c7)oc7ccccc78)nc(-n7c8ccccc8c8ccccc87)n6)cc5)nc4c3n2)cc1 ; c1ccc(-c2ccc3ccc4ccc(-c5cccc(-c6nc(-c7ccc8oc9ccccc9c8c7)nc(-n7c8ccccc8c8ccccc87)n6)c5)nc4c3n2)cc1 ; c1ccc(-c2ccc3ccc4ccc(-c5ccc(-c6nc(-c7cccc8oc9ccccc9c78)nc(-n7c8ccccc8c8ccccc87)n6)cc5)nc4c3n2)cc1]

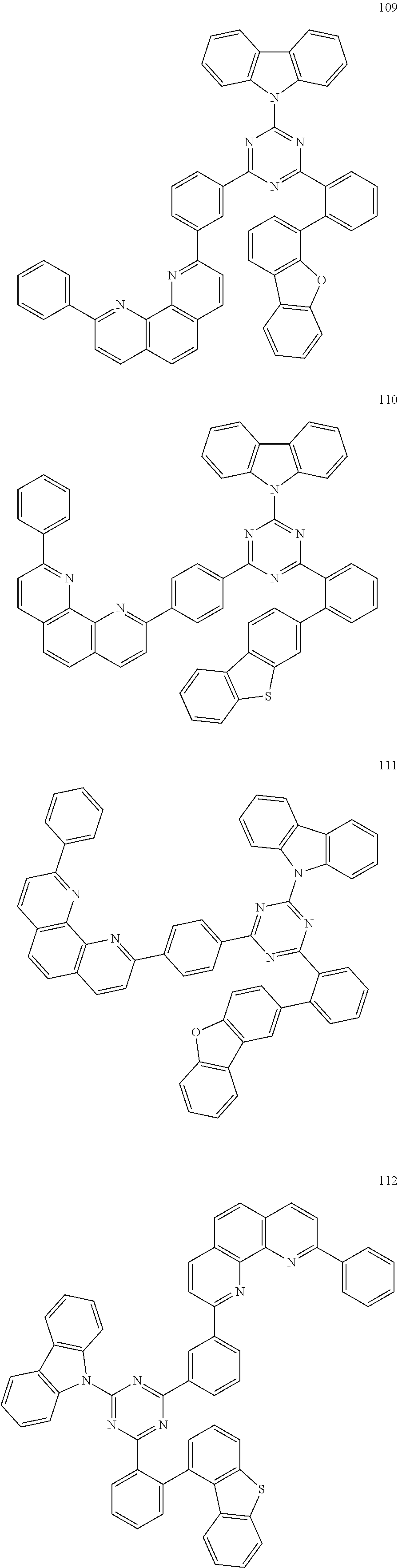 [SMILES: c1ccc(-c2ccc3ccc4ccc(-c5cccc(-c6nc(-c7ccccc7-c7cccc8c7oc7ccccc78)nc(-n7c8ccccc8c8ccccc87)n6)c5)nc4c3n2)cc1 ; c1ccc(-c2ccc3ccc4ccc(-c5ccc(-c6nc(-c7ccccc7-c7ccc8c(c7)sc7ccccc78)nc(-n7c8ccccc8c8ccccc87)n6)cc5)nc4c3n2)cc1 ; c1ccc(-c2ccc3ccc4ccc(-c5ccc(-c6nc(-c7ccccc7-c7ccc8oc9ccccc9c8c7)nc(-n7c8ccccc8c8ccccc87)n6)cc5)nc4c3n2)cc1 ; c1ccc(-c2ccc3ccc4ccc(-c5cccc(-c6nc(-c7ccccc7-c7cccc8sc9ccccc9c78)nc(-n7c8ccccc8c8ccccc87)n6)c5)nc4c3n2)cc1]

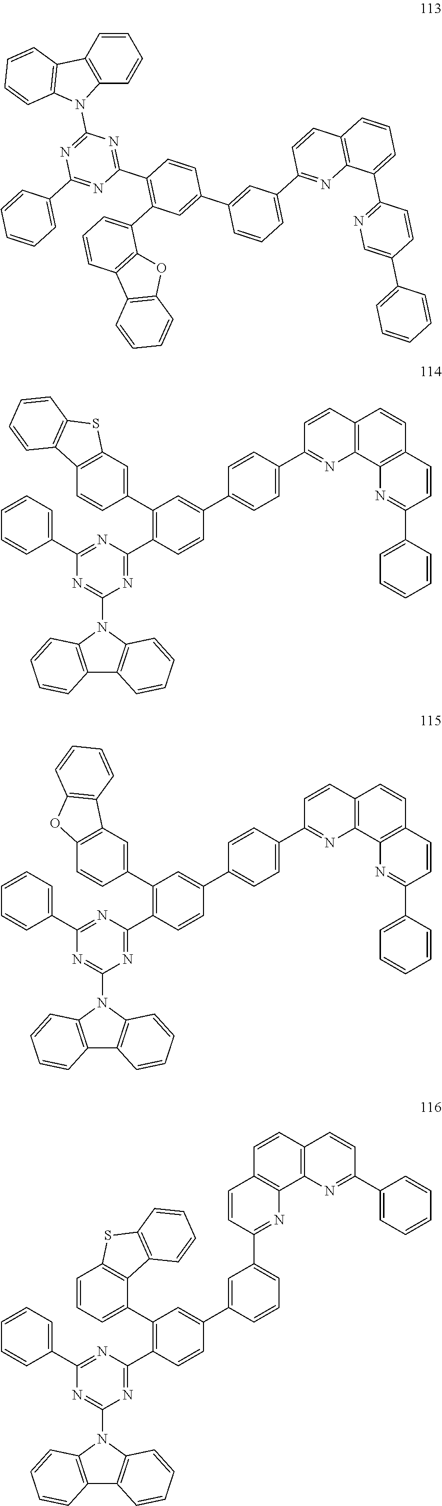 [SMILES: c1ccc(-c2ccc(-c3cccc4ccc(-c5cccc(-c6ccc(-c7nc(-c8ccccc8)nc(-n8c9ccccc9c9ccccc98)n7)c(-c7cccc8c7oc7ccccc78)c6)c5)nc34)nc2)cc1 ; c1ccc(-c2ccc3ccc4ccc(-c5ccc(-c6ccc(-c7nc(-c8ccccc8)nc(-n8c9ccccc9c9ccccc98)n7)c(-c7ccc8c(c7)sc7ccccc78)c6)cc5)nc4c3n2)cc1 ; c1ccc(-c2ccc3ccc4ccc(-c5ccc(-c6ccc(-c7nc(-c8ccccc8)nc(-n8c9ccccc9c9ccccc98)n7)c(-c7ccc8oc9ccccc9c8c7)c6)cc5)nc4c3n2)cc1 ; c1ccc(-c2ccc3ccc4ccc(-c5cccc(-c6ccc(-c7nc(-c8ccccc8)nc(-n8c9ccccc9c9ccccc98)n7)c(-c7cccc8sc9ccccc9c78)c6)c5)nc4c3n2)cc1]

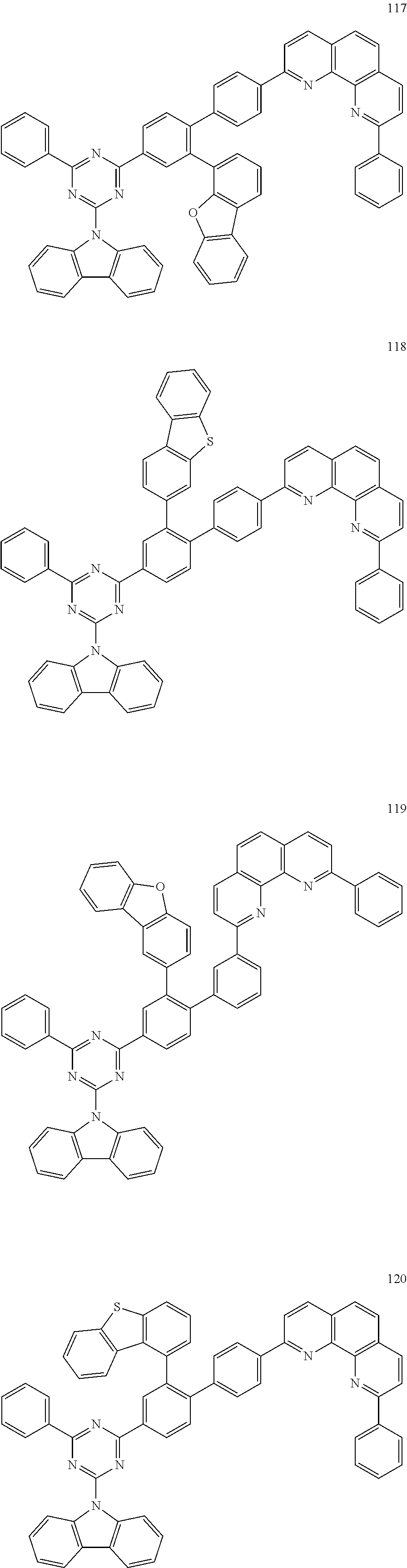 [SMILES: c1ccc(-c2ccc3ccc4ccc(-c5ccc(-c6ccc(-c7nc(-c8ccccc8)nc(-n8c9ccccc9c9ccccc98)n7)cc6-c6cccc7c6oc6ccccc67)cc5)nc4c3n2)cc1 ; c1ccc(-c2ccc3ccc4ccc(-c5ccc(-c6ccc(-c7nc(-c8ccccc8)nc(-n8c9ccccc9c9ccccc98)n7)cc6-c6ccc7c(c6)sc6ccccc67)cc5)nc4c3n2)cc1 ; c1ccc(-c2ccc3ccc4ccc(-c5cccc(-c6ccc(-c7nc(-c8ccccc8)nc(-n8c9ccccc9c9ccccc98)n7)cc6-c6ccc7oc8ccccc8c7c6)c5)nc4c3n2)cc1 ; c1ccc(-c2ccc3ccc4ccc(-c5ccc(-c6ccc(-c7nc(-c8ccccc8)nc(-n8c9ccccc9c9ccccc98)n7)cc6-c6cccc7sc8ccccc8c67)cc5)nc4c3n2)cc1]

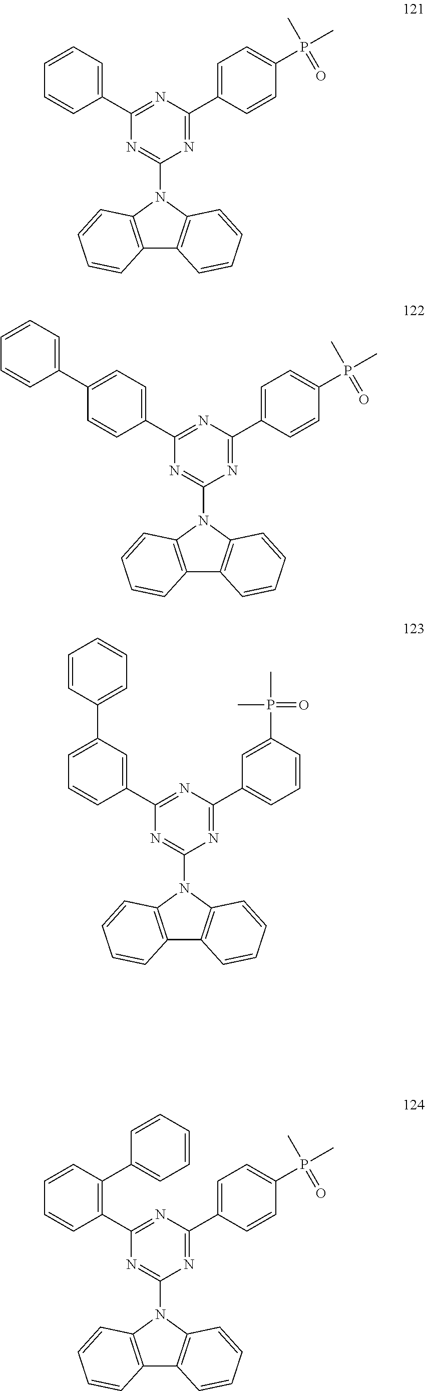 [SMILES: CP(C)(=O)c1ccc(-c2nc(-c3ccccc3)nc(-n3c4ccccc4c4ccccc43)n2)cc1 ; CP(C)(=O)c1ccc(-c2nc(-c3ccc(-c4ccccc4)cc3)nc(-n3c4ccccc4c4ccccc43)n2)cc1 ; CP(C)(=O)c1cccc(-c2nc(-c3cccc(-c4ccccc4)c3)nc(-n3c4ccccc4c4ccccc43)n2)c1 ; CP(C)(=O)c1ccc(-c2nc(-c3ccccc3-c3ccccc3)nc(-n3c4ccccc4c4ccccc43)n2)cc1]

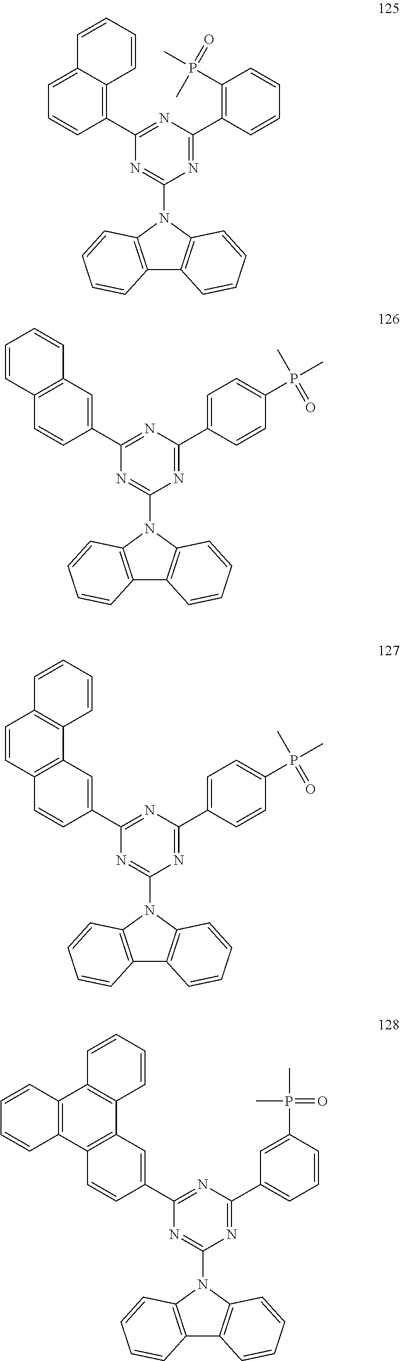 [SMILES: CP(C)(=O)c1ccccc1-c1nc(-c2cccc3ccccc23)nc(-n2c3ccccc3c3ccccc32)n1 ; CP(C)(=O)c1ccc(-c2nc(-c3ccc4ccccc4c3)nc(-n3c4ccccc4c4ccccc43)n2)cc1 ; CP(C)(=O)c1ccc(-c2nc(-c3ccc4ccc5ccccc5c4c3)nc(-n3c4ccccc4c4ccccc43)n2)cc1 ; CP(C)(=O)c1cccc(-c2nc(-c3ccc4c5ccccc5c5ccccc5c4c3)nc(-n3c4ccccc4c4ccccc43)n2)c1]

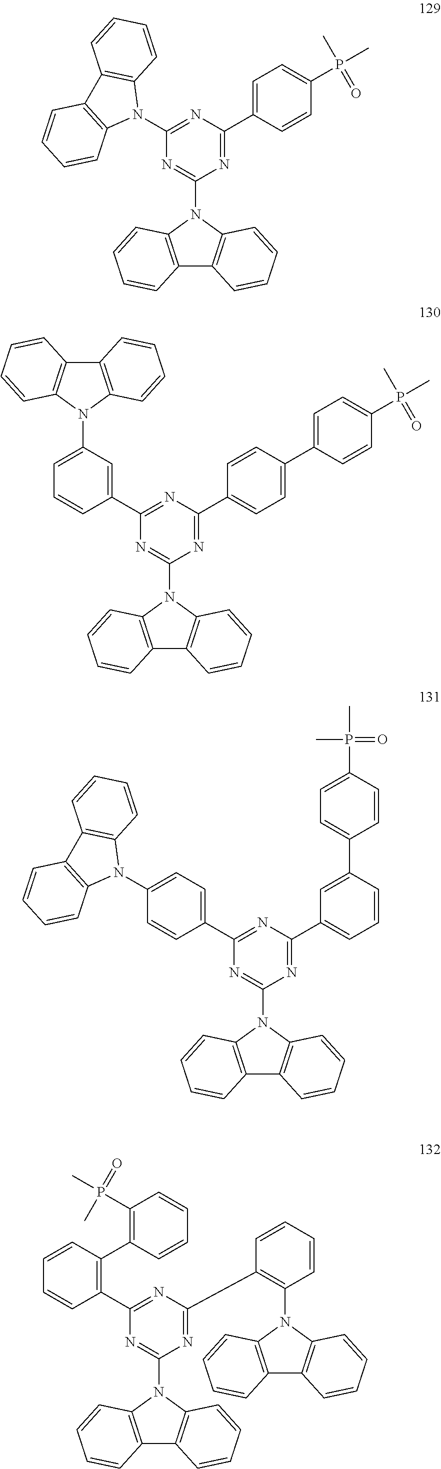 [SMILES: CP(C)(=O)c1ccc(-c2nc(-n3c4ccccc4c4ccccc43)nc(-n3c4ccccc4c4ccccc43)n2)cc1 ; CP(C)(=O)c1ccc(-c2ccc(-c3nc(-c4cccc(-n5c6ccccc6c6ccccc65)c4)nc(-n4c5ccccc5c5ccccc54)n3)cc2)cc1 ; CP(C)(=O)c1ccc(-c2cccc(-c3nc(-c4ccc(-n5c6ccccc6c6ccccc65)cc4)nc(-n4c5ccccc5c5ccccc54)n3)c2)cc1 ; CP(C)(=O)c1ccccc1-c1ccccc1-c1nc(-c2ccccc2-n2c3ccccc3c3ccccc32)nc(-n2c3ccccc3c3ccccc32)n1]

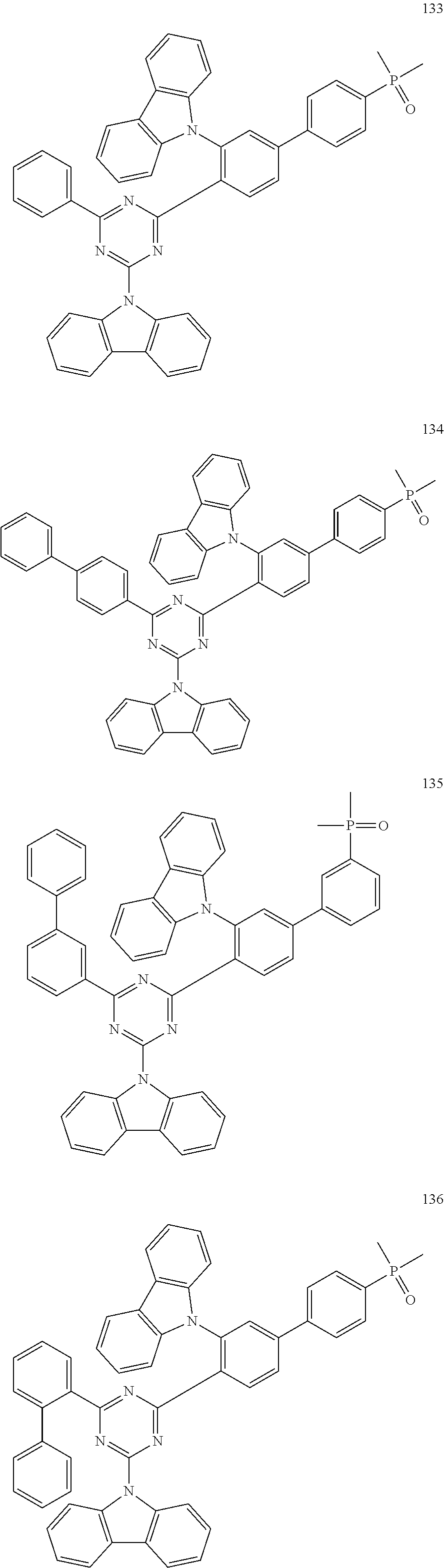 [SMILES: CP(C)(=O)c1ccc(-c2ccc(-c3nc(-c4ccccc4)nc(-n4c5ccccc5c5ccccc54)n3)c(-n3c4ccccc4c4ccccc43)c2)cc1 ; CP(C)(=O)c1ccc(-c2ccc(-c3nc(-c4ccc(-c5ccccc5)cc4)nc(-n4c5ccccc5c5ccccc54)n3)c(-n3c4ccccc4c4ccccc43)c2)cc1 ; CP(C)(=O)c1cccc(-c2ccc(-c3nc(-c4cccc(-c5ccccc5)c4)nc(-n4c5ccccc5c5ccccc54)n3)c(-n3c4ccccc4c4ccccc43)c2)c1 ; CP(C)(=O)c1ccc(-c2ccc(-c3nc(-c4ccccc4-c4ccccc4)nc(-n4c5ccccc5c5ccccc54)n3)c(-n3c4ccccc4c4ccccc43)c2)cc1]

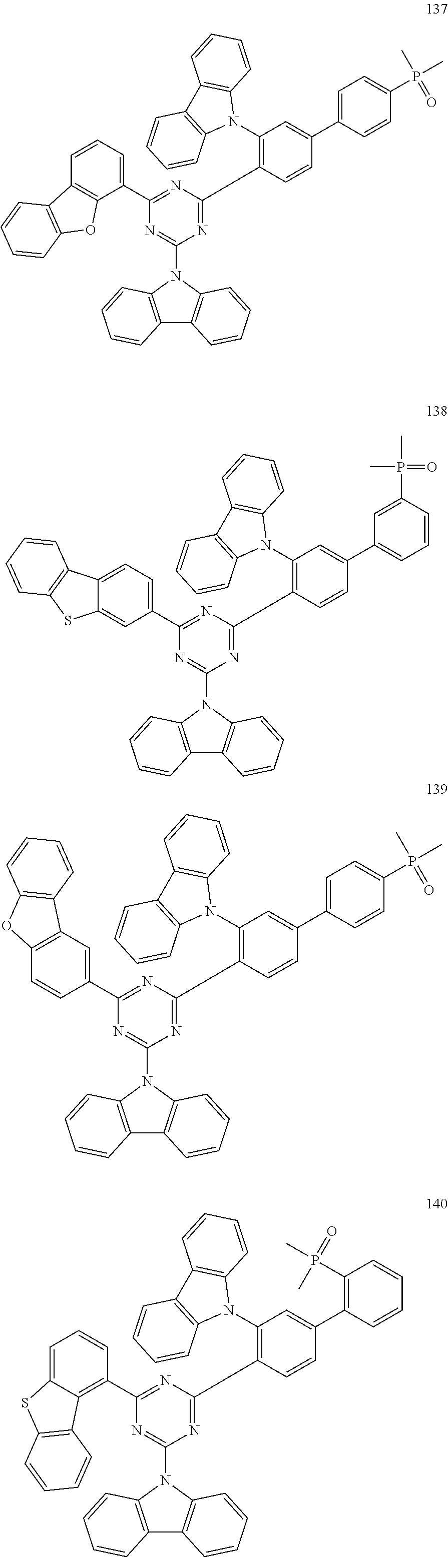

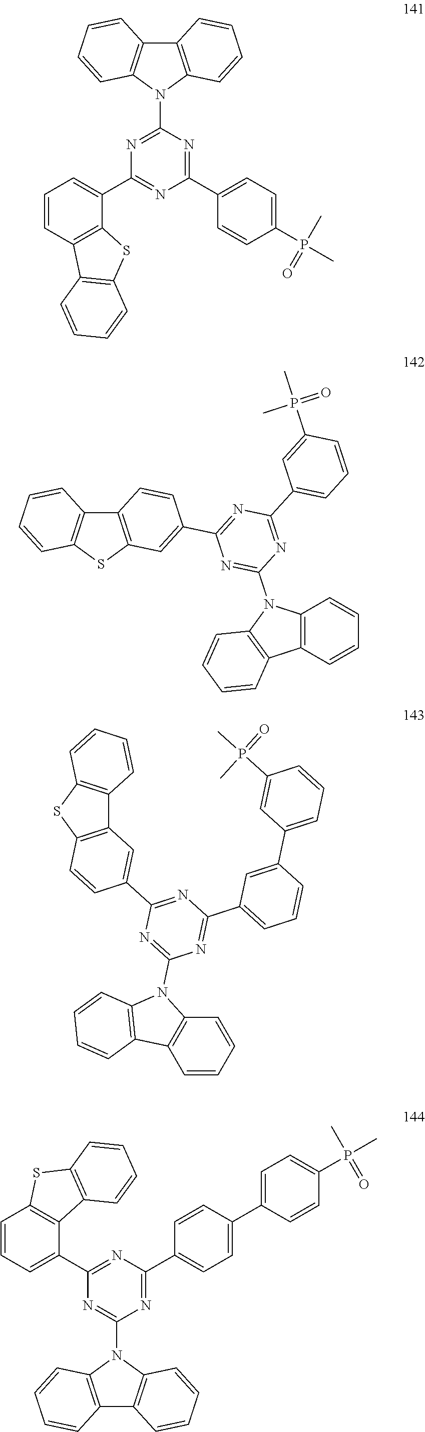

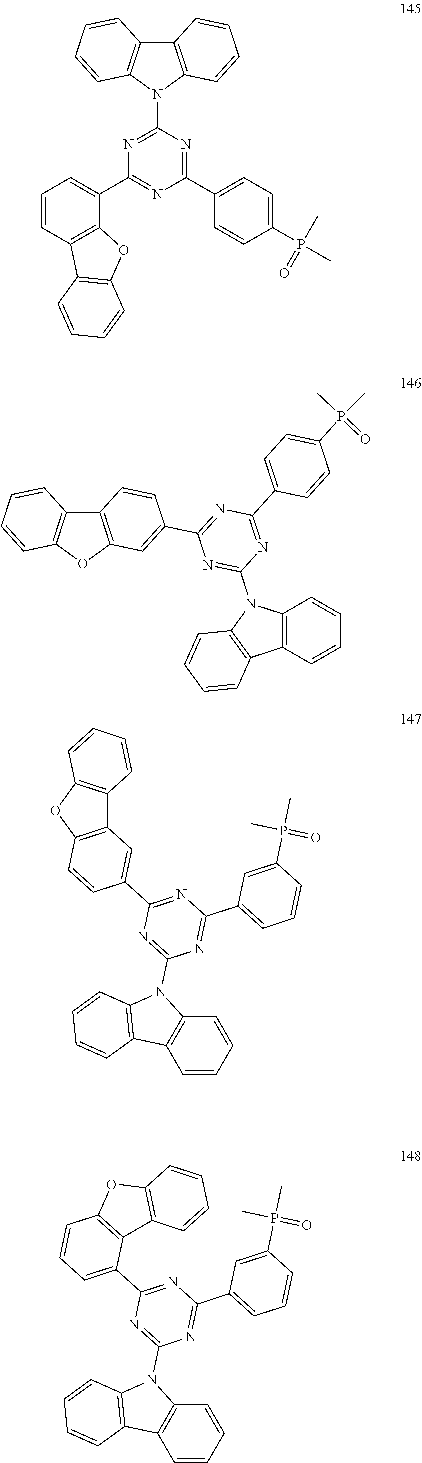 [SMILES: CP(C)(=O)c1ccc(-c2nc(-c3cccc4c3oc3ccccc34)nc(-n3c4ccccc4c4ccccc43)n2)cc1 ; CP(C)(=O)c1ccc(-c2nc(-c3ccc4c(c3)oc3ccccc34)nc(-n3c4ccccc4c4ccccc43)n2)cc1 ; CP(C)(=O)c1cccc(-c2nc(-c3ccc4oc5ccccc5c4c3)nc(-n3c4ccccc4c4ccccc43)n2)c1 ; CP(C)(=O)c1cccc(-c2nc(-c3cccc4oc5ccccc5c34)nc(-n3c4ccccc4c4ccccc43)n2)c1]

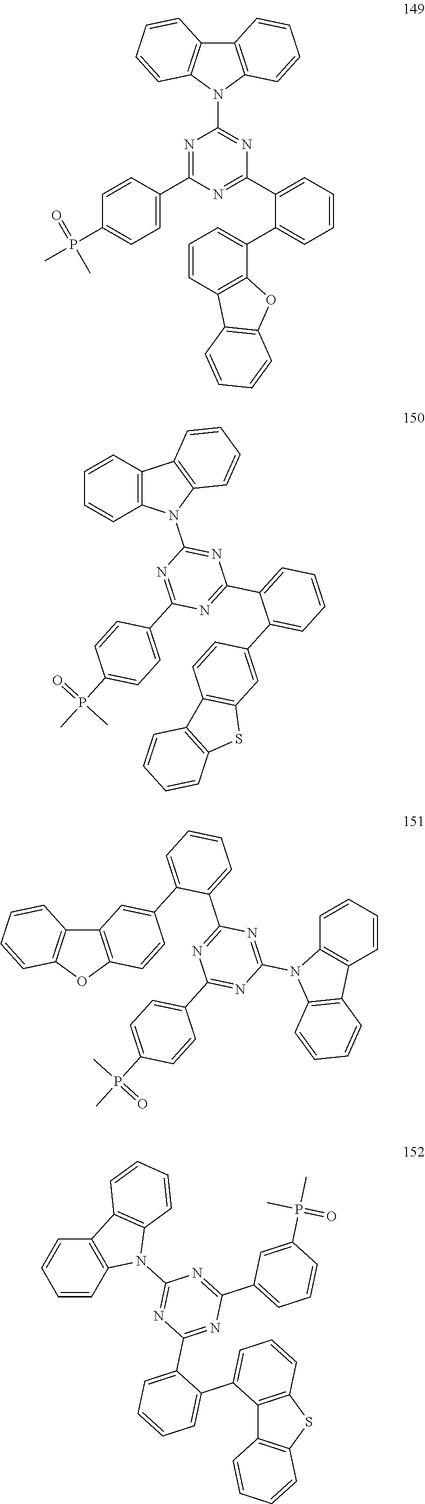

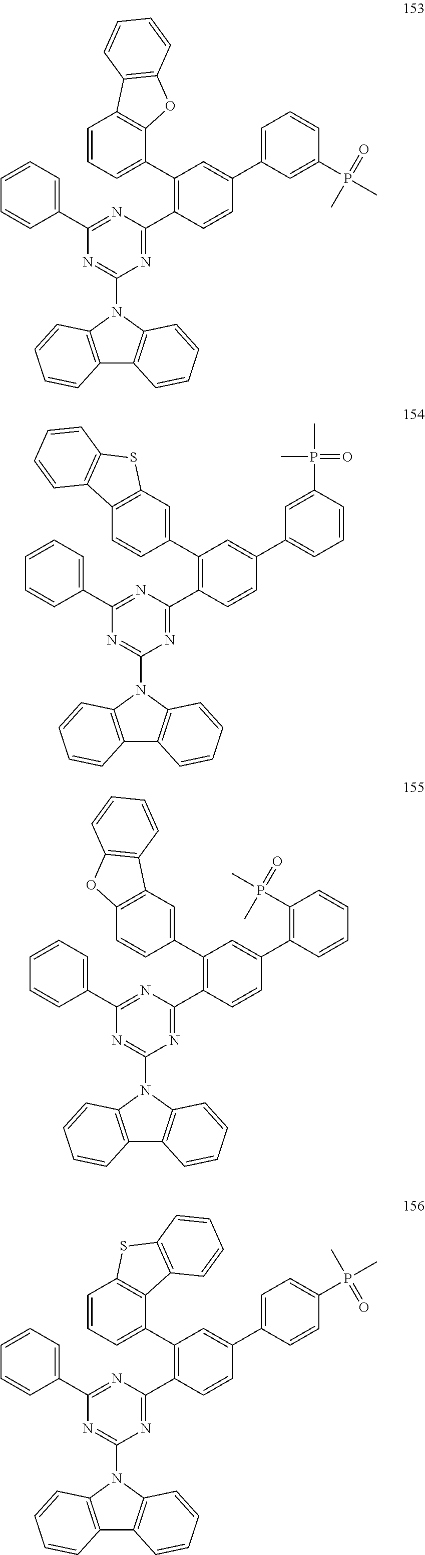

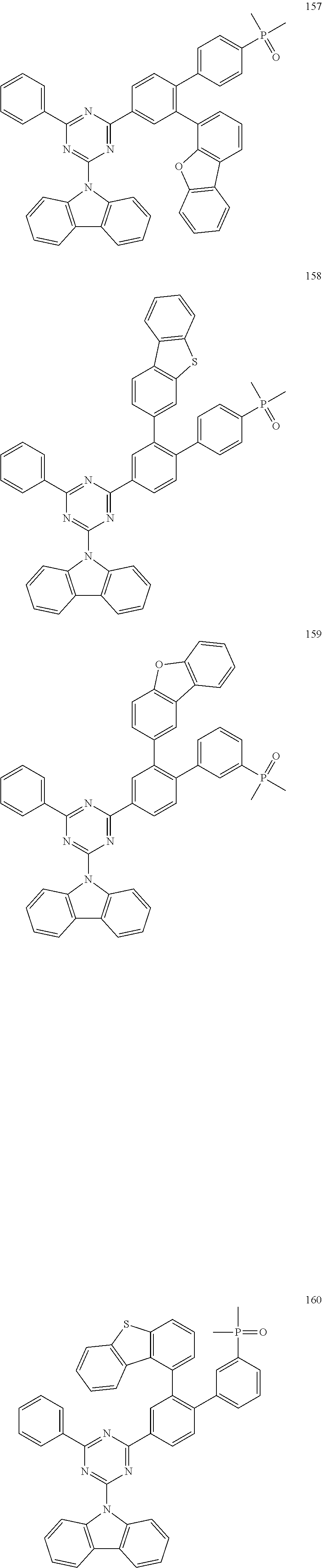 [SMILES: CP(C)(=O)c1ccc(-c2ccc(-c3nc(-c4ccccc4)nc(-n4c5ccccc5c5ccccc54)n3)cc2-c2cccc3c2oc2ccccc23)cc1 ; CP(C)(=O)c1ccc(-c2ccc(-c3nc(-c4ccccc4)nc(-n4c5ccccc5c5ccccc54)n3)cc2-c2ccc3c(c2)sc2ccccc23)cc1 ; CP(C)(=O)c1cccc(-c2ccc(-c3nc(-c4ccccc4)nc(-n4c5ccccc5c5ccccc54)n3)cc2-c2ccc3oc4ccccc4c3c2)c1 ; CP(C)(=O)c1cccc(-c2ccc(-c3nc(-c4ccccc4)nc(-n4c5ccccc5c5ccccc54)n3)cc2-c2cccc3sc4ccccc4c23)c1]

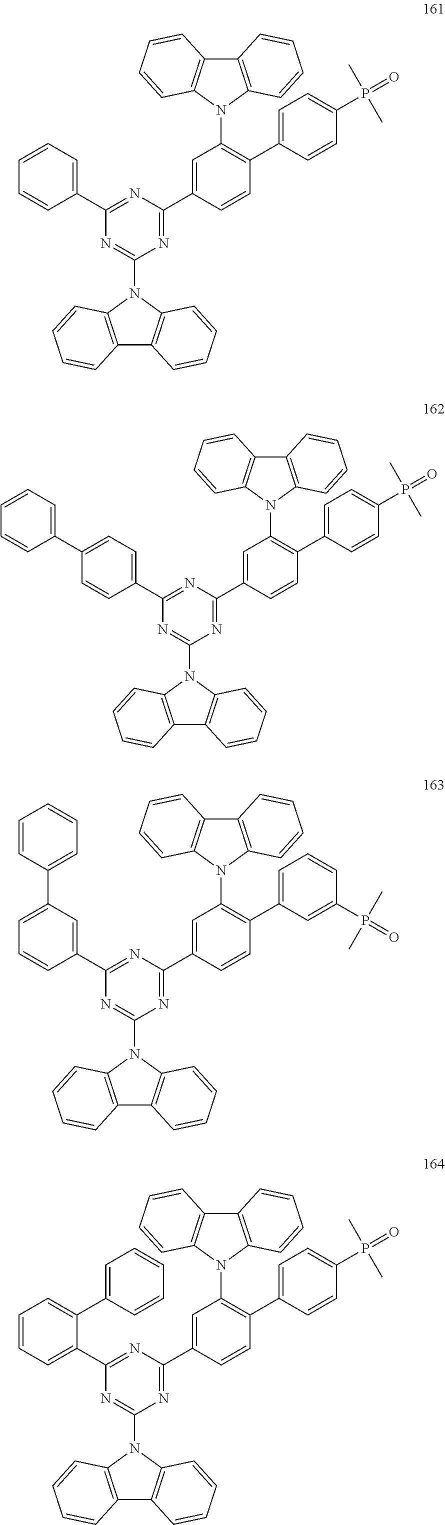 [SMILES: CP(C)(=O)c1ccc(-c2ccc(-c3nc(-c4ccccc4)nc(-n4c5ccccc5c5ccccc54)n3)cc2-n2c3ccccc3c3ccccc32)cc1 ; CP(C)(=O)c1ccc(-c2ccc(-c3nc(-c4ccc(-c5ccccc5)cc4)nc(-n4c5ccccc5c5ccccc54)n3)cc2-n2c3ccccc3c3ccccc32)cc1 ; CP(C)(=O)c1cccc(-c2ccc(-c3nc(-c4cccc(-c5ccccc5)c4)nc(-n4c5ccccc5c5ccccc54)n3)cc2-n2c3ccccc3c3ccccc32)c1 ; CP(C)(=O)c1ccc(-c2ccc(-c3nc(-c4ccccc4-c4ccccc4)nc(-n4c5ccccc5c5ccccc54)n3)cc2-n2c3ccccc3c3ccccc32)cc1]

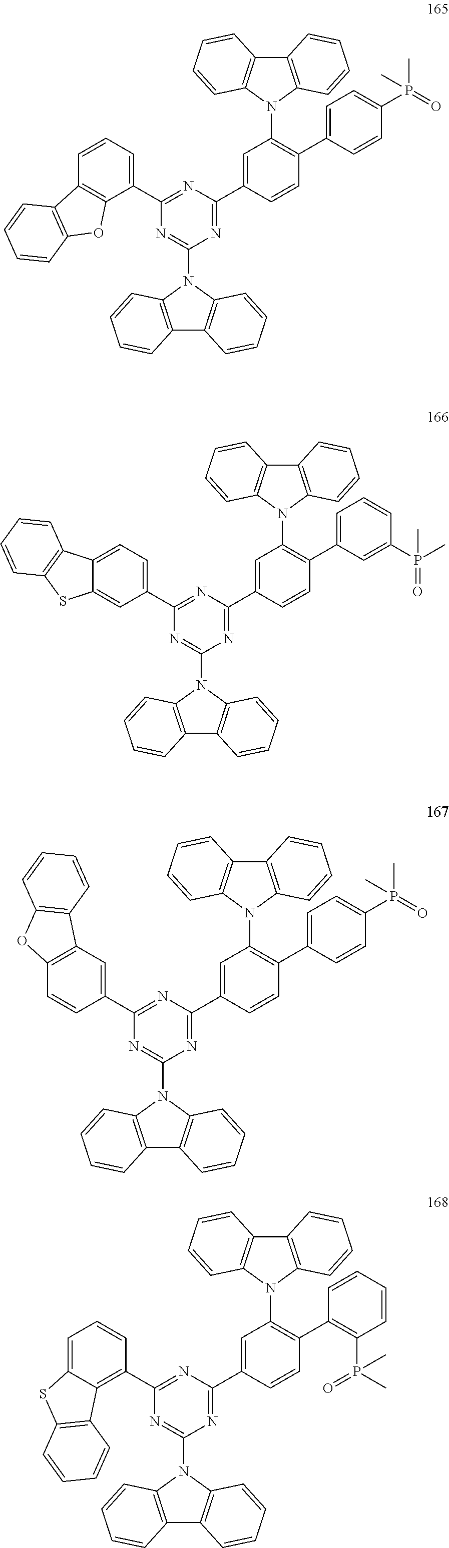 [SMILES: CP(C)(=O)c1ccc(-c2ccc(-c3nc(-c4cccc5c4oc4ccccc45)nc(-n4c5ccccc5c5ccccc54)n3)cc2-n2c3ccccc3c3ccccc32)cc1 ; CP(C)(=O)c1cccc(-c2ccc(-c3nc(-c4ccc5c(c4)sc4ccccc45)nc(-n4c5ccccc5c5ccccc54)n3)cc2-n2c3ccccc3c3ccccc32)c1 ; CP(C)(=O)c1ccc(-c2ccc(-c3nc(-c4ccc5oc6ccccc6c5c4)nc(-n4c5ccccc5c5ccccc54)n3)cc2-n2c3ccccc3c3ccccc32)cc1 ; CP(C)(=O)c1ccccc1-c1ccc(-c2nc(-c3cccc4sc5ccccc5c34)nc(-n3c4ccccc4c4ccccc43)n2)cc1-n1c2ccccc2c2ccccc21]

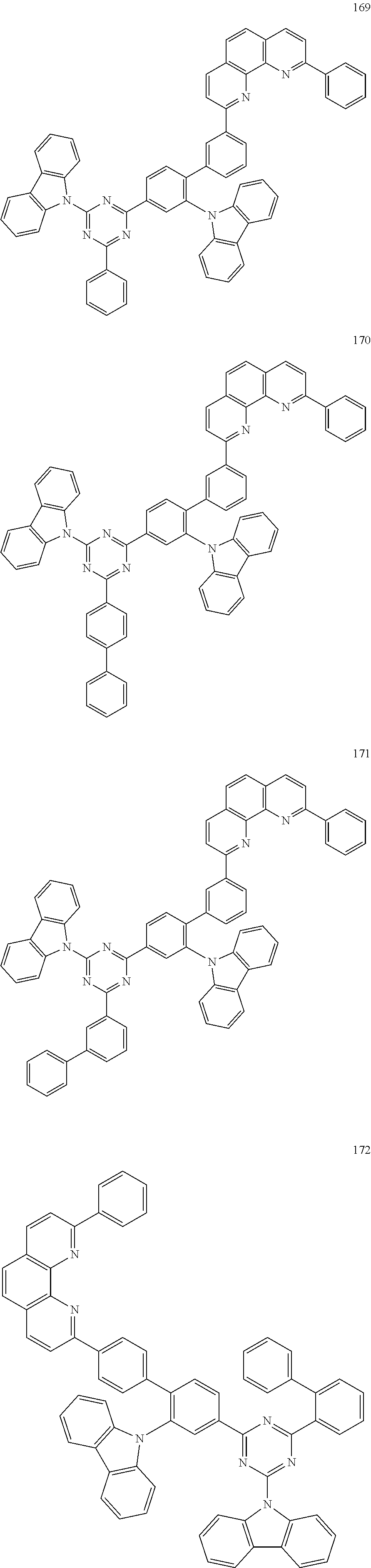

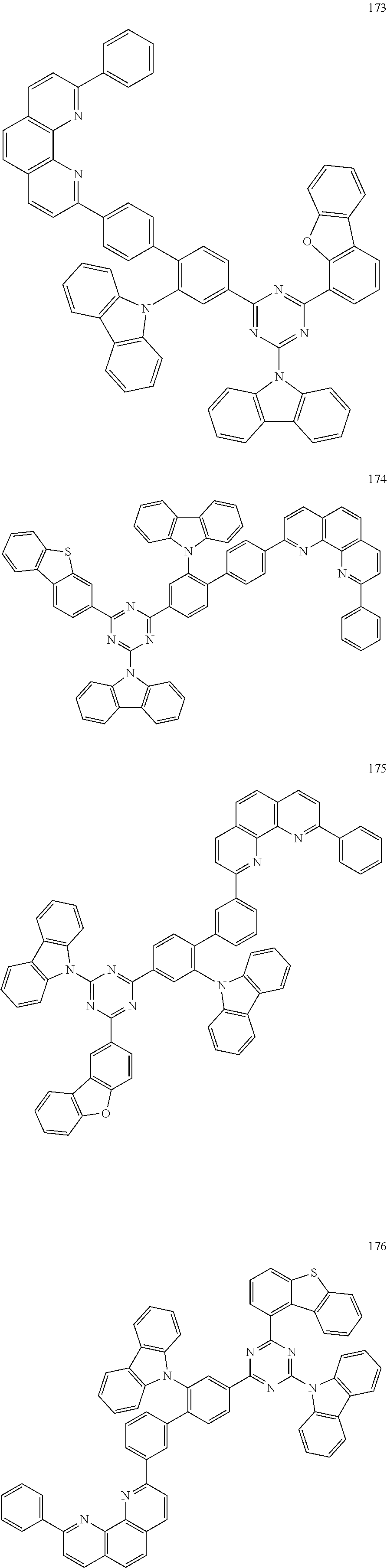

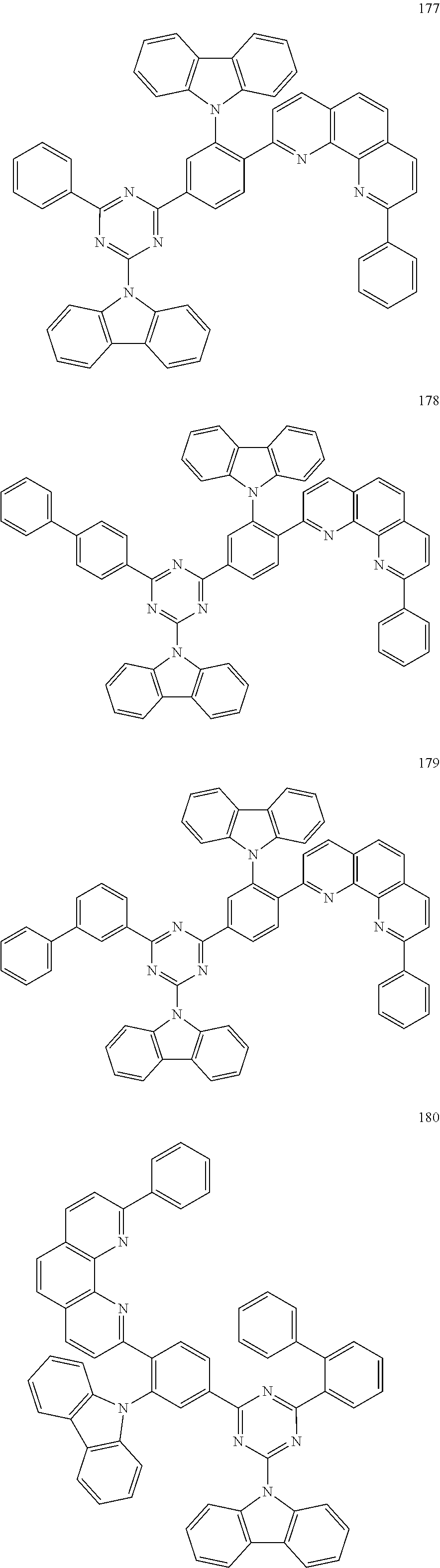 [SMILES: c1ccc(-c2ccc3ccc4ccc(-c5ccc(-c6nc(-c7ccccc7)nc(-n7c8ccccc8c8ccccc87)n6)cc5-n5c6ccccc6c6ccccc65)nc4c3n2)cc1 ; c1ccc(-c2ccc(-c3nc(-c4ccc(-c5ccc6ccc7ccc(-c8ccccc8)nc7c6n5)c(-n5c6ccccc6c6ccccc65)c4)nc(-n4c5ccccc5c5ccccc54)n3)cc2)cc1 ; c1ccc(-c2cccc(-c3nc(-c4ccc(-c5ccc6ccc7ccc(-c8ccccc8)nc7c6n5)c(-n5c6ccccc6c6ccccc65)c4)nc(-n4c5ccccc5c5ccccc54)n3)c2)cc1 ; c1ccc(-c2ccc3ccc4ccc(-c5ccc(-c6nc(-c7ccccc7-c7ccccc7)nc(-n7c8ccccc8c8ccccc87)n6)cc5-n5c6ccccc6c6ccccc65)nc4c3n2)cc1]

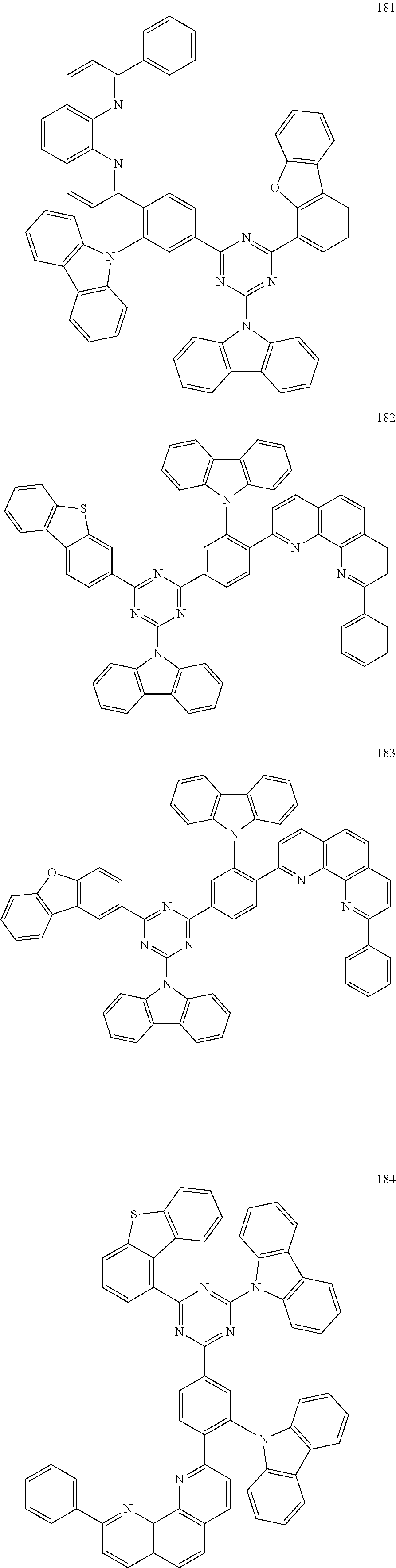 [SMILES: c1ccc(-c2ccc3ccc4ccc(-c5ccc(-c6nc(-c7cccc8c7oc7ccccc78)nc(-n7c8ccccc8c8ccccc87)n6)cc5-n5c6ccccc6c6ccccc65)nc4c3n2)cc1 ; c1ccc(-c2ccc3ccc4ccc(-c5ccc(-c6nc(-c7ccc8c(c7)sc7ccccc78)nc(-n7c8ccccc8c8ccccc87)n6)cc5-n5c6ccccc6c6ccccc65)nc4c3n2)cc1 ; c1ccc(-c2ccc3ccc4ccc(-c5ccc(-c6nc(-c7ccc8oc9ccccc9c8c7)nc(-n7c8ccccc8c8ccccc87)n6)cc5-n5c6ccccc6c6ccccc65)nc4c3n2)cc1 ; c1ccc(-c2ccc3ccc4ccc(-c5ccc(-c6nc(-c7cccc8sc9ccccc9c78)nc(-n7c8ccccc8c8ccccc87)n6)cc5-n5c6ccccc6c6ccccc65)nc4c3n2)cc1]

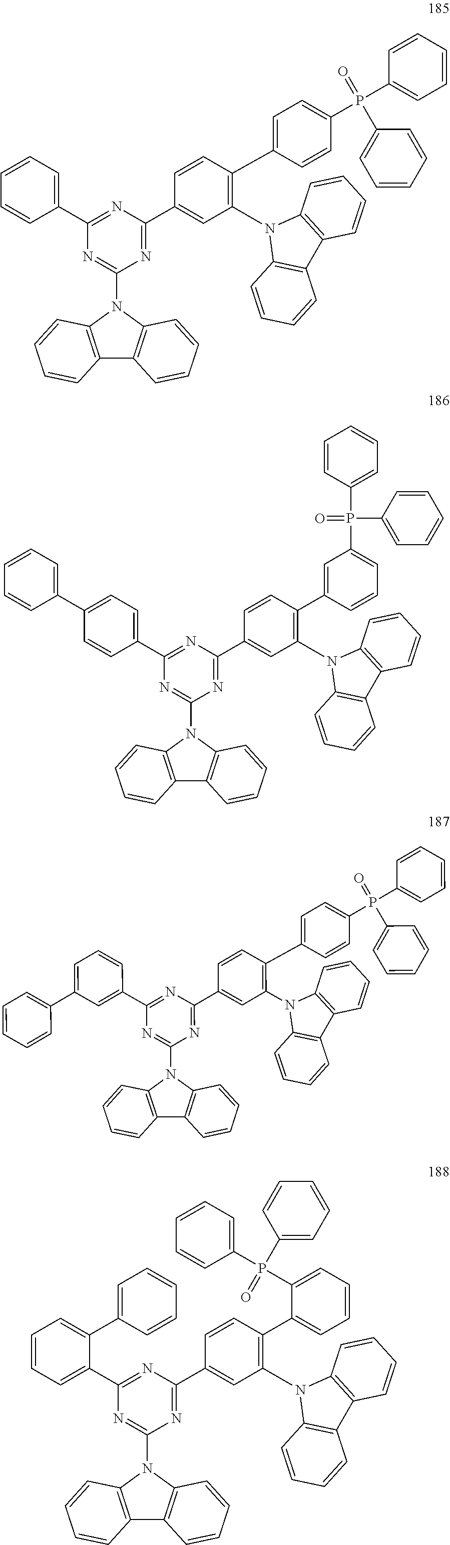 [SMILES: O=P(c1ccccc1)(c1ccccc1)c1ccc(-c2ccc(-c3nc(-c4ccccc4)nc(-n4c5ccccc5c5ccccc54)n3)cc2-n2c3ccccc3c3ccccc32)cc1 ; O=P(c1ccccc1)(c1ccccc1)c1cccc(-c2ccc(-c3nc(-c4ccc(-c5ccccc5)cc4)nc(-n4c5ccccc5c5ccccc54)n3)cc2-n2c3ccccc3c3ccccc32)c1 ; O=P(c1ccccc1)(c1ccccc1)c1ccc(-c2ccc(-c3nc(-c4cccc(-c5ccccc5)c4)nc(-n4c5ccccc5c5ccccc54)n3)cc2-n2c3ccccc3c3ccccc32)cc1 ; O=P(c1ccccc1)(c1ccccc1)c1ccccc1-c1ccc(-c2nc(-c3ccccc3-c3ccccc3)nc(-n3c4ccccc4c4ccccc43)n2)cc1-n1c2ccccc2c2ccccc21]

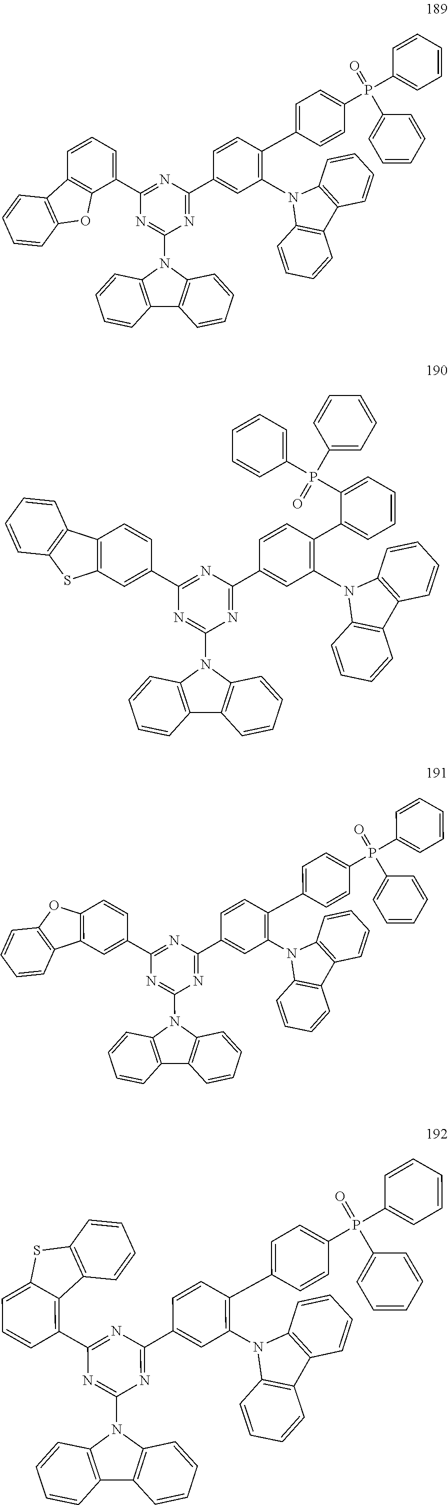 [SMILES: O=P(c1ccccc1)(c1ccccc1)c1ccc(-c2ccc(-c3nc(-c4cccc5c4oc4ccccc45)nc(-n4c5ccccc5c5ccccc54)n3)cc2-n2c3ccccc3c3ccccc32)cc1 ; O=P(c1ccccc1)(c1ccccc1)c1ccccc1-c1ccc(-c2nc(-c3ccc4c(c3)sc3ccccc34)nc(-n3c4ccccc4c4ccccc43)n2)cc1-n1c2ccccc2c2ccccc21 ; O=P(c1ccccc1)(c1ccccc1)c1ccc(-c2ccc(-c3nc(-c4ccc5oc6ccccc6c5c4)nc(-n4c5ccccc5c5ccccc54)n3)cc2-n2c3ccccc3c3ccccc32)cc1 ; O=P(c1ccccc1)(c1ccccc1)c1ccc(-c2ccc(-c3nc(-c4cccc5sc6ccccc6c45)nc(-n4c5ccccc5c5ccccc54)n3)cc2-n2c3ccccc3c3ccccc32)cc1]

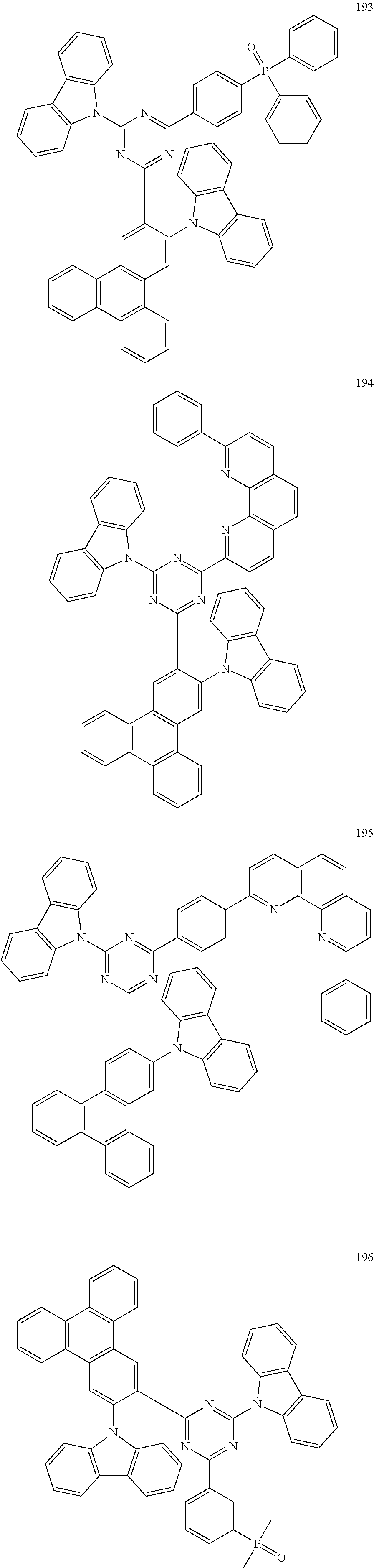 [SMILES: O=P(c1ccccc1)(c1ccccc1)c1ccc(-c2nc(-c3cc4c5ccccc5c5ccccc5c4cc3-n3c4ccccc4c4ccccc43)nc(-n3c4ccccc4c4ccccc43)n2)cc1 ; c1ccc(-c2ccc3ccc4ccc(-c5nc(-c6cc7c8ccccc8c8ccccc8c7cc6-n6c7ccccc7c7ccccc76)nc(-n6c7ccccc7c7ccccc76)n5)nc4c3n2)cc1 ; c1ccc(-c2ccc3ccc4ccc(-c5ccc(-c6nc(-c7cc8c9ccccc9c9ccccc9c8cc7-n7c8ccccc8c8ccccc87)nc(-n7c8ccccc8c8ccccc87)n6)cc5)nc4c3n2)cc1 ; CP(C)(=O)c1cccc(-c2nc(-c3cc4c5ccccc5c5ccccc5c4cc3-n3c4ccccc4c4ccccc43)nc(-n3c4ccccc4c4ccccc43)n2)c1]

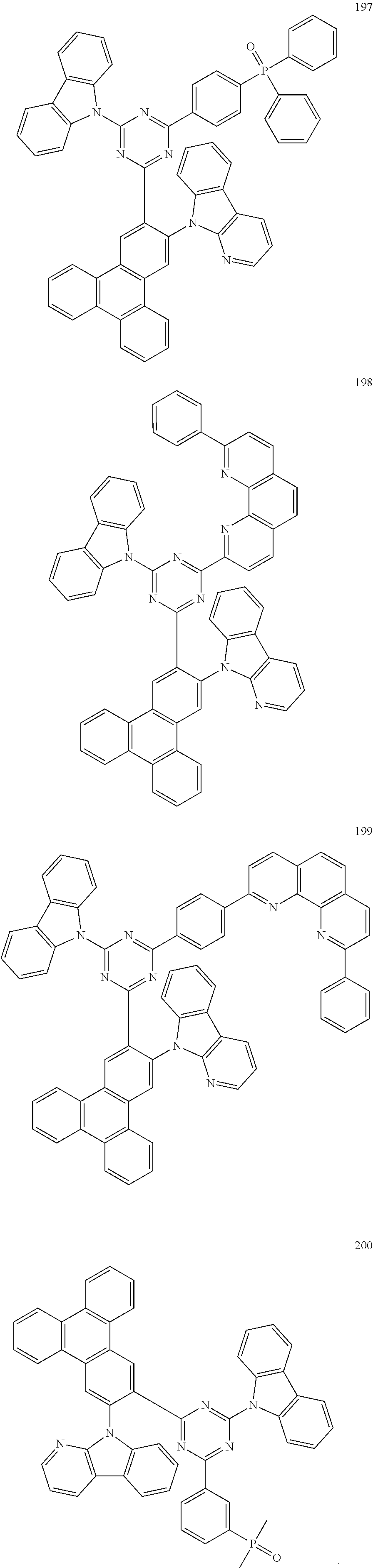 [SMILES: O=P(c1ccccc1)(c1ccccc1)c1ccc(-c2nc(-c3cc4c5ccccc5c5ccccc5c4cc3-n3c4ccccc4c4cccnc43)nc(-n3c4ccccc4c4ccccc43)n2)cc1 ; c1ccc(-c2ccc3ccc4ccc(-c5nc(-c6cc7c8ccccc8c8ccccc8c7cc6-n6c7ccccc7c7cccnc76)nc(-n6c7ccccc7c7ccccc76)n5)nc4c3n2)cc1 ; c1ccc(-c2ccc3ccc4ccc(-c5ccc(-c6nc(-c7cc8c9ccccc9c9ccccc9c8cc7-n7c8ccccc8c8cccnc87)nc(-n7c8ccccc8c8ccccc87)n6)cc5)nc4c3n2)cc1 ; CP(C)(=O)c1cccc(-c2nc(-c3cc4c5ccccc5c5ccccc5c4cc3-n3c4ccccc4c4cccnc43)nc(-n3c4ccccc4c4ccccc43)n2)c1]

6. An organic light emitting device comprising:
a first electrode;
a second electrode provided opposite to the first electrode; and
one or more organic material layers provided between the first electrode and the second electrode,
wherein the organic material layers include one or more types of the heterocyclic compound of any one of claims 1 to 5.

7. The organic light emitting device of claim 6, wherein the organic material layer includes a light emitting layer, and the light emitting layer includes the heterocyclic compound.

8. The organic light emitting device of claim 6, wherein the organic material layer includes an electron injection layer or an electron transport layer, and the electron injection layer or the electron transport layer includes the heterocyclic compound.

9. The organic light emitting device of claim 6, wherein the organic material layer includes an electron blocking layer or a hole blocking layer, and the electron blocking layer or the hole blocking layer includes the heterocyclic compound.

10. The organic light emitting device of claim 6, further comprising one, or two or more layers selected from the group consisting of a light emitting layer, a hole injection layer, a hole transport layer, an electron injection layer, an electron transport layer, an electron blocking layer and a hole blocking layer.

11. The organic light emitting device of claim 6, comprising:
a first electrode;
a first stack provided on the first electrode and including a first light emitting layer;
a charge generation layer provided on the first stack;
a second stack provided on the charge generation layer and including a second light emitting layer; and
a second electrode provided on the second stack.

12. The organic light emitting device of claim 11, wherein the charge generation layer includes the heterocyclic compound.

13. The organic light emitting device of claim 11, wherein the charge generation layer is an N-type charge generation layer, and the charge generation layer includes the heterocyclic compound.
